(19) United States
(12) Patent Application Publication
(10) Pub. No.: US 2021/0098706 A1
(43) Pub. Date: Apr. 1, 2021

(54) ORGANIC ELECTRIC ELEMENT COMPRISING COMPOUND FOR ORGANIC ELECTRIC ELEMENT AND ELECTRONIC DEVICE THEREOF

(71) Applicant: DUK SAN NEOLUX CO., LTD., Cheonan-si, Chungcheongnam-do (KR)

(72) Inventors: Soung Yun MUN, Cheonan-si, Chungcheongnam-do (KR); Sun Hee LEE, Hwaseong-si, Gyeonggi-do (KR); Jong Gwang PARK, Cheonan-si, Chungcheongnam-do (KR); Yun Suk LEE, Seongnam-si, Gyeonggi-do (KR); Jung Hwan PARK, Hwaseong-si, Gyeonggi-do (KR); Bum Sung LEE, Hwaseong-si, Gyeonggi-do (KR); Chi Hyun PARK, Cheonan-si, Chungcheongnam-do (KR)

(73) Assignee: DUK SAN NEOLUX CO., LTD., Cheonan-si, Chungcheongnam-do (KR)

(21) Appl. No.: 16/955,349

(22) PCT Filed: Dec. 17, 2018
(86) PCT No.: PCT/KR2018/016001

§ 371 Date: Jun. 18, 2020

(30) Foreign Application Priority Data
Dec. 18, 2017 (KR) 10-2017-0173891

(51) Int. Cl. H01L 51/00; C07D 333/76; C09K 11/06; C07D 409/14; C07D 409/12; C07D 495/04

(52) U.S. Cl. CPC H01L 51/0061; C07D 333/76; C09K 11/06; C07D 409/14; C07D 409/12; C07D 495/04; H01L 51/5012; H01L 51/0067; H01L 51/0072; H01L 51/0074; H01L 51/006; H01L 51/0073; H01L 51/0071

(57) ABSTRACT

The present invention provides an organic electric element and an electronic device thereof, wherein at least three of organic material layers of the organic electric element comprise the compound capable of increasing the luminous efficiency, lowering the driving voltage, increasing the heat resistance, improving color purity and lifetime of the organic electric element.

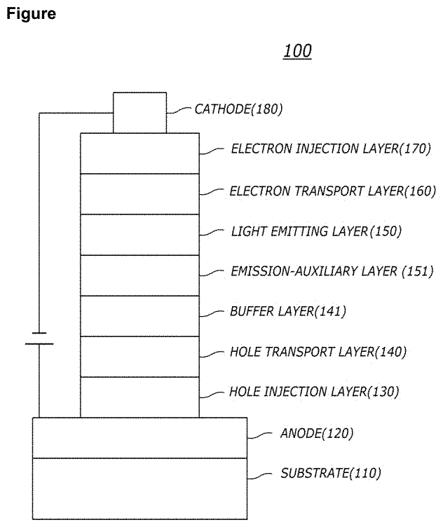

### CROSS-REFERENCE TO RELATED APPLICATION

This patent application claims priority from and the benefit under 35 U.S.C. § 119 to § 121, and § 365 of Korean Patent Application No. 10-2017-0173891, filed on Dec. 18, 2017 which is hereby incorporated by reference for all purposes as if fully set forth herein. Further, this application claims the benefit of priority in countries other than U.S., which is hereby incorporated by reference herein.

### BACKGROUND

### Technical Field

The present invention relates to an organic electric element employing the compound for an organic electric element and an electronic device thereof.

### Background Art

In general, an organic light emitting phenomenon refers to a phenomenon in which electric energy is converted into light energy of an organic material. An organic electric element utilizing the organic light emitting phenomenon usually has a structure including an anode, a cathode, and an organic material layer interposed therebetween. In many cases, the organic material layer has a multi-layered structure having respectively different materials in order to improve efficiency and stability of an organic electric element, and for example, may include a hole injection layer, a hole transport layer, a light emitting layer, an electron transport layer, an electron injection layer, or the like.

Materials used as an organic material layer in an organic electric element may be classified into a light emitting material and a charge transport material, for example, a hole injection material, a hole transport material, an electron transport material, an electron injection material, and the like according to its function. Further, the light emitting material may be divided into a high molecular weight type and a low molecular weight type according to its molecular weight, and may also be divided into a fluorescent material derived from excited singlet states of electron and a phosphorescent material derived from excited triplet states of electron according to its light emitting mechanism. Further, the light emitting material may be divided into blue, green, and red light emitting material and yellow and orange light emitting material required for better natural color reproduction according to its light emitting color.

Currently, the power consumption is required more than more as size of display becomes larger and larger in the portable display market. Therefore, the power consumption is a very important factor in the portable display with a limited power source of the battery, and efficiency and life span issue also is solved.

Efficiency, life span, driving voltage, and the like are correlated with each other. For example, if efficiency is increased, then driving voltage is relatively lowered, and the crystallization of an organic material due to Joule heating generated during operation is reduced as driving voltage is lowered, as a result of which life span shows a tendency to increase. However, efficiency cannot be maximized only by simply improving the organic material layer. This is because long life span and high efficiency can be simultaneously achieved when an optimal combination of energy levels and Ti values, inherent material properties (mobility, interfacial properties, etc.), and the like among the respective layers included in the organic material layer is given.

Therefore, there is a need for research and development for the optimal combination of the compounds used for each organic material layer that has high thermal stability and can efficiently achieve charge balance in the light emitting layer. In particular, it is necessary to maximize the properties of the organic electric device by appropriately selecting each material for a hole transport layer, an emission-auxiliary layer and a light emitting layer.

### Object, Technical Solution and Effects of the Invention

An object of the present invention is to provide an organic electric element and an electronic device thereof, wherein at least three of organic material layers of the organic electric element comprise the compound capable of increasing the luminous efficiency, lowering the driving voltage, increasing the heat resistance, improving color purity and lifetime of the organic electric element.

In an aspect of the present invention, the present invention provides an organic electric element comprising a first electrode, a second electrode, and an organic material layer. Here, at least three layers among the layers forming the organic material layer comprise a single compound or two or more compounds represented by Formula 1.

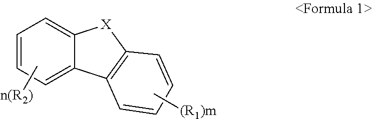

In another aspect of the present invention, the present invention provides an electronic device comprising the organic electric element.

By employing the compound according to the embodiment of the present invention as a material for at least three layers of the organic material layer of an organic electric element, the driving voltage of the organic electric element can be lowered, and the luminous efficiency, color purity and lifespan of the organic electric element can be significantly improved.

### BRIEF DESCRIPTION OF THE DRAWINGS

FIGURE illustrate an example of an organic electric element according to the present invention: 100 is an organic electric element, 110 is a substrate, 120 is a first electrode, 130 is a hole injection layer, 140 is a hole transport layer, 141 is a buffer layer, 150 is a light emitting layer, 151 is an emission-auxiliary layer, 160 is an electron transport layer, 170 is an electron injection layer, and 180 is a second electrode.

### DETAILED DESCRIPTION

In this specification, a ‘group name’ corresponding to an aryl group, an arylene group, a heterocyclic group, and the like exemplified for each symbol and its substituent may be written in the name of functional group reflecting the valence, and may also be described as the name of a parent compound. For example, in the case of phenanthrene which is a kind of aryl group, it may be described by distinguishing valence such as ‘phenanthryl’ when it is ‘monovalent group’, and as ‘phenanthrylene’ when it is ‘divalent group’, and it may also be described as a parent compound name, ‘phenanthrene’, regardless of its valence. Similarly, in the case of pyrimidine, it may be described as ‘pyrimidine’ regardless of its valence, and it may also be described as the name of corresponding functional group such as pyrimidinyl when it is ‘monovalent group’, and as ‘pyrimidylene’ when it is ‘divalent group’.

Unless otherwise stated, the term “fluorenyl group” or “fluorenylene group” as used herein means univalent or bivalent functional group in which R, R′ and R″ are all hydrogen in the following structure, “substituted fluorenyl group” or “substituted fluorenylene group” means that at least any one of R, R′ and R″ is a substituent other than hydrogen, and it comprises the case where R and R′ are bonded to each other to form the spiro compound together with the carbon to which they are bonded.

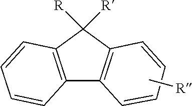

The term “spiro compound” as used herein has, a spiro union which means union having one atom as the only common member of two rings. The common atom is designated as ‘spiro atom’. The compounds are defined as ‘monospiro-’, ‘dispiro-’ or ‘trispiro-’ depending on the number of spiro atoms in one compound.

The term “heterocyclic group” as used herein means a ring comprising a heteroatom like N, O, S, P, Si or the like instead of carbon consisting of, it comprises a non-aromatic ring as well as an aromatic ring like “heteroaryl group” or “heteroarylene group” and the compound comprising heteroatom group like SO₂, P═O or the like instead of carbon consisting of a ring such as the following compound.

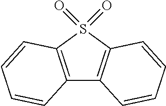 [SMILES: O=S1(=O)c2ccccc2-c2ccccc21]

Also, otherwise specified, the formulas used in the present invention are as defined in the index definition of the substituent of the following formula.

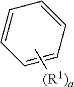

Here, the substituent R¹ is absent when a is an integer of zero, the sole R¹ is bonded to any one of the carbon atoms constituting the benzene ring when a is an integer of 1, when a is an integer of 2 or 3, the substituent R¹s may be bonded as follows and the substituents R¹s may be the same or different each other, and the substituent R¹s may be bonded to the carbon of the benzene ring in a similar manner when a is an integer of 4 to 6. Herein, the indication of the hydrogen bonded to the carbon which forms the benzene ring is omitted.

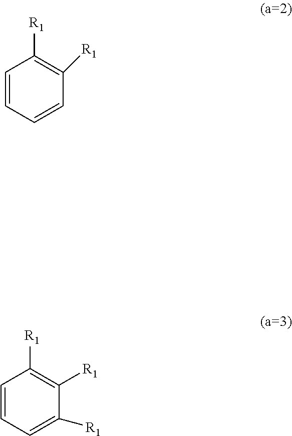

Hereinafter, a laminated structure of the electric element comprising the compound of the present invention will be described with reference to FIGURE.

The FIGURE illustrates a laminated structure of an organic electric element according to an embodiment of the present invention.

Referring to the FIGURE, an organic electric element 100 according to an embodiment of the present invention includes a first electrode 120 formed on a substrate 110, a second electrode 180, and an organic material layer formed between the first electrode 120 and the second electrode 180 and comprising the compound of the present invention. Here, the first electrode 120 may be an anode (positive electrode), and the second electrode 180 may be a cathode (negative electrode). In the case of an inverted organic electroluminescent element, the first electrode may be a cathode, and the second electrode may be an anode.

The organic material layer may include a hole injection layer 130, a hole transport layer 140, a light emitting layer 150, an electron transport layer 160, and an electron injection layer 170 formed in sequence on the first electrode 120. Here, at least one layer of the organic material layer may be omitted, or the organic material layer may further include a hole blocking layer, an electron blocking layer, an emission-auxiliary layer 151, a buffer layer 141, etc., and the electron transport layer 160 or the like may serve as a hole blocking layer.

In addition, although not shown, the organic electric element according to an embodiment of the present invention may further include a protective layer or a layer for improving luminous efficiency formed on at least one side of sides of the first electrode and the second electrode, wherein at least one side is not facing the organic material layer.

The inventive compound employed in the organic material layer may be used as a material of a hole injection layer 130, a hole transport layer 140, electron transport layer 160, an electron injection layer 170, a light emitting layer 150, a layer for improving luminous efficiency, an emission-auxiliary layer and so on. For example, the inventive compound may be used as material of a light emitting layer 150, preferably, as host material of a light emitting layer.

The organic electric element according to an embodiment of the present invention may be manufactured using various deposition methods. The organic electric element according to an embodiment of the present invention may be manufactured using a PVD (physical vapor deposition) method or CVD (chemical vapor deposition) method. For example, the organic electric element may be manufactured by depositing a metal, a conductive metal oxide, or a mixture thereof on the substrate to form the anode 120, forming the organic material layer including the hole injection layer 130, the hole transport layer 140, the light emitting layer 150, the electron transport layer 160, and the electron injection layer 170 thereon, and then depositing a material, which can be used as the cathode 180, thereon. Also, an emitting auxiliary layer 151 may be formed between a hole transport layer 140 and a light emitting layer 150.

Also, the organic material layer may be manufactured in such a manner that a smaller number of layers are formed using various polymer materials by a soluble process or solvent process, for example, spin coating, nozzle printing, inkjet printing, slot coating, dip coating, roll-to-roll, doctor blading, screen printing, or thermal transfer, instead of deposition. Since the organic material layer according to the present invention may be formed in various ways, the scope of protection of the present invention is not limited by a method of forming the organic material layer.

The organic electric element according to the present invention may be one of an organic light emitting device (OLED), an organic solar cell, an organic photo conductor (OPC), an organic transistor, an element for monochromatic or white illumination and an element quantum dot display.

Another embodiment of the present invention provides an electronic device including a display device which includes the above described organic electric element, and a control unit for controlling the display device. Here, the electronic device may be a wired/wireless communication terminal which is currently used or will be used in the future, and covers all kinds of electronic devices including a mobile communication terminal such as a cellular phone, a personal digital assistant (PDA), an electric dictionary, a point-to-multipoint (PMP), a remote controller, a navigation unit, a game player, various kinds of TVs, and various kinds of computers, and the display device may comprise an electroluminescent display, a quantum dot display and so on.

An organic electric element according to one aspect of the present invention comprises a first electrode, a second electrode, and an organic material layer formed between the first electrode and the second electrode and comprising a light emitting layer, wherein the organic material layer comprises an emission-auxiliary layer formed between the light emitting layer and the first electrode, a hole transport layer formed between the emission-auxiliary layer and the first electrode, an electron transport-auxiliary layer formed between the light emitting layer and the second electrode, and an electron transport layer formed between the electron transport-auxiliary layer and the second electrode, and at least three layers among the layers forming the organic material layer comprise a single compound or two or more compounds represented by Formula 1.

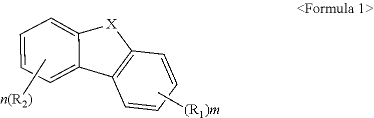

In the formula 1, each of symbols may be defined as follows.

X is 0 or S.

R₁ and R₂ are each independently selected from the group consisting of hydrogen, deuterium, halogen, a C₆-C₆₀ aryl group, a fluorenyl group, a C₂-C₆₀ heterocyclic group containing at least one heteroatom of O, N, S, Si, and P, a C₃-C₆₀ aliphatic ring, a fused ring group formed by a C₃-C₆₀ aliphatic ring with a C₆-C₆₀ aromatic ring, a C₁-C₅₀ alkyl group, a C₂-C₂₀ alkenyl group, a C₂-C₂₀ alkynyl group, a C₁-C₃₀ alkoxyl group, a C₆-C₃₀ aryloxy group and -L′-N(Rₐ)(R_{b}), and adjacent R¹ groups or adjacent R² groups together may be bonded to each other to form a ring, wherein the ring is a C₆-C₆₀ aromatic ring, a C₃-C₆₀ aliphatic ring, a C₂-C₆₀ heterocyclic ring containing at least one heteroatom of O, N, S, Si, and P, or a combination thereof.

m and n are each an integer of 0 to 4, when m is an integer of 2 or more, a plurality of R₁s are the same or different from each other, and when n is an integer of 2 or more, a plurality of Res are the same or different from each other.

Where R₁ and R₂ are an aryl group, the aryl group may be preferably a C₆-C₃₀, more preferably a C₆-C₁₈ aryl group, for example, phenyl, naphthyl, biphenyl and the like. Where R₁ and R₂ are a heterocyclic group, the heterocyclic group may be preferably a C₂-C₃₀, more preferably a C₂-C₁₆ heterocyclic group, for example, carbazole, benzocarbazole, pyridoindole and the like.

Where adjacent R₁ groups or adjacent R₂ groups together may be bonded to each other to form an aromatic ring, the aromatic ring is preferably a C₆-C₃₀ aromatic ring, more preferably a C₆-C₁₄ aromatic ring may be formed, for example, benzene, naphthalene, phenanthrene and the like. Where adjacent R₁ groups or adjacent R₂ groups together may be bonded to each other to form a heterocycle, the heterocycle is preferably a C₂-C₃₀ heterocycle, more preferably a C₂-C₁₆ heterocycle, for example, indole, benzoindole, dibenzoindole, benzothiophene, naphthothiophene, benzofuran and the like.

L′ is selected from the group consisting of a single bond, a C₆-C₆₀ arylene group, a fluorenylene group, a C₃-C₆₀ aliphatic ring, a C₂-C₆₀ heterocyclic group containing at least one heteroatom of O, N, S, Si, and P, and a combination thereof.

Where L′ are an arylene group, the arylene group may be preferably a C₆-C₃₀, more preferably a C₆-C₁₈ arylene group, for example, phenyl, naphthalene, biphenyl and the like.

Rₐ and R_{b} are each independently selected from the group consisting of a C₆-C₆₀ aryl group, a fluorenyl group, a C₂-C₆₀ heterocyclic group containing at least one heteroatom of O, N, S, Si, and P, a C₃-C₆₀ aliphatic ring, and a fused ring group formed by a C₃-C₆₀ aliphatic ring with a C₆-C₆₀ aromatic ring.

Where Rₐ and R_{b} are an aryl group, the aryl group may be preferably a C₆-C₃₀, more preferably a C₆-C₁₈ aryl group, for example, phenyl, biphenyl, naphthalene, terphenyl, phenanthrene, triphenylene and the like. Where Rₐ and R_{b} are a heterocyclic group, the heterocyclic group may be preferably a C₂-C₃₀, more preferably a C₂-C₂₂ heterocyclic group, for example, dibenzofuran, dibenzothiophene, benzothienopyrimidine, benzonaphthofuran, carbazole, phenylcarbazole, naphthylcarbazole and the like. Where Rₐ and R_{b} are a fluorenyl group, the fluorenyl group may be diphenylfluorene, spyrobifluorene, dimethylfluorene, dimethylbenzofluorene and the like.

R₁, R₂, the ring formed by adjacent R₁ groups, the ring formed by adjacent R₂ groups, L′, Rₐ and R_{b} may be each optionally substituted with one or more substituents selected from the group consisting of deuterium, halogen, a silane group unsubstituted or substituted with a C₁-C₂₀ alkyl group or a C₆-C₂₀ aryl group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group containing at least one heteroatom of O, N, S, Si, and P, a C₃-C₂₀ cycloalkyl group, a C₇-C₂₀ arylalkyl group, a C₈-C₂₀ arylalkenyl group, -L′-N(Rₐ)(R_{b}) and a combination thereof, wherein L′, Rₐ and R_{b} are the same as defined above.

Preferably, the compound represented by Formula 1 may be comprised in at least a light emitting layer.

Also, as another example, the compound represented by Formula 1 may be comprised in a light emitting layer and at least one layer of the organic material layer between a first electrode and the light emitting layer.

Also, as another example, the compound represented by Formula 1 may be comprised in a light emitting layer and at least two layers of the organic material layer between a first electrode and the light emitting layer.

Preferably, the compound represented by Formula 1 may be comprised in in a hole transport layer, an emission-auxiliary layer and a light emitting layer, more preferably, at least one of the compounds represented by Formula 1 comprised in the adjacent layers among the hole transport layer, the emission-auxiliary layer and the light emitting layer is different from each other.

Formula 1 may be represented by one of Formula 2 to Formula 4.

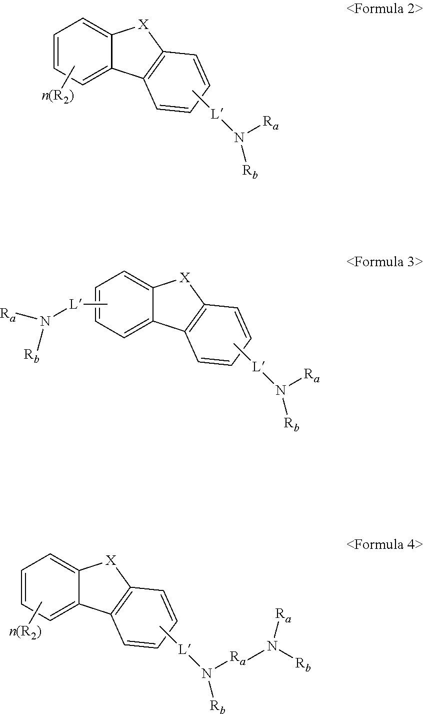

In Formulas 2 to 4, X, R₂, L′, Rₐ, R_{b} and n are the same as defined for Formula 1, and Rₐs may be the same or different from each other and R_{b}s may be the same or different from each other.

Preferably, the hole transport layer, the emission-auxiliary layer and the light emitting layer may comprise the compound represented by one of Formulas 2 to 4.

The light emitting layer may comprise a single compound or two or more compounds represented by Formula 5 or Formula 6, preferably, Formulas 5 and 6 may be comprised in Formula 1.

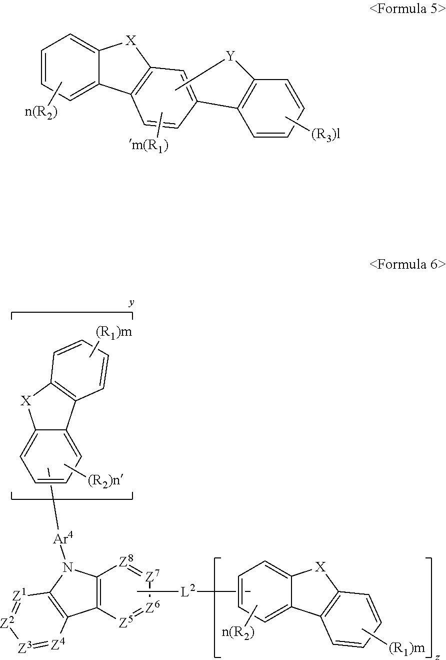

In Formulas 5 and 6, X, R₁, R₂, m and n are the same as defined for Formula 1, m′ is an integer of 0 to 2, n′ is an integer of 0 to 3, y and z are each an integer of 0 or 1, y+z is 1 or 2. Therefore, in Formula 6, it is preferable that at least one of 3-membered rings comprising X is present.

Y is O, S or N(Ar¹).

Ar¹ is selected from the group consisting of a C₆-C₆₀ aryl group, a fluorenyl group, a C₂-C₆₀ heterocyclic group containing at least one heteroatom selected from the group consisting of O, N, S, Si, a C₃-C₆₀ aliphatic ring, -L′-N(Rₐ)(R_{b}) and a combination thereof. Preferably, Ar¹ is selected from the group consisting of a C₆-C₂₀ aryl group, a fluorenyl group, a C₂-C₂₀ heterocyclic group containing at least one heteroatom selected from the group consisting of O, N, S, Si, a C₃-C₂₀ aliphatic ring, -L′-N(Rₐ)(R_{b}) and a combination thereof, wherein L′, Rₐ and R_{b} are the same as defined above.

Where Ar¹ is an aryl group, the aryl group may be preferably a C₆-C₃₀, more preferably a C₆-C₁₈ aryl group, for example, phenyl, naphthalene, biphenyl, terphenyl, triphenylene, phenanthrene and the like. Where Ar¹ is a heterocyclic group, the heterocyclic group may be preferably a C₂-C₃₀, more preferably a C₂-C₁₈ heterocyclic group, for example, triazine, pyrimidine, pyridine, quinazoline, quinoxaline, benzothienopyrimidine, benzoquinazoline, carbazole, phenylcarbazole, dibenzothiophene, dibenzofuran and the like. Where Ar¹ is a fluorenyl group, the fluorenyl group may be dimethylfluorene, diphenylfluorene, spyrofluorene and the like.

R₃ is each independently selected from the group consisting of hydrogen, deuterium, halogen, a C₆-C₆₀ aryl group, a fluorenyl group, a C₂-C₆₀ heterocyclic group containing at least one heteroatom of O, N, S, Si, and P, a C₃-C₆₀ aliphatic ring, a fused ring group formed by a C₃-C₆₀ aliphatic ring with a C₆-C₆₀ aromatic ring, a C₁-C₅₀ alkyl group, a C₂-C₂₀ alkenyl group, a C₂-C₂₀ alkynyl group, a C₁-C₃₀ alkoxyl group, a C₆-C₃₀ aryloxy group and -L′-N(Rₐ)(R_{b}). Preferably, R₃ is each independently selected from the group consisting of hydrogen, deuterium, halogen, a C₆-C₂₀ aryl group, a fluorenyl group, a C₂-C₂₀ heterocyclic group containing at least one heteroatom of O, N, S, Si, and P, a C₃-C₂₀ aliphatic ring, a C₁-C₂₀ alkyl group, a C₂-C₂₀ alkenyl group, a C₂-C₂₀ alkynyl group and -L′—N(Rₐ)(R_{b}), wherein L′, Rₐ and R_{b} are the same as defined above.

l is an integer of 0 to 4, and when l is an integer of 2 or more, a plurality of R₃s are the same or different from each other, and adjacent R₃ groups may be optionally linked to each other to form a ring, wherein the ring is a C₆-C₆₀ aromatic ring, a C₃-C₆₀ aliphatic ring, a C₂-C₆₀ heterocyclic group containing at least one heteroatom selected from the group consisting of O, N, S, Si, and P, or a combination thereof.

In Formula 6, Z¹ to Z⁸ are each independently C(R) or N.

Ar⁴ is selected from the group consisting of a C₆-C₆₀ aryl group, a C₆-C₆₀ arylene group, a C₂-C₆₀ heterocyclic group containing at least one heteroatom selected from the group consisting of O, N, S, Si, and P, a C₃-C₆₀ aliphatic ring, a fused ring group formed by a C₃-C₆₀ aliphatic ring with a C₆-C₆₀ aromatic ring, a C₁-C₅₀ alkyl group, a C₁-C₅₀ alkylene group, a C₆-C₆₀ arylamine group, a fluorenyl group, and a fluorenylene group. Preferably, Ar⁴ is selected from the group consisting of a C₆-C₂₀ aryl group, a C₆-C₂₀ arylene group, a C₂-C₂₀ heterocyclic group containing at least one heteroatom selected from the group consisting of O, N, S, Si, and P, a C₃-C₂₀ aliphatic ring, a C₁-C₂₀ alkyl group, a C₁-C₂₀ alkylene group, a fluorenyl group, and a fluorenylene group.

Where Ar⁴ is an aryl or arylene group, the aryl or arylene group may be preferably a C₆-C₃₀, more preferably a C₆-C₁₈ aryl or arylene group, for example, phenyl, naphthalene, biphenyl, terphenyl and the like. Where Ar⁴ is a heterocyclic group, the heterocyclic group may be preferably a C₂-C₃₀, more preferably a C₂-C₁₂ heterocyclic group, for example, pyridine, pyrimidine, triazine, dibenzofuran, dibenzothiophene and the like.

L² is selected from the group consisting of a single bond, a C₆-C₆₀ aryl group, a C₆-C₆₀ arylene group, a C₂-C₆₀ heterocyclic group containing at least one heteroatom selected from the group consisting of O, N, S, Si, and P, a C₃-C₆₀ aliphatic ring, a fluorenyl group, a fluorenylene group, and a combination thereof. Preferably, L² is selected from the group consisting of a single bond, a C₆-C₂₀ aryl group, a C₆-C₂₀ arylene group, a C₂-C₂₀ heterocyclic group containing at least one heteroatom selected from the group consisting of O, N, S, Si, and P, a C₃-C₂₀ aliphatic ring, a fluorenyl group, a fluorenylene group, and a combination thereof.

R is selected from the group consisting of hydrogen, a C₆-C₆₀ aryl group, a C₂-C₆₀ heterocyclic group containing at least one heteroatom selected from the group consisting of O, N, S, Si, a fluorenyl group, a C₁-C₅₀ alkyl group and a C₆-C₆₀ arylamine group, and adjacent R groups may be optionally linked to each other to form a ring. Preferably, R is selected from the group consisting of hydrogen, a C₆-C₂₀ aryl group, a C₂-C₂₀ heterocyclic group containing at least one heteroatom selected from the group consisting of O, N, S, Si, a fluorenyl group and a C₁-C₂₀ alkyl group.

Preferably, Formula 5 may be represented by one of Formulas 7 to 42.

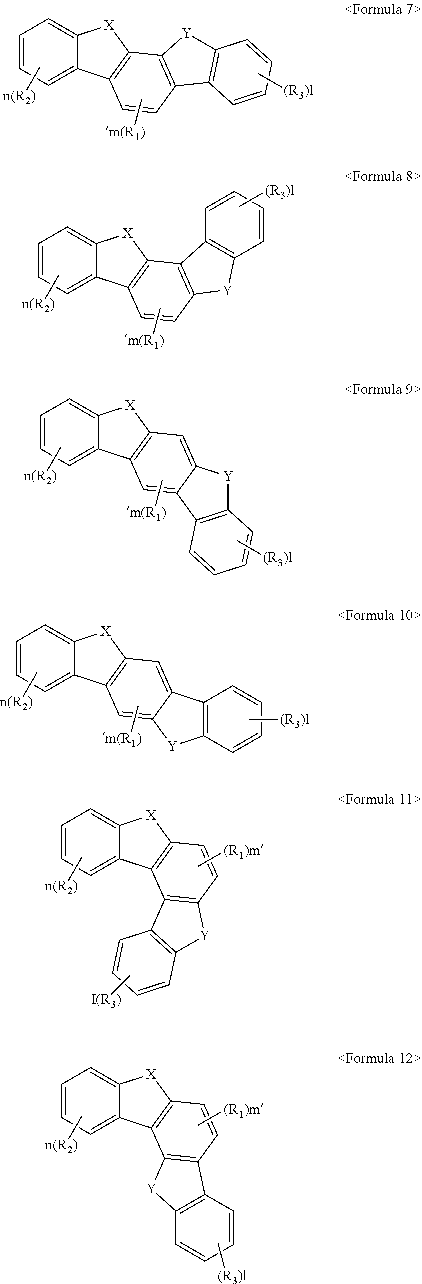

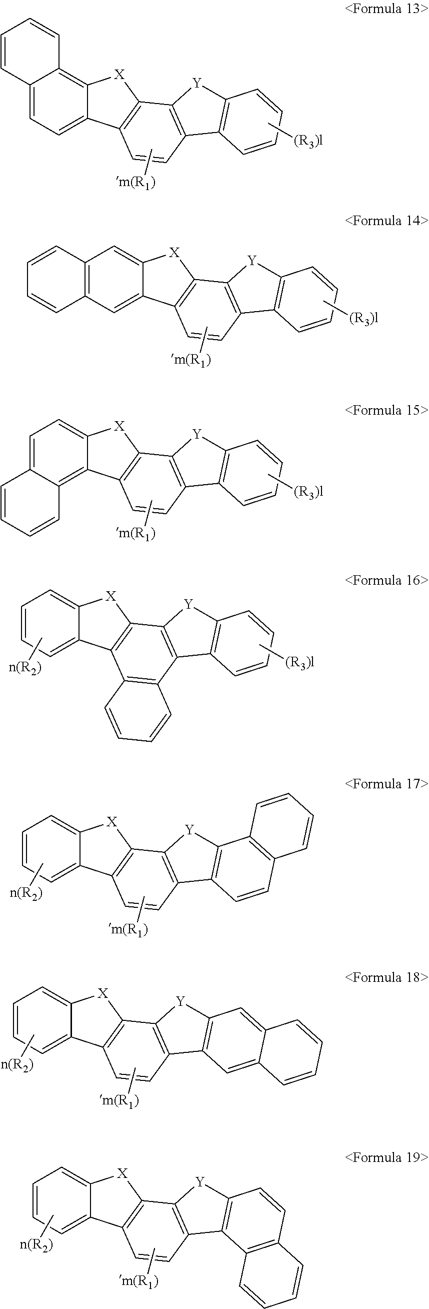

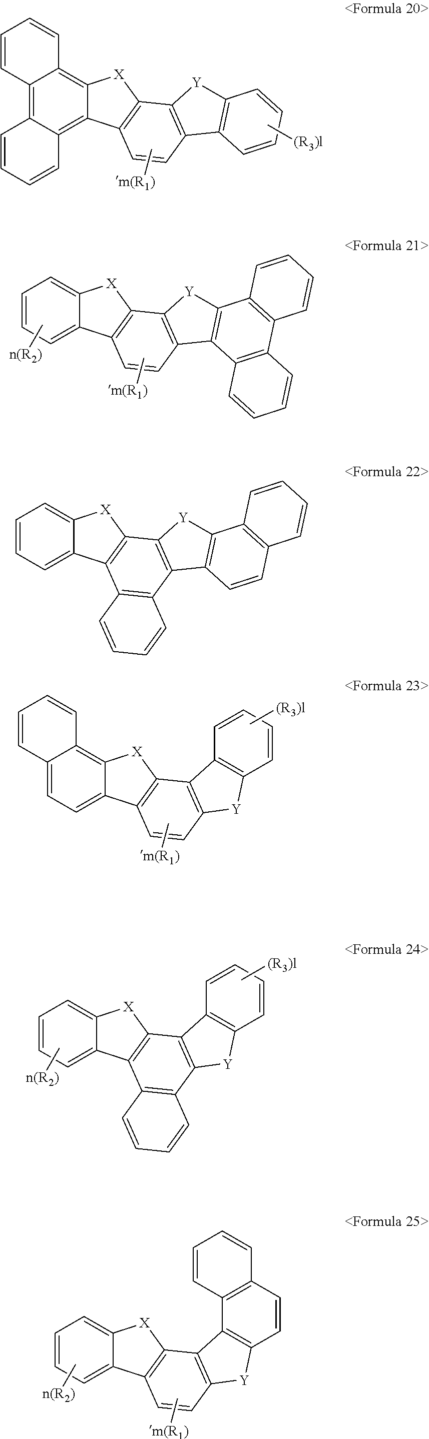

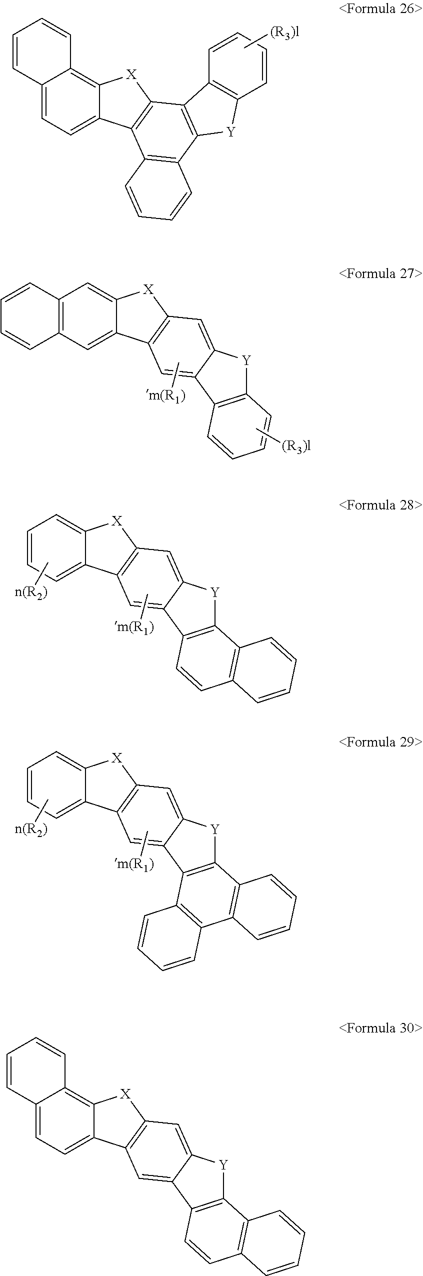

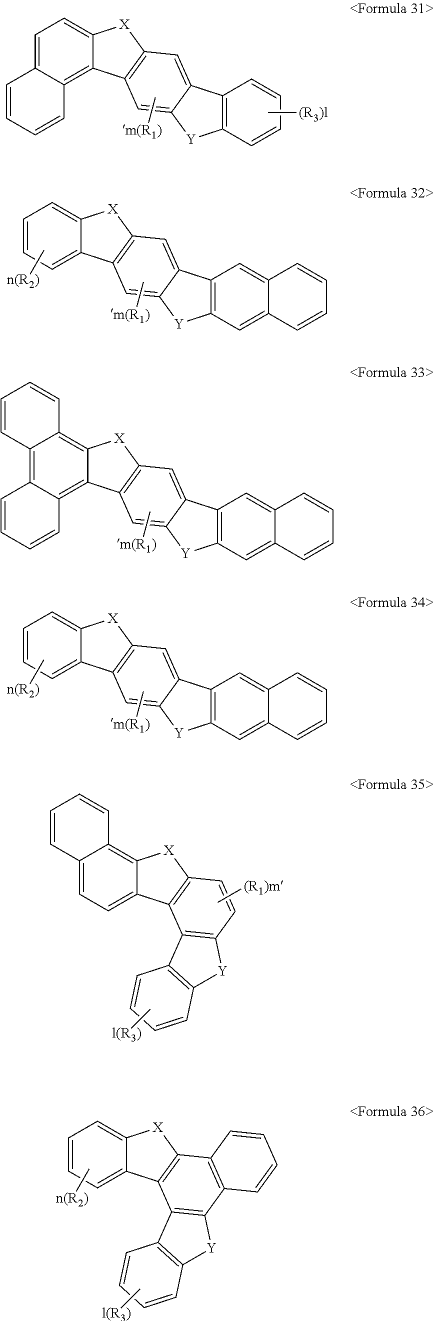

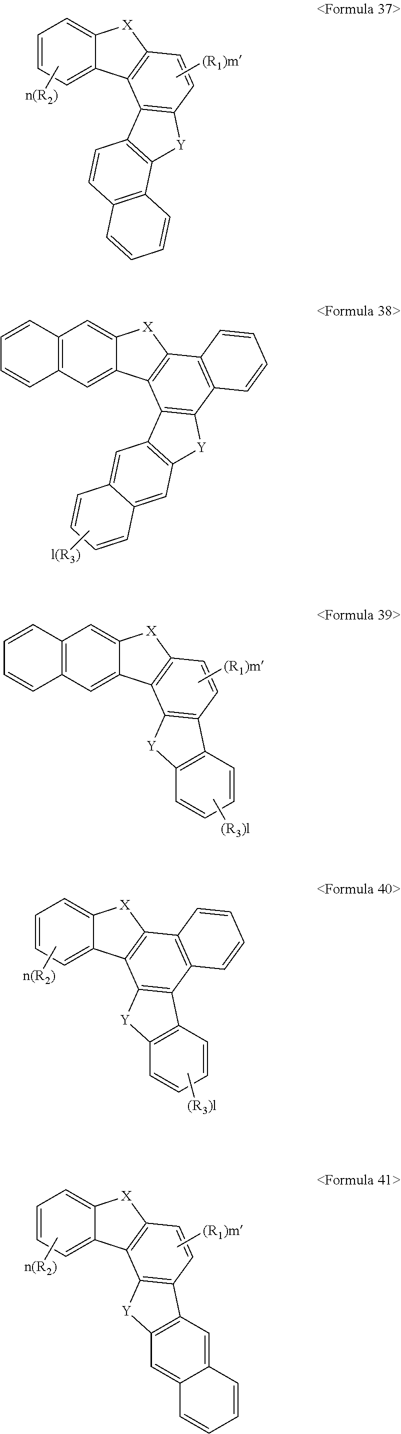

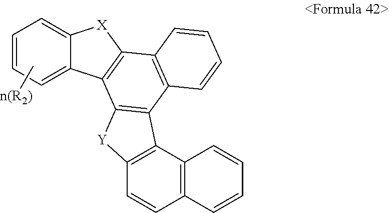

In Formulas 7 to 42, the symbols such as X, Y, R₁, R₂, R₃, n, m, l and so on are the same as defined for Formula 5.

Preferably, in Formula 5, where Y is N(Ar¹), Ar¹ may be represented by one of Formulas (A-1) to (A-7).

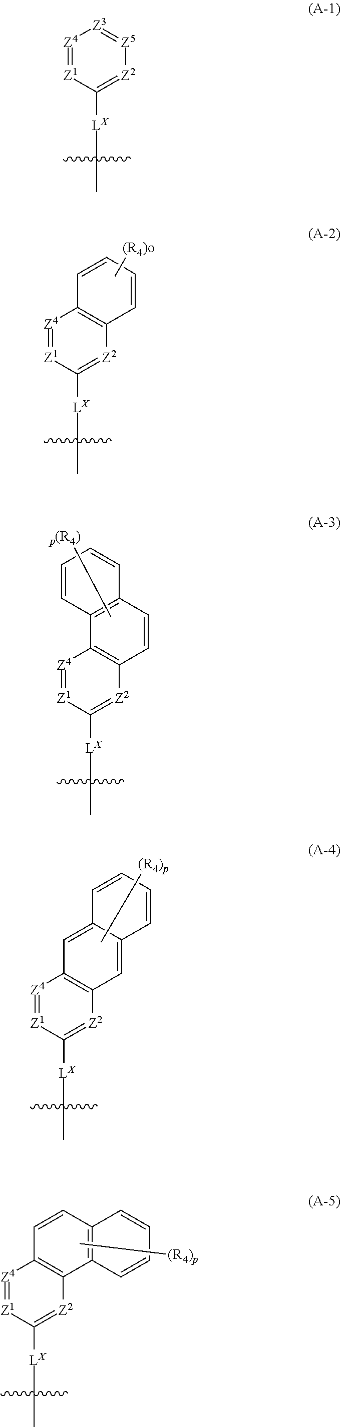

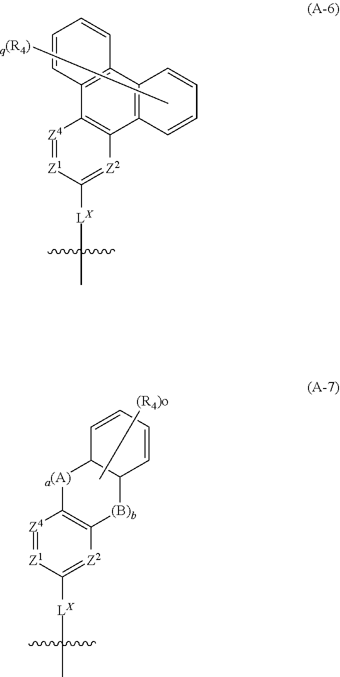

In Formulas A-1 to A-7, each of symbols may be defined as follows.

Z¹ to Z⁵ are N or C(R^{X}), and one or more of Z¹ to Z⁵ is N.

R_{X} and R₄ are each independently selected from the group consisting of hydrogen, deuterium, halogen, a C₆-C₆₀ aryl group, a fluorenyl group, a C₂-C₆₀ heterocyclic group containing at least one heteroatom of O, N, S, Si, and P, a fused ring group formed by a C₃-C₆₀ aliphatic ring with a C₆-C₆₀ aromatic ring, a C₁-C₅₀ alkyl group, a C₂-C₂₀ alkenyl group, a C₂-C₂₀ alkynyl group, a C₁-C₃₀ alkoxyl group, a C₆-C₃₀ aryloxy group and -L′-N(Rₐ)(R_{b}). Preferably, R^{X} and R₄ are each independently selected from the group consisting of hydrogen, deuterium, halogen, a C₆-C₂₀ aryl group, a fluorenyl group, a C₂-C₂₀ heterocyclic group containing at least one heteroatom of O, N, S, Si, and P, a C₃-C₂₀ aliphatic ring, a C₁-C₂₀ alkyl group, a C₂-C₂₀ alkenyl group, a C₂-C₂₀ alkynyl group and -L′-N(Rₐ)(R_{b}), wherein L′, Rₐ and R_{b} are the same as defined above.

Also, adjacent R^{X} groups or adjacent R₄ groups together may be bonded to each other to form a C₆-C₆₀ aromatic ring or a C₂-C₆₀ heterocyclic ring, preferably, a C₆-C₂₀ aromatic ring or a C₂-C₂₀ heterocyclic ring.

o is an integer of 0-4, p is an integer of 0-6, q is an integer of 0-8, and when each of them is an integer of 2 or more, a plurality of R₄s are the same or different from each other.

L^{X} is selected from the group consisting of a single bond, a C₆-C₆₀ arylene group, a fluorenylene group, a C₂-C₆₀ heterocyclic group containing at least one heteroatom of O, N, S, Si, and P, a C₃-C₆₀ aliphatic ring and a combination thereof. Preferably, L^{X} is selected from the group consisting of a single bond, a C₆-C₂₀ arylene group, a fluorenylene group, a C₂-C₂₀ heterocyclic group containing at least one heteroatom of O, N, S, Si, and P, a C₃-C₂₀ aliphatic ring and a combination thereof.

L′, Rₐ and R_{b} are the same as defined for Formula 1.

Preferably, the light emitting layer may comprise at least one of the compounds represented by Formulas 2 to 4.

Preferably, the hole transport layer and the emission-auxiliary layer may comprise the compound represented by Formulas 2 to 4 and the light emitting layer may comprise the compound represented by Formula 5 or Formula 6.

More preferably, the light emitting layer may comprise the compound represented by Formula 5 and the compound represented by one of Formulas 2 to 4.

Preferably, the emission-auxiliary layer may comprise the compound represented by Formula 3 and light emitting layer may comprise the compound represented by Formula 5.

More preferably, the hole transport layer may comprise the compound represented by Formula 4 and the emission-auxiliary layer may comprise the compound represented by Formula 3 and light emitting layer may comprise the compound represented by Formula 5.

Preferably, in Formulas 3 to 5, all of X may be O.

Also, preferably, in Formulas 3 to 5, all of X may be S.

Also, preferably, X may be O in Formula 4, and X may be S in Formulas 3 and 5.

Also, preferably, X may be O in Formula 4, and X may be different from each other in Formulas 3 and 5.

Also, preferably, X may be S in Formula 4, and X may be O in Formulas 3 and 5.

Also, preferably, X may be S in Formula 4, and X may be different from each other in Formulas 3 and 5.

Specifically, the compound represented by formula 1 may be one of the following compounds.

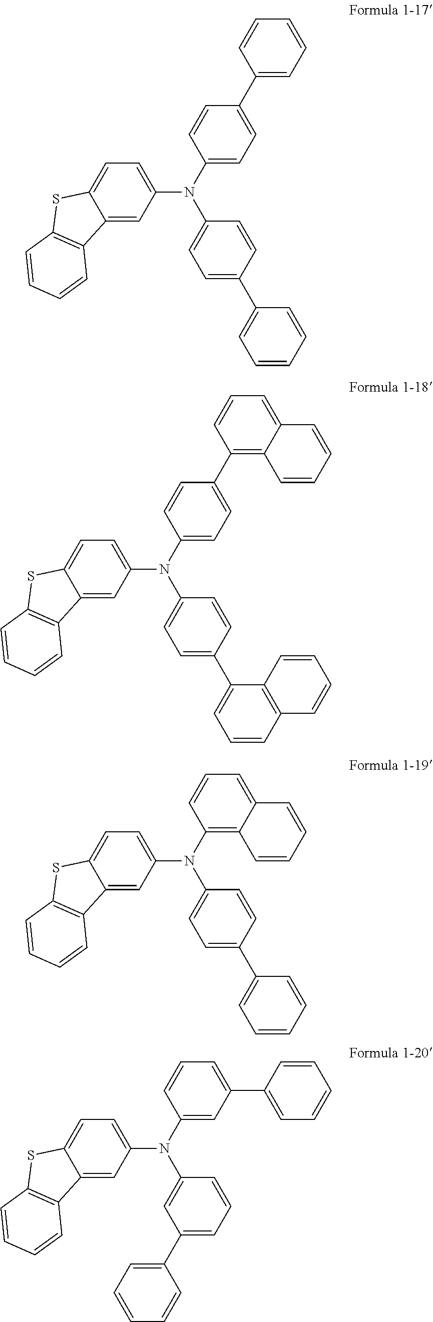

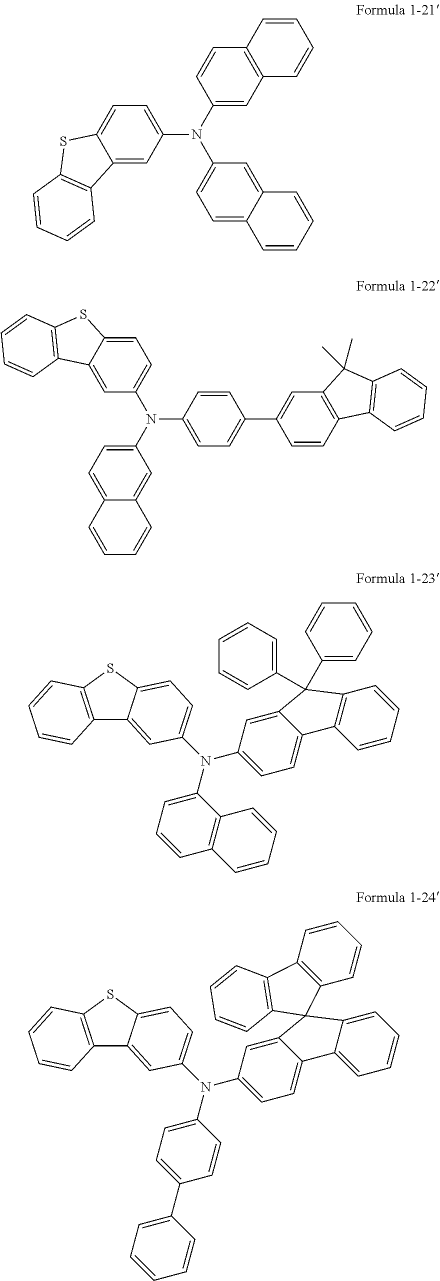 [SMILES: c1ccc2cc(N(c3ccc4ccccc4c3)c3ccc4sc5ccccc5c4c3)ccc2c1 ; CC1(C)c2ccccc2-c2ccc(-c3ccc(N(c4ccc5ccccc5c4)c4ccc5sc6ccccc6c5c4)cc3)cc21 ; c1ccc(C2(c3ccccc3)c3ccccc3-c3ccc(N(c4ccc5sc6ccccc6c5c4)c4cccc5ccccc45)cc32)cc1 ; c1ccc(-c2ccc(N(c3ccc4c(c3)C3(c5ccccc5-c5ccccc53)c3ccccc3-4)c3ccc4sc5ccccc5c4c3)cc2)cc1]

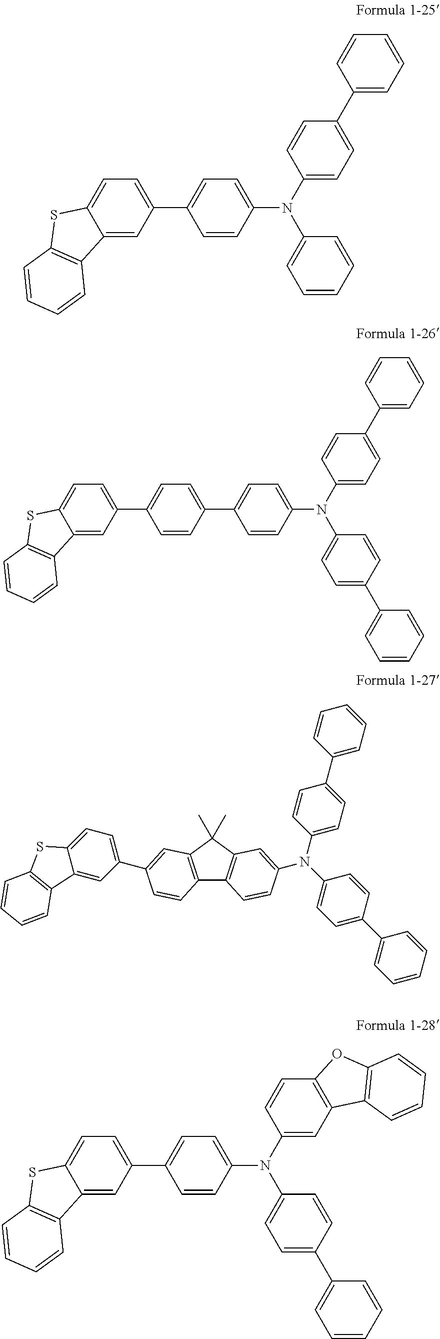 [SMILES: c1ccc(-c2ccc(N(c3ccccc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-c5ccc6sc7ccccc7c6c5)cc4)cc3)cc2)cc1 ; CC1(C)c2cc(-c3ccc4sc5ccccc5c4c3)ccc2-c2ccc(N(c3ccc(-c4ccccc4)cc3)c3ccc(-c4ccccc4)cc3)cc21 ; c1ccc(-c2ccc(N(c3ccc(-c4ccc5sc6ccccc6c5c4)cc3)c3ccc4oc5ccccc5c4c3)cc2)cc1]

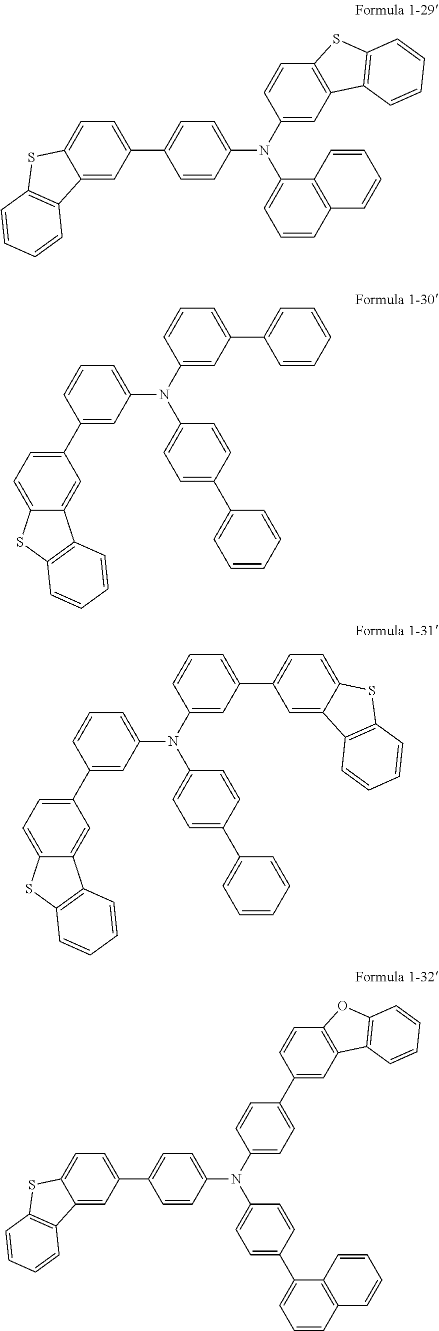 [SMILES: c1ccc2c(N(c3ccc(-c4ccc5sc6ccccc6c5c4)cc3)c3ccc4sc5ccccc5c4c3)cccc2c1 ; c1ccc(-c2ccc(N(c3cccc(-c4ccccc4)c3)c3cccc(-c4ccc5sc6ccccc6c5c4)c3)cc2)cc1 ; c1ccc(-c2ccc(N(c3cccc(-c4ccc5sc6ccccc6c5c4)c3)c3cccc(-c4ccc5sc6ccccc6c5c4)c3)cc2)cc1 ; c1ccc2c(-c3ccc(N(c4ccc(-c5ccc6oc7ccccc7c6c5)cc4)c4ccc(-c5ccc6sc7ccccc7c6c5)cc4)cc3)cccc2c1]

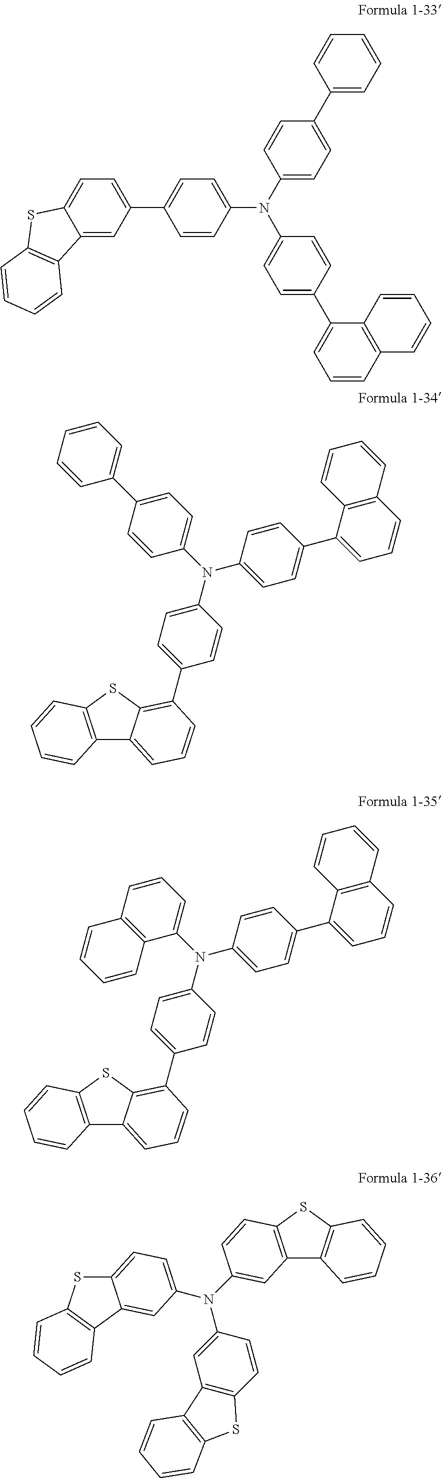 [SMILES: c1ccc(-c2ccc(N(c3ccc(-c4ccc5sc6ccccc6c5c4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5c4sc4ccccc45)cc3)cc2)cc1 ; c1ccc2c(-c3ccc(N(c4ccc(-c5cccc6c5sc5ccccc56)cc4)c4cccc5ccccc45)cc3)cccc2c1 ; c1ccc2c(c1)sc1ccc(N(c3ccc4sc5ccccc5c4c3)c3ccc4sc5ccccc5c4c3)cc12]

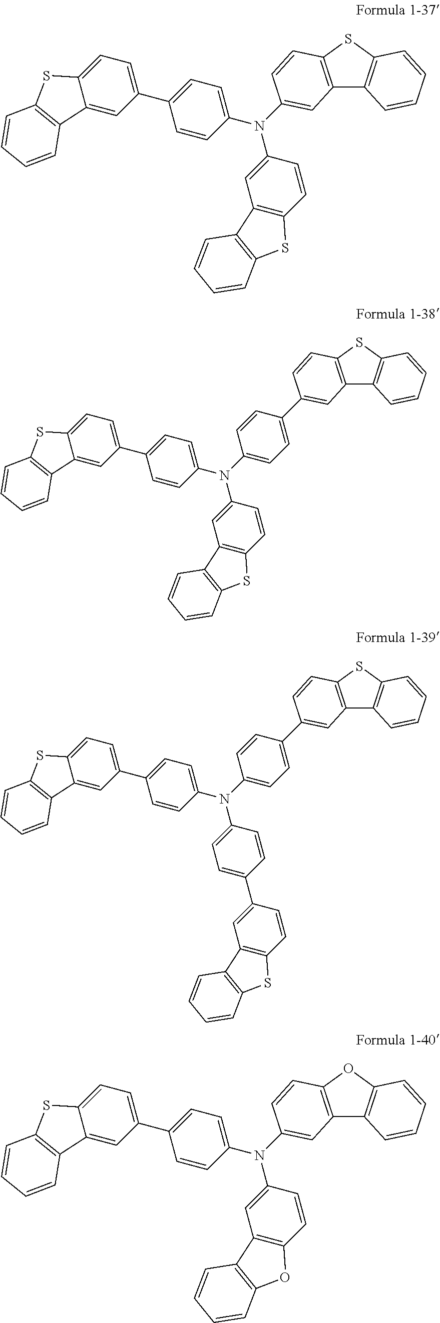 [SMILES: c1ccc2c(c1)sc1ccc(-c3ccc(N(c4ccc5sc6ccccc6c5c4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc(-c5ccc6sc7ccccc7c6c5)cc4)cc3)cc12 ; c1ccc2c(c1)oc1ccc(N(c3ccc(-c4ccc5sc6ccccc6c5c4)cc3)c3ccc4oc5ccccc5c4c3)cc12]

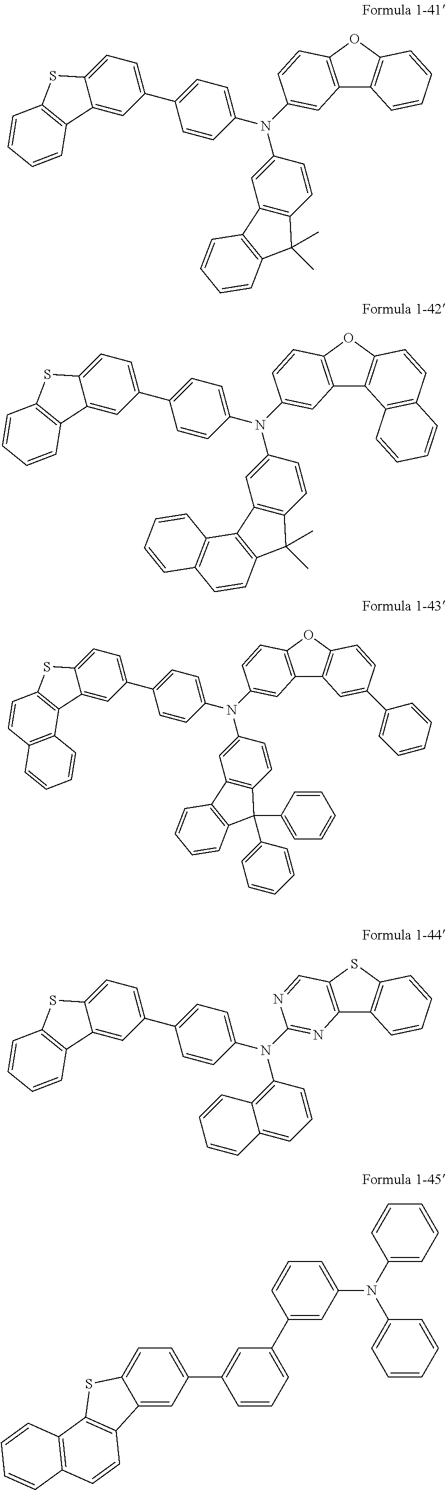 [SMILES: CC1(C)c2ccccc2-c2cc(N(c3ccc(-c4ccc5sc6ccccc6c5c4)cc3)c3ccc4oc5ccccc5c4c3)ccc21 ; CC1(C)c2ccc(N(c3ccc(-c4ccc5sc6ccccc6c5c4)cc3)c3ccc4oc5ccc6ccccc6c5c4c3)cc2-c2c1ccc1ccccc21 ; c1ccc(-c2ccc3oc4ccc(N(c5ccc(-c6ccc7sc8ccc9ccccc9c8c7c6)cc5)c5ccc6c(c5)-c5ccccc5C6(c5ccccc5)c5ccccc5)cc4c3c2)cc1 ; c1ccc2c(N(c3ccc(-c4ccc5sc6ccccc6c5c4)cc3)c3ncc4sc5ccccc5c4n3)cccc2c1 ; c1ccc(N(c2ccccc2)c2cccc(-c3cccc(-c4ccc5sc6c7ccccc7ccc6c5c4)c3)c2)cc1]

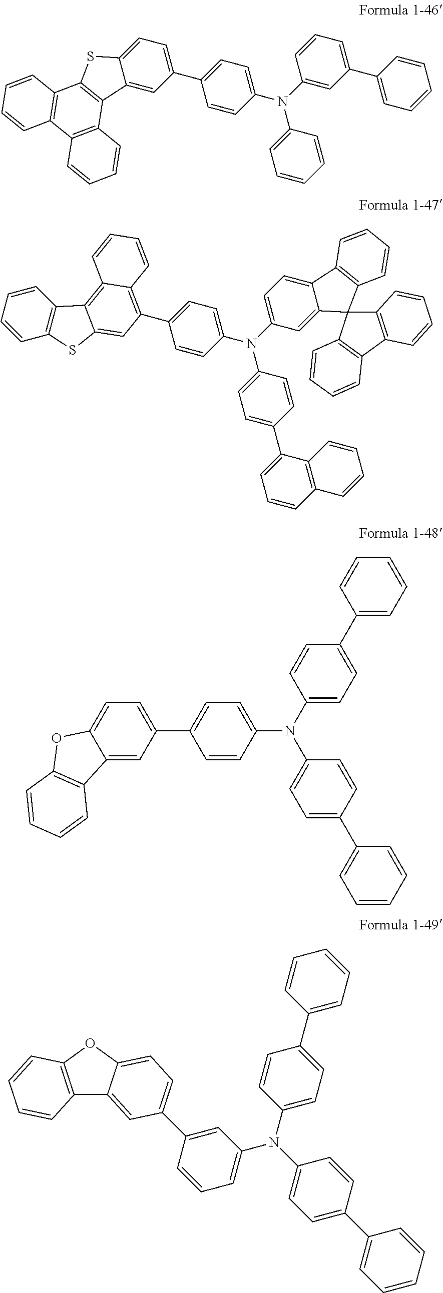 [SMILES: c1ccc(-c2cccc(N(c3ccccc3)c3ccc(-c4ccc5sc6c7ccccc7c7ccccc7c6c5c4)cc3)c2)cc1 ; c1ccc2c(c1)-c1ccccc1C21c2ccccc2-c2ccc(N(c3ccc(-c4cccc5ccccc45)cc3)c3ccc(-c4cc5sc6ccccc6c5c5ccccc45)cc3)cc21 ; c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3cccc(-c4ccc5oc6ccccc6c5c4)c3)cc2)cc1]

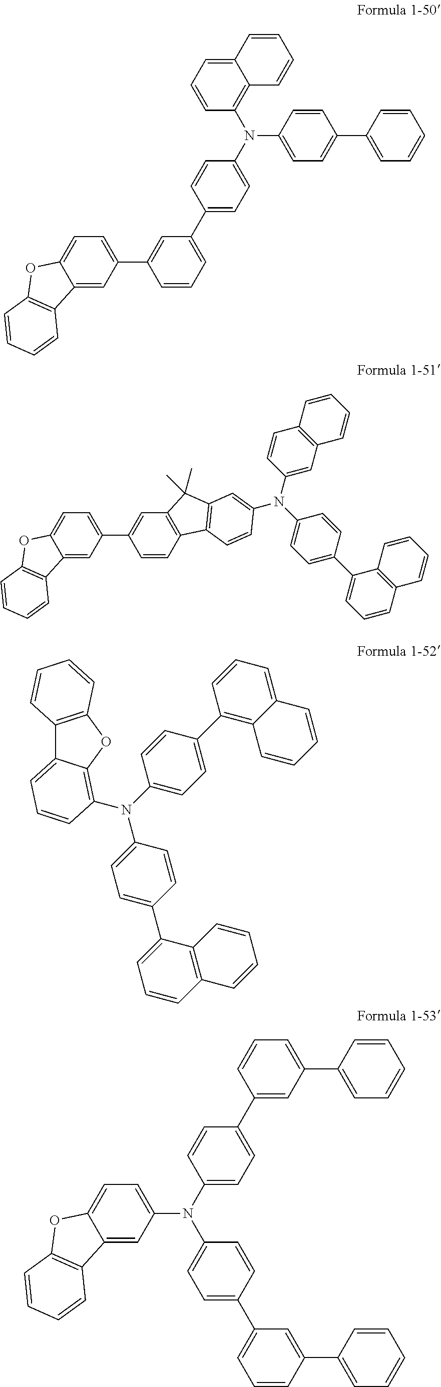 [SMILES: c1ccc(-c2ccc(N(c3ccc(-c4cccc(-c5ccc6oc7ccccc7c6c5)c4)cc3)c3cccc4ccccc34)cc2)cc1 ; CC1(C)c2cc(-c3ccc4oc5ccccc5c4c3)ccc2-c2ccc(N(c3ccc(-c4cccc5ccccc45)cc3)c3ccc4ccccc4c3)cc21 ; c1ccc2c(-c3ccc(N(c4ccc(-c5cccc6ccccc56)cc4)c4cccc5c4oc4ccccc45)cc3)cccc2c1 ; c1ccc(-c2cccc(-c3ccc(N(c4ccc(-c5cccc(-c6ccccc6)c5)cc4)c4ccc5oc6ccccc6c5c4)cc3)c2)cc1]

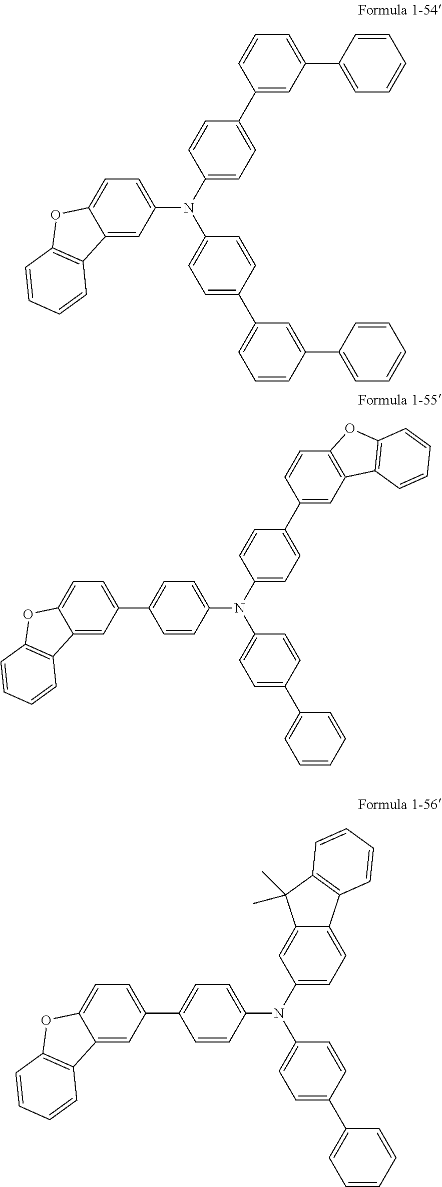 [SMILES: c1ccc(-c2cccc(-c3ccc(N(c4ccc(-c5cccc(-c6ccccc6)c5)cc4)c4ccc5oc6ccccc6c5c4)cc3)c2)cc1 ; c1ccc(-c2ccc(N(c3ccc(-c4ccc5oc6ccccc6c5c4)cc3)c3ccc(-c4ccc5oc6ccccc6c5c4)cc3)cc2)cc1 ; CC1(C)c2ccccc2-c2ccc(N(c3ccc(-c4ccccc4)cc3)c3ccc(-c4ccc5oc6ccccc6c5c4)cc3)cc21]

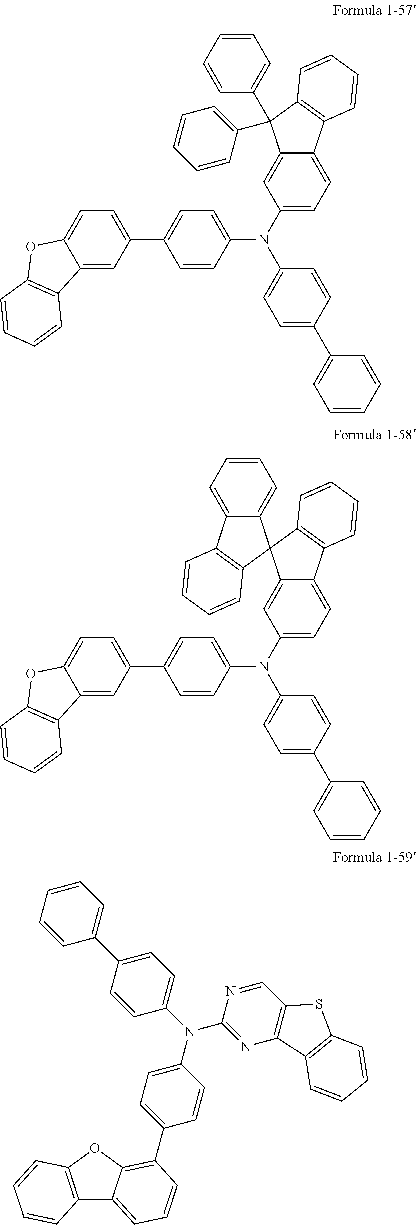 [SMILES: c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4cccc5c4oc4ccccc45)cc3)c3ncc4sc5ccccc5c4n3)cc2)cc1]

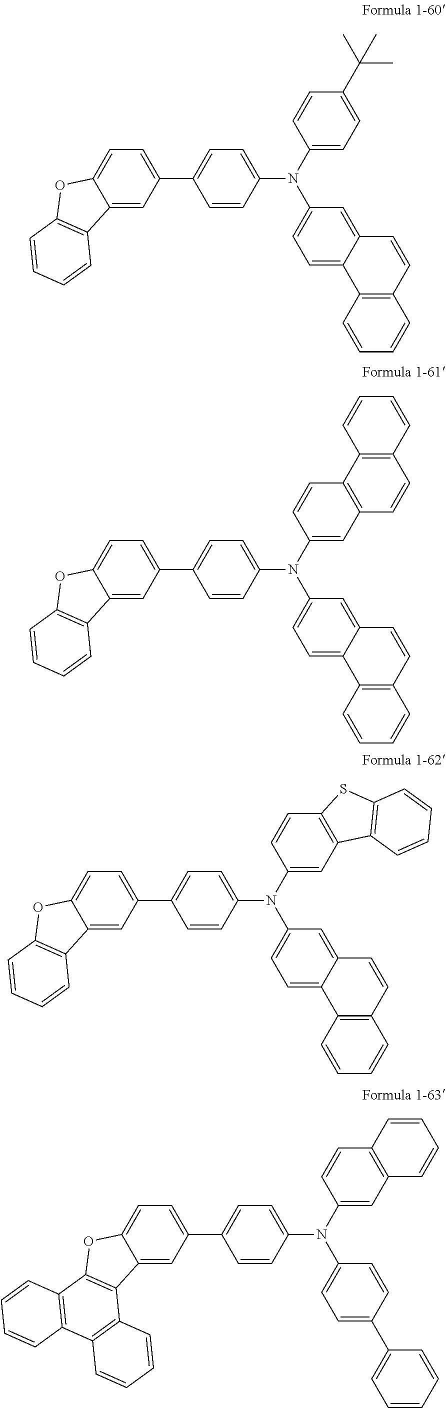

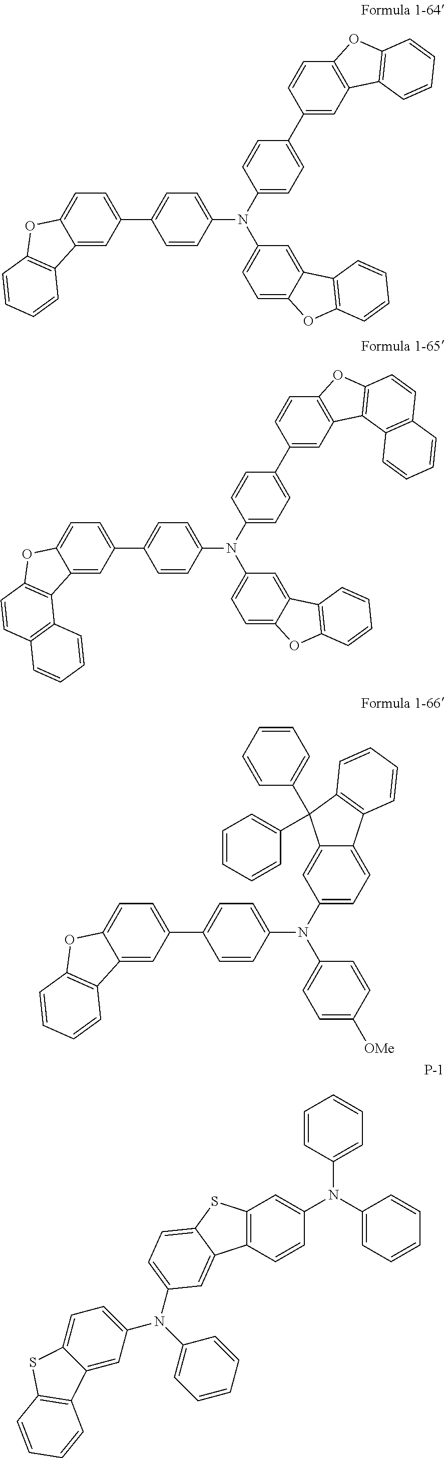 [SMILES: c1ccc2c(c1)oc1ccc(-c3ccc(N(c4ccc(-c5ccc6oc7ccccc7c6c5)cc4)c4ccc5oc6ccccc6c5c4)cc3)cc12 ; c1ccc2c(c1)ccc1oc3ccc(-c4ccc(N(c5ccc(-c6ccc7oc8ccc9ccccc9c8c7c6)cc5)c5ccc6oc7ccccc7c6c5)cc4)cc3c12 ; COc1ccc(N(c2ccc(-c3ccc4oc5ccccc5c4c3)cc2)c2ccc3c(c2)C(c2ccccc2)(c2ccccc2)c2ccccc2-3)cc1 ; c1ccc(N(c2ccccc2)c2ccc3c(c2)sc2ccc(N(c4ccccc4)c4ccc5sc6ccccc6c5c4)cc23)cc1]

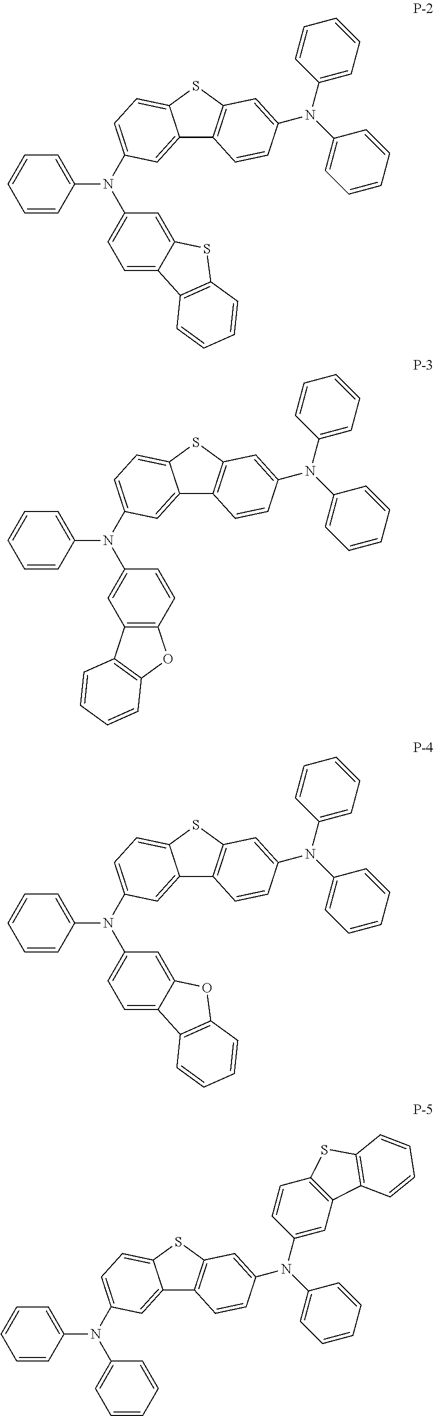 [SMILES: c1ccc(N(c2ccccc2)c2ccc3c(c2)sc2ccc(N(c4ccccc4)c4ccc5c(c4)sc4ccccc45)cc23)cc1 ; c1ccc(N(c2ccccc2)c2ccc3c(c2)sc2ccc(N(c4ccccc4)c4ccc5oc6ccccc6c5c4)cc23)cc1 ; c1ccc(N(c2ccccc2)c2ccc3c(c2)sc2ccc(N(c4ccccc4)c4ccc5c(c4)oc4ccccc45)cc23)cc1 ; c1ccc(N(c2ccc3c(c2)sc2ccc(N(c4ccccc4)c4ccccc4)cc23)c2ccc3sc4ccccc4c3c2)cc1]

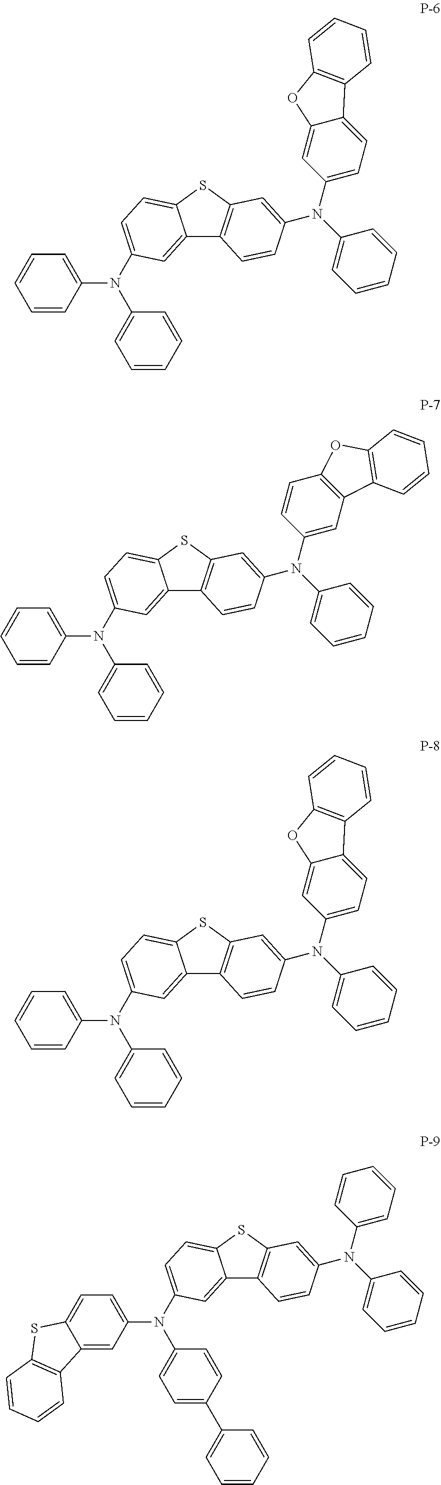 [SMILES: c1ccc(N(c2ccc3c(c2)oc2ccccc23)c2ccc3c(c2)sc2ccc(N(c4ccccc4)c4ccccc4)cc23)cc1 ; c1ccc(N(c2ccc3c(c2)sc2ccc(N(c4ccccc4)c4ccccc4)cc23)c2ccc3oc4ccccc4c3c2)cc1 ; c1ccc(N(c2ccc3c(c2)oc2ccccc23)c2ccc3c(c2)sc2ccc(N(c4ccccc4)c4ccccc4)cc23)cc1 ; c1ccc(-c2ccc(N(c3ccc4sc5ccccc5c4c3)c3ccc4sc5cc(N(c6ccccc6)c6ccccc6)ccc5c4c3)cc2)cc1]

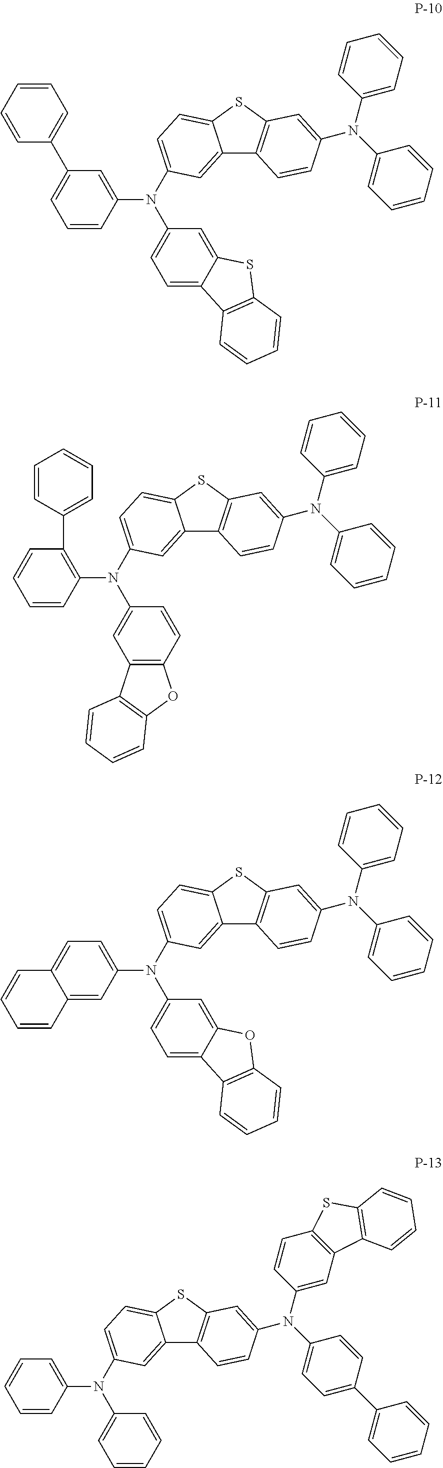 [SMILES: c1ccc(-c2cccc(N(c3ccc4c(c3)sc3ccccc34)c3ccc4sc5cc(N(c6ccccc6)c6ccccc6)ccc5c4c3)c2)cc1 ; c1ccc(-c2ccccc2N(c2ccc3oc4ccccc4c3c2)c2ccc3sc4cc(N(c5ccccc5)c5ccccc5)ccc4c3c2)cc1 ; c1ccc(N(c2ccccc2)c2ccc3c(c2)sc2ccc(N(c4ccc5ccccc5c4)c4ccc5c(c4)oc4ccccc45)cc23)cc1 ; c1ccc(-c2ccc(N(c3ccc4c(c3)sc3ccc(N(c5ccccc5)c5ccccc5)cc34)c3ccc4sc5ccccc5c4c3)cc2)cc1]

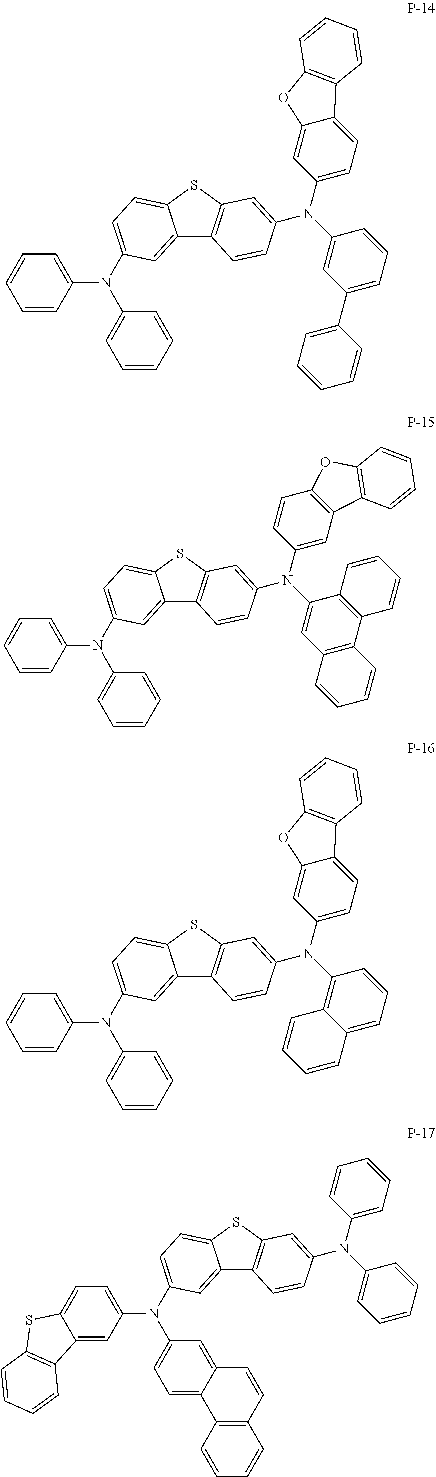 [SMILES: c1ccc(-c2cccc(N(c3ccc4c(c3)oc3ccccc34)c3ccc4c(c3)sc3ccc(N(c5ccccc5)c5ccccc5)cc34)c2)cc1 ; c1ccc(N(c2ccccc2)c2ccc3sc4cc(N(c5ccc6oc7ccccc7c6c5)c5cc6ccccc6c6ccccc56)ccc4c3c2)cc1 ; c1ccc(N(c2ccccc2)c2ccc3sc4cc(N(c5ccc6c(c5)oc5ccccc56)c5cccc6ccccc56)ccc4c3c2)cc1 ; c1ccc(N(c2ccccc2)c2ccc3c(c2)sc2ccc(N(c4ccc5c(ccc6ccccc65)c4)c4ccc5sc6ccccc6c5c4)cc23)cc1]

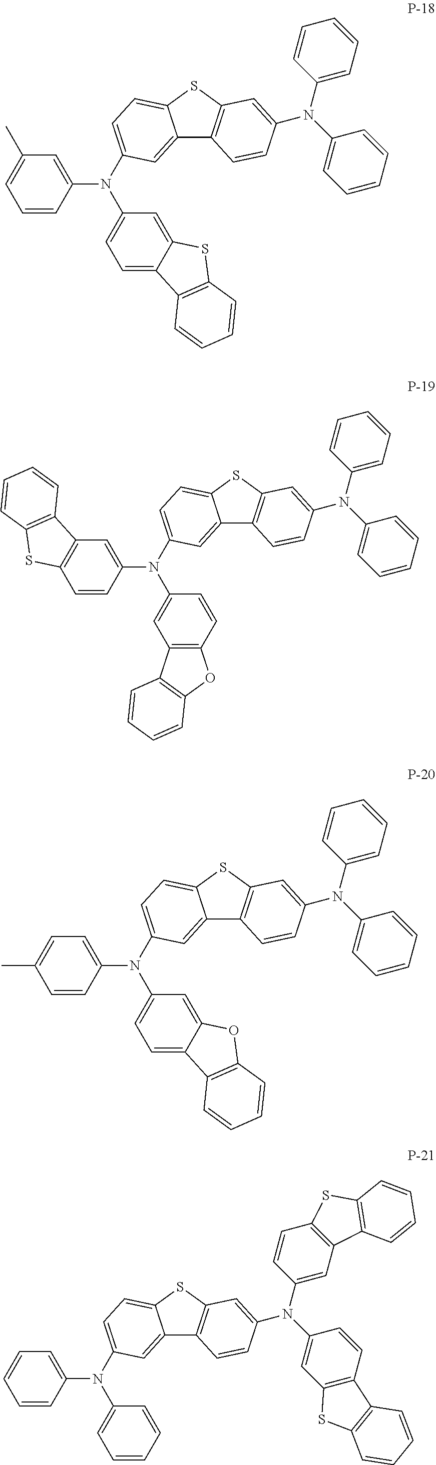 [SMILES: Cc1cccc(N(c2ccc3c(c2)sc2ccccc23)c2ccc3sc4cc(N(c5ccccc5)c5ccccc5)ccc4c3c2)c1 ; c1ccc(N(c2ccccc2)c2ccc3c(c2)sc2ccc(N(c4ccc5oc6ccccc6c5c4)c4ccc5sc6ccccc6c5c4)cc23)cc1 ; Cc1ccc(N(c2ccc3c(c2)oc2ccccc23)c2ccc3sc4cc(N(c5ccccc5)c5ccccc5)ccc4c3c2)cc1 ; c1ccc(N(c2ccccc2)c2ccc3sc4cc(N(c5ccc6c(c5)sc5ccccc56)c5ccc6sc7ccccc7c6c5)ccc4c3c2)cc1]

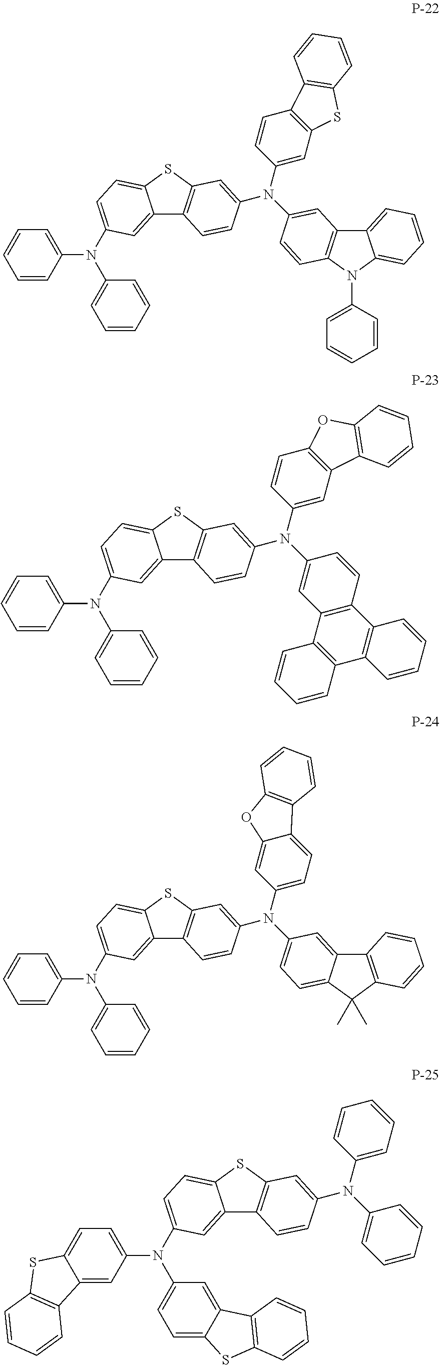 [SMILES: c1ccc(N(c2ccccc2)c2ccc3sc4cc(N(c5ccc6c(c5)sc5ccccc56)c5ccc6c(c5)c5ccccc5n6-c5ccccc5)ccc4c3c2)cc1 ; c1ccc(N(c2ccccc2)c2ccc3sc4cc(N(c5ccc6oc7ccccc7c6c5)c5ccc6c7ccccc7c7ccccc7c6c5)ccc4c3c2)cc1 ; CC1(C)c2ccccc2-c2cc(N(c3ccc4c(c3)oc3ccccc34)c3ccc4c(c3)sc3ccc(N(c5ccccc5)c5ccccc5)cc34)ccc21 ; c1ccc(N(c2ccccc2)c2ccc3c(c2)sc2ccc(N(c4ccc5sc6ccccc6c5c4)c4ccc5sc6ccccc6c5c4)cc23)cc1]

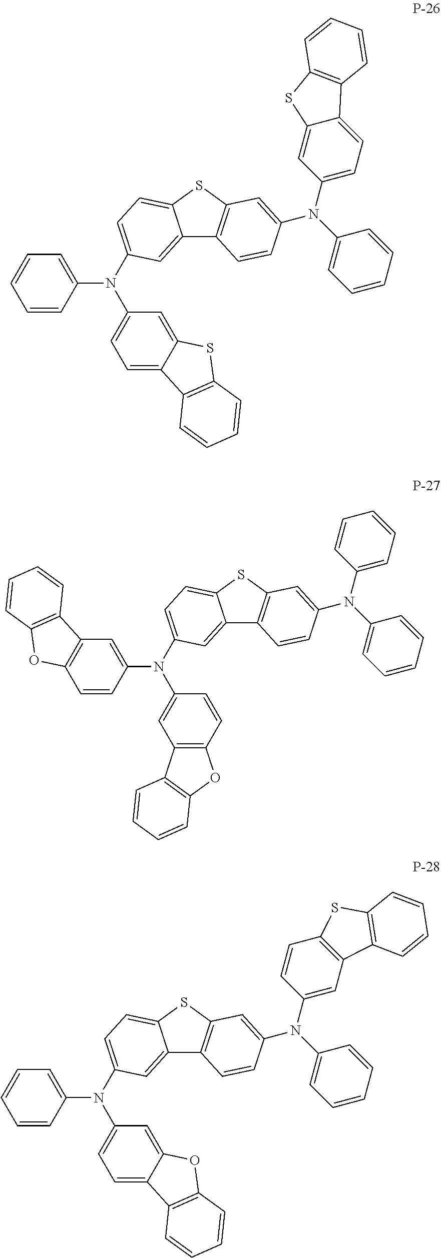 [SMILES: c1ccc(N(c2ccc3c(c2)sc2ccccc23)c2ccc3c(c2)sc2ccc(N(c4ccccc4)c4ccc5c(c4)sc4ccccc45)cc23)cc1 ; c1ccc(N(c2ccccc2)c2ccc3c(c2)sc2ccc(N(c4ccc5oc6ccccc6c5c4)c4ccc5oc6ccccc6c5c4)cc23)cc1 ; c1ccc(N(c2ccc3c(c2)sc2ccc(N(c4ccccc4)c4ccc5c(c4)oc4ccccc45)cc23)c2ccc3sc4ccccc4c3c2)cc1]

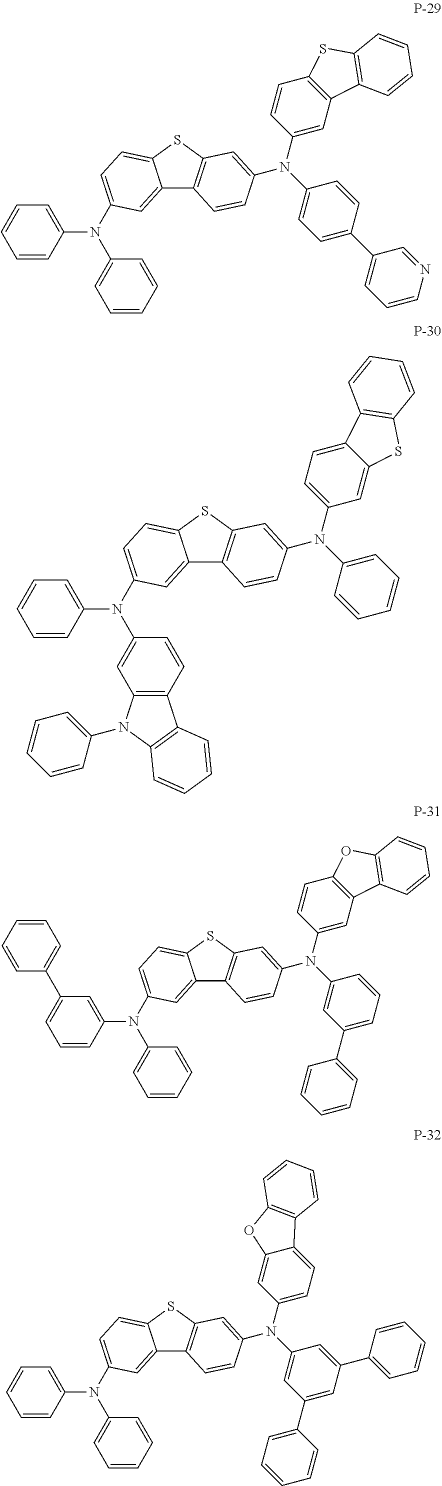 [SMILES: c1ccc(N(c2ccccc2)c2ccc3sc4cc(N(c5ccc(-c6cccnc6)cc5)c5ccc6sc7ccccc7c6c5)ccc4c3c2)cc1 ; c1ccc(N(c2ccc3c(c2)sc2ccccc23)c2ccc3c(c2)sc2ccc(N(c4ccccc4)c4ccc5c6ccccc6n(-c6ccccc6)c5c4)cc23)cc1 ; c1ccc(-c2cccc(N(c3ccc4c(c3)sc3ccc(N(c5ccccc5)c5cccc(-c6ccccc6)c5)cc34)c3ccc4oc5ccccc5c4c3)c2)cc1 ; c1ccc(-c2cc(-c3ccccc3)cc(N(c3ccc4c(c3)oc3ccccc34)c3ccc4c(c3)sc3ccc(N(c5ccccc5)c5ccccc5)cc34)c2)cc1]

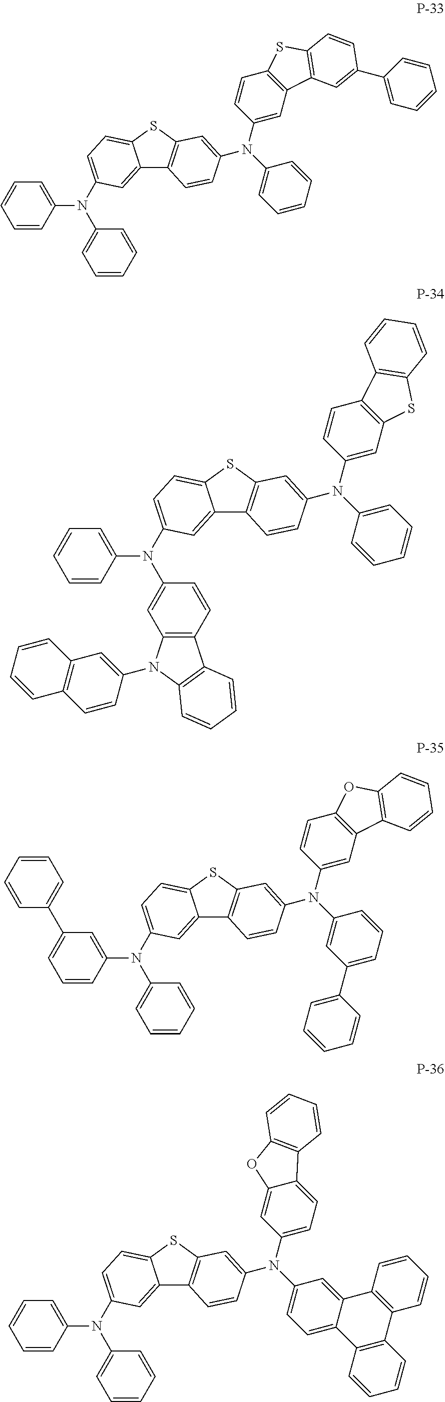 [SMILES: c1ccc(-c2ccc3sc4ccc(N(c5ccccc5)c5ccc6c(c5)sc5ccc(N(c7ccccc7)c7ccccc7)cc56)cc4c3c2)cc1 ; c1ccc(N(c2ccc3c(c2)sc2ccccc23)c2ccc3c(c2)sc2ccc(N(c4ccccc4)c4ccc5c6ccccc6n(-c6ccc7ccccc7c6)c5c4)cc23)cc1 ; c1ccc(-c2cccc(N(c3ccc4c(c3)sc3ccc(N(c5ccccc5)c5cccc(-c6ccccc6)c5)cc34)c3ccc4oc5ccccc5c4c3)c2)cc1 ; c1ccc(N(c2ccccc2)c2ccc3sc4cc(N(c5ccc6c(c5)oc5ccccc56)c5ccc6c7ccccc7c7ccccc7c6c5)ccc4c3c2)cc1]

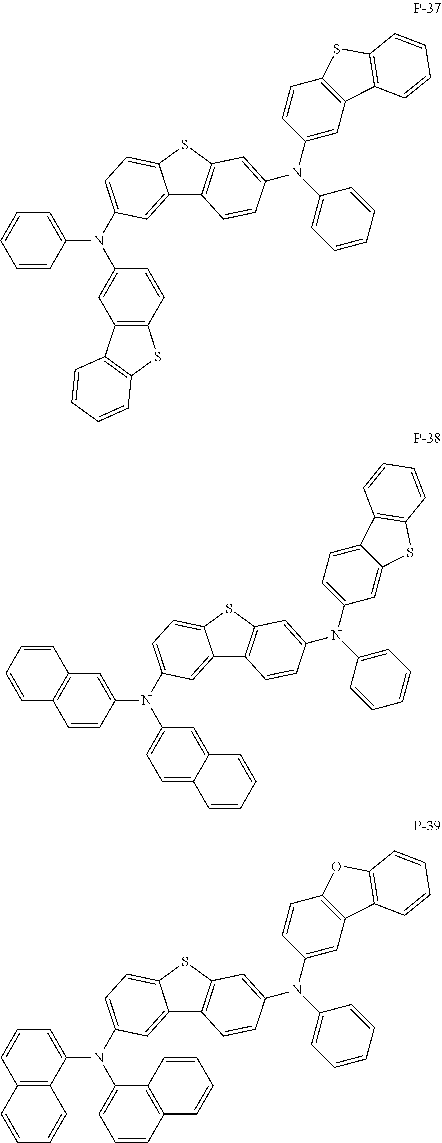 [SMILES: c1ccc(N(c2ccc3c(c2)sc2ccc(N(c4ccccc4)c4ccc5sc6ccccc6c5c4)cc23)c2ccc3sc4ccccc4c3c2)cc1 ; c1ccc(N(c2ccc3c(c2)sc2ccccc23)c2ccc3c(c2)sc2ccc(N(c4ccc5ccccc5c4)c4ccc5ccccc5c4)cc23)cc1 ; c1ccc(N(c2ccc3c(c2)sc2ccc(N(c4cccc5ccccc45)c4cccc5ccccc45)cc23)c2ccc3oc4ccccc4c3c2)cc1]

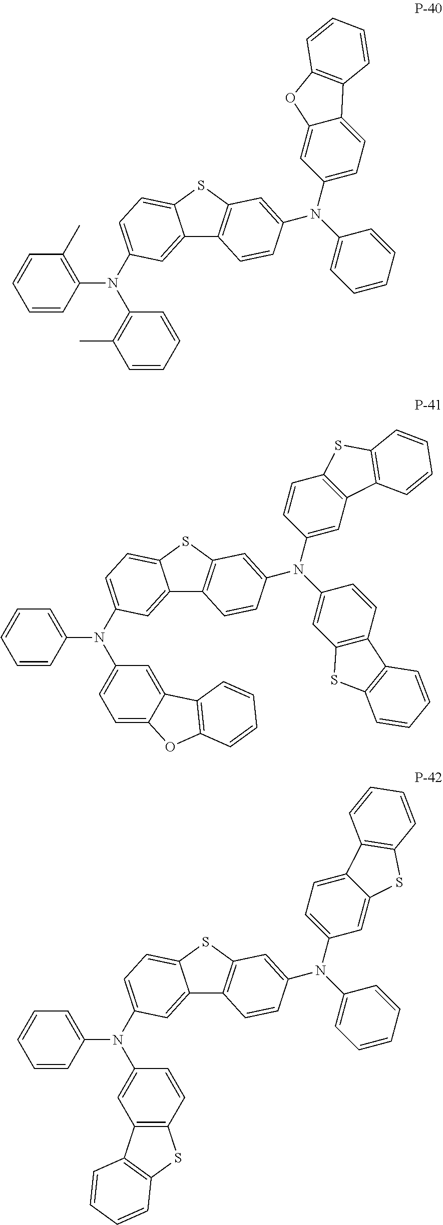 [SMILES: Cc1ccccc1N(c1ccc2sc3cc(N(c4ccccc4)c4ccc5c(c4)oc4ccccc45)ccc3c2c1)c1ccccc1C ; c1ccc(N(c2ccc3oc4ccccc4c3c2)c2ccc3sc4cc(N(c5ccc6c(c5)sc5ccccc56)c5ccc6sc7ccccc7c6c5)ccc4c3c2)cc1 ; c1ccc(N(c2ccc3c(c2)sc2ccccc23)c2ccc3c(c2)sc2ccc(N(c4ccccc4)c4ccc5sc6ccccc6c5c4)cc23)cc1]

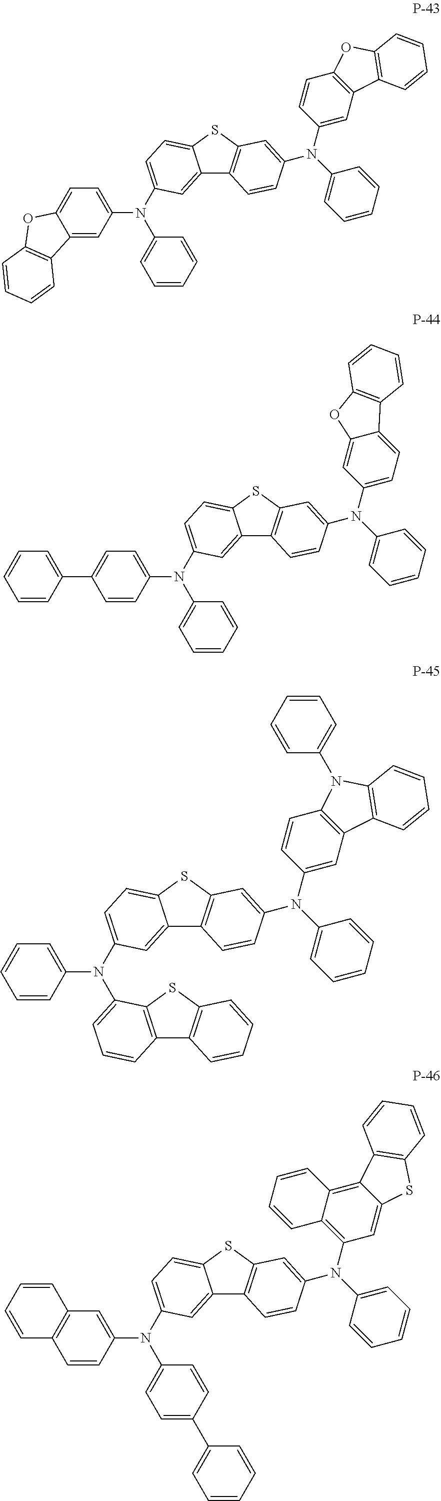 [SMILES: c1ccc(N(c2ccc3c(c2)sc2ccc(N(c4ccccc4)c4ccc5oc6ccccc6c5c4)cc23)c2ccc3oc4ccccc4c3c2)cc1 ; c1ccc(-c2ccc(N(c3ccccc3)c3ccc4sc5cc(N(c6ccccc6)c6ccc7c(c6)oc6ccccc67)ccc5c4c3)cc2)cc1 ; c1ccc(N(c2ccc3c(c2)sc2ccc(N(c4ccccc4)c4cccc5c4sc4ccccc45)cc23)c2ccc3c(c2)c2ccccc2n3-c2ccccc2)cc1 ; c1ccc(-c2ccc(N(c3ccc4ccccc4c3)c3ccc4sc5cc(N(c6ccccc6)c6cc7sc8ccccc8c7c7ccccc67)ccc5c4c3)cc2)cc1]

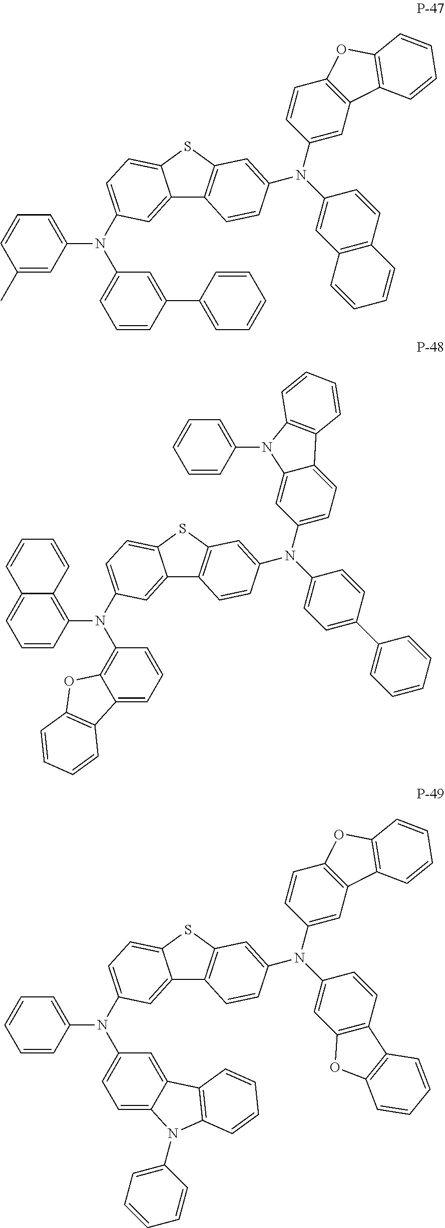 [SMILES: Cc1cccc(N(c2cccc(-c3ccccc3)c2)c2ccc3sc4cc(N(c5ccc6ccccc6c5)c5ccc6oc7ccccc7c6c5)ccc4c3c2)c1 ; c1ccc(-c2ccc(N(c3ccc4c(c3)sc3ccc(N(c5cccc6ccccc56)c5cccc6c5oc5ccccc56)cc34)c3ccc4c5ccccc5n(-c5ccccc5)c4c3)cc2)cc1 ; c1ccc(N(c2ccc3sc4cc(N(c5ccc6c(c5)oc5ccccc56)c5ccc6oc7ccccc7c6c5)ccc4c3c2)c2ccc3c(c2)c2ccccc2n3-c2ccccc2)cc1]

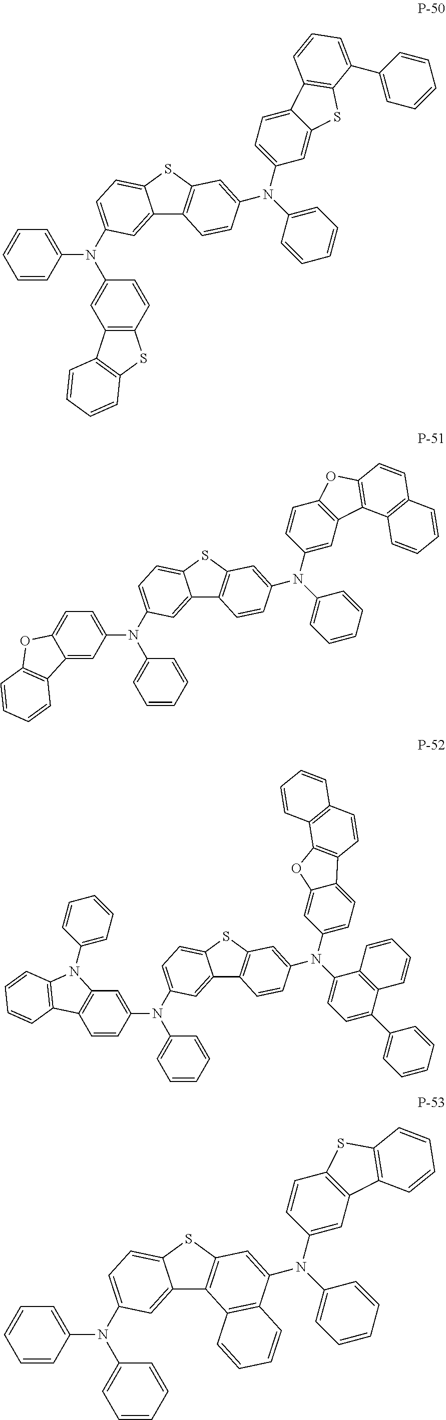 [SMILES: c1ccc(-c2cccc3c2sc2cc(N(c4ccccc4)c4ccc5c(c4)sc4ccc(N(c6ccccc6)c6ccc7sc8ccccc8c7c6)cc45)ccc23)cc1 ; c1ccc(N(c2ccc3oc4ccccc4c3c2)c2ccc3sc4cc(N(c5ccccc5)c5ccc6oc7ccc8ccccc8c7c6c5)ccc4c3c2)cc1 ; c1ccc(-c2ccc(N(c3ccc4c(c3)oc3c5ccccc5ccc43)c3ccc4c(c3)sc3ccc(N(c5ccccc5)c5ccc6c7ccccc7n(-c7ccccc7)c6c5)cc34)c3ccccc23)cc1 ; c1ccc(N(c2ccccc2)c2ccc3sc4cc(N(c5ccccc5)c5ccc6sc7ccccc7c6c5)c5ccccc5c4c3c2)cc1]

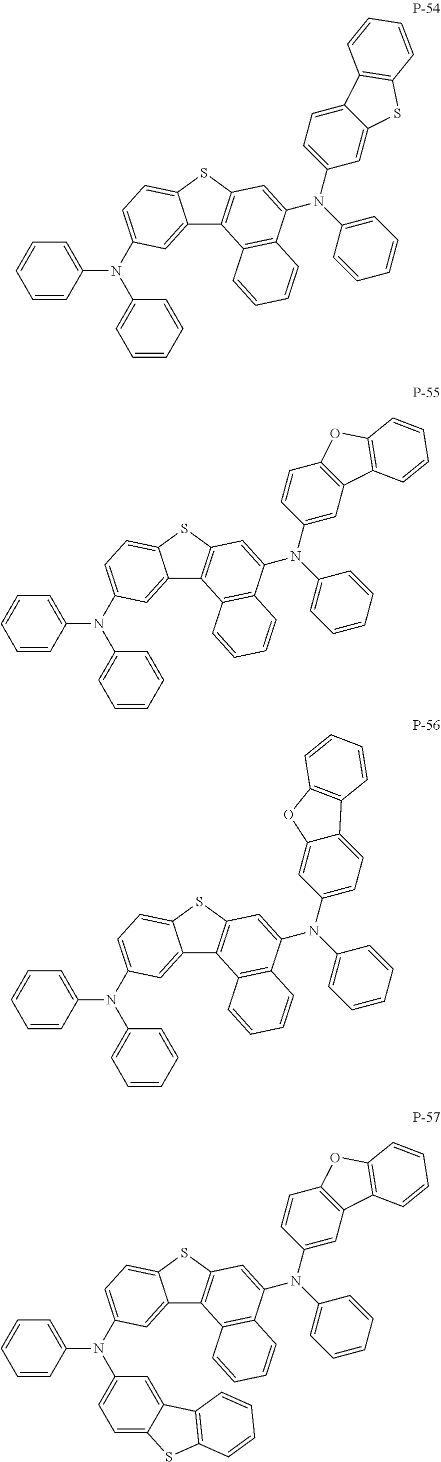 [SMILES: c1ccc(N(c2ccccc2)c2ccc3sc4cc(N(c5ccccc5)c5ccc6c(c5)sc5ccccc56)c5ccccc5c4c3c2)cc1 ; c1ccc(N(c2ccccc2)c2ccc3sc4cc(N(c5ccccc5)c5ccc6oc7ccccc7c6c5)c5ccccc5c4c3c2)cc1 ; c1ccc(N(c2ccccc2)c2ccc3sc4cc(N(c5ccccc5)c5ccc6c(c5)oc5ccccc56)c5ccccc5c4c3c2)cc1 ; c1ccc(N(c2ccc3sc4ccccc4c3c2)c2ccc3sc4cc(N(c5ccccc5)c5ccc6oc7ccccc7c6c5)c5ccccc5c4c3c2)cc1]

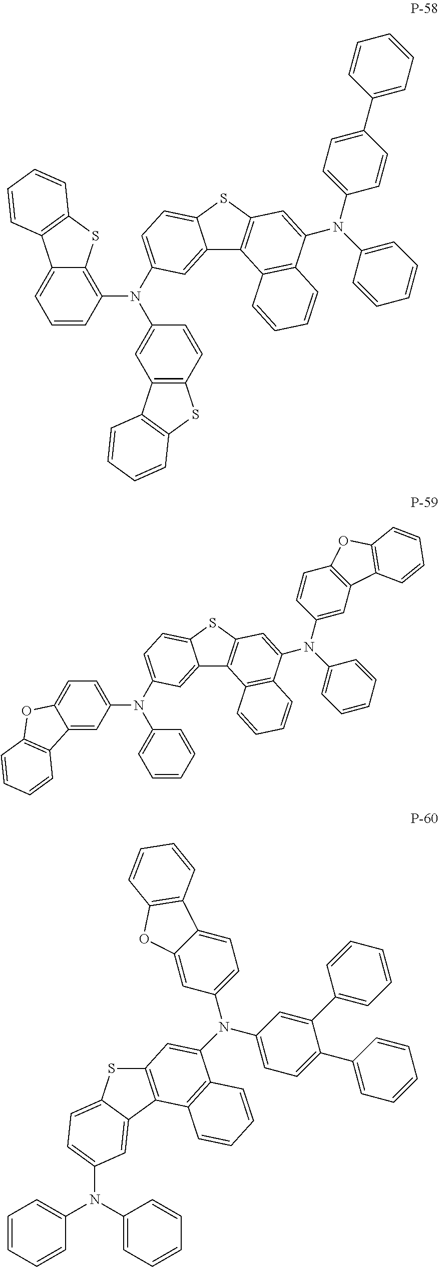 [SMILES: c1ccc(-c2ccc(N(c3ccccc3)c3cc4sc5ccc(N(c6ccc7sc8ccccc8c7c6)c6cccc7c6sc6ccccc67)cc5c4c4ccccc34)cc2)cc1 ; c1ccc(N(c2ccc3oc4ccccc4c3c2)c2ccc3sc4cc(N(c5ccccc5)c5ccc6oc7ccccc7c6c5)c5ccccc5c4c3c2)cc1 ; c1ccc(-c2ccc(N(c3ccc4c(c3)oc3ccccc34)c3cc4sc5ccc(N(c6ccccc6)c6ccccc6)cc5c4c4ccccc34)cc2-c2ccccc2)cc1]

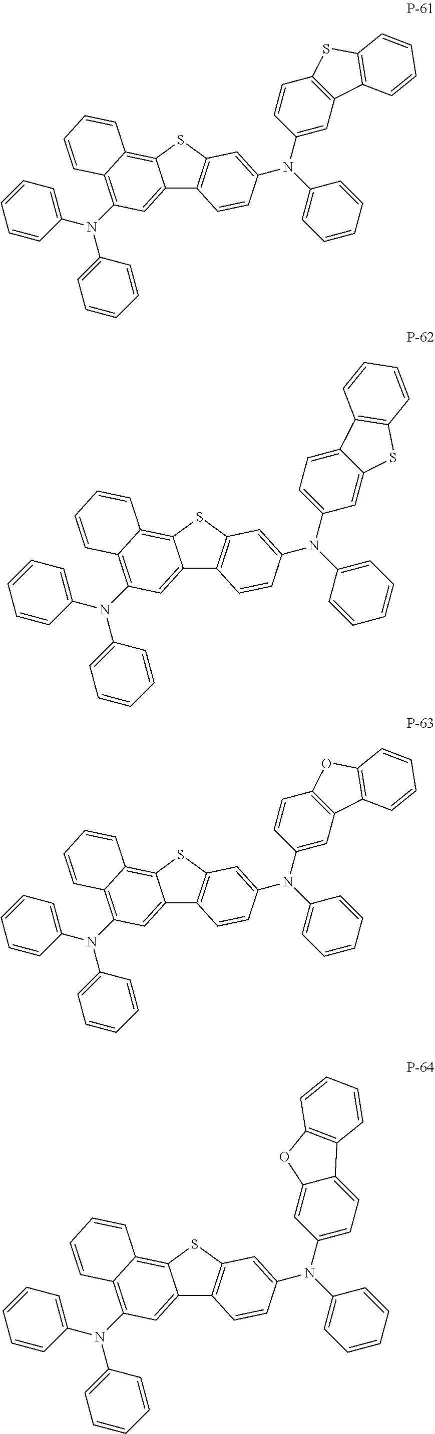 [SMILES: c1ccc(N(c2ccc3c(c2)sc2c4ccccc4c(N(c4ccccc4)c4ccccc4)cc32)c2ccc3sc4ccccc4c3c2)cc1 ; c1ccc(N(c2ccc3c(c2)sc2ccccc23)c2ccc3c(c2)sc2c4ccccc4c(N(c4ccccc4)c4ccccc4)cc32)cc1 ; c1ccc(N(c2ccc3c(c2)sc2c4ccccc4c(N(c4ccccc4)c4ccccc4)cc32)c2ccc3oc4ccccc4c3c2)cc1 ; c1ccc(N(c2ccc3c(c2)oc2ccccc23)c2ccc3c(c2)sc2c4ccccc4c(N(c4ccccc4)c4ccccc4)cc32)cc1]

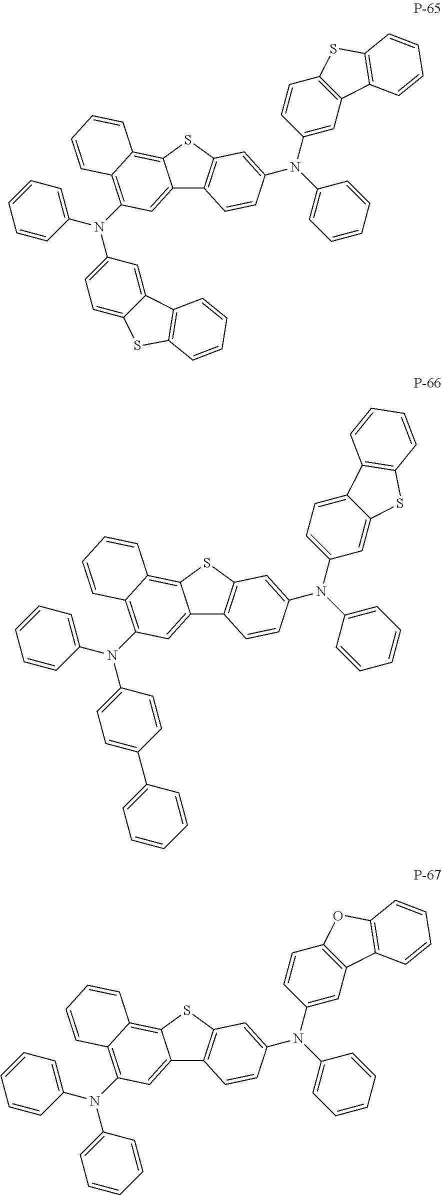 [SMILES: c1ccc(N(c2ccc3c(c2)sc2c4ccccc4c(N(c4ccccc4)c4ccc5sc6ccccc6c5c4)cc32)c2ccc3sc4ccccc4c3c2)cc1 ; c1ccc(-c2ccc(N(c3ccccc3)c3cc4c5ccc(N(c6ccccc6)c6ccc7c(c6)sc6ccccc67)cc5sc4c4ccccc34)cc2)cc1 ; c1ccc(N(c2ccc3c(c2)sc2c4ccccc4c(N(c4ccccc4)c4ccccc4)cc32)c2ccc3oc4ccccc4c3c2)cc1]

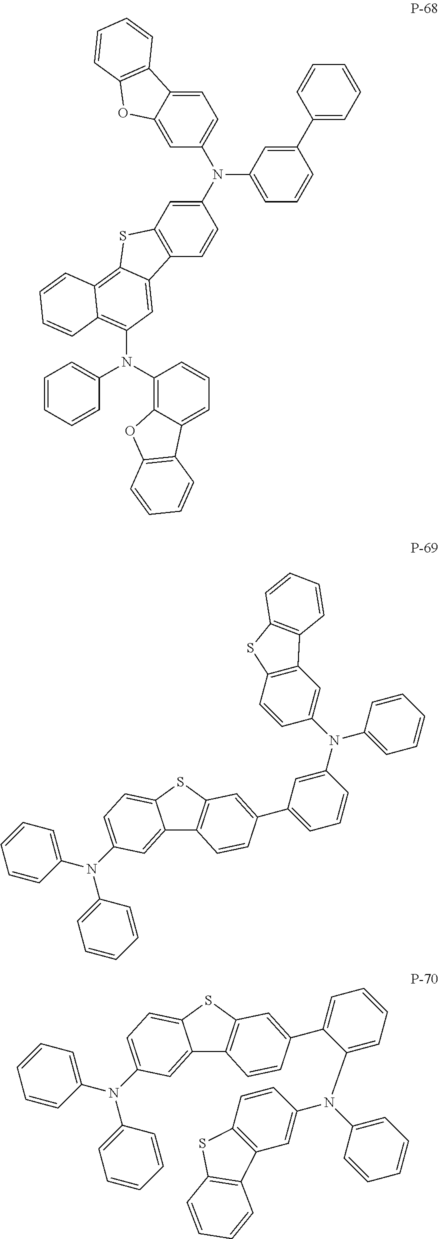 [SMILES: c1ccc(-c2cccc(N(c3ccc4c(c3)oc3ccccc34)c3ccc4c(c3)sc3c5ccccc5c(N(c5ccccc5)c5cccc6c5oc5ccccc56)cc43)c2)cc1 ; c1ccc(N(c2cccc(-c3ccc4c(c3)sc3ccc(N(c5ccccc5)c5ccccc5)cc34)c2)c2ccc3sc4ccccc4c3c2)cc1 ; c1ccc(N(c2ccccc2)c2ccc3sc4cc(-c5ccccc5N(c5ccccc5)c5ccc6sc7ccccc7c6c5)ccc4c3c2)cc1]

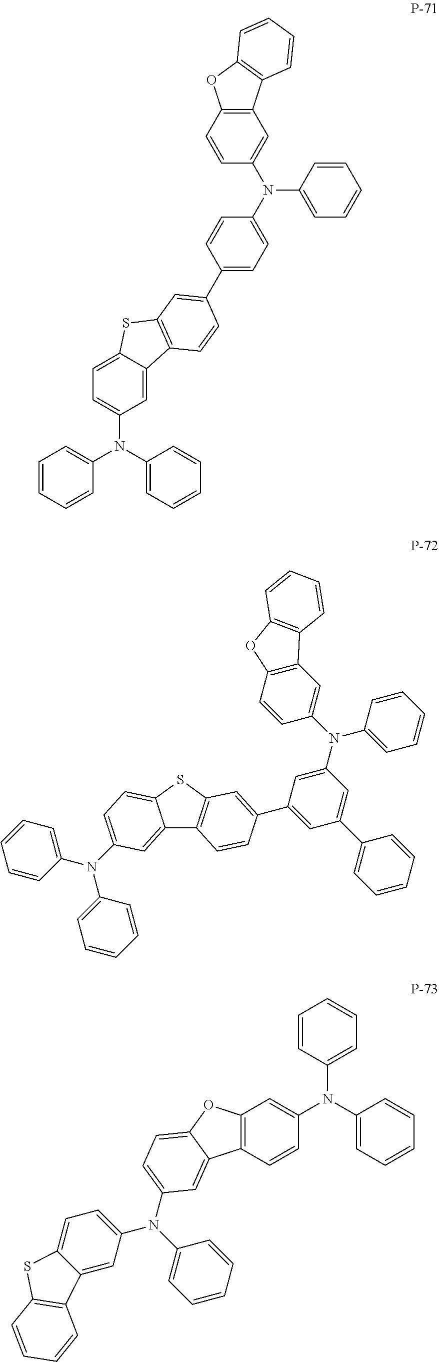 [SMILES: c1ccc(N(c2ccc(-c3ccc4c(c3)sc3ccc(N(c5ccccc5)c5ccccc5)cc34)cc2)c2ccc3oc4ccccc4c3c2)cc1 ; c1ccc(-c2cc(-c3ccc4c(c3)sc3ccc(N(c5ccccc5)c5ccccc5)cc34)cc(N(c3ccccc3)c3ccc4oc5ccccc5c4c3)c2)cc1 ; c1ccc(N(c2ccccc2)c2ccc3c(c2)oc2ccc(N(c4ccccc4)c4ccc5sc6ccccc6c5c4)cc23)cc1]

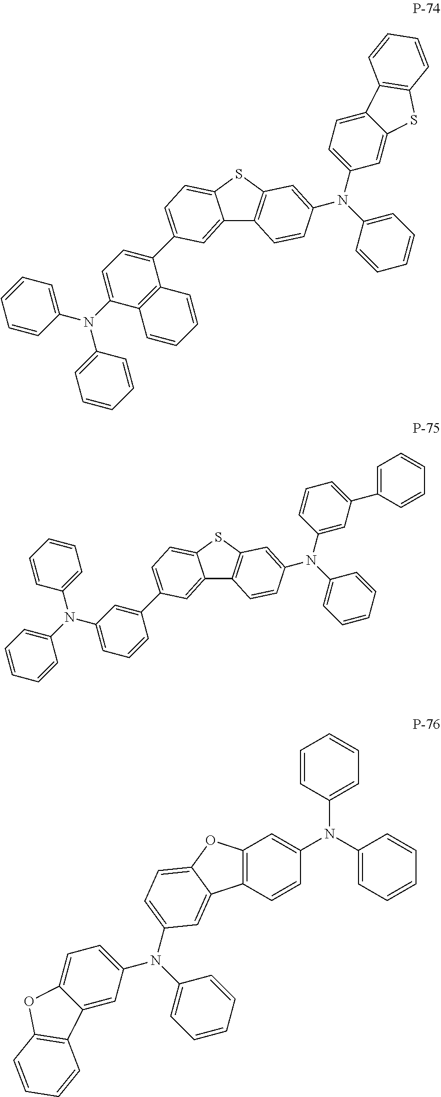 [SMILES: c1ccc(N(c2ccc3c(c2)sc2ccccc23)c2ccc3c(c2)sc2ccc(-c4ccc(N(c5ccccc5)c5ccccc5)c5ccccc45)cc23)cc1 ; c1ccc(-c2cccc(N(c3ccccc3)c3ccc4c(c3)sc3ccc(-c5cccc(N(c6ccccc6)c6ccccc6)c5)cc34)c2)cc1 ; c1ccc(N(c2ccccc2)c2ccc3c(c2)oc2ccc(N(c4ccccc4)c4ccc5oc6ccccc6c5c4)cc23)cc1]

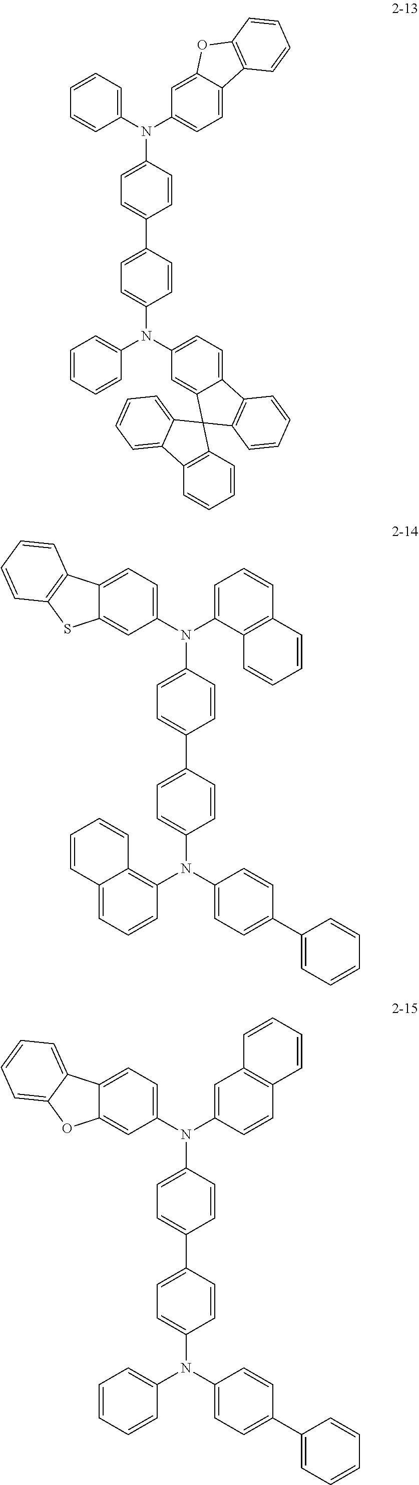 [SMILES: c1ccc(N(c2ccc(-c3ccc(N(c4ccccc4)c4ccc5c(c4)oc4ccccc45)cc3)cc2)c2ccc3c(c2)C2(c4ccccc4-c4ccccc42)c2ccccc2-3)cc1 ; c1ccc(-c2ccc(N(c3ccc(-c4ccc(N(c5ccc6c(c5)sc5ccccc56)c5cccc6ccccc56)cc4)cc3)c3cccc4ccccc34)cc2)cc1 ; c1ccc(-c2ccc(N(c3ccccc3)c3ccc(-c4ccc(N(c5ccc6ccccc6c5)c5ccc6c(c5)oc5ccccc56)cc4)cc3)cc2)cc1]

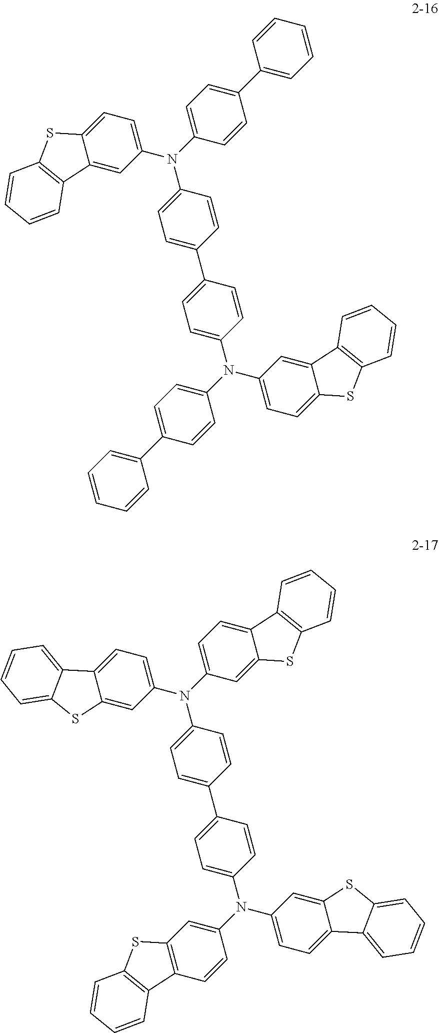 [SMILES: c1ccc(-c2ccc(N(c3ccc(-c4ccc(N(c5ccc(-c6ccccc6)cc5)c5ccc6sc7ccccc7c6c5)cc4)cc3)c3ccc4sc5ccccc5c4c3)cc2)cc1 ; c1ccc2c(c1)sc1cc(N(c3ccc(-c4ccc(N(c5ccc6c(c5)sc5ccccc56)c5ccc6c(c5)sc5ccccc56)cc4)cc3)c3ccc4c(c3)sc3ccccc34)ccc12]

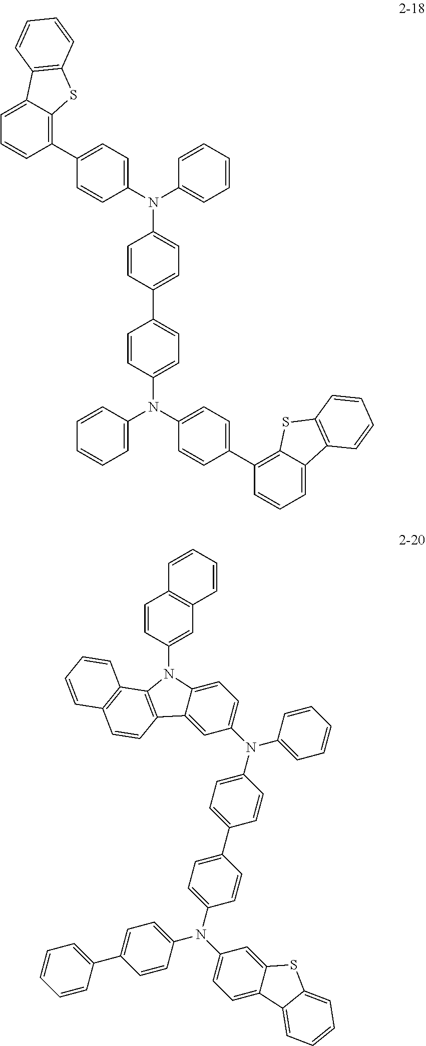 [SMILES: c1ccc(N(c2ccc(-c3ccc(N(c4ccccc4)c4ccc(-c5cccc6c5sc5ccccc56)cc4)cc3)cc2)c2ccc(-c3cccc4c3sc3ccccc34)cc2)cc1 ; c1ccc(-c2ccc(N(c3ccc(-c4ccc(N(c5ccccc5)c5ccc6c(c5)c5ccc7ccccc7c5n6-c5ccc6ccccc6c5)cc4)cc3)c3ccc4c(c3)sc3ccccc34)cc2)cc1]

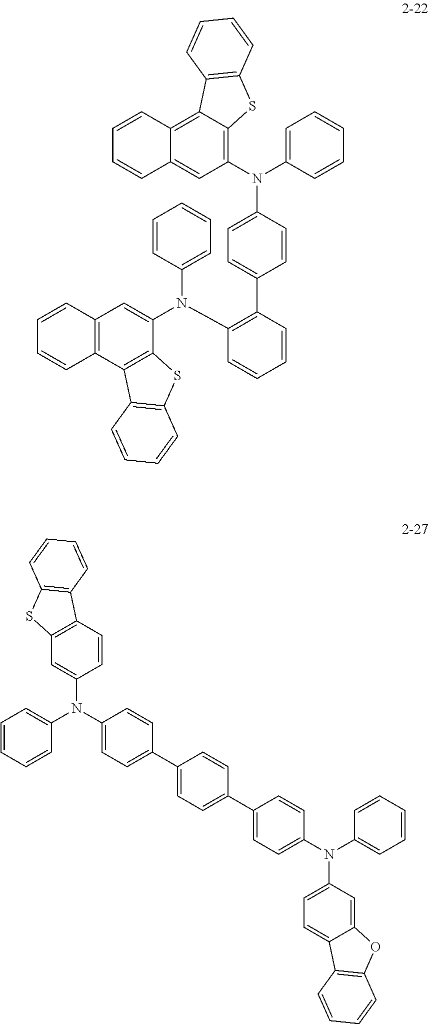 [SMILES: c1ccc(N(c2ccc(-c3ccccc3N(c3ccccc3)c3cc4ccccc4c4c3sc3ccccc34)cc2)c2cc3ccccc3c3c2sc2ccccc23)cc1 ; c1ccc(N(c2ccc(-c3ccc(-c4ccc(N(c5ccccc5)c5ccc6c(c5)sc5ccccc56)cc4)cc3)cc2)c2ccc3c(c2)oc2ccccc23)cc1]

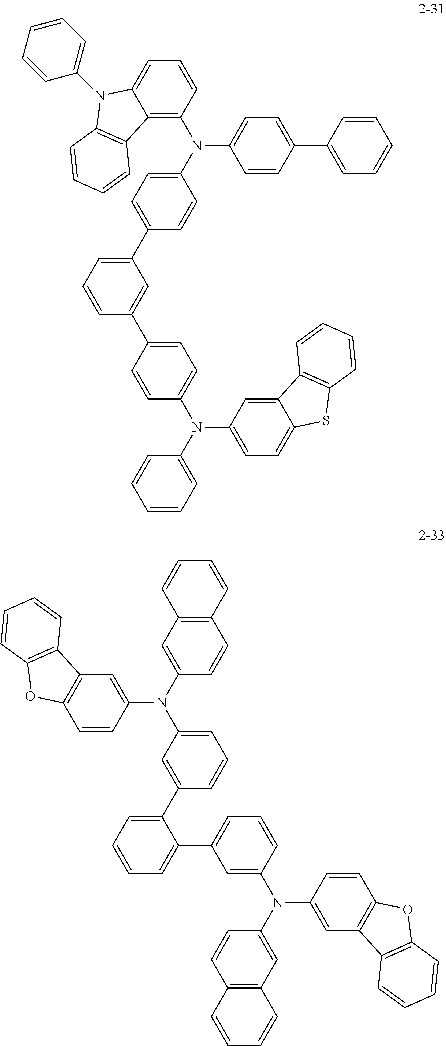 [SMILES: c1ccc(-c2ccc(N(c3ccc(-c4cccc(-c5ccc(N(c6ccccc6)c6ccc7sc8ccccc8c7c6)cc5)c4)cc3)c3cccc4c3c3ccccc3n4-c3ccccc3)cc2)cc1 ; c1cc(-c2ccccc2-c2cccc(N(c3ccc4ccccc4c3)c3ccc4oc5ccccc5c4c3)c2)cc(N(c2ccc3ccccc3c2)c2ccc3oc4ccccc4c3c2)c1]

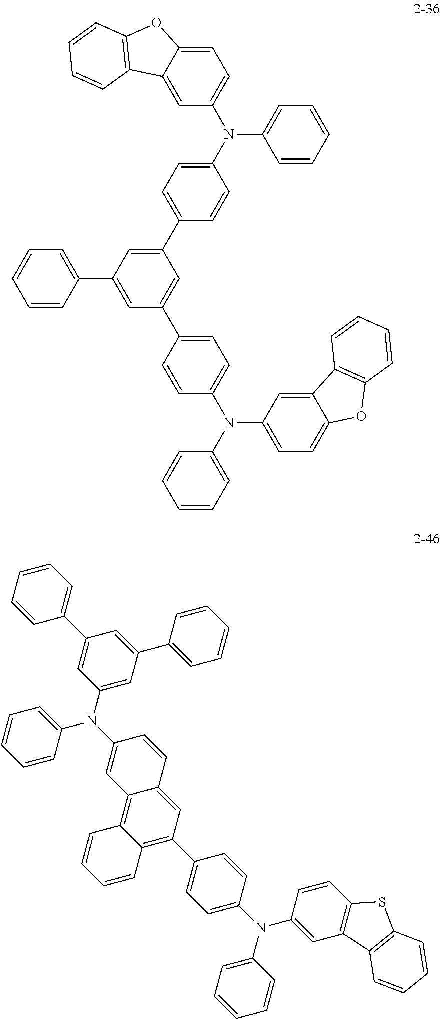 [SMILES: c1ccc(-c2cc(-c3ccc(N(c4ccccc4)c4ccc5oc6ccccc6c5c4)cc3)cc(-c3ccc(N(c4ccccc4)c4ccc5oc6ccccc6c5c4)cc3)c2)cc1 ; c1ccc(-c2cc(-c3ccccc3)cc(N(c3ccccc3)c3ccc4cc(-c5ccc(N(c6ccccc6)c6ccc7sc8ccccc8c7c6)cc5)c5ccccc5c4c3)c2)cc1]

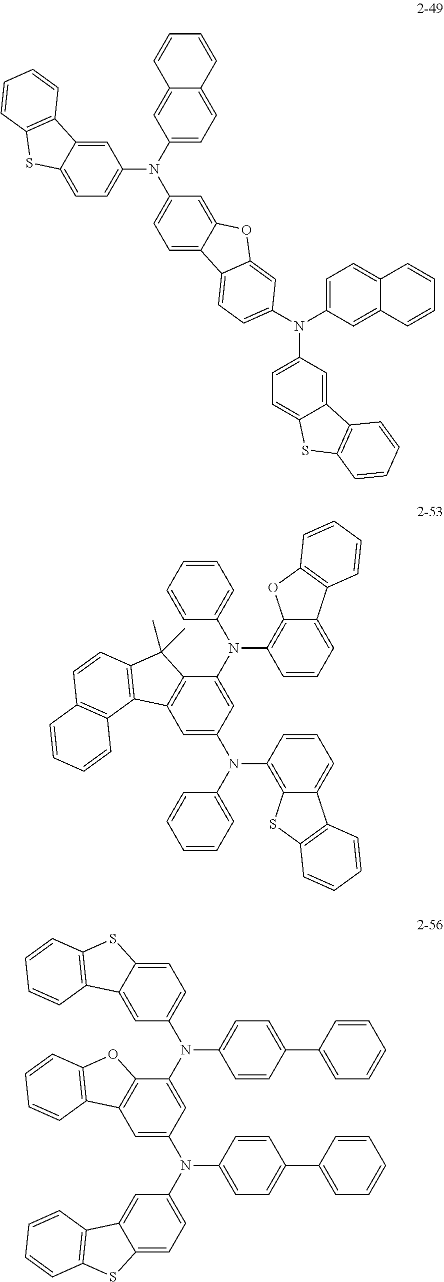 [SMILES: c1ccc2cc(N(c3ccc4c(c3)oc3cc(N(c5ccc6ccccc6c5)c5ccc6sc7ccccc7c6c5)ccc34)c3ccc4sc5ccccc5c4c3)ccc2c1 ; CC1(C)c2ccc3ccccc3c2-c2cc(N(c3ccccc3)c3cccc4c3sc3ccccc34)cc(N(c3ccccc3)c3cccc4c3oc3ccccc34)c21 ; c1ccc(-c2ccc(N(c3ccc4sc5ccccc5c4c3)c3cc(N(c4ccc(-c5ccccc5)cc4)c4ccc5sc6ccccc6c5c4)c4oc5ccccc5c4c3)cc2)cc1]

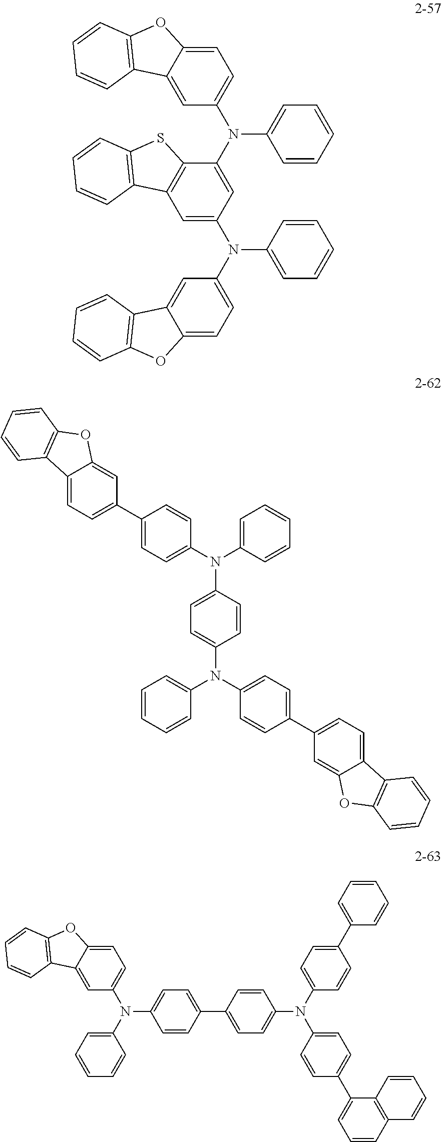 [SMILES: c1ccc(N(c2ccc3oc4ccccc4c3c2)c2cc(N(c3ccccc3)c3ccc4oc5ccccc5c4c3)c3sc4ccccc4c3c2)cc1 ; c1ccc(N(c2ccc(-c3ccc4c(c3)oc3ccccc34)cc2)c2ccc(N(c3ccccc3)c3ccc(-c4ccc5c(c4)oc4ccccc45)cc3)cc2)cc1 ; c1ccc(-c2ccc(N(c3ccc(-c4ccc(N(c5ccccc5)c5ccc6oc7ccccc7c6c5)cc4)cc3)c3ccc(-c4cccc5ccccc45)cc3)cc2)cc1]

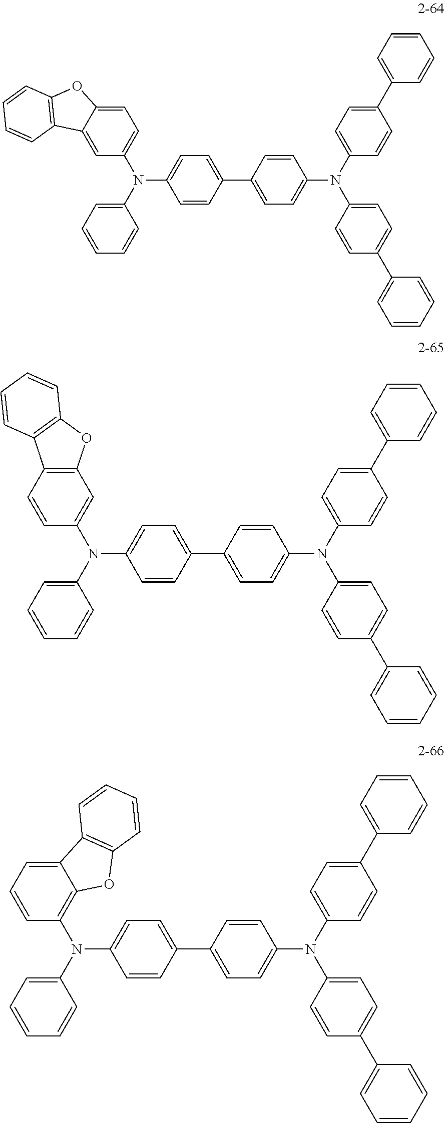 [SMILES: c1ccc(-c2ccc(N(c3ccc(-c4ccccc4)cc3)c3ccc(-c4ccc(N(c5ccccc5)c5ccc6oc7ccccc7c6c5)cc4)cc3)cc2)cc1 ; c1ccc(-c2ccc(N(c3ccc(-c4ccccc4)cc3)c3ccc(-c4ccc(N(c5ccccc5)c5ccc6c(c5)oc5ccccc56)cc4)cc3)cc2)cc1 ; c1ccc(-c2ccc(N(c3ccc(-c4ccccc4)cc3)c3ccc(-c4ccc(N(c5ccccc5)c5cccc6c5oc5ccccc56)cc4)cc3)cc2)cc1]

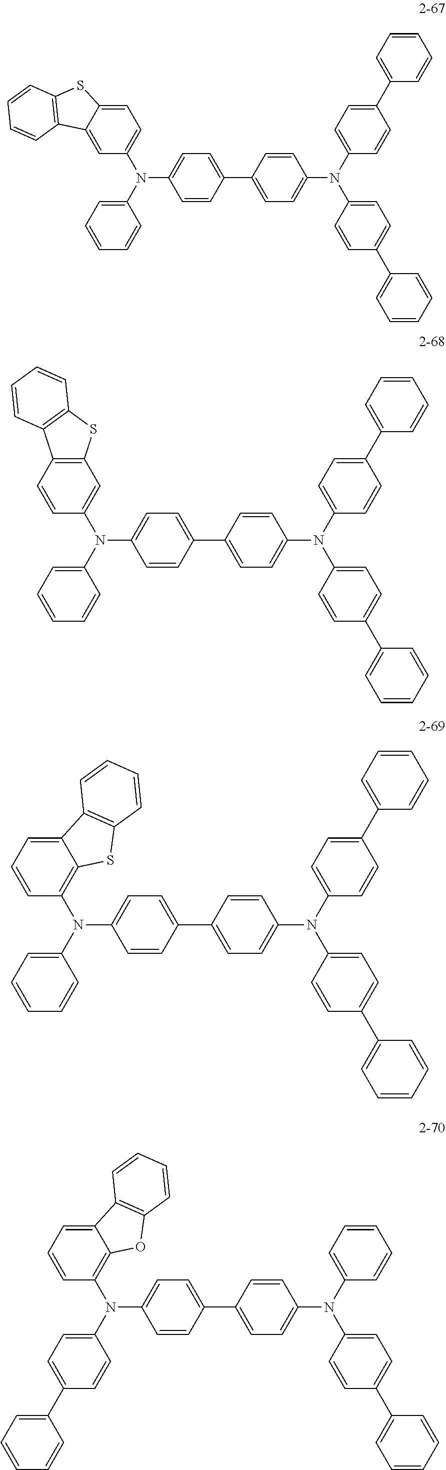 [SMILES: c1ccc(-c2ccc(N(c3ccc(-c4ccccc4)cc3)c3ccc(-c4ccc(N(c5ccccc5)c5ccc6sc7ccccc7c6c5)cc4)cc3)cc2)cc1 ; c1ccc(-c2ccc(N(c3ccc(-c4ccccc4)cc3)c3ccc(-c4ccc(N(c5ccccc5)c5ccc6c(c5)sc5ccccc56)cc4)cc3)cc2)cc1 ; c1ccc(-c2ccc(N(c3ccc(-c4ccccc4)cc3)c3ccc(-c4ccc(N(c5ccccc5)c5cccc6c5sc5ccccc56)cc4)cc3)cc2)cc1 ; c1ccc(-c2ccc(N(c3ccccc3)c3ccc(-c4ccc(N(c5ccc(-c6ccccc6)cc5)c5cccc6c5oc5ccccc56)cc4)cc3)cc2)cc1]

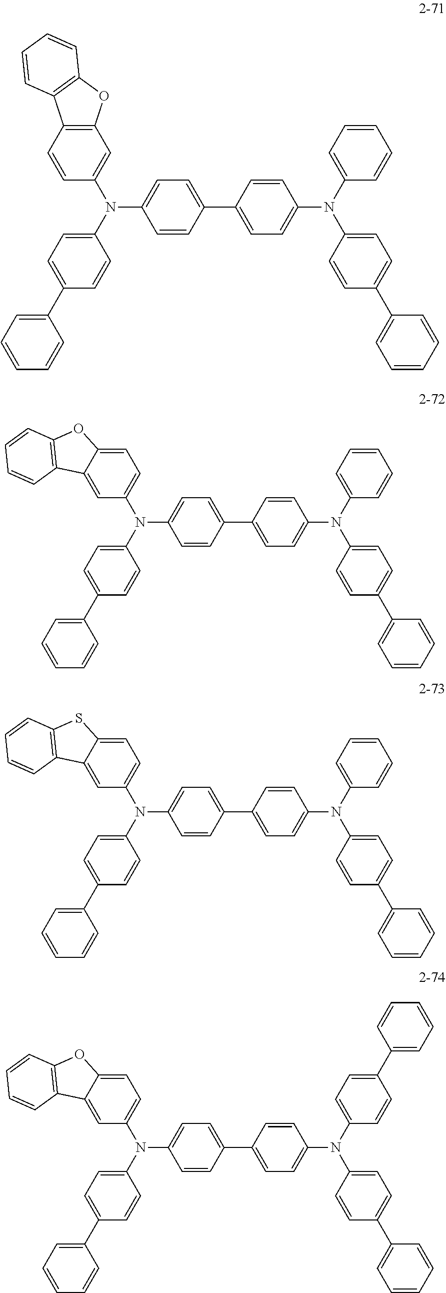 [SMILES: c1ccc(-c2ccc(N(c3ccccc3)c3ccc(-c4ccc(N(c5ccc(-c6ccccc6)cc5)c5ccc6c(c5)oc5ccccc56)cc4)cc3)cc2)cc1 ; c1ccc(-c2ccc(N(c3ccccc3)c3ccc(-c4ccc(N(c5ccc(-c6ccccc6)cc5)c5ccc6oc7ccccc7c6c5)cc4)cc3)cc2)cc1 ; c1ccc(-c2ccc(N(c3ccccc3)c3ccc(-c4ccc(N(c5ccc(-c6ccccc6)cc5)c5ccc6sc7ccccc7c6c5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc6oc7ccccc7c6c5)cc4)cc3)cc2)cc1]

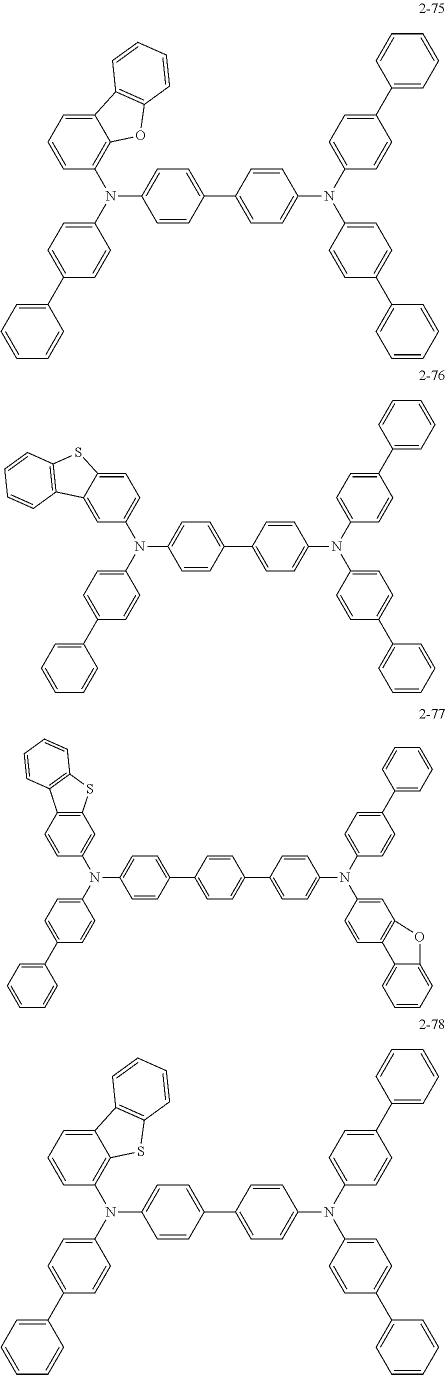 [SMILES: c1ccc(-c2ccc(N(c3ccc(-c4ccccc4)cc3)c3ccc(-c4ccc(N(c5ccc(-c6ccccc6)cc5)c5cccc6c5oc5ccccc56)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc6sc7ccccc7c6c5)cc4)cc3)cc2)cc1 ; c1ccc(-c2ccc(N(c3ccc(-c4ccc(-c5ccc(N(c6ccc(-c7ccccc7)cc6)c6ccc7c(c6)sc6ccccc67)cc5)cc4)cc3)c3ccc4c(c3)oc3ccccc34)cc2)cc1 ; c1ccc(-c2ccc(N(c3ccc(-c4ccccc4)cc3)c3ccc(-c4ccc(N(c5ccc(-c6ccccc6)cc5)c5cccc6c5sc5ccccc56)cc4)cc3)cc2)cc1]

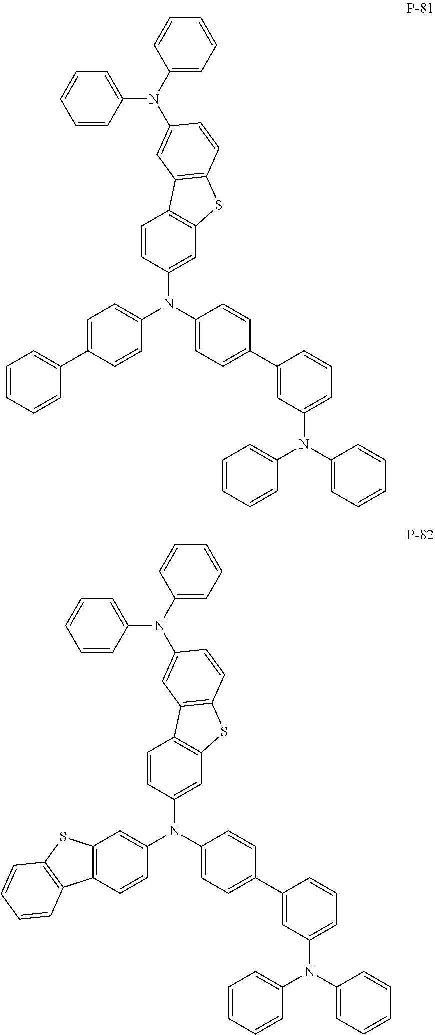 [SMILES: c1ccc(-c2ccc(N(c3ccc(-c4cccc(N(c5ccccc5)c5ccccc5)c4)cc3)c3ccc4c(c3)sc3ccc(N(c5ccccc5)c5ccccc5)cc34)cc2)cc1 ; c1ccc(N(c2ccccc2)c2cccc(-c3ccc(N(c4ccc5c(c4)sc4ccccc45)c4ccc5c(c4)sc4ccc(N(c6ccccc6)c6ccccc6)cc45)cc3)c2)cc1]

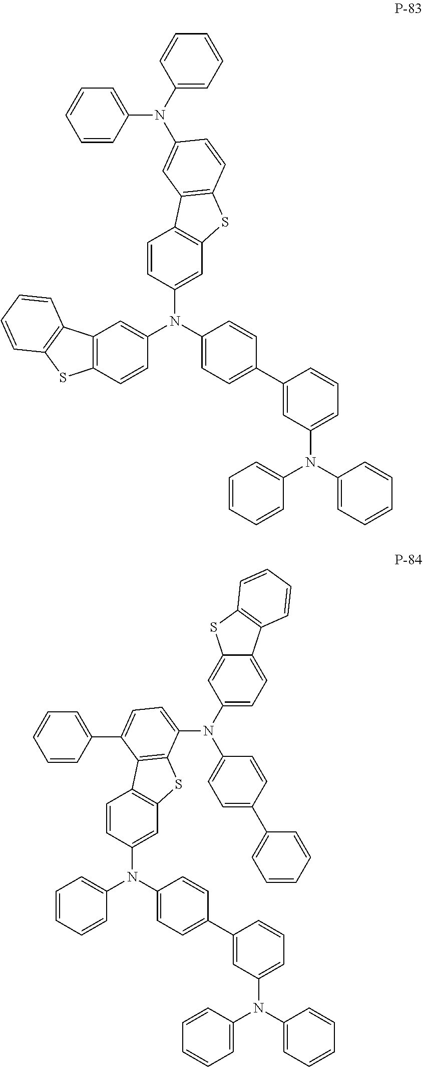 [SMILES: c1ccc(N(c2ccccc2)c2cccc(-c3ccc(N(c4ccc5c(c4)sc4ccc(N(c6ccccc6)c6ccccc6)cc45)c4ccc5sc6ccccc6c5c4)cc3)c2)cc1 ; c1ccc(-c2ccc(N(c3ccc4c(c3)sc3ccccc34)c3ccc(-c4ccccc4)c4c3sc3cc(N(c5ccccc5)c5ccc(-c6cccc(N(c7ccccc7)c7ccccc7)c6)cc5)ccc34)cc2)cc1]

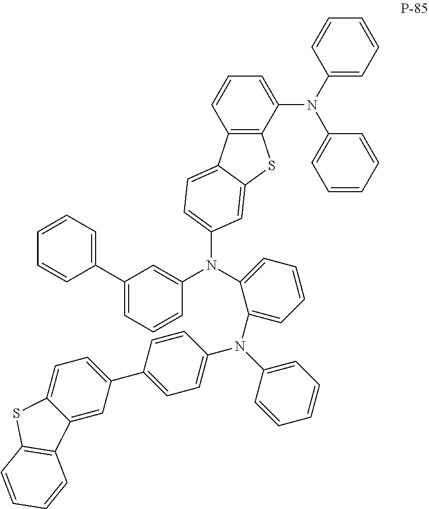 [SMILES: c1ccc(-c2cccc(N(c3ccc4c(c3)sc3c(N(c5ccccc5)c5ccccc5)cccc34)c3ccccc3N(c3ccccc3)c3ccc(-c4ccc5sc6ccccc6c5c4)cc3)c2)cc1]

Also, specifically, the compound represented by formula 5 may be one of the following compounds.

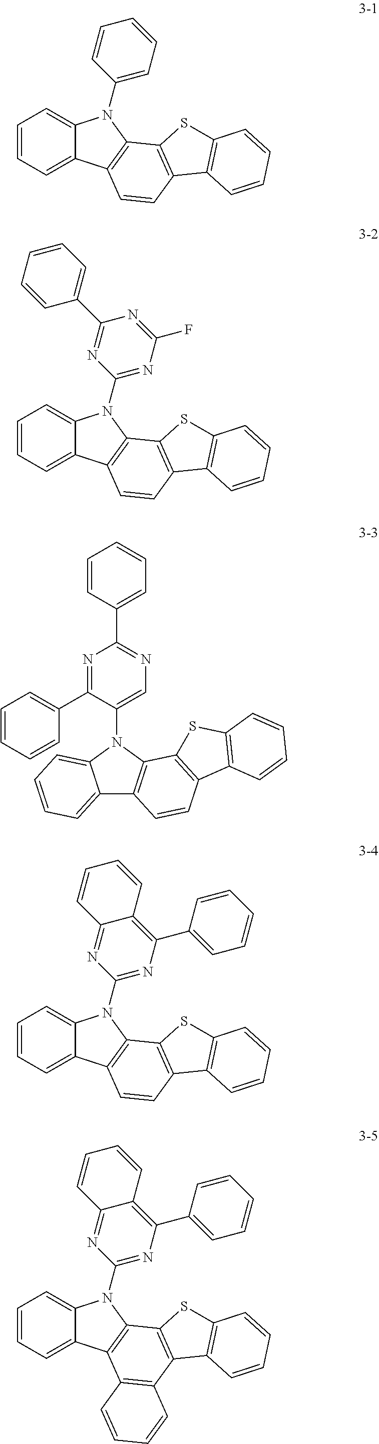 [SMILES: c1ccc(-n2c3ccccc3c3ccc4c5ccccc5sc4c32)cc1 ; Fc1nc(-c2ccccc2)nc(-n2c3ccccc3c3ccc4c5ccccc5sc4c32)n1 ; c1ccc(-c2ncc(-n3c4ccccc4c4ccc5c6ccccc6sc5c43)c(-c3ccccc3)n2)cc1 ; c1ccc(-c2nc(-n3c4ccccc4c4ccc5c6ccccc6sc5c43)nc3ccccc23)cc1 ; c1ccc(-c2nc(-n3c4ccccc4c4c5ccccc5c5c6ccccc6sc5c43)nc3ccccc23)cc1]

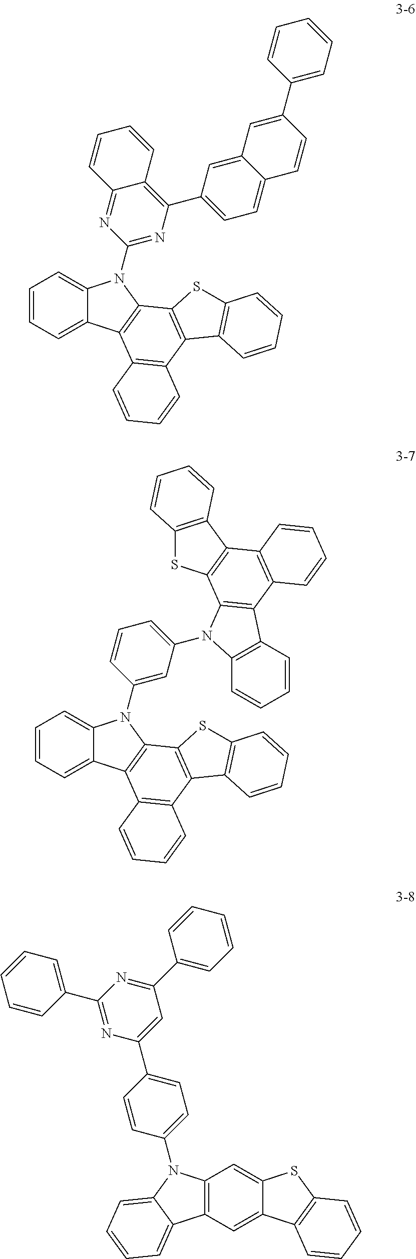 [SMILES: c1ccc(-c2ccc3ccc(-c4nc(-n5c6ccccc6c6c7ccccc7c7c8ccccc8sc7c65)nc5ccccc45)cc3c2)cc1 ; c1cc(-n2c3ccccc3c3c4ccccc4c4c5ccccc5sc4c32)cc(-n2c3ccccc3c3c4ccccc4c4c5ccccc5sc4c32)c1 ; c1ccc(-c2cc(-c3ccc(-n4c5ccccc5c5cc6c(cc54)sc4ccccc46)cc3)nc(-c3ccccc3)n2)cc1]

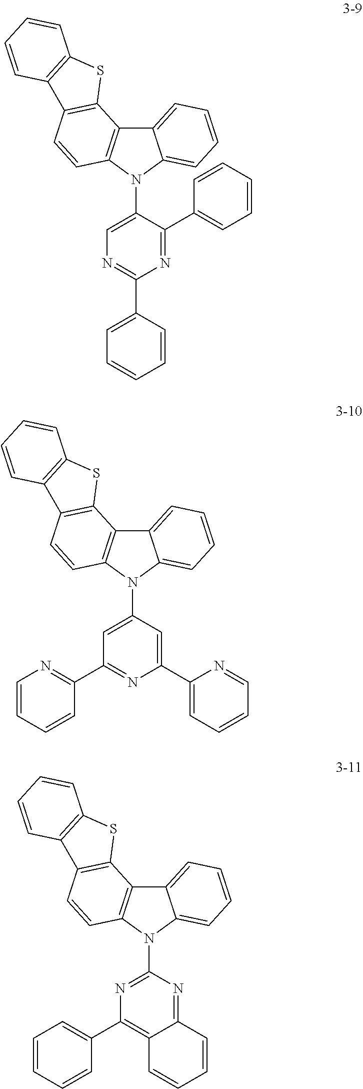

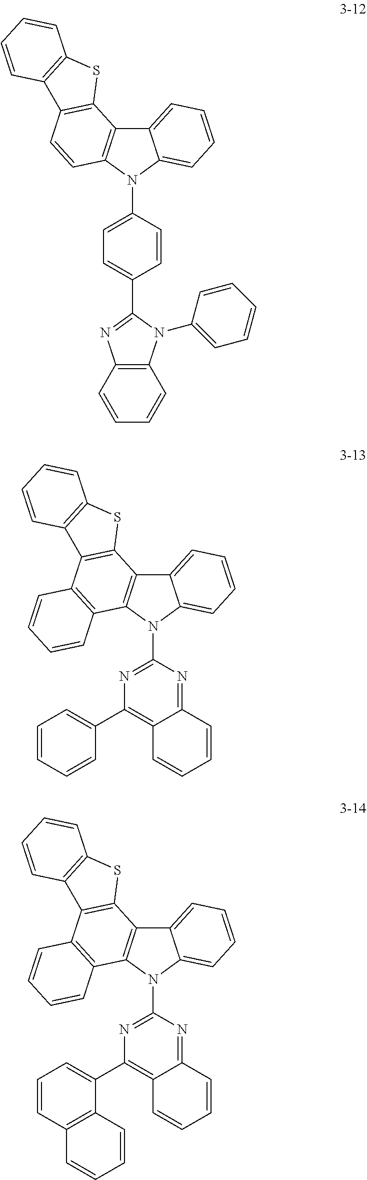 [SMILES: c1ccc(-n2c(-c3ccc(-n4c5ccccc5c5c6sc7ccccc7c6ccc54)cc3)nc3ccccc32)cc1 ; c1ccc(-c2nc(-n3c4ccccc4c4c5sc6ccccc6c5c5ccccc5c43)nc3ccccc23)cc1 ; c1ccc2c(-c3nc(-n4c5ccccc5c5c6sc7ccccc7c6c6ccccc6c54)nc4ccccc34)cccc2c1]

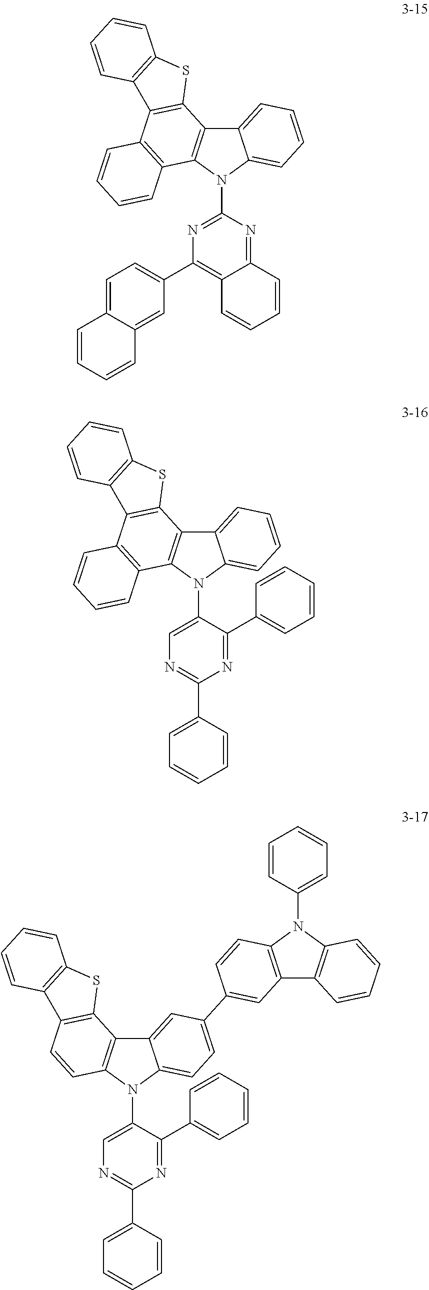

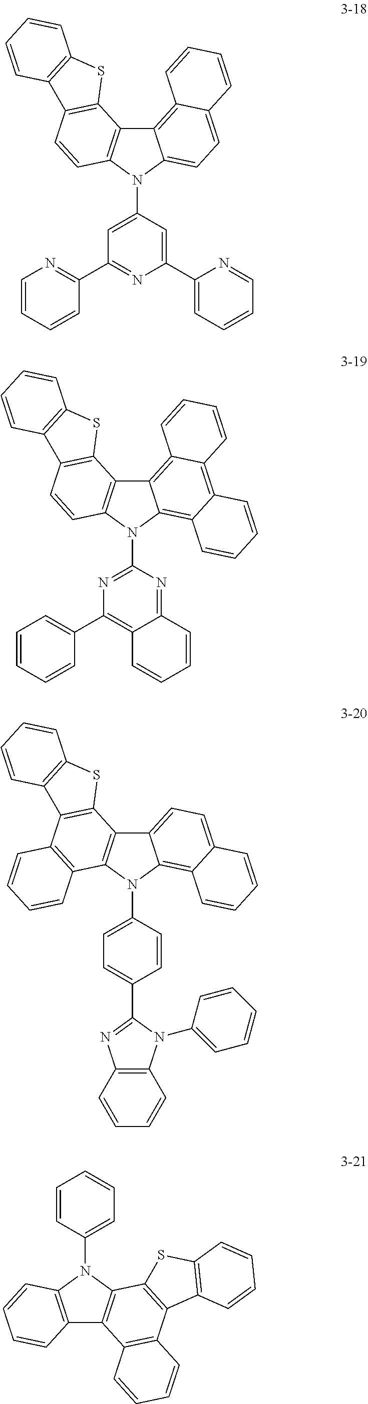

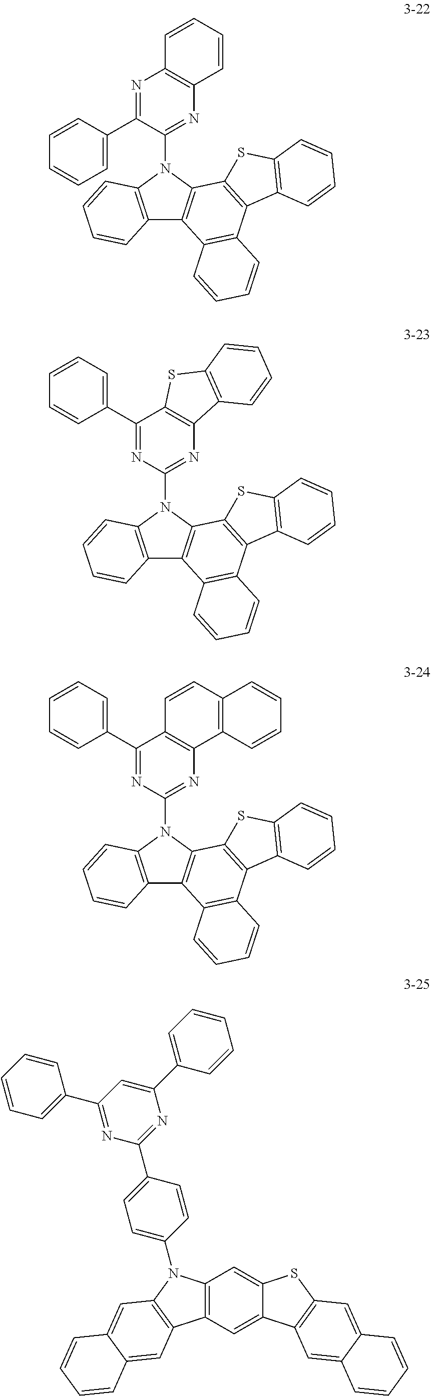

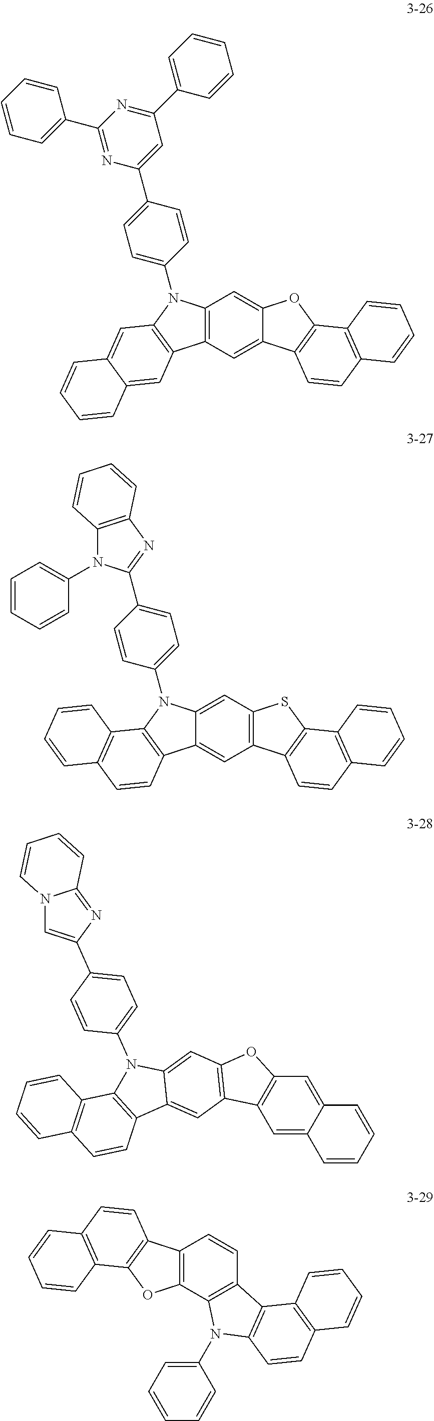 [SMILES: c1ccc(-c2cc(-c3ccc(-n4c5cc6ccccc6cc5c5cc6c(cc54)oc4c5ccccc5ccc64)cc3)nc(-c3ccccc3)n2)cc1 ; c1ccc(-n2c(-c3ccc(-n4c5cc6sc7c8ccccc8ccc7c6cc5c5ccc6ccccc6c54)cc3)nc3ccccc32)cc1 ; c1ccc2cc3c(cc2c1)oc1cc2c(cc13)c1ccc3ccccc3c1n2-c1ccc(-c2cn3ccccc3n2)cc1 ; c1ccc(-n2c3ccc4ccccc4c3c3ccc4c5ccc6ccccc6c5oc4c32)cc1]

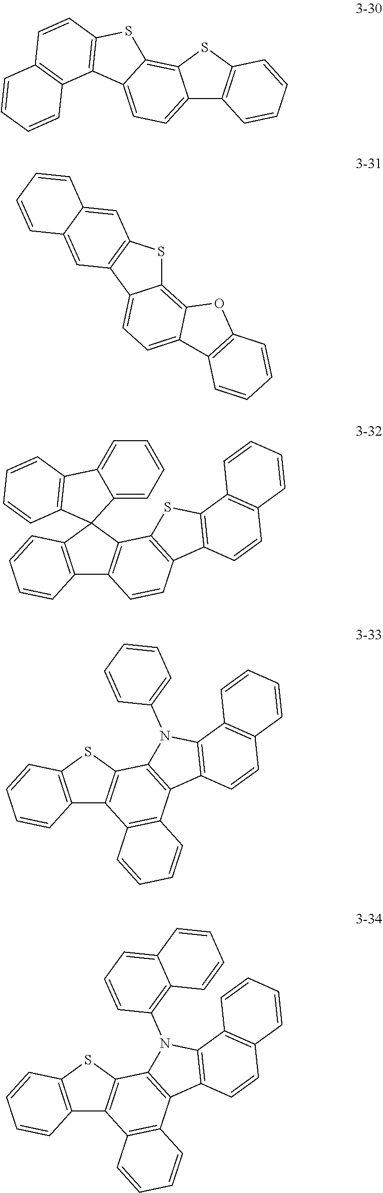

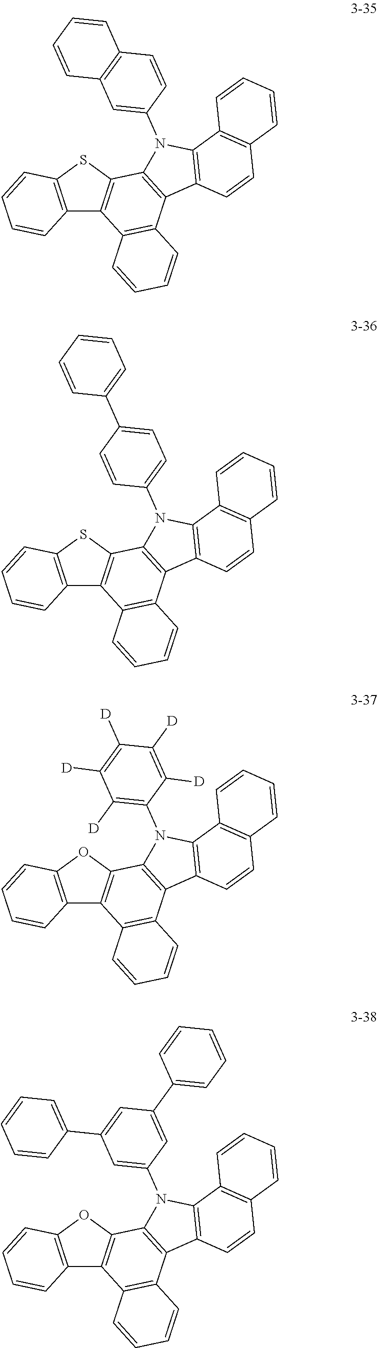 [SMILES: c1ccc2cc(-n3c4c5ccccc5ccc4c4c5ccccc5c5c6ccccc6sc5c43)ccc2c1 ; c1ccc(-c2ccc(-n3c4c5ccccc5ccc4c4c5ccccc5c5c6ccccc6sc5c43)cc2)cc1 ; [2H]c1c([2H])c([2H])c(-n2c3c4ccccc4ccc3c3c4ccccc4c4c5ccccc5oc4c32)c([2H])c1[2H] ; c1ccc(-c2cc(-c3ccccc3)cc(-n3c4c5ccccc5ccc4c4c5ccccc5c5c6ccccc6oc5c43)c2)cc1]

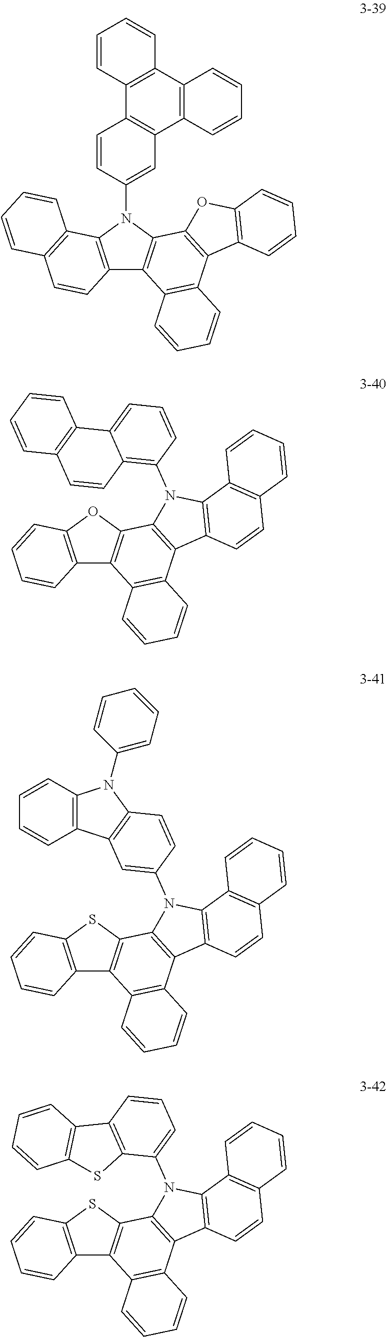

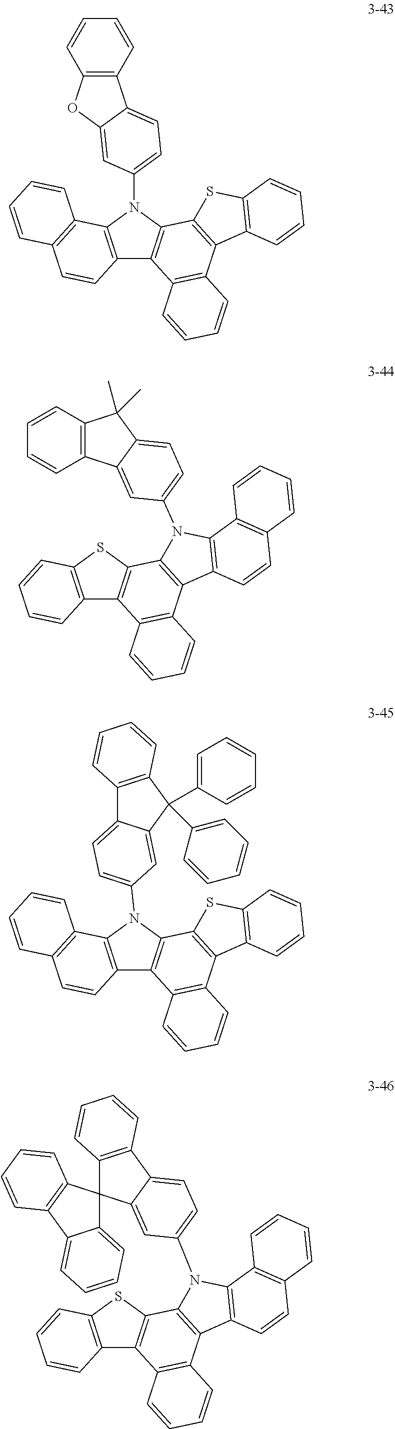 [SMILES: c1ccc2c(c1)ccc1c3c4ccccc4c4c5ccccc5sc4c3n(-c3ccc4c(c3)oc3ccccc34)c21 ; CC1(C)c2ccccc2-c2cc(-n3c4c5ccccc5ccc4c4c5ccccc5c5c6ccccc6sc5c43)ccc21 ; c1ccc(C2(c3ccccc3)c3ccccc3-c3ccc(-n4c5c6ccccc6ccc5c5c6ccccc6c6c7ccccc7sc6c54)cc32)cc1 ; c1ccc2c(c1)-c1ccccc1C21c2ccccc2-c2ccc(-n3c4c5ccccc5ccc4c4c5ccccc5c5c6ccccc6sc5c43)cc21]

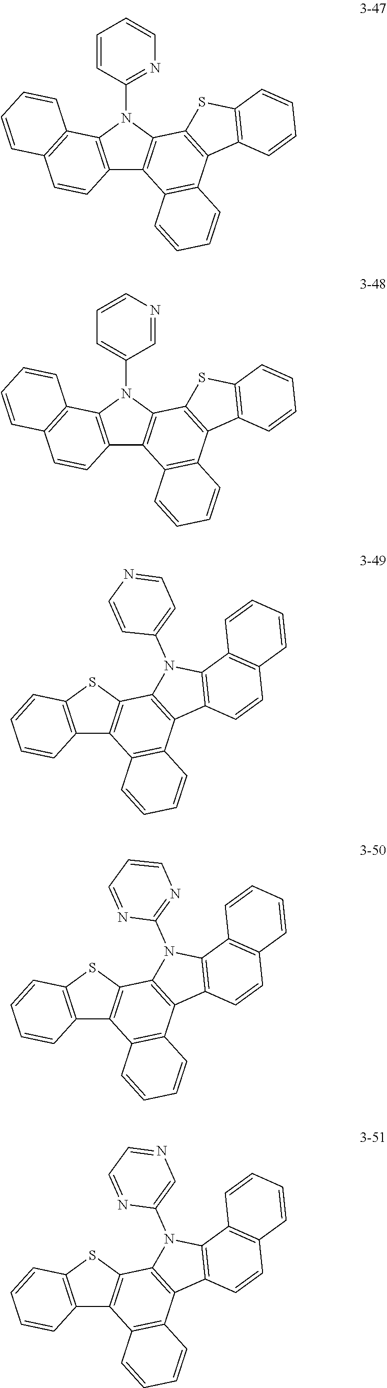 [SMILES: c1ccc(-n2c3c4ccccc4ccc3c3c4ccccc4c4c5ccccc5sc4c32)nc1 ; c1cncc(-n2c3c4ccccc4ccc3c3c4ccccc4c4c5ccccc5sc4c32)c1 ; c1ccc2c(c1)ccc1c3c4ccccc4c4c5ccccc5sc4c3n(-c3ccncc3)c21 ; c1cnc(-n2c3c4ccccc4ccc3c3c4ccccc4c4c5ccccc5sc4c32)nc1 ; c1ccc2c(c1)ccc1c3c4ccccc4c4c5ccccc5sc4c3n(-c3cnccn3)c21]

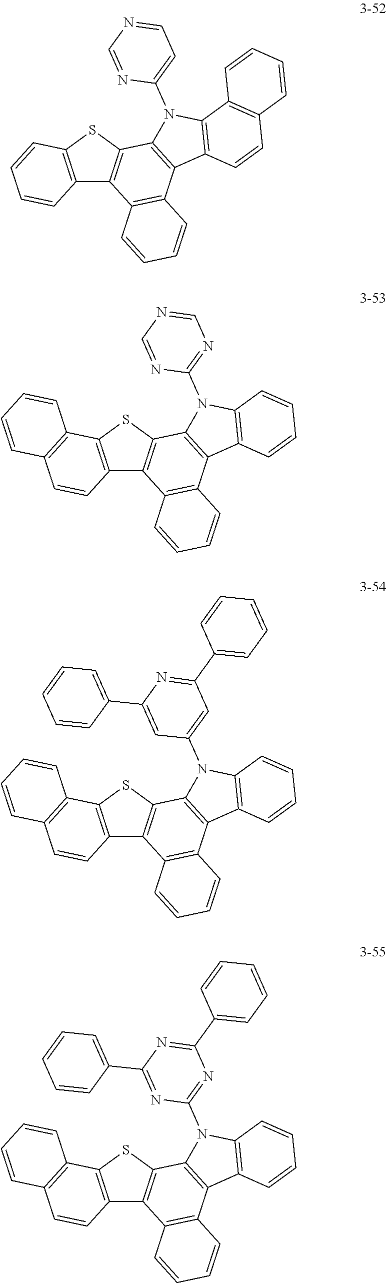 [SMILES: c1ccc2c(c1)ccc1c3c4ccccc4c4c5ccccc5sc4c3n(-c3ccncn3)c21 ; c1ccc2c(c1)ccc1c2sc2c1c1ccccc1c1c3ccccc3n(-c3ncncn3)c21 ; c1ccc(-c2cc(-n3c4ccccc4c4c5ccccc5c5c6ccc7ccccc7c6sc5c43)cc(-c3ccccc3)n2)cc1 ; c1ccc(-c2nc(-c3ccccc3)nc(-n3c4ccccc4c4c5ccccc5c5c6ccc7ccccc7c6sc5c43)n2)cc1]

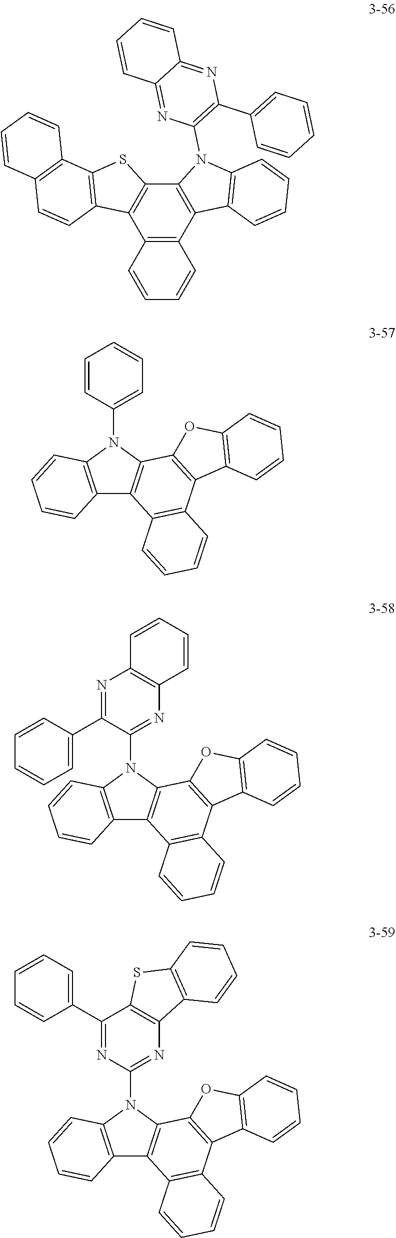

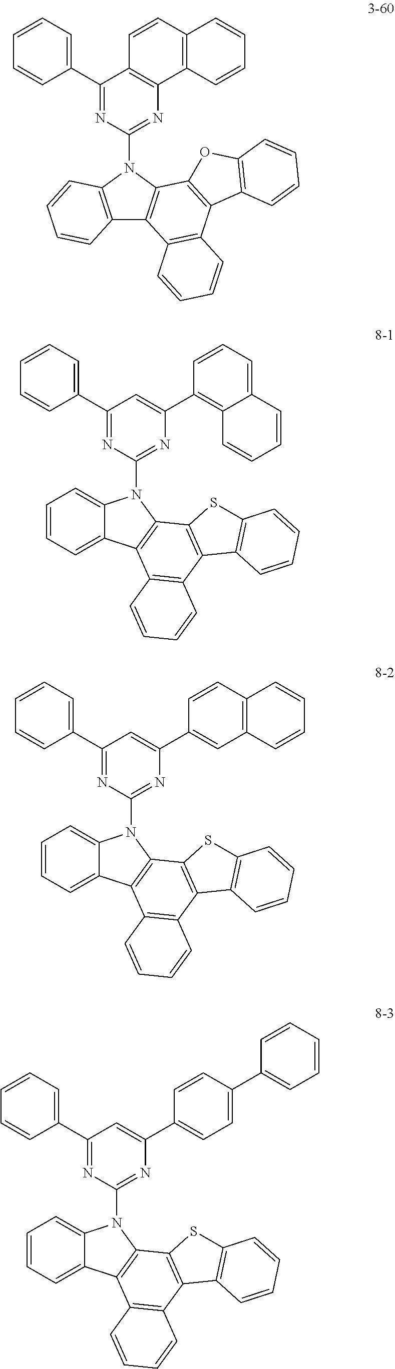 [SMILES: c1ccc(-c2nc(-n3c4ccccc4c4c5ccccc5c5c6ccccc6oc5c43)nc3c2ccc2ccccc23)cc1 ; c1ccc(-c2cc(-c3cccc4ccccc34)nc(-n3c4ccccc4c4c5ccccc5c5c6ccccc6sc5c43)n2)cc1 ; c1ccc(-c2cc(-c3ccc4ccccc4c3)nc(-n3c4ccccc4c4c5ccccc5c5c6ccccc6sc5c43)n2)cc1 ; c1ccc(-c2ccc(-c3cc(-c4ccccc4)nc(-n4c5ccccc5c5c6ccccc6c6c7ccccc7sc6c54)n3)cc2)cc1]

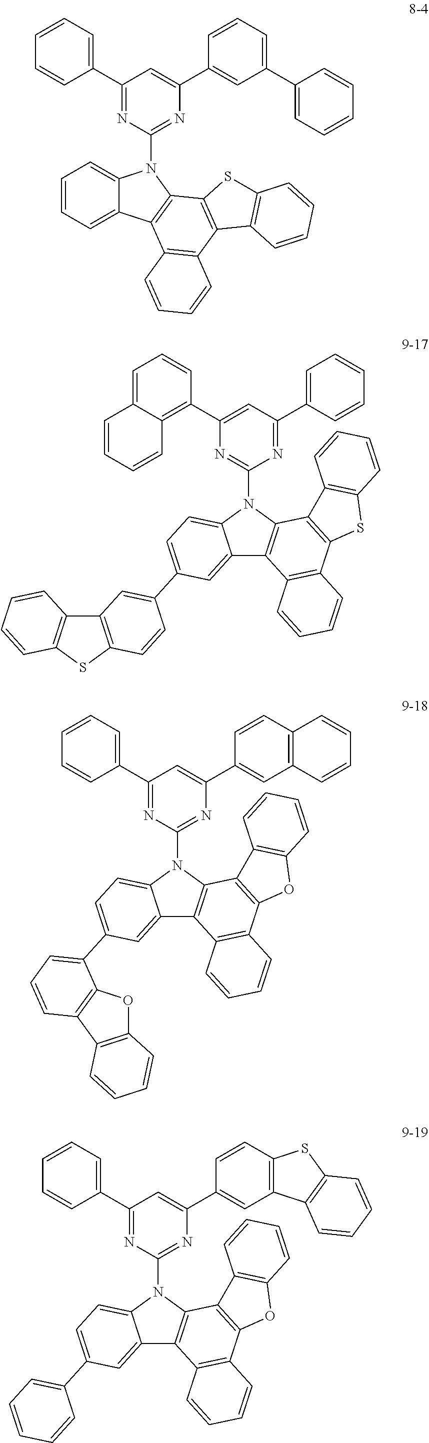

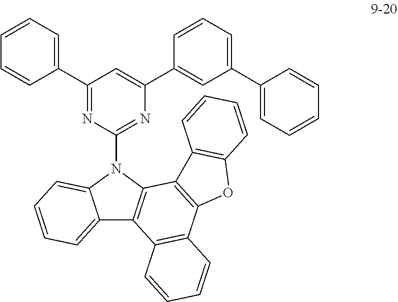 [SMILES: c1ccc(-c2cccc(-c3cc(-c4ccccc4)nc(-n4c5ccccc5c5c6ccccc6c6oc7ccccc7c6c54)n3)c2)cc1]

Specifically, the compound represented by formula 6 may be one of the following compounds, but there is no limitation thereto.

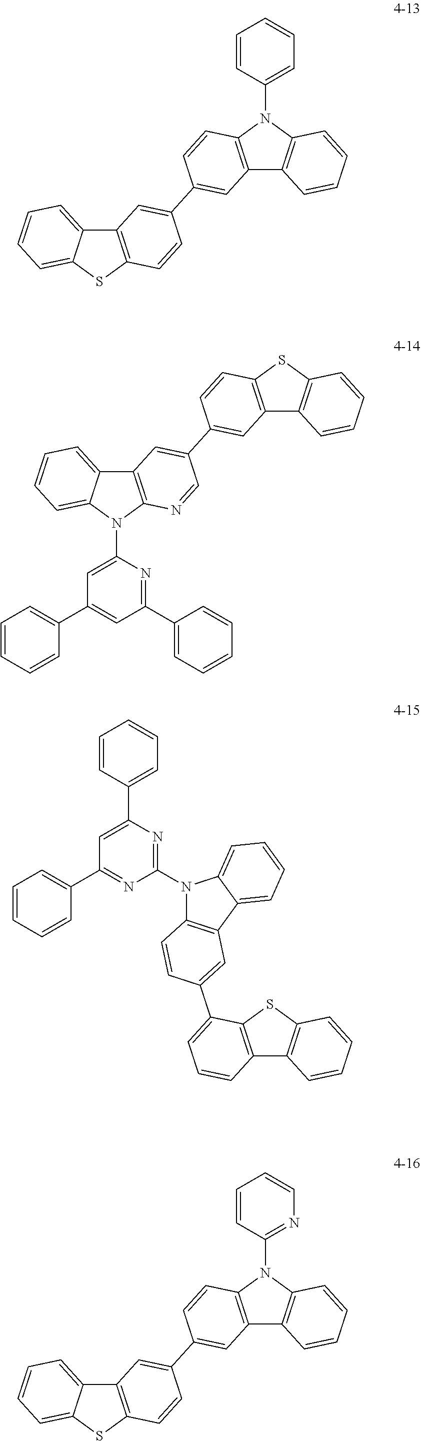 [SMILES: c1ccc(-n2c3ccccc3c3cc(-c4ccc5sc6ccccc6c5c4)ccc32)cc1 ; c1ccc(-c2cc(-c3ccccc3)nc(-n3c4ccccc4c4cc(-c5ccc6sc7ccccc7c6c5)cnc43)c2)cc1 ; c1ccc(-c2cc(-c3ccccc3)nc(-n3c4ccccc4c4cc(-c5cccc6c5sc5ccccc56)ccc43)n2)cc1 ; c1ccc(-n2c3ccccc3c3cc(-c4ccc5sc6ccccc6c5c4)ccc32)nc1]

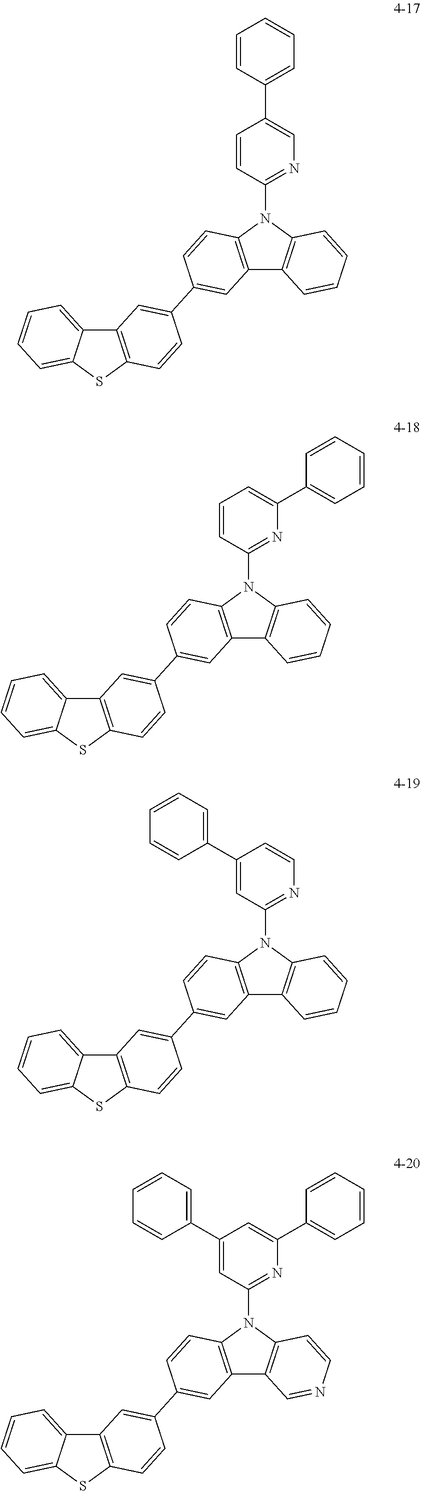 [SMILES: c1ccc(-c2ccc(-n3c4ccccc4c4cc(-c5ccc6sc7ccccc7c6c5)ccc43)nc2)cc1 ; c1ccc(-c2cccc(-n3c4ccccc4c4cc(-c5ccc6sc7ccccc7c6c5)ccc43)n2)cc1 ; c1ccc(-c2ccnc(-n3c4ccccc4c4cc(-c5ccc6sc7ccccc7c6c5)ccc43)c2)cc1 ; c1ccc(-c2cc(-c3ccccc3)nc(-n3c4ccncc4c4cc(-c5ccc6sc7ccccc7c6c5)ccc43)c2)cc1]

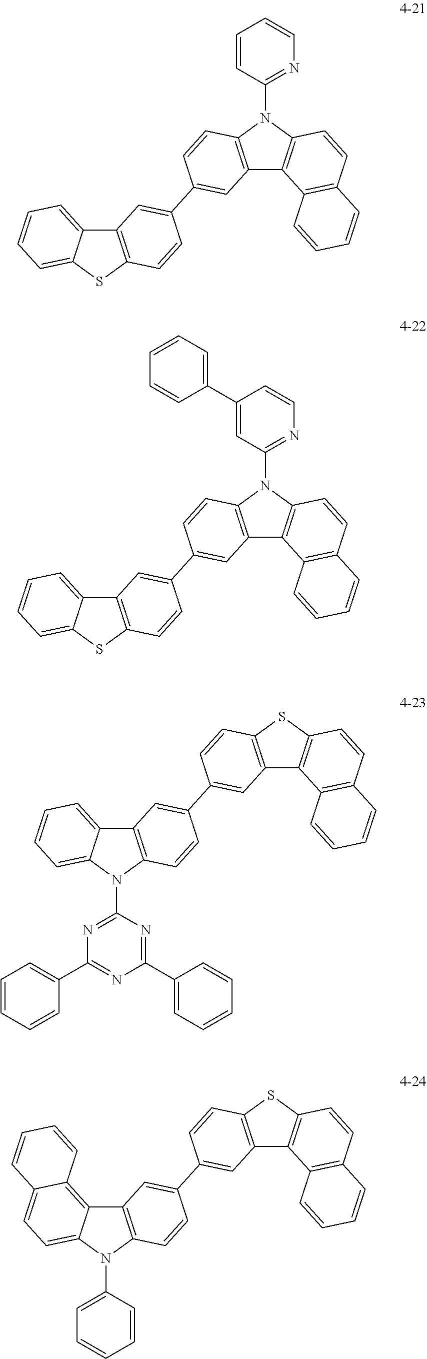 [SMILES: c1ccc(-n2c3ccc(-c4ccc5sc6ccccc6c5c4)cc3c3c4ccccc4ccc32)nc1 ; c1ccc(-c2ccnc(-n3c4ccc(-c5ccc6sc7ccccc7c6c5)cc4c4c5ccccc5ccc43)c2)cc1 ; c1ccc(-c2nc(-c3ccccc3)nc(-n3c4ccccc4c4cc(-c5ccc6sc7ccc8ccccc8c7c6c5)ccc43)n2)cc1 ; c1ccc(-n2c3ccc(-c4ccc5sc6ccc7ccccc7c6c5c4)cc3c3c4ccccc4ccc32)cc1]

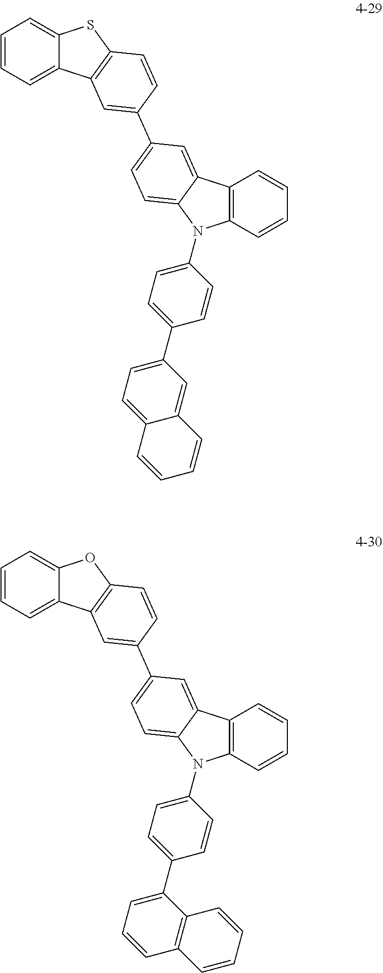 [SMILES: c1ccc2cc(-c3ccc(-n4c5ccccc5c5cc(-c6ccc7sc8ccccc8c7c6)ccc54)cc3)ccc2c1 ; c1ccc2c(-c3ccc(-n4c5ccccc5c5cc(-c6ccc7oc8ccccc8c7c6)ccc54)cc3)cccc2c1]

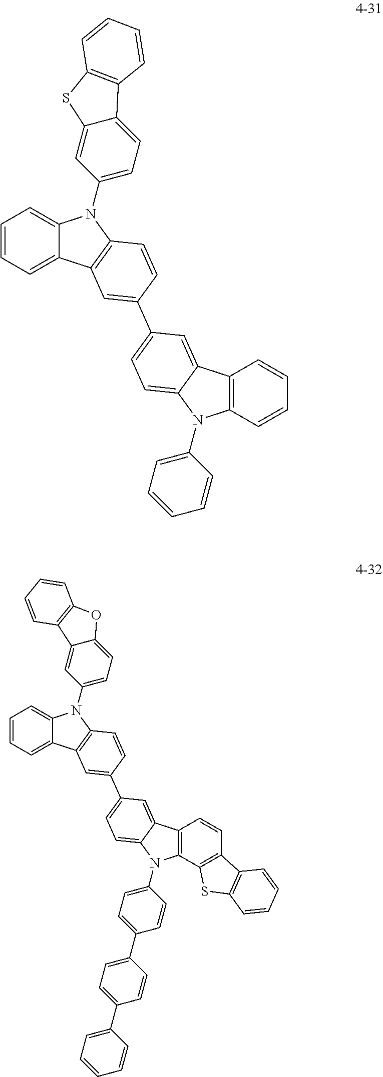 [SMILES: c1ccc(-n2c3ccccc3c3cc(-c4ccc5c(c4)c4ccccc4n5-c4ccc5c(c4)sc4ccccc45)ccc32)cc1 ; c1ccc(-c2ccc(-c3ccc(-n4c5ccc(-c6ccc7c(c6)c6ccccc6n7-c6ccc7oc8ccccc8c7c6)cc5c5ccc6c7ccccc7sc6c54)cc3)cc2)cc1]

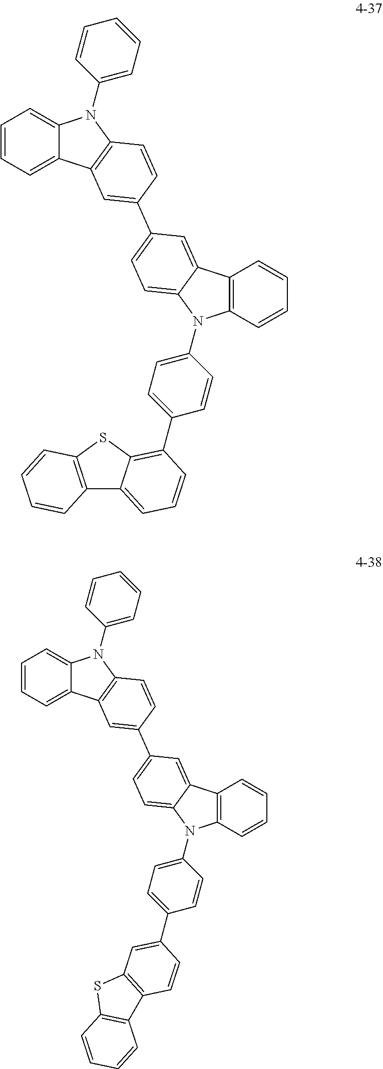 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc(-c5ccc6c(c5)sc5ccccc56)cc4)ccc32)cc1]

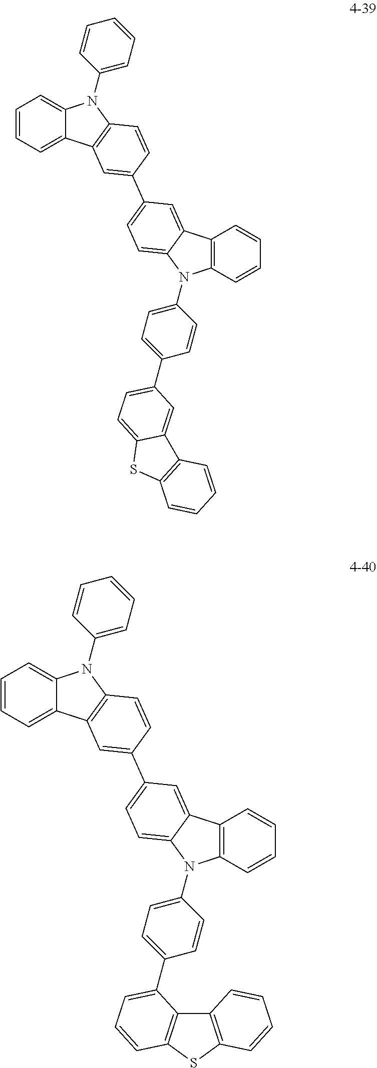 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; c1ccc(-n2c3ccccc3c3cc(-c4ccc5c(c4)c4ccccc4n5-c4ccc(-c5cccc6sc7ccccc7c56)cc4)ccc32)cc1]

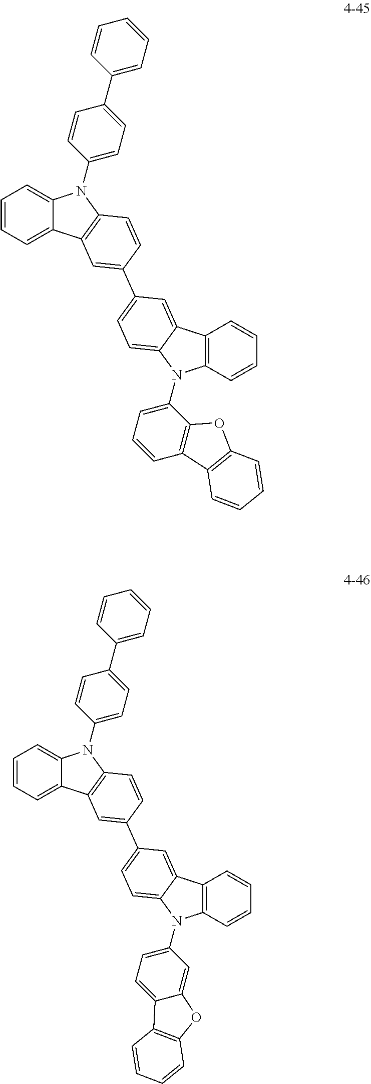 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-c2ccc(-n3c4ccccc4c4cc(-c5ccc6c(c5)c5ccccc5n6-c5ccc6c(c5)oc5ccccc56)ccc43)cc2)cc1]

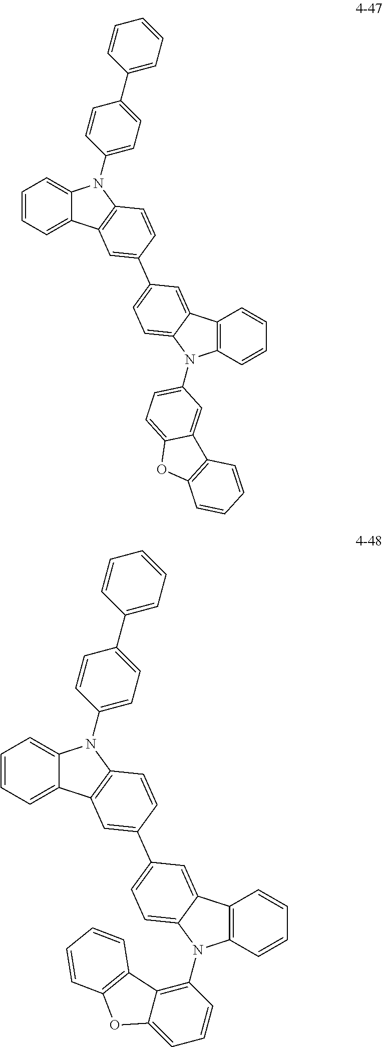 [SMILES: c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2ccc(-n3c4ccccc4c4cc(-c5ccc6c(c5)c5ccccc5n6-c5cccc6oc7ccccc7c56)ccc43)cc2)cc1]

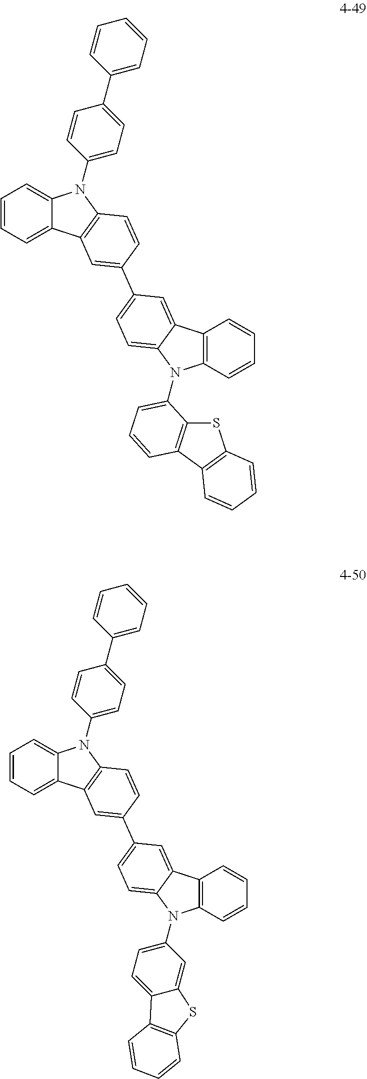 [SMILES: c1ccc(-c2ccc(-n3c4ccccc4c4cc(-c5ccc6c(c5)c5ccccc5n6-c5cccc6c5sc5ccccc56)ccc43)cc2)cc1 ; c1ccc(-c2ccc(-n3c4ccccc4c4cc(-c5ccc6c(c5)c5ccccc5n6-c5ccc6c(c5)sc5ccccc56)ccc43)cc2)cc1]

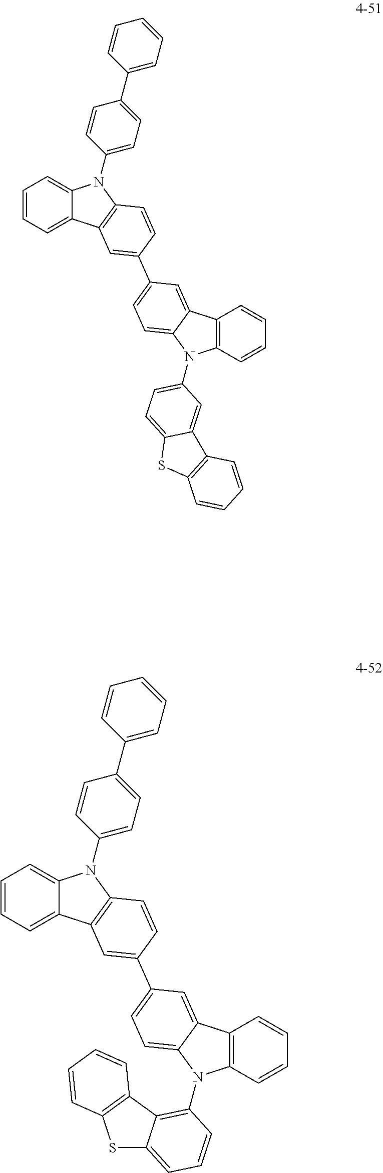 [SMILES: c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2ccc(-n3c4ccccc4c4cc(-c5ccc6c(c5)c5ccccc5n6-c5cccc6sc7ccccc7c56)ccc43)cc2)cc1]

Hereinafter, synthesis example of the compound represented by Formula and preparation method of an organic electric element according to one embodiment of the present invention will be described in detail by way of examples. However, the present invention is not limited to the following examples.

### SYNTHESIS EXAMPLE

The following final products were synthesized with reference to the synthetic method disclosed in Korean Patent Registration No. 10-1530049 filed by the present applicant (registration-published on Jun. 18, 2015), Korean Patent Publication No. 10-2017-0112865 (published on Oct. 12, 2017), Korean Patent Publication No. 10-2017-0112913 (published on Oct. 12, 2017).

Synthesis of 1-19′

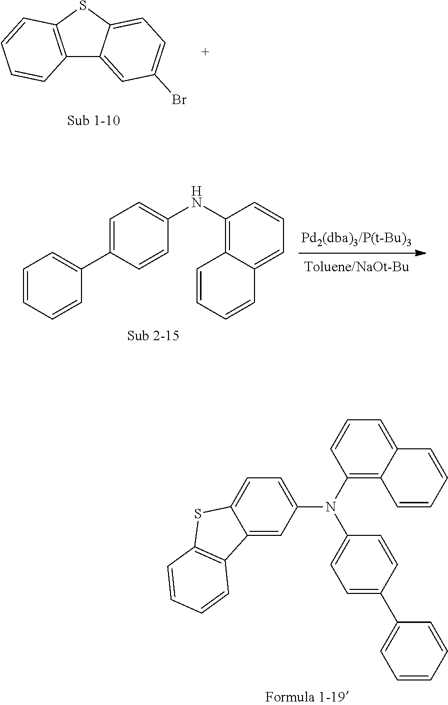 [SMILES: Brc1ccc2sc3ccccc3c2c1 ; c1ccc(-c2ccc(Nc3cccc4ccccc34)cc2)cc1 ; c1ccc(-c2ccc(N(c3ccc4sc5ccccc5c4c3)c3cccc4ccccc34)cc2)cc1]

After putting N-([1,1′-biphenyl]-4-yl)naphthalen-1-amine (10 g, 33.6 mmol), 2-bromodibenzo[b,d]thiophene (9.8 g, 37.2 mmol), Pd₂(dba)₃ (1.55 g, 1.7 mmol), P(t-Bu)₃ (0.68 g, 3.38 mmol), NaOt-Bu (10.76 g, 112 mmol) and toluene (355 mL) into a round bottom flask, the reaction was carried out at 100° C. When the reaction was completed, the reaction product was extracted with CH₂Cl₂ and water. The organic layer was dried with MgSO₄ and concentrated. The concentrate was passed through a silica gel column and recrystallized to obtain 12.3 g (yield: 76%) of the product.

Synthesis of 1-20′

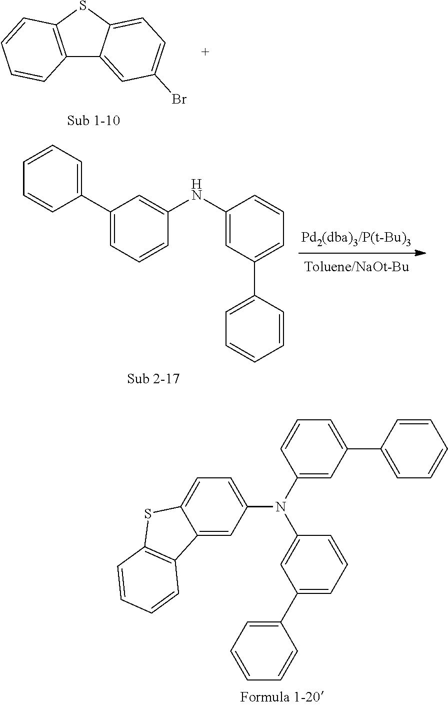 [SMILES: Brc1ccc2sc3ccccc3c2c1 ; c1ccc(-c2cccc(Nc3cccc(-c4ccccc4)c3)c2)cc1 ; c1ccc(-c2cccc(N(c3cccc(-c4ccccc4)c3)c3ccc4sc5ccccc5c4c3)c2)cc1]

12.2 g (yield: 78%) of the product was obtained in the same manner as the synthesis method of 1-19′ by using Di([1,1′-biphenyl]-3-yl)amine (10 g, 31.1 mmol), 2-bromodibenzo[b,d]thiophene (9 g, 34.2 mmol), Pd₂(dba)₃ (1.42 g, 1.56 mmol), P(t-Bu)₃ (0.63 g, 3.11 mmol), NaOt-Bu (9.87 g, 102.7 mmol) and toluene (327 mL).

Synthesis of 1-23′

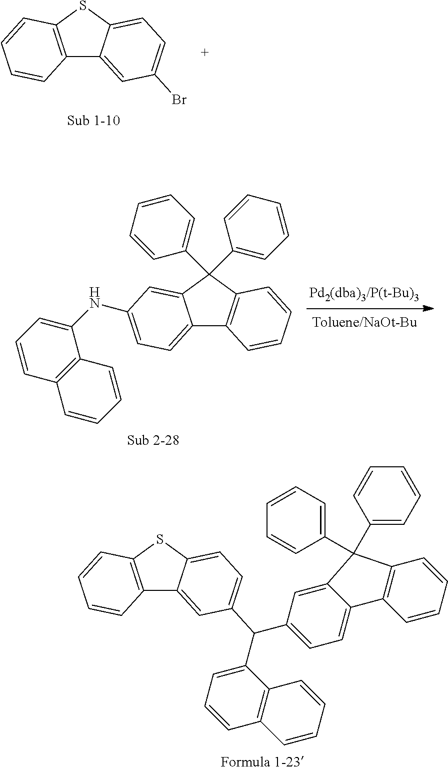 [SMILES: Brc1ccc2sc3ccccc3c2c1 ; c1ccc(C2(c3ccccc3)c3ccccc3-c3ccc(Nc4cccc5ccccc45)cc32)cc1 ; c1ccc(C2(c3ccccc3)c3ccccc3-c3ccc(C(c4ccc5sc6ccccc6c5c4)c4cccc5ccccc45)cc32)cc1]

10.2 g (yield: 73%) of the product was obtained in the same manner as the synthesis method of 1-19′ by using N-(naphthalen-1-yl)-9,9-diphenyl-9H-fluoren-2-amine (10 g, 21.8 mmol), 2-bromodibenzo[b,d]thiophene (6.3 g, 23.9 mmol), Pd₂(dba)₃ (1 g, 1.09 mmol), P(t-Bu)₃ (0.44 g, 2.2 mmol), NaOt-Bu (6.9 g, 71.8 mmol) and toluene (230 mL).

Synthesis of 1-24′

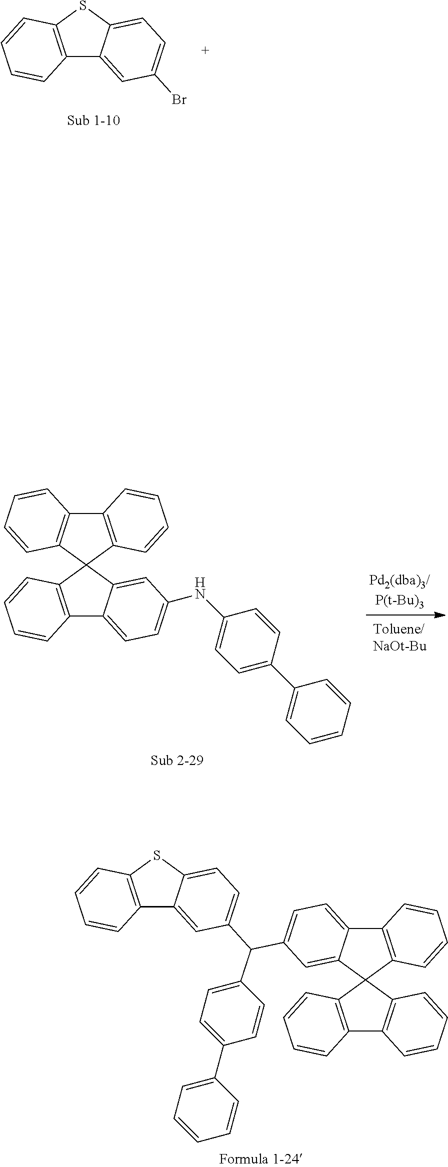 [SMILES: Brc1ccc2sc3ccccc3c2c1 ; c1ccc(-c2ccc(Nc3ccc4c(c3)C3(c5ccccc5-c5ccccc53)c3ccccc3-4)cc2)cc1 ; c1ccc(-c2ccc(C(c3ccc4c(c3)C3(c5ccccc5-c5ccccc53)c3ccccc3-4)c3ccc4sc5ccccc5c4c3)cc2)cc1]

10.2 g (yield: 74%) of the product was obtained in the same manner as the synthesis method of 1-19′ by using N-([1,1′-biphenyl]-4-yl)-9,9′-spirobi[fluoren]-2-amine (10 g, 20.7 mmol), 2-bromodibenzo[b,d]thiophene (6 g, 22.7 mmol), Pd₂(dba)₃ (0.95 g, 1.03 mmol), P(t-Bu)₃ (0.42 g, 2.07 mmol), NaOt-Bu (6.55 g, 68.2 mmol), toluene (220 mL).

Synthesis of 1-29′

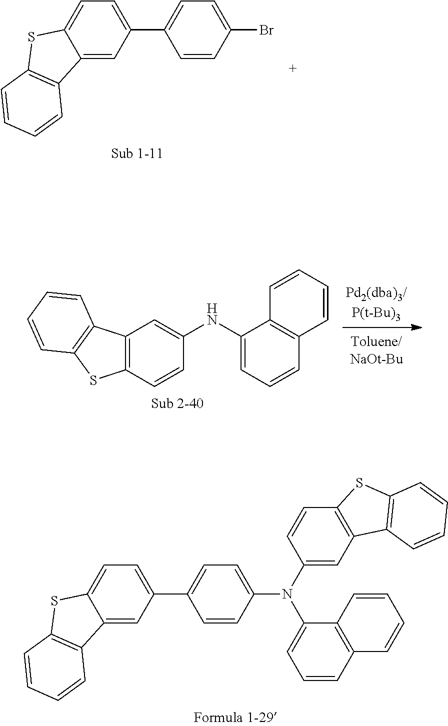 [SMILES: Brc1ccc(-c2ccc3sc4ccccc4c3c2)cc1 ; c1ccc2c(Nc3ccc4sc5ccccc5c4c3)cccc2c1 ; c1ccc2c(N(c3ccc(-c4ccc5sc6ccccc6c5c4)cc3)c3ccc4sc5ccccc5c4c3)cccc2c1]

12.9 g (yield: 72%) of the product was obtained in the same manner as the synthesis method of 1-19′ by using N-(naphthalen-1-yl)dibenzo[b,d]thiophen-2-amine (10 g, 30.7 mmol), 2-(4-bromophenyl)dibenzo[b,d]thiophene (11.5 g, 33.8 mmol), Pd₂(dba)₃ (1.41 g, 1.54 mmol), P(t-Bu)₃ (0.62 g, 3.07 mmol), NaOt-Bu (9.75 g, 101.4 mmol) and toluene (325 mL).

Synthesis of 1-30′

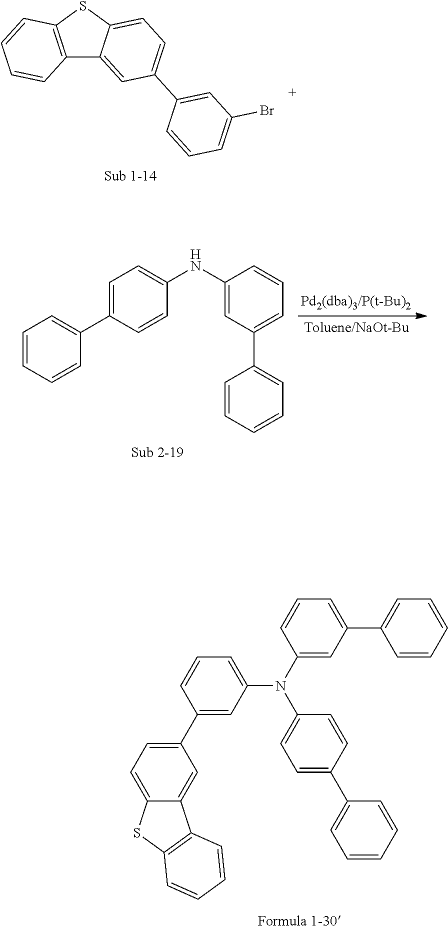 [SMILES: Brc1cccc(-c2ccc3sc4ccccc4c3c2)c1 ; c1ccc(-c2ccc(Nc3cccc(-c4ccccc4)c3)cc2)cc1 ; c1ccc(-c2ccc(N(c3cccc(-c4ccccc4)c3)c3cccc(-c4ccc5sc6ccccc6c5c4)c3)cc2)cc1]

12.8 g (yield: 71%) of the product was obtained in the same manner as the synthesis method of 1-19′ by using N-([1,1′-biphenyl]-4-yl)-[1,1′-biphenyl]-3-amine (10 g, 31.1 mmol), 2-(3-bromophenyl)dibenzo[b,d]thiophene (11.6 g, 34.2 mmol), Pd₂(dba)₃ (1.42 g, 1.55 mmol), P(t-Bu)₃ (0.63 g, 3.11 mmol), NaOt-Bu (9.9 g, 103 mmol) and toluene (330 mL).

Synthesis of 1-36′

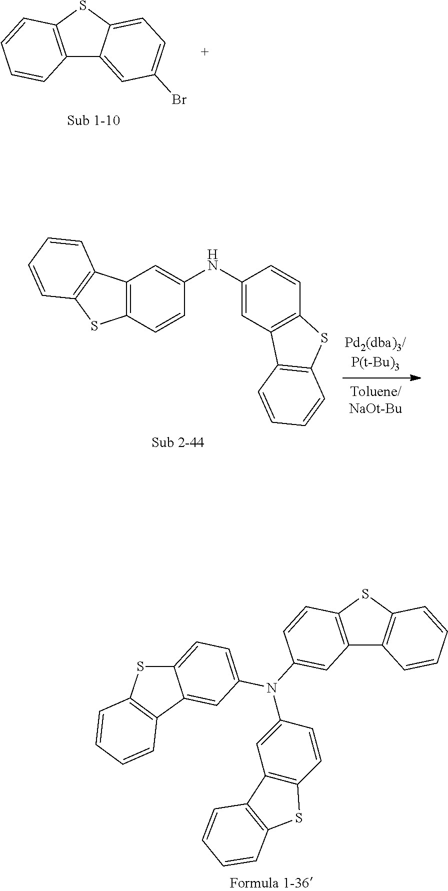 [SMILES: Brc1ccc2sc3ccccc3c2c1 ; c1ccc2c(c1)sc1ccc(Nc3ccc4sc5ccccc5c4c3)cc12 ; c1ccc2c(c1)sc1ccc(N(c3ccc4sc5ccccc5c4c3)c3ccc4sc5ccccc5c4c3)cc12]

11.4 g (yield: 77%) of the product was obtained in the same manner as the synthesis method of 1-19′ by using bis(dibenzo[b,d]thiophen-2-yl)amine (10 g, 26.2 mmol), 2-bromodibenzo[b,d]thiophene (7.59 g, 28.8 mmol), Pd₂(dba)₃ (1.2 g, 1.31 mmol), P(t-Bu)₃ (0.53 g, 2.62 mmol), NaOt-Bu (8.31 g, 86.5 mmol) and toluene (275 mL).

Synthesis of 1-49′

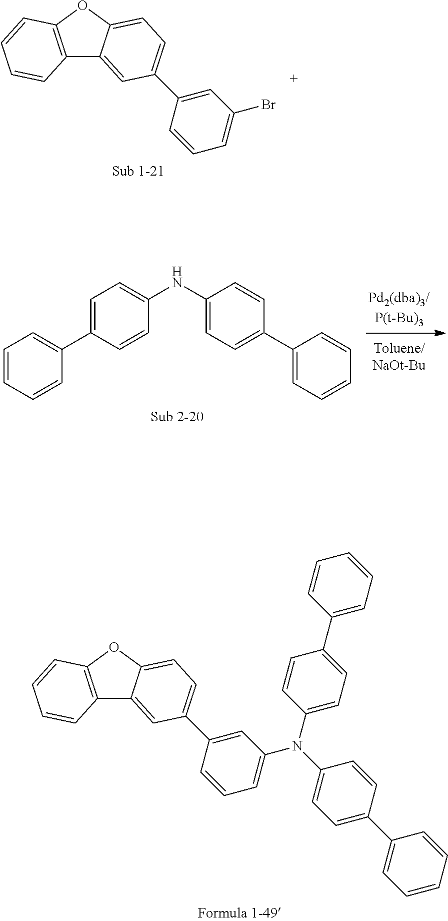 [SMILES: Brc1cccc(-c2ccc3oc4ccccc4c3c2)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5oc6ccccc6c5c4)c3)cc2)cc1]

13.3 g (yield: 76%) of the product was obtained in the same manner as the synthesis method of 1-19′ by using Di([1,1′-biphenyl]-4-yl)amine (10 g, 31.1 mmol), 2-(3-bromophenyl)dibenzo[b,d]furan (11.1 g, 34.2 mmol), Pd₂(dba)₃ (1.42 g, 1.56 mmol), P(t-Bu)₃ (0.63 g, 3.11 mmol), NaOt-Bu (9.9 g, 103 mmol) and toluene (330 mL).

Synthesis of 1-51′

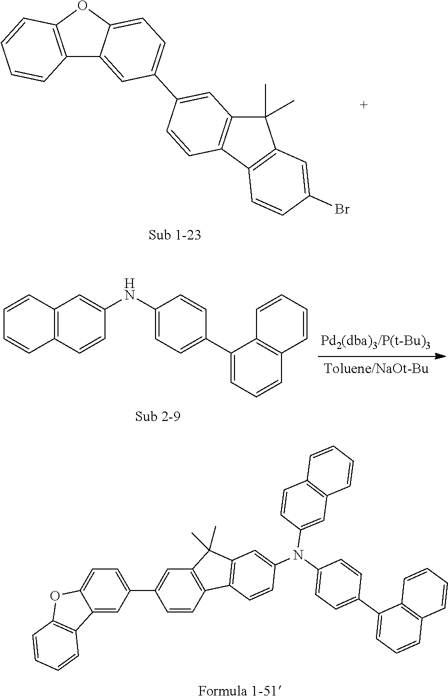 [SMILES: CC1(C)c2cc(Br)ccc2-c2ccc(-c3ccc4oc5ccccc5c4c3)cc21 ; c1ccc2cc(Nc3ccc(-c4cccc5ccccc45)cc3)ccc2c1 ; CC1(C)c2cc(-c3ccc4oc5ccccc5c4c3)ccc2-c2ccc(N(c3ccc(-c4cccc5ccccc45)cc3)c3ccc4ccccc4c3)cc21]

14.5 g (yield: 71%) of the product was obtained in the same manner as the synthesis method of 1-19′ by using N-(4-(naphthalen-1-yl)phenyl)naphthalen-2-amine (10 g, 28.9 mmol), 2-(7-bromo-9,9-dimethyl-9H-fluoren-2-yl)dibenzo[b,d]furan (14 g, 32 mmol), Pd₂(dba)₃ (1.33 g, 1.45 mmol), P(t-Bu)₃ (0.59 g, 2.9 mmol), NaOt-Bu (9.2 g, 95.5 mmol) and toluene (310 mL).

Synthesis of 1-59′

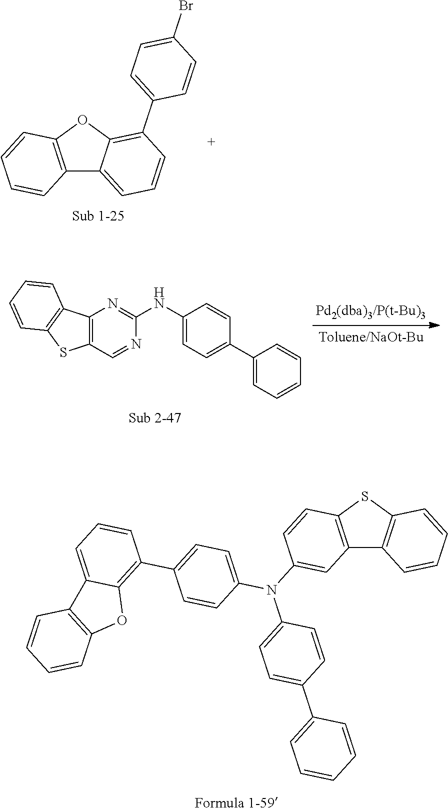 [SMILES: Brc1ccc(-c2cccc3c2oc2ccccc23)cc1 ; "+" ; c1ccc(-c2ccc(Nc3ncc4sc5ccccc5c4n3)cc2)cc1 ; c1ccc(-c2ccc(N(c3ccc(-c4cccc5c4oc4ccccc45)cc3)c3ccc4sc5ccccc5c4c3)cc2)cc1]

12.3 g (yield: 73%) of the product was obtained in the same manner as the synthesis method of 1-19′ by using N-([1,1′-biphenyl]-4-yl)benzo[4,5]thieno[3,2-d]pyrimidin-2-amine (10 g, 28.3 mmol), 4-(4-bromophenyl)dibenzo[b,d]furan (10.1 g, 31.1 mmol), Pd₂(dba)₃ (1.3 g, 1.41 mmol), P(t-Bu)₃ (0.57 g, 2.83 mmol), NaOt-Bu (8.98 g, 93.4 mmol) and toluene (300 mL).

Synthesis of Synthesis of P-1

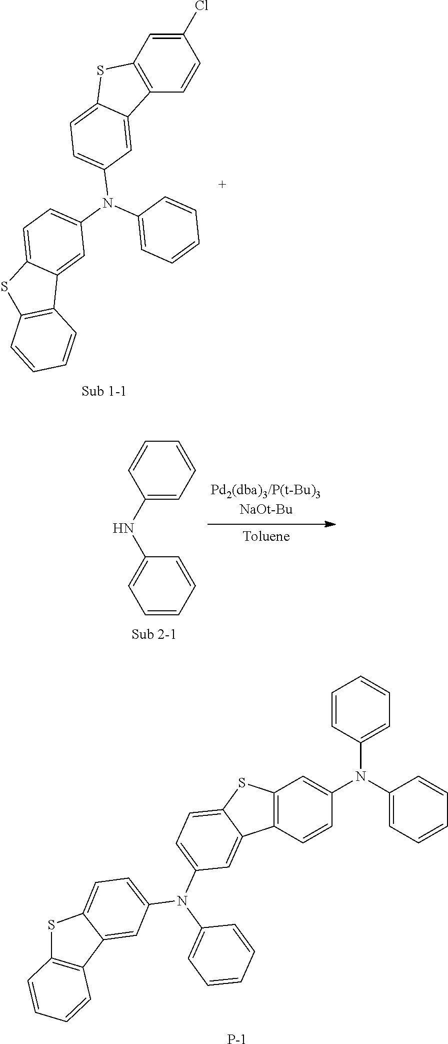

After Sub 1-1 (7.8 g, 15.94 mmol) was dissolved in toluene (150 ml) in a round bottom flask, Sub 2-1 (2.7 g, 15.94 mmol), Pd₂(dba)₃ (0.44 g, 0.47 mmol), 50% P(t-Bu)₃ (1.95 ml, 0.97 mmol) and NaOt-Bu (4.6 g, 47.84 mmol) were added thereto and the mixture was stirred at 130° C. When the reaction was completed, the reaction product was extracted with CH₂Cl₂ and water. The organic layer was dried with MgSO₄ and concentrated. Thereafter, the concentrate was passed through a silica gel column and recrystallized to obtain 8.1 g (yield: 80%) of Product P-1.

Synthesis of Synthesis of P-13

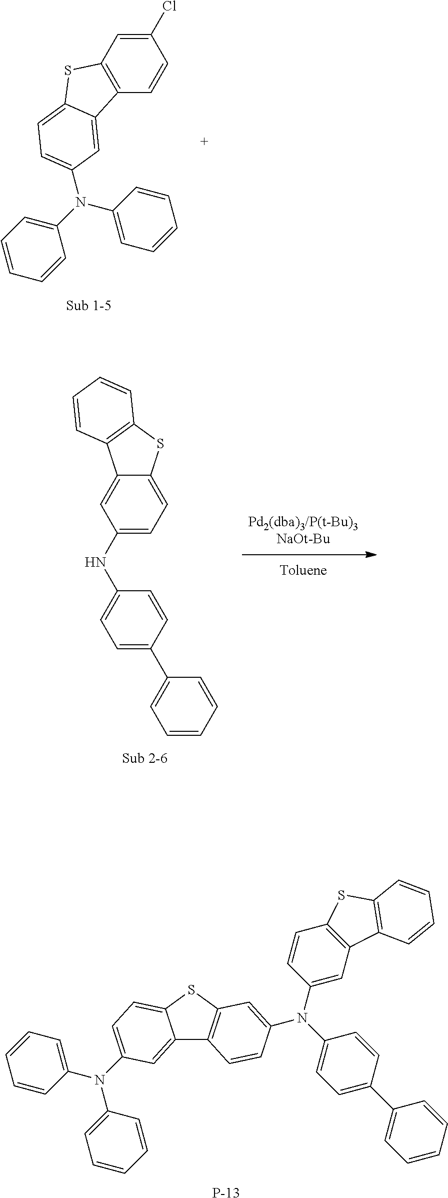

11 g (yield: 61%) of the product P-13 was obtained in the same manner as the synthesis method of P-1 by using Sub 1-5 (10 g, 25.91 mmol), Sub 2-6 (9.1 g, 25.91 mmol), Pd₂(dba)₃ (0.72 g, 0.0077 mmol), P(t-Bu)₃ (0.6 mL, 0.016 mmol), NaOt-Bu (7.5 g, 77.7 mmol).

Synthesis of Synthesis of P-22

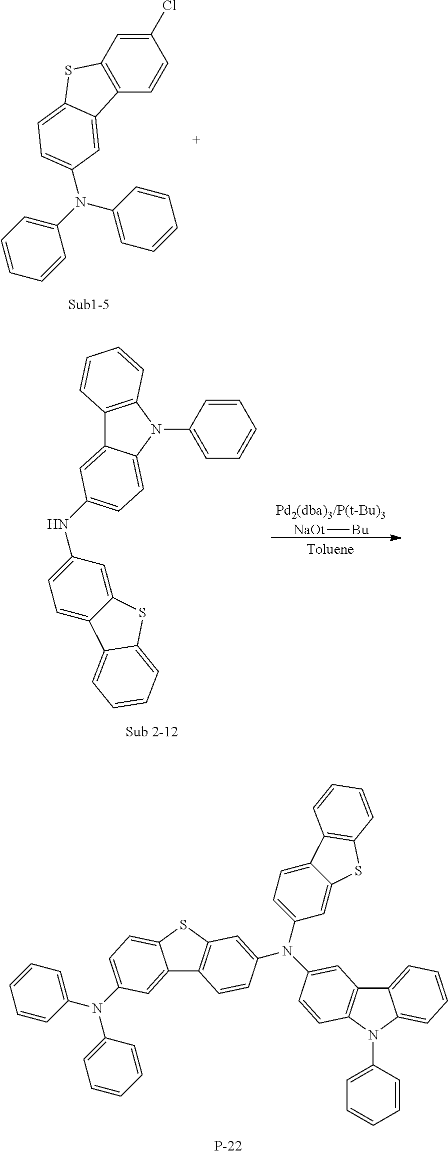

15 g (yield: 73%) of the product P-22 was obtained in the same manner as the synthesis method of P-1 by using Sub 1-5 (10 g, 25.91 mmol), Sub 2-12 (11.4 g, 25.91 mmol), Pd₂(dba)₃ (0.72 g, 0.0077 mmol), P(t-Bu)₃ (0.6 mL, 0.016 mmol), NaOt-Bu (7.5 g, 77.7 mmol).

Synthesis of Synthesis of 2-77

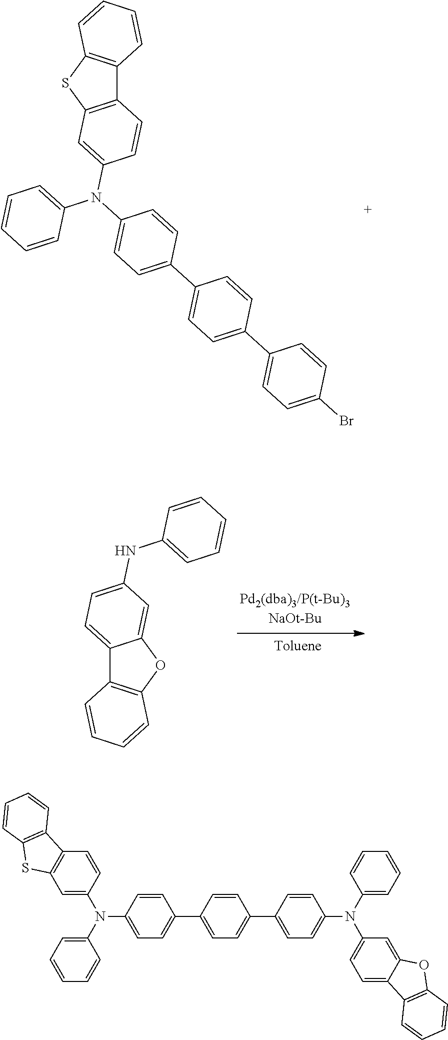 [SMILES: Brc1ccc(-c2ccc(-c3ccc(N(c4ccccc4)c4ccc5c(c4)sc4ccccc45)cc3)cc2)cc1 ; c1ccc(Nc2ccc3c(c2)oc2ccccc23)cc1 ; c1ccc(N(c2ccc(-c3ccc(-c4ccc(N(c5ccccc5)c5ccc6c(c5)sc5ccccc56)cc4)cc3)cc2)c2ccc3c(c2)oc2ccccc23)cc1]

10.49 g (yield: 73%) of the product was obtained in the same manner as the synthesis method of P-1 by using N-([1,1′-biphenyl]-4-yl)-N-(4″-bromo-[1,1′:4′,1″-terphenyl]-4-yl)dibenzo[b,d]thiophen-3-amine (11 g, 18.88 mmol), toluene (198 ml), N-([1,1′-biphenyl]-4-yl)dibenzo[b,d]furan-3-amine (5.39 g, 20.77 mmol), Pd₂(dba)₃ (0.52 g, 0.57 mmol), P(t-Bu)₃ (0.38 g, 1.89 mmol), NaOt-Bu (5.44 g, 56.65 mmol).

Synthesis of Synthesis of 3-9

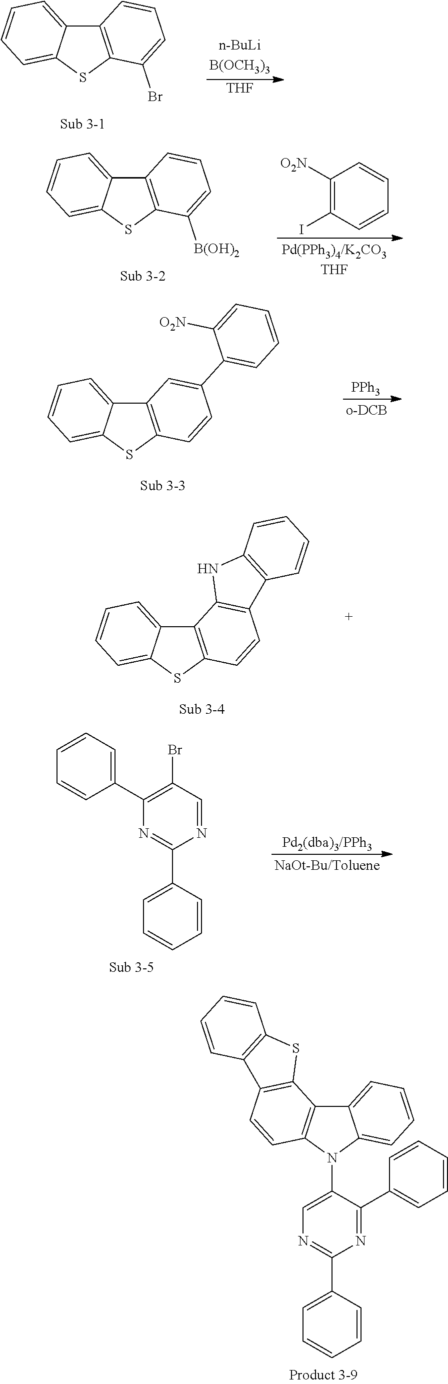

(1) Synthesis Method of Sub 3-2

After dissolving Sub 3-1 in anhydrous THF, the temperature was lowered to −78° C. and n-BuLi (2.5 M in hexane) was slowly added thereto. Then, mixture was stirred at 0° C. for 1 hour. Thereafter, the temperature of the reactant was lowered to −78° C., trimethyl borate was added dropwise, and the mixture was stirred at room temperature for 12 hours. When the reaction was completed, 2N-HCl aqueous solution was added and the mixture was stirred for 30 minutes. Then, the mixture was extracted with ether and water in the extract was removed with anhydrous MgSO₄. Then, the resultant was filtered under reduced pressure and was concentrated to remove the organic solvent. The concentrate was separated by column chromatography to obtain Sub 3-2.

(2) Synthesis Method of Sub 3-3

After Sub 3-2, 1-iodo-2-nitrobenzene, Pd(PPh₃)₄ and K₂CO₃ were dissolved in anhydrous THF and a small amount of water, the mixture was refluxed for 24 hours. When the reaction was completed, the temperature of the reaction product was cooled to room temperature and extracted with CH₂Cl₂. The extract was washed with water and the water was removed with anhydrous MgSO₄. Then, the resultant was filtered under reduced pressure and was concentrated to remove the organic solvent. The concentrate was separated by column chromatography to obtain Sub 3-3.

(3) Synthesis Method of Sub 3-4

After Sub 3-3 and triphenylphosphine were dissolved in o-dichlorobenzene, the mixture was refluxed for 24 hours. When the reaction was completed, the solvent was removed by distillation under reduced pressure. Thereafter, the concentrate was separated by column chromatography to obtain Sub 3-4.

(4) Synthesis method of Product 3-9

After adding Sub 3-4 (1 eq.) and Sub 5-6 (1.1 eq.) to toluene, Pd₂(dba)₃ (0.05 eq.), PPh₃ (0.1 eq.) and NaOt-Bu (3 eq.) were added thereto and the mixture was stirred at 100° C. for 24 hours under reflux. Thereafter, the resultant was extracted with ether and water, and the organic layer was dried with MgSO₄ and concentrated. Thereafter, the concentrate was passed through a silica gel column and recrystallized to obtain Product 3-9.

Synthesis of 3-5

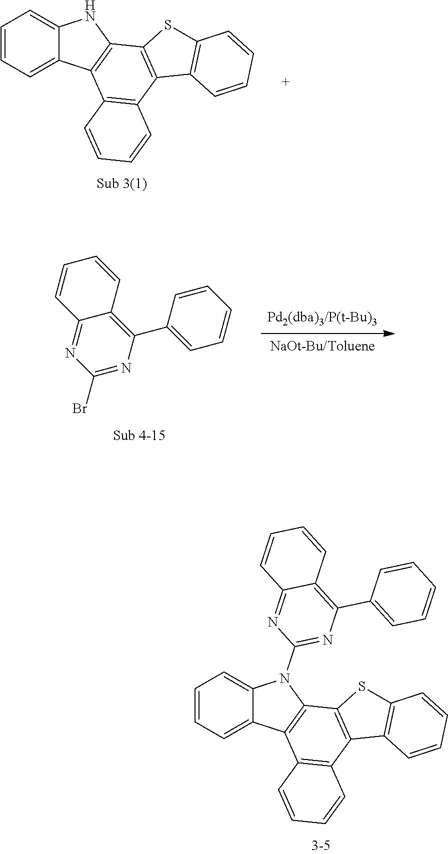 [SMILES: c1ccc2c(c1)[nH]c1c3sc4ccccc4c3c3ccccc3c21 ; Brc1nc(-c2ccccc2)c2ccccc2n1 ; c1ccc(-c2nc(-n3c4ccccc4c4c5ccccc5c5c6ccccc6sc5c43)nc3ccccc23)cc1]

After dissolving Sub 3(1) (15.3 g, 47.3 mmol) in toluene (500 mL) in round bottom flask, Sub 4-15 (14.8 g, 52.0 mmol), Pd₂(dba)₃ (2.4 g, 2.6 mmol), P(t-Bu)₃ (1.1 g, 5.2 mmol) and NaOt-Bu (15 g, 156.1 mmol) were added thereto and the mixture was stirred at 100° C. When the reaction was completed, the resultant was extracted with CH₂Cl₂ and water, and the organic layer was dried with MgSO₄ and concentrated. Thereafter, the concentrate was passed through a silica gel column and recrystallized to obtain 17.0 g (yield: 68%) of product.

Synthesis of 4-31

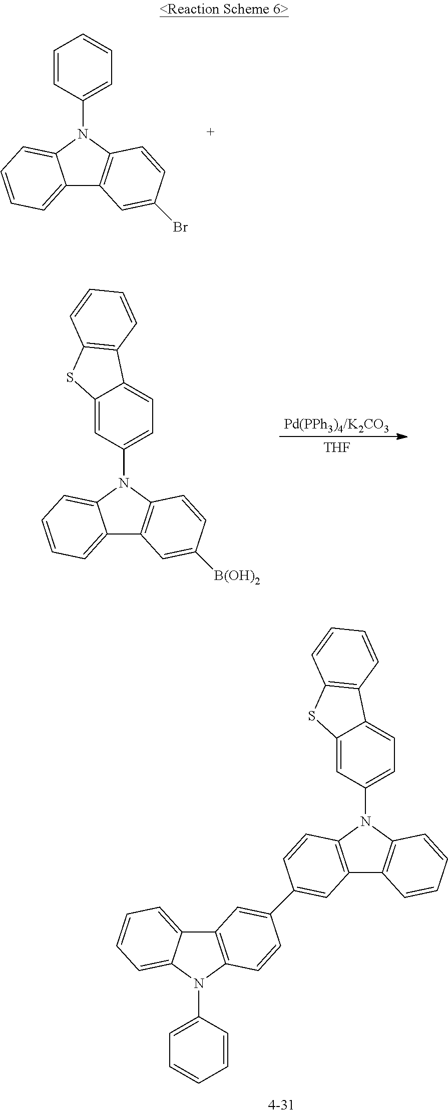 [SMILES: Brc1ccc2c(c1)c1ccccc1n2-c1ccccc1 ; OB(O)c1ccc2c(c1)c1ccccc1n2-c1ccc2c(c1)sc1ccccc12 ; c1ccc(-n2c3ccccc3c3cc(-c4ccc5c(c4)c4ccccc4n5-c4ccc5c(c4)sc4ccccc45)ccc32)cc1 ; "THF"]

After dissolving 3-bromo-9-phenyl-9H-carbazole (6.4 g, 20 mmol) in THF, (9-(dibenzo[b,d]thiophen-3-yl)-9H-carbazol-3-yl)boronic acid (7.8 g, 20 mmol), Pd(PPh₃)₄ (0.03 eq.), K₂CO₃(3 eq.) and water were added thereto and the mixture was stirred under reflux. When the reaction was completed, the reaction product was extracted with ether and water. The organic layer was dried with MgSO₄ and concentrated. Thereafter, the concentrate was passed through a silica gel column and recrystallized to obtain 8.5 g (yield: 72%) of product.

### Fabrication and Evaluation of Organic Electric Element

### [Example 1] Red OLED (Host)

After vacuum-depositing N¹-(naphthalen-2-yl)-N⁴,N⁴-bis(4-(naphthalen-2-yl(phenyl)amino)phenyl)-N¹-phenylbenzene-1,4-diamine (hereinafter, “2-TNATA”) on an ITO layer (anode) formed on a glass substrate to form a hole injection layer with a thickness of 60 nm, a hole transport layer with a thickness of 60 nm was formed by vacuum-depositing the compound 1-33′ of the present invention on the hole injection layer.

Thereafter, on the hole transport layer, the compound 1-26′ of the present invention was vacuum-deposited to a thickness of 20 nm to form an emission-auxiliary layer.

Next, on the emission-auxiliary layer, the compound 3-5 of the present invention as a host material and bis-(1-phenylisoquinolyl)iridium(III)acetylacetonate (hereinafter, “(piq)₂Ir(acac)”) as a dopant material in a weight ratio of 95:5 were deposited on the hole transport layer to form a light emitting layer with a thickness of 30 nm.

Next, 1,1′-bisphenyl-4-olato)bis(2-methyl-8-quinolinolato)aluminum (hereinafter, “BAlq”) was vacuum-deposited to a thickness of 10 nm on the light emitting layer to form a hole blocking layer, and tris(8-quinolinolato)aluminum (hereinafter, “Alq3”) was vacuum-deposited to a thickness of 40 nm on the hole blocking layer to form a an electron transport layer.

Thereafter, LiF was deposited to a thickness of 0.2 nm to form an electron injection layer on the electron transport layer, and then Al was deposited to a thickness of 150 nm to form a cathode on the electron injection layer.

### Example 2 to Example 207

An organic electroluminescent element was manufactured in the same manner as in Example 1, using the compounds shown in Table 1 below as a material for the hole transport layer, the emission-auxiliary layer and the light emitting layer.

### Comparative Example 1

An organic electroluminescent element was manufactured in the same manner as in Example 1 except that N,N′-Bis(1-naphthalenyl)-N,N′-bis-phenyl-(1,1′-biphenyl)-4,4′-diamine (hereinafter abbreviated as “NPB”) was used as a material for the hole transport layer, an emission-auxiliary layer r was not formed and 4,4′-N,N′-dicarbazole-biphenyl (hereinafter referred to as “CBP”) was used as the host of a light-emitting layer.

### Comparative Example 2

An organic electroluminescent element was manufactured in the same manner as in Example 1 except that NPB was used as a material for the hole transport layer, the compound P-73 was used as a material for an emission-auxiliary layer and CBP was used as the host of a light-emitting layer.

### Comparative Example 3

An organic electroluminescent element was manufactured in the same manner as in Example 1 except that the compound 2-75 was used as a material for the hole transport layer, the compound P-73 was used as a material for an emission-auxiliary layer and CBP was used as the host of a light-emitting layer.

### Comparative Example 4

An organic electroluminescent element was manufactured in the same manner as in Example 1 except that NPB was used as a material for the hole transport layer, the compound P-73 was used as a material for an emission-auxiliary layer and the compound 3-5 was used as the host of a light-emitting layer.

Electroluminescence (EL) characteristics were measured with a PR-650 (Photoresearch) by applying a forward bias DC voltage to the OLEDs prepared in Examples 1 to 207 of the present invention and Comparative Examples 1 to 4. And, the T95 life time was measured using a life time measuring apparatus manufactured by ms science Inc. at reference brightness of 2500 cd/m². The measurement results are shown in Tables 2 below.

TABLE 1

| | Hole transport | Emission-auxiliary | Light-emitting |
|---|---|---|---|
| | layer compd. | layer compd. | layer compd. |
| comp. Ex (1) | NPB | — | CBP |
| comp. Ex (2) | NPB | P-73 | CBP |
| comp. Ex (3) | 2-75 | P-73 | CBP |
| comp. Ex (4) | NPB | P-73 | 3-5 |
| Ex. (1) | 1-33′ | 1-26′ | 3-5 |
| Ex. (2) | | | 3-13 |
| Ex. (3) | | | 3-22 |
| Ex. (4) | | | 3-23 |
| Ex. (5) | | | 3-24 |
| Ex. (6) | | | 3-26 |
| Ex. (7) | | | 3-58 |
| Ex. (8) | | | 3-59 |
| Ex. (9) | | | 8-1 |
| Ex. (10) | | 1-52′ | 3-5 |
| Ex. (11) | | | 3-13 |
| Ex. (12) | | | 3-22 |
| Ex. (13) | | | 3-23 |
| Ex. (14) | | | 3-24 |
| Ex. (15) | | | 3-26 |
| Ex. (16) | | | 3-58 |
| Ex. (17) | | | 3-59 |
| Ex. (18) | | | 8-1 |
| Ex. (19) | | 1-64′ | 3-5 |
| Ex. (20) | | | 3-13 |
| Ex. (21) | | | 3-22 |
| Ex. (22) | | | 3-23 |
| Ex. (23) | | | 3-24 |
| Ex. (24) | | | 3-26 |
| Ex. (25) | | | 3-58 |
| Ex. (26) | | | 3-59 |
| Ex. (27) | | | 8-1 |
| Ex. (28) | | 2-75 | 3-5 |
| Ex. (29) | | | 3-13 |
| Ex. (30) | | | 3-22 |
| Ex. (31) | | | 3-23 |
| Ex. (32) | | | 3-24 |
| Ex. (33) | | | 3-26 |
| Ex. (34) | | | 3-58 |
| Ex. (35) | | | 3-59 |
| Ex. (36) | | | 8-1 |
| Ex. (37) | | P-5 | 3-5 |
| Ex. (38) | | | 3-13 |
| Ex. (39) | | | 3-22 |
| Ex. (40) | | | 3-23 |
| Ex. (41) | | | 3-24 |
| Ex. (42) | | | 3-26 |
| Ex. (43) | | | 3-58 |
| Ex. (44) | | | 3-59 |
| Ex. (45) | | | 8-1 |
| Ex. (46) | P-73 | 1-26′ | 3-5 |
| Ex. (47) | | | 3-13 |
| Ex. (48) | | | 3-22 |
| Ex. (49) | | | 3-23 |
| Ex. (50) | | | 3-24 |
| Ex. (51) | | | 3-26 |
| Ex. (52) | | | 3-58 |
| Ex. (53) | | | 3-59 |
| Ex. (54) | | | 8-1 |
| Ex. (55) | | 1-52′ | 3-5 |
| Ex. (56) | | | 3-13 |
| Ex. (57) | | | 3-22 |
| Ex. (58) | | | 3-23 |
| Ex. (59) | | | 3-24 |
| Ex. (60) | | | 3-26 |
| Ex. (61) | | | 3-58 |
| Ex. (62) | | | 3-59 |
| Ex. (63) | | | 8-1 |
| Ex. (64) | | 1-64′ | 3-5 |
| Ex. (65) | | | 3-13 |
| Ex. (66) | | | 3-22 |
| Ex. (67) | | | 3-23 |
| Ex. (68) | | | 3-24 |
| Ex. (69) | | | 3-26 |
| Ex. (70) | | | 3-58 |
| Ex. (71) | | | 3-59 |
| Ex. (72) | | | 8-1 |
| Ex. (73) | | P-82 | 3-5 |
| Ex. (74) | | | 3-13 |
| Ex. (75) | | | 3-22 |
| Ex. (76) | | | 3-23 |
| Ex. (77) | | | 3-24 |
| Ex. (78) | | | 3-26 |
| Ex. (79) | | | 3-58 |
| Ex. (80) | | | 3-59 |
| Ex. (81) | | | 8-1 |
| Ex. (82) | | 2-64 | 3-5 |
| Ex. (83) | | | 3-13 |
| Ex. (84) | | | 3-22 |
| Ex. (85) | | | 3-23 |
| Ex. (86) | | | 3-24 |
| Ex. (87) | | | 3-26 |
| Ex. (88) | | | 3-58 |
| Ex. (89) | | | 3-59 |
| Ex. (90) | | | 8-1 |
| Ex. (91) | | 2-75 | 3-5 |
| Ex. (92) | | | 3-13 |
| Ex. (93) | | | 3-22 |
| Ex. (94) | | | 3-23 |
| Ex. (95) | | | 3-24 |
| Ex. (96) | | | 3-26 |
| Ex. (97) | | | 3-58 |
| Ex. (98) | | | 3-59 |
| Ex. (99) | | | 8-1 |
| Ex. (100) | 2-64 | 1-26′ | 3-5 |
| Ex. (101) | | | 3-13 |
| Ex. (102) | | | 3-22 |
| Ex. (103) | | | 3-23 |
| Ex. (104) | | | 3-24 |
| Ex. (105) | | | 3-26 |
| Ex. (106) | | | 3-58 |
| Ex. (107) | | | 3-59 |
| Ex. (108) | | | 8-1 |
| Ex. (109) | | 1-52′ | 3-5 |
| Ex. (110) | | | 3-13 |
| Ex. (111) | | | 3-22 |
| Ex. (112) | | | 3-23 |
| Ex. (113) | | | 3-24 |
| Ex. (114) | | | 3-26 |
| Ex. (115) | | | 3-58 |
| Ex. (116) | | | 3-59 |
| Ex. (117) | | | 8-1 |
| Ex. (118) | | 1-64′ | 3-5 |
| Ex. (119) | | | 3-13 |
| Ex. (120) | | | 3-22 |
| Ex. (121) | | | 3-23 |
| Ex. (122) | | | 3-24 |
| Ex. (123) | | | 3-26 |
| Ex. (124) | | | 3-58 |
| Ex. (125) | | | 3-59 |
| Ex. (126) | | | 8-1 |
| Ex. (127) | | P-5 | 3-5 |
| Ex. (128) | | | 3-13 |
| Ex. (129) | | | 3-22 |
| Ex. (130) | | | 3-23 |
| Ex. (131) | | | 3-24 |
| Ex. (132) | | | 3-26 |
| Ex. (133) | | | 3-58 |
| Ex. (134) | | | 3-59 |
| Ex. (135) | | | 8-1 |
| Ex. (136) | | P-73 | 3-5 |
| Ex. (137) | | | 3-13 |
| Ex. (138) | | | 3-22 |
| Ex. (139) | | | 3-23 |
| Ex. (140) | | | 3-24 |
| Ex. (141) | | | 3-26 |
| Ex. (142) | | | 3-58 |
| Ex. (143) | | | 3-59 |
| Ex. (144) | | | 8-1 |
| Ex. (145) | | P-82 | 3-5 |
| Ex. (146) | | | 3-13 |
| Ex. (147) | | | 3-22 |
| Ex. (148) | | | 3-23 |
| Ex. (149) | | | 3-24 |
| Ex. (150) | | | 3-26 |
| Ex. (151) | | | 3-58 |
| Ex. (152) | | | 3-59 |
| Ex. (153) | | | 8-1 |
| Ex. (154) | 2-75 | 1-26′ | 3-5 |
| Ex. (155) | | | 3-13 |
| Ex. (156) | | | 3-22 |
| Ex. (157) | | | 3-23 |
| Ex. (158) | | | 3-24 |
| Ex. (159) | | | 3-26 |
| Ex. (160) | | | 3-58 |
| Ex. (161) | | | 3-59 |
| Ex. (162) | | | 8-1 |
| Ex. (163) | | 1-52′ | 3-5 |
| Ex. (164) | | | 3-13 |
| Ex. (165) | | | 3-22 |
| Ex. (166) | | | 3-23 |
| Ex. (167) | | | 3-24 |
| Ex. (168) | | | 3-26 |
| Ex. (169) | | | 3-58 |
| Ex. (170) | | | 3-59 |
| Ex. (171) | | | 8-1 |
| Ex. (172) | | 1-64′ | 3-5 |
| Ex. (173) | | | 3-13 |
| Ex. (174) | | | 3-22 |
| Ex. (175) | | | 3-23 |
| Ex. (176) | | | 3-24 |
| Ex. (177) | | | 3-26 |
| Ex. (178) | | | 3-58 |
| Ex. (179) | | | 3-59 |
| Ex. (180) | | | 8-1 |
| Ex. (181) | | P-5 | 3-5 |
| Ex. (182) | | | 3-13 |
| Ex. (183) | | | 3-22 |
| Ex. (184) | | | 3-23 |
| Ex. (185) | | | 3-24 |
| Ex. (186) | | | 3-26 |
| Ex. (187) | | | 3-58 |
| Ex. (188) | | | 3-59 |
| Ex. (189) | | | 8-1 |
| Ex. (190) | | P-73 | 3-5 |
| Ex. (191) | | | 3-13 |
| Ex. (192) | | | 3-22 |
| Ex. (193) | | | 3-23 |
| Ex. (194) | | | 3-24 |
| Ex. (195) | | | 3-26 |
| Ex. (196) | | | 3-58 |
| Ex. (197) | | | 3-59 |
| Ex. (198) | | | 8-1 |
| Ex. (199) | | P-82 | 3-5 |
| Ex. (200) | | | 3-13 |
| Ex. (201) | | | 3-22 |
| Ex. (202) | | | 3-23 |
| Ex. (203) | | | 3-24 |
| Ex. (204) | | | 3-26 |
| Ex. (205) | | | 3-58 |
| Ex. (206) | | | 3-59 |
| Ex. (207) | | | 8-1 |

TABLE 2

| | | Current | Bright- | Effi- | Life- | | |
|---|---|---|---|---|---|---|---|
| | Voltage | Density | ness | ciency | time | CIE | |
| | (V) | (mA/cm²) | (cd/m²) | (cd/A) | T(95) | x | y |
| comp. Ex(1) | 6.0 | 32.9 | 2500.0 | 7.6 | 61.8 | 0.66 | 0.32 |
| comp. Ex(2) | 5.9 | 24.8 | 2500.0 | 10.1 | 90.4 | 0.60 | 0.33 |
| comp. Ex(3) | 5.5 | 19.4 | 2500.0 | 12.9 | 98.7 | 0.62 | 0.33 |
| comp. Ex(4) | 5.7 | 16.3 | 2500.0 | 15.3 | 95.3 | 0.62 | 0.34 |
| Ex. (1) | 5.0 | 11.0 | 2500.0 | 22.6 | 111.4 | 0.62 | 0.32 |
| Ex. (2) | 5.0 | 11.2 | 2500.0 | 22.3 | 111.1 | 0.61 | 0.33 |
| Ex. (3) | 5.0 | 11.1 | 2500.0 | 22.5 | 112.5 | 0.60 | 0.32 |
| Ex. (4) | 4.9 | 10.9 | 2500.0 | 23.0 | 112.3 | 0.64 | 0.34 |
| Ex. (5) | 5.0 | 11.0 | 2500.0 | 22.6 | 113.4 | 0.63 | 0.31 |
| Ex. (6) | 5.0 | 11.3 | 2500.0 | 22.2 | 113.2 | 0.61 | 0.31 |
| Ex. (7) | 4.9 | 11.2 | 2500.0 | 22.4 | 113.6 | 0.61 | 0.32 |
| Ex. (8) | 4.9 | 10.9 | 2500.0 | 23.0 | 113.3 | 0.62 | 0.30 |
| Ex. (9) | 4.9 | 11.2 | 2500.0 | 22.3 | 111.2 | 0.65 | 0.33 |
| Ex. (10) | 4.9 | 11.2 | 2500.0 | 22.4 | 112.0 | 0.62 | 0.32 |
| Ex. (11) | 4.9 | 11.1 | 2500.0 | 22.5 | 113.0 | 0.60 | 0.30 |
| Ex. (12) | 5.0 | 11.3 | 2500.0 | 22.1 | 110.9 | 0.64 | 0.31 |
| Ex. (13) | 4.9 | 11.2 | 2500.0 | 22.4 | 110.7 | 0.63 | 0.33 |
| Ex. (14) | 5.0 | 11.2 | 2500.0 | 22.3 | 112.2 | 0.63 | 0.30 |
| Ex. (15) | 5.0 | 11.0 | 2500.0 | 22.7 | 112.5 | 0.62 | 0.34 |
| Ex. (16) | 4.9 | 10.9 | 2500.0 | 22.9 | 113.0 | 0.64 | 0.33 |
| Ex. (17) | 5.0 | 11.1 | 2500.0 | 22.5 | 111.9 | 0.60 | 0.35 |
| Ex. (18) | 5.0 | 11.2 | 2500.0 | 22.3 | 113.1 | 0.61 | 0.31 |
| Ex. (19) | 4.9 | 11.2 | 2500.0 | 22.3 | 114.0 | 0.65 | 0.35 |
| Ex. (20) | 5.0 | 11.1 | 2500.0 | 22.6 | 112.1 | 0.62 | 0.31 |
| Ex. (21) | 5.0 | 11.3 | 2500.0 | 22.2 | 113.5 | 0.63 | 0.32 |
| Ex. (22) | 4.9 | 11.0 | 2500.0 | 22.8 | 110.6 | 0.63 | 0.32 |
| Ex. (23) | 4.9 | 11.3 | 2500.0 | 22.0 | 111.2 | 0.62 | 0.33 |
| Ex. (24) | 4.9 | 11.0 | 2500.0 | 22.8 | 113.1 | 0.64 | 0.30 |
| Ex. (25) | 5.0 | 11.3 | 2500.0 | 22.2 | 114.9 | 0.60 | 0.32 |
| Ex. (26) | 5.0 | 11.1 | 2500.0 | 22.5 | 112.6 | 0.60 | 0.34 |
| Ex. (27) | 4.9 | 11.3 | 2500.0 | 22.1 | 113.1 | 0.62 | 0.33 |
| Ex. (28) | 5.2 | 12.2 | 2500.0 | 20.5 | 113.2 | 0.64 | 0.34 |
| Ex. (29) | 5.2 | 12.1 | 2500.0 | 20.7 | 114.2 | 0.60 | 0.33 |
| Ex. (30) | 5.1 | 12.3 | 2500.0 | 20.3 | 111.1 | 0.65 | 0.31 |
| Ex. (31) | 5.2 | 12.1 | 2500.0 | 20.7 | 112.6 | 0.62 | 0.34 |
| Ex. (32) | 5.2 | 12.4 | 2500.0 | 20.2 | 110.4 | 0.60 | 0.31 |
| Ex. (33) | 5.2 | 12.4 | 2500.0 | 20.2 | 114.1 | 0.65 | 0.31 |
| Ex. (34) | 5.1 | 12.1 | 2500.0 | 20.6 | 113.8 | 0.61 | 0.30 |
| Ex. (35) | 5.1 | 12.2 | 2500.0 | 20.5 | 111.9 | 0.60 | 0.33 |
| Ex. (36) | 5.2 | 12.4 | 2500.0 | 20.2 | 113.5 | 0.64 | 0.31 |
| Ex. (37) | 4.8 | 10.1 | 2500.0 | 24.7 | 114.2 | 0.65 | 0.32 |
| Ex. (38) | 4.8 | 10.0 | 2500.0 | 25.0 | 111.0 | 0.61 | 0.32 |
| Ex. (39) | 4.9 | 10.1 | 2500.0 | 24.8 | 112.8 | 0.62 | 0.34 |
| Ex. (40) | 4.8 | 10.2 | 2500.0 | 24.5 | 112.6 | 0.64 | 0.34 |
| Ex. (41) | 4.9 | 10.3 | 2500.0 | 24.2 | 110.5 | 0.63 | 0.34 |
| Ex. (42) | 4.8 | 10.3 | 2500.0 | 24.2 | 113.5 | 0.62 | 0.35 |
| Ex. (43) | 4.9 | 10.2 | 2500.0 | 24.4 | 110.9 | 0.64 | 0.32 |
| Ex. (44) | 4.8 | 10.4 | 2500.0 | 24.0 | 114.3 | 0.63 | 0.32 |
| Ex. (45) | 4.8 | 10.0 | 2500.0 | 24.9 | 111.4 | 0.61 | 0.32 |
| Ex. (46) | 5.0 | 11.2 | 2500.0 | 22.4 | 109.9 | 0.64 | 0.32 |
| Ex. (47) | 5.0 | 11.3 | 2500.0 | 22.2 | 105.1 | 0.64 | 0.33 |
| Ex. (48) | 5.0 | 11.2 | 2500.0 | 22.3 | 108.4 | 0.63 | 0.33 |
| Ex. (49) | 4.9 | 11.2 | 2500.0 | 22.3 | 108.1 | 0.62 | 0.34 |
| Ex. (50) | 4.9 | 11.0 | 2500.0 | 22.8 | 109.1 | 0.62 | 0.30 |
| Ex. (51) | 5.0 | 11.0 | 2500.0 | 22.7 | 106.5 | 0.62 | 0.35 |
| Ex. (52) | 4.9 | 11.2 | 2500.0 | 22.4 | 108.8 | 0.60 | 0.31 |
| Ex. (53) | 4.9 | 11.4 | 2500.0 | 22.0 | 105.8 | 0.61 | 0.32 |
| Ex. (54) | 5.0 | 11.0 | 2500.0 | 22.7 | 107.4 | 0.63 | 0.32 |
| Ex. (55) | 4.9 | 11.3 | 2500.0 | 22.1 | 107.8 | 0.61 | 0.34 |
| Ex. (56) | 4.9 | 11.0 | 2500.0 | 22.7 | 105.5 | 0.61 | 0.32 |
| Ex. (57) | 4.9 | 10.9 | 2500.0 | 22.9 | 108.4 | 0.62 | 0.30 |
| Ex. (58) | 5.0 | 11.1 | 2500.0 | 22.5 | 108.5 | 0.61 | 0.35 |
| Ex. (59) | 4.9 | 11.3 | 2500.0 | 22.1 | 106.1 | 0.64 | 0.32 |
| Ex. (60) | 5.0 | 11.0 | 2500.0 | 22.7 | 107.3 | 0.61 | 0.32 |
| Ex. (61) | 4.9 | 11.0 | 2500.0 | 22.7 | 107.5 | 0.61 | 0.30 |
| Ex. (62) | 4.9 | 11.4 | 2500.0 | 22.0 | 109.3 | 0.62 | 0.30 |
| Ex. (63) | 5.0 | 11.0 | 2500.0 | 22.7 | 105.4 | 0.61 | 0.32 |
| Ex. (64) | 4.9 | 10.9 | 2500.0 | 23.0 | 107.5 | 0.63 | 0.33 |
| Ex. (65) | 4.9 | 11.1 | 2500.0 | 22.6 | 106.2 | 0.65 | 0.33 |
| Ex. (66) | 4.9 | 11.0 | 2500.0 | 22.8 | 105.5 | 0.60 | 0.35 |
| Ex. (67) | 4.9 | 10.9 | 2500.0 | 22.9 | 109.0 | 0.63 | 0.33 |
| Ex. (68) | 5.0 | 11.1 | 2500.0 | 22.5 | 108.2 | 0.62 | 0.35 |
| Ex. (69) | 5.0 | 11.2 | 2500.0 | 22.4 | 106.3 | 0.63 | 0.30 |
| Ex. (70) | 4.9 | 11.2 | 2500.0 | 22.3 | 109.4 | 0.61 | 0.33 |
| Ex. (71) | 5.0 | 11.3 | 2500.0 | 22.1 | 108.1 | 0.60 | 0.32 |
| Ex. (72) | 4.9 | 10.9 | 2500.0 | 22.8 | 108.2 | 0.64 | 0.34 |
| Ex. (73) | 4.9 | 10.3 | 2500.0 | 24.4 | 105.3 | 0.61 | 0.32 |
| Ex. (74) | 4.9 | 10.4 | 2500.0 | 24.1 | 109.1 | 0.62 | 0.31 |
| Ex. (75) | 4.9 | 10.4 | 2500.0 | 24.0 | 105.5 | 0.61 | 0.32 |
| Ex. (76) | 4.8 | 10.4 | 2500.0 | 24.1 | 108.0 | 0.64 | 0.35 |
| Ex. (77) | 4.8 | 10.2 | 2500.0 | 24.5 | 105.7 | 0.60 | 0.34 |
| Ex. (78) | 4.8 | 10.1 | 2500.0 | 24.7 | 106.0 | 0.61 | 0.31 |
| Ex. (79) | 4.9 | 10.3 | 2500.0 | 24.2 | 106.5 | 0.64 | 0.34 |
| Ex. (80) | 4.8 | 10.4 | 2500.0 | 24.1 | 106.5 | 0.63 | 0.32 |
| Ex. (81) | 4.8 | 10.4 | 2500.0 | 24.1 | 106.7 | 0.61 | 0.34 |
| Ex. (82) | 5.2 | 12.3 | 2500.0 | 20.4 | 105.9 | 0.62 | 0.30 |
| Ex. (83) | 5.2 | 12.5 | 2500.0 | 20.1 | 108.9 | 0.61 | 0.34 |
| Ex. (84) | 5.2 | 12.4 | 2500.0 | 20.1 | 107.6 | 0.65 | 0.34 |
| Ex. (85) | 5.1 | 12.0 | 2500.0 | 20.9 | 108.1 | 0.65 | 0.32 |
| Ex. (86) | 5.1 | 12.4 | 2500.0 | 20.2 | 106.4 | 0.64 | 0.32 |
| Ex. (87) | 5.2 | 12.4 | 2500.0 | 20.2 | 105.5 | 0.62 | 0.34 |
| Ex. (88) | 5.2 | 12.2 | 2500.0 | 20.5 | 109.1 | 0.63 | 0.34 |
| Ex. (89) | 5.2 | 12.5 | 2500.0 | 20.0 | 108.7 | 0.62 | 0.30 |
| Ex. (90) | 5.2 | 12.1 | 2500.0 | 20.7 | 109.3 | 0.64 | 0.30 |
| Ex. (91) | 5.1 | 12.3 | 2500.0 | 20.3 | 109.5 | 0.62 | 0.30 |
| Ex. (92) | 5.1 | 12.4 | 2500.0 | 20.2 | 105.9 | 0.64 | 0.33 |
| Ex. (93) | 5.1 | 12.5 | 2500.0 | 20.1 | 108.4 | 0.60 | 0.30 |
| Ex. (94) | 5.2 | 12.4 | 2500.0 | 20.2 | 106.1 | 0.62 | 0.32 |
| Ex. (95) | 5.1 | 11.9 | 2500.0 | 20.9 | 107.4 | 0.62 | 0.32 |
| Ex. (96) | 5.2 | 12.4 | 2500.0 | 20.1 | 105.5 | 0.61 | 0.34 |
| Ex. (97) | 5.1 | 12.5 | 2500.0 | 20.0 | 107.3 | 0.65 | 0.34 |
| Ex. (98) | 5.1 | 12.3 | 2500.0 | 20.3 | 110.0 | 0.63 | 0.32 |
| Ex. (99) | 5.1 | 12.2 | 2500.0 | 20.6 | 108.9 | 0.62 | 0.34 |
| Ex. (100) | 4.5 | 9.2 | 2500.0 | 27.3 | 118.9 | 0.63 | 0.33 |
| Ex. (101) | 4.5 | 9.0 | 2500.0 | 27.6 | 115.3 | 0.65 | 0.34 |
| Ex. (102) | 4.5 | 9.1 | 2500.0 | 27.5 | 119.8 | 0.65 | 0.31 |
| Ex. (103) | 4.6 | 9.1 | 2500.0 | 27.4 | 118.8 | 0.63 | 0.33 |
| Ex. (104) | 4.5 | 9.2 | 2500.0 | 27.1 | 118.0 | 0.65 | 0.30 |
| Ex. (105) | 4.6 | 9.0 | 2500.0 | 27.7 | 119.1 | 0.61 | 0.35 |
| Ex. (106) | 4.5 | 8.9 | 2500.0 | 28.0 | 119.8 | 0.62 | 0.34 |
| Ex. (107) | 4.6 | 9.0 | 2500.0 | 27.8 | 118.4 | 0.62 | 0.34 |
| Ex. (108) | 4.6 | 9.0 | 2500.0 | 27.8 | 118.7 | 0.63 | 0.30 |
| Ex. (109) | 4.6 | 9.1 | 2500.0 | 27.5 | 115.9 | 0.65 | 0.30 |
| Ex. (110) | 4.5 | 9.0 | 2500.0 | 27.8 | 118.0 | 0.65 | 0.33 |
| Ex. (111) | 4.6 | 9.0 | 2500.0 | 27.8 | 119.3 | 0.64 | 0.34 |
| Ex. (112) | 4.5 | 9.0 | 2500.0 | 27.6 | 119.8 | 0.64 | 0.35 |
| Ex. (113) | 4.6 | 9.2 | 2500.0 | 27.2 | 119.6 | 0.63 | 0.30 |
| Ex. (114) | 4.6 | 9.0 | 2500.0 | 27.9 | 116.0 | 0.62 | 0.34 |
| Ex. (115) | 4.5 | 9.2 | 2500.0 | 27.3 | 116.8 | 0.63 | 0.34 |
| Ex. (116) | 4.5 | 9.2 | 2500.0 | 27.3 | 116.0 | 0.64 | 0.34 |
| Ex. (117) | 4.5 | 9.2 | 2500.0 | 27.3 | 118.2 | 0.64 | 0.34 |
| Ex. (118) | 4.6 | 9.1 | 2500.0 | 27.5 | 117.8 | 0.62 | 0.32 |
| Ex. (119) | 4.5 | 8.9 | 2500.0 | 27.9 | 119.0 | 0.61 | 0.31 |
| Ex. (120) | 4.5 | 9.1 | 2500.0 | 27.6 | 116.2 | 0.63 | 0.35 |
| Ex. (121) | 4.6 | 9.0 | 2500.0 | 27.8 | 118.1 | 0.64 | 0.33 |
| Ex. (122) | 4.5 | 9.1 | 2500.0 | 27.5 | 117.4 | 0.62 | 0.33 |
| Ex. (123) | 4.6 | 9.1 | 2500.0 | 27.5 | 115.8 | 0.61 | 0.30 |
| Ex. (124) | 4.5 | 9.1 | 2500.0 | 27.4 | 116.1 | 0.62 | 0.33 |
| Ex. (125) | 4.6 | 9.2 | 2500.0 | 27.1 | 118.1 | 0.60 | 0.32 |
| Ex. (126) | 4.5 | 9.2 | 2500.0 | 27.2 | 118.4 | 0.62 | 0.33 |
| Ex. (127) | 4.4 | 8.5 | 2500.0 | 29.4 | 116.0 | 0.63 | 0.30 |
| Ex. (128) | 4.3 | 8.3 | 2500.0 | 30.0 | 118.1 | 0.65 | 0.31 |
| Ex. (129) | 4.3 | 8.0 | 2500.0 | 31.3 | 123.5 | 0.62 | 0.31 |
| Ex. (130) | 4.4 | 8.5 | 2500.0 | 29.5 | 116.6 | 0.64 | 0.32 |
| Ex. (131) | 4.3 | 8.4 | 2500.0 | 29.8 | 119.4 | 0.63 | 0.34 |
| Ex. (132) | 4.3 | 8.4 | 2500.0 | 29.8 | 119.1 | 0.62 | 0.30 |
| Ex. (133) | 4.4 | 8.4 | 2500.0 | 29.7 | 115.8 | 0.62 | 0.34 |
| Ex. (134) | 4.3 | 8.4 | 2500.0 | 29.7 | 115.7 | 0.62 | 0.33 |
| Ex. (135) | 4.4 | 8.5 | 2500.0 | 29.3 | 117.9 | 0.62 | 0.33 |
| Ex. (136) | 4.3 | 8.3 | 2500.0 | 30.0 | 117.2 | 0.65 | 0.30 |
| Ex. (137) | 4.4 | 8.5 | 2500.0 | 29.6 | 115.5 | 0.64 | 0.33 |
| Ex. (138) | 4.3 | 8.5 | 2500.0 | 29.4 | 119.8 | 0.64 | 0.32 |
| Ex. (139) | 4.3 | 8.6 | 2500.0 | 29.1 | 118.6 | 0.63 | 0.34 |
| Ex. (140) | 4.3 | 8.5 | 2500.0 | 29.5 | 116.2 | 0.63 | 0.31 |
| Ex. (141) | 4.3 | 8.6 | 2500.0 | 29.2 | 115.6 | 0.61 | 0.34 |
| Ex. (142) | 4.3 | 8.6 | 2500.0 | 29.0 | 119.4 | 0.64 | 0.31 |
| Ex. (143) | 4.3 | 8.5 | 2500.0 | 29.3 | 120.0 | 0.65 | 0.34 |
| Ex. (144) | 4.4 | 8.6 | 2500.0 | 29.1 | 117.7 | 0.64 | 0.33 |
| Ex. (145) | 4.3 | 8.5 | 2500.0 | 29.4 | 119.1 | 0.63 | 0.35 |
| Ex. (146) | 4.3 | 8.5 | 2500.0 | 29.5 | 115.4 | 0.62 | 0.32 |
| Ex. (147) | 4.3 | 8.5 | 2500.0 | 29.5 | 118.6 | 0.62 | 0.35 |
| Ex. (148) | 4.3 | 8.6 | 2500.0 | 29.2 | 118.0 | 0.62 | 0.31 |
| Ex. (149) | 4.4 | 8.5 | 2500.0 | 29.5 | 119.7 | 0.61 | 0.30 |
| Ex. (150) | 4.3 | 8.5 | 2500.0 | 29.3 | 118.2 | 0.61 | 0.31 |
| Ex. (151) | 4.3 | 8.5 | 2500.0 | 29.5 | 119.1 | 0.63 | 0.35 |
| Ex. (152) | 4.3 | 8.4 | 2500.0 | 29.8 | 115.7 | 0.63 | 0.32 |
| Ex. (153) | 4.4 | 8.5 | 2500.0 | 29.5 | 115.8 | 0.64 | 0.33 |
| Ex. (154) | 4.5 | 9.3 | 2500.0 | 26.8 | 119.8 | 0.64 | 0.35 |
| Ex. (155) | 4.5 | 9.4 | 2500.0 | 26.7 | 116.5 | 0.62 | 0.32 |
| Ex. (156) | 4.6 | 9.3 | 2500.0 | 26.8 | 116.2 | 0.64 | 0.35 |
| Ex. (157) | 4.6 | 9.4 | 2500.0 | 26.6 | 116.9 | 0.64 | 0.32 |
| Ex. (158) | 4.6 | 9.3 | 2500.0 | 26.8 | 118.5 | 0.64 | 0.30 |
| Ex. (159) | 4.6 | 9.3 | 2500.0 | 26.9 | 119.7 | 0.63 | 0.34 |
| Ex. (160) | 4.6 | 9.6 | 2500.0 | 26.2 | 115.5 | 0.63 | 0.35 |
| Ex. (161) | 4.6 | 9.3 | 2500.0 | 26.9 | 116.0 | 0.64 | 0.33 |
| Ex. (162) | 4.6 | 9.4 | 2500.0 | 26.5 | 116.4 | 0.61 | 0.31 |
| Ex. (163) | 4.6 | 9.4 | 2500.0 | 26.5 | 117.0 | 0.64 | 0.33 |
| Ex. (164) | 4.5 | 9.5 | 2500.0 | 26.5 | 116.6 | 0.63 | 0.33 |
| Ex. (165) | 4.5 | 9.4 | 2500.0 | 26.5 | 118.5 | 0.65 | 0.32 |
| Ex. (166) | 4.6 | 9.5 | 2500.0 | 26.4 | 119.5 | 0.64 | 0.33 |
| Ex. (167) | 4.6 | 9.5 | 2500.0 | 26.3 | 117.0 | 0.62 | 0.32 |
| Ex. (168) | 4.6 | 9.5 | 2500.0 | 26.4 | 117.3 | 0.64 | 0.31 |
| Ex. (169) | 4.5 | 9.3 | 2500.0 | 27.0 | 116.9 | 0.60 | 0.32 |
| Ex. (170) | 4.6 | 9.4 | 2500.0 | 26.7 | 119.1 | 0.62 | 0.31 |
| Ex. (171) | 4.5 | 9.5 | 2500.0 | 26.4 | 117.7 | 0.60 | 0.34 |
| Ex. (172) | 4.5 | 9.4 | 2500.0 | 26.7 | 119.0 | 0.63 | 0.30 |
| Ex. (173) | 4.5 | 9.6 | 2500.0 | 26.1 | 119.7 | 0.63 | 0.31 |
| Ex. (174) | 4.6 | 9.5 | 2500.0 | 26.4 | 115.2 | 0.61 | 0.33 |
| Ex. (175) | 4.6 | 9.3 | 2500.0 | 27.0 | 117.7 | 0.63 | 0.33 |
| Ex. (176) | 4.6 | 9.5 | 2500.0 | 26.3 | 118.7 | 0.65 | 0.31 |
| Ex. (177) | 4.5 | 9.4 | 2500.0 | 26.6 | 115.4 | 0.63 | 0.33 |
| Ex. (178) | 4.5 | 9.5 | 2500.0 | 26.2 | 115.7 | 0.61 | 0.34 |
| Ex. (179) | 4.5 | 9.4 | 2500.0 | 26.6 | 118.4 | 0.62 | 0.32 |
| Ex. (180) | 4.6 | 9.4 | 2500.0 | 26.6 | 119.1 | 0.63 | 0.31 |
| Ex. (181) | 4.3 | 9.4 | 2500.0 | 26.7 | 118.5 | 0.62 | 0.33 |
| Ex. (182) | 4.3 | 8.8 | 2500.0 | 28.5 | 116.9 | 0.64 | 0.34 |
| Ex. (183) | 4.3 | 8.7 | 2500.0 | 28.7 | 115.6 | 0.64 | 0.35 |
| Ex. (184) | 4.3 | 8.6 | 2500.0 | 28.9 | 115.3 | 0.62 | 0.32 |
| Ex. (185) | 4.4 | 8.6 | 2500.0 | 28.9 | 119.3 | 0.61 | 0.32 |
| Ex. (186) | 4.3 | 8.7 | 2500.0 | 28.6 | 116.3 | 0.60 | 0.33 |
| Ex. (187) | 4.4 | 8.8 | 2500.0 | 28.3 | 116.0 | 0.63 | 0.34 |
| Ex. (188) | 4.4 | 8.8 | 2500.0 | 28.3 | 119.0 | 0.60 | 0.31 |
| Ex. (189) | 4.3 | 8.8 | 2500.0 | 28.6 | 116.3 | 0.64 | 0.33 |
| Ex. (190) | 4.4 | 8.7 | 2500.0 | 28.7 | 118.1 | 0.63 | 0.35 |
| Ex. (191) | 4.4 | 8.9 | 2500.0 | 28.2 | 115.8 | 0.63 | 0.31 |
| Ex. (192) | 4.4 | 8.8 | 2500.0 | 28.5 | 118.2 | 0.64 | 0.33 |
| Ex. (193) | 4.3 | 8.7 | 2500.0 | 28.6 | 117.7 | 0.63 | 0.34 |
| Ex. (194) | 4.3 | 8.7 | 2500.0 | 28.7 | 115.3 | 0.61 | 0.34 |
| Ex. (195) | 4.4 | 8.8 | 2500.0 | 28.3 | 119.0 | 0.65 | 0.33 |
| Ex. (196) | 4.4 | 8.9 | 2500.0 | 28.0 | 118.5 | 0.64 | 0.34 |
| Ex. (197) | 4.3 | 8.7 | 2500.0 | 28.7 | 116.6 | 0.63 | 0.34 |
| Ex. (198) | 4.4 | 8.7 | 2500.0 | 28.6 | 118.4 | 0.64 | 0.34 |
| Ex. (199) | 4.3 | 8.9 | 2500.0 | 28.1 | 115.6 | 0.64 | 0.32 |
| Ex. (200) | 4.3 | 8.9 | 2500.0 | 28.2 | 119.5 | 0.64 | 0.31 |
| Ex. (201) | 4.3 | 8.9 | 2500.0 | 28.2 | 118.5 | 0.65 | 0.31 |
| Ex. (202) | 4.4 | 8.7 | 2500.0 | 28.9 | 119.2 | 0.62 | 0.34 |
| Ex. (203) | 4.4 | 8.9 | 2500.0 | 28.1 | 115.3 | 0.62 | 0.33 |
| Ex. (204) | 4.3 | 8.8 | 2500.0 | 28.3 | 117.6 | 0.63 | 0.32 |
| Ex. (205) | 4.3 | 8.8 | 2500.0 | 28.4 | 116.4 | 0.62 | 0.30 |
| Ex. (206) | 4.4 | 8.8 | 2500.0 | 28.3 | 118.3 | 0.64 | 0.30 |
| Ex. (207) | 4.3 | 8.9 | 2500.0 | 28.1 | 116.7 | 0.62 | 0.30 |

As can be seen from the results of Table 2, the organic electroluminescent element employing the compound represented by Formula 1 to at least three layers, such as a hole transport layer, an emission auxiliary layer and a light emitting layer, has higher luminous efficiency, a low driving voltage and significantly improved lifespan than Comparative Examples 1 to 4.

The results of elements of Comparative Examples 2 to 4 using the compound P-73 represented by Formula 1 as material for an emission auxiliary layer were superior to Comparative Example 1 using NPB as material for a hole transport layer and CBP as material for the light emitting layer. The results of elements of Comparative Example 3 and Comparative Example 4 were superior to those of Comparative Example 2, and the results of elements of the present invention were the best, wherein Comparative Examples 2 employs the compound represented by Formula 1 to only one layer, Comparative Examples 3 and 4 employ the compound to two layers and the present invention employs the compound to three layers.

This is because where compound represented by Formula 1 is used in at least three layers, the combination of these compounds cause electrochemically synergy, as a result, the performance of the entire element like charge balance is improved.

### Example 208 to Example 225

An organic electroluminescent element was manufactured in the same manner as in Example 1 except that two types of compounds represented by Formula 1 were used as material for the light emitting layer. Here, the compounds used in the hole transport layer, the emission-auxiliary layer and the light emitting layer were shown in Table 3.

TABLE 3

| | Hole transport | Emission-auxiliary | Light-emitting | |
|---|---|---|---|---|
| | layer compd. | layer compd. | layer compd. | |
| Ex.(208) | 2-64 | P-5 | 3-5 | P-5 |
| Ex.(209) | | | 3-13 | |
| Ex.(210) | | | 3-22 | |
| Ex.(211) | | | 3-23 | |
| Ex.(212) | | | 3-24 | |
| Ex.(213) | | | 3-26 | |
| Ex.(214) | | | 3-58 | |
| Ex.(215) | | | 3-59 | |
| Ex.(216) | | | 8-1 | |
| Ex.(217) | 2-64 | P-5 | 3-5 | 4-31 |
| Ex.(218) | | | 3-13 | |
| Ex.(219) | | | 3-22 | |
| Ex.(220) | | | 3-23 | |
| Ex.(221) | | | 3-24 | |
| Ex.(222) | | | 3-26 | |
| Ex.(223) | | | 3-58 | |
| Ex.(224) | | | 3-59 | |
| Ex.(225) | | | 8-1 | |

Also, electroluminescence (EL) characteristics were measured with a PR-650 (Photoresearch) by applying a forward bias DC voltage to the OLEDs prepared in Examples 1 to 19 of the present invention and Comparative Examples 208 to 225. And, the T95 life time was measured using a life time measuring apparatus manufactured by ms science Inc. at reference brightness of 2500 cd/m². The measurement results are shown in Tables 4 below.

TABLE 4

| | | Current | Bright- | Effi- | | | |
|---|---|---|---|---|---|---|---|
| | Voltage | Density | ness | ciency | Lifetime | CIE | |
| | (V) | (mA/cm²) | (cd/m²) | (cd/A) | T(95) | x | y |
| Ex. (208) | 4.2 | 7.1 | 2500.0 | 35.1 | 128.0 | 0.64 | 0.33 |
| Ex. (209) | 4.2 | 7.0 | 2500.0 | 35.7 | 127.7 | 0.61 | 0.33 |
| Ex. (210) | 4.1 | 6.8 | 2500.0 | 36.9 | 127.5 | 0.63 | 0.32 |
| Ex. (211) | 4.2 | 6.8 | 2500.0 | 36.9 | 128.2 | 0.61 | 0.31 |
| Ex. (212) | 4.2 | 6.9 | 2500.0 | 36.2 | 128.9 | 0.62 | 0.34 |
| Ex. (213) | 4.2 | 7.1 | 2500.0 | 35.4 | 127.9 | 0.64 | 0.31 |
| Ex. (214) | 4.1 | 7.0 | 2500.0 | 35.9 | 129.3 | 0.63 | 0.31 |
| Ex. (215) | 4.1 | 7.1 | 2500.0 | 35.4 | 127.7 | 0.60 | 0.34 |
| Ex. (216) | 4.1 | 7.0 | 2500.0 | 35.9 | 127.3 | 0.62 | 0.31 |
| Ex. (217) | 4.2 | 7.5 | 2500.0 | 33.1 | 125.5 | 0.61 | 0.33 |
| Ex. (218) | 4.2 | 7.5 | 2500.0 | 33.4 | 125.7 | 0.64 | 0.33 |
| Ex. (219) | 4.2 | 7.5 | 2500.0 | 33.4 | 125.1 | 0.63 | 0.31 |
| Ex. (220) | 4.2 | 7.6 | 2500.0 | 33.0 | 125.2 | 0.63 | 0.35 |
| Ex. (221) | 4.2 | 7.5 | 2500.0 | 33.1 | 125.9 | 0.65 | 0.34 |
| Ex. (222) | 4.3 | 7.5 | 2500.0 | 33.5 | 125.9 | 0.61 | 0.31 |
| Ex. (223) | 4.3 | 7.4 | 2500.0 | 33.9 | 125.5 | 0.64 | 0.33 |
| Ex. (224) | 4.3 | 7.4 | 2500.0 | 33.7 | 125.4 | 0.64 | 0.34 |
| Ex. (225) | 4.2 | 7.5 | 2500.0 | 33.2 | 125.1 | 0.63 | 0.33 |

As can be seen from the results in Table 4, when the compound represented by Formula 1 is used in at least three layers such as a hole transport layer, an emitting auxiliary layer and a light-emitting layer and two different types of compounds represented by Formula 1 are used as the host material of the light emitting layer, the driving voltage was slightly improved and efficiency and lifespan were significantly improved than Examples 1 to 207 in which only one type of compound was used as a host material for the light emitting layer.

This is because when the mixture of the compound of the present invention is employed to the light emitting layer, not only electrons and holes are moved through the energy level of each material, but also electrons and holes are moved and energy is transferred through exciplex with a new energy level formed by mixing, resulting in efficiency and lifetime are increased.

Although the exemplary embodiments of the present invention have been described for illustrative purposes, those skilled in the art to which the present invention pertains will be capable of various modifications without departing from the essential characteristics of the present invention. Therefore, the embodiment disclosed herein is intended to illustrate the scope of the technical idea of the present invention, and the spirit and scope of the present invention are not limited by the embodiments. The scope of the present invention shall be construed on the basis of the accompanying claims, and it shall be construed that all of the technical ideas included within the scope equivalent to the claims belong to the present invention.

1. An organic electric element comprising a first electrode, a second electrode, and an organic material layer formed between the first electrode and the second electrode and comprising a light emitting layer, wherein the organic material layer comprises an emission-auxiliary layer formed between the light emitting layer and the first electrode, a hole transport layer formed between the emission-auxiliary layer and the first electrode, an electron transport-auxiliary layer formed between the light emitting layer and the second electrode, and an electron transport layer formed between the electron transport-auxiliary layer and the second electrode, and at least three layers among the layers forming the organic material layer comprise a single compound or two or more compounds represented by Formula 1: 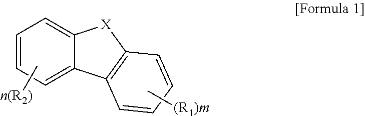
wherein:
X is O or S,
R₁ and R₂ are each independently selected from the group consisting of hydrogen, deuterium, halogen, a C₆-C₆₀ aryl group, a fluorenyl group, a C₂-C₆₀ heterocyclic group containing at least one heteroatom of O, N, S, Si, and P, a C₃-C₆₀ aliphatic ring, a fused ring group formed by a C₃-C₆₀ aliphatic ring with a C₆-C₆₀ aromatic ring, a C₁-C₅₀ alkyl group, a C₂-C₂₀ alkenyl group, a C₂-C₂₀ alkynyl group, a C₁-C₃₀ alkoxyl group, a C₆-C₃₀ aryloxy group and -L′-N(Rₐ)(R_{b}), and adjacent R₁ groups or adjacent R₂ groups together may be bonded to each other to form a ring, wherein the ring is a C₆-C₆₀ aromatic ring, a C₃-C₆₀ aliphatic ring, a C₂-C₆₀ heterocyclic ring containing at least one heteroatom of O, N, S, Si, and P, or a combination thereof,
m and n are each an integer of 0 to 4, when m is an integer of 2 or more, a plurality of R₁s are the same or different from each other, and when n is an integer of 2 or more, a plurality of Res are the same or different from each other,
L′ is selected from the group consisting of a single bond, a C₆-C₆₀ arylene group, a fluorenylene group, a C₃-C₆₀ aliphatic ring, a C₂-C₆₀ heterocyclic group containing at least one heteroatom of O, N, S, Si, and P, and a combination thereof,
Rₐ and R_{b} are each independently selected from the group consisting of a C₆-C₆₀ aryl group, a fluorenyl group, a C₂-C₆₀ heterocyclic group containing at least one heteroatom of O, N, S, Si, and P, a C₃-C₆₀ aliphatic ring, and a fused ring group formed by a C₃-C₆₀ aliphatic ring with a C₆-C₆₀ aromatic ring, and
the R₁, R₂, the ring formed by adjacent R₁ groups, the ring formed by adjacent R₂ groups, L′, Rₐ and R_{b} may be each optionally substituted with one or more substituents selected from the group consisting of deuterium, halogen, a silane group unsubstituted or substituted with a C₁-C₂₀ alkyl group or a C₆-C₂₀ aryl group, a siloxane group, a boron group, a germanium group, a cyano group, a nitro group, a C₁-C₂₀ alkylthio group, a C₁-C₂₀ alkoxyl group, a C₁-C₂₀ alkyl group, a C₂-C₂₀ alkenyl group, a C₂-C₂₀ alkynyl group, a C₆-C₂₀ aryl group, a C₆-C₂₀ aryl group substituted with deuterium, a fluorenyl group, a C₂-C₂₀ heterocyclic group containing at least one heteroatom of O, N, S, Si, and P, a C₃-C₂₀ aliphatic ring, a C₇-C₂₀ arylalkyl group, a C₈-C₂₀ arylalkenyl group, -L′-N(Rₐ)(R_{b}) and a combination thereof.

2. The organic electric element of claim 1, wherein the compound represented by Formula 1 is comprised in at least the light emitting layer.

3. The organic electric element of claim 2, wherein the compound represented by Formula 1 is comprised in at least one layer of the organic material layer between the first electrode and the light emitting layer.

4. The organic electric element of claim 2, wherein the compound represented by Formula 1 is comprised in at least two layers of the organic material layer between the first electrode and the light emitting layer.

5. The organic electric element of claim 1, wherein the compound represented by Formula 1 is comprised in the hole transport layer, the emission-auxiliary layer and the light emitting layer.

6. The organic electric element of claim 5, wherein the layers adjacent to each other among the hole transport layer, the emission-auxiliary layer and the light emitting layer comprise at least one different compound.

7. The organic electric element of claim 1, wherein Formula 1 is represented by one of Formula 2 to Formula 4: 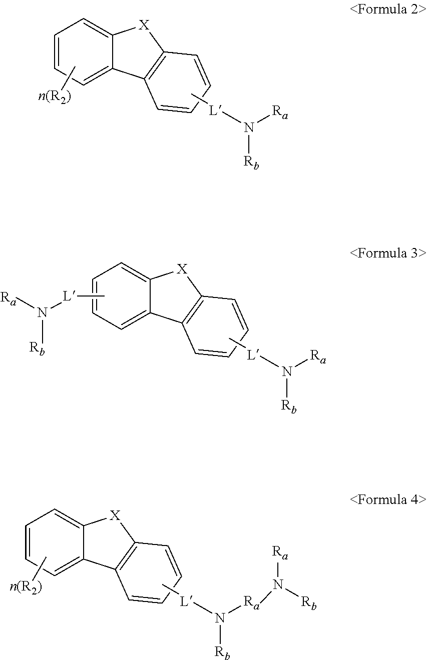
wherein X, R₂, L′, Rₐ, R_{b} and n are the same as defined in claim 1, and Rₐs are the same or different from each other and R_{b}s are the same or different from each other.

8. The organic electric element of claim 7, wherein the hole transport layer, the emission-auxiliary layer and the light emitting layer comprise the compound represented by one of Formulas 2 to 4.

9. The organic electric element of claim 1, wherein the light emitting layer comprises a single compound or two or more compounds represented by Formula 5 or Formula 6: 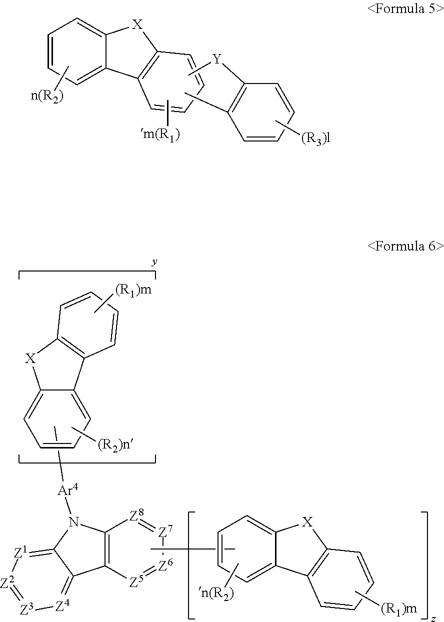
wherein, X, R₁, R₂, m and n are the same as defined in claim 1, m′ is an integer of 0 to 2, n′ is an integer of 0 to 3, y and z are each an integer of 0 or 1, y+z is 1 or 2,
Y is O, S or N(Ar¹),
R₃ is each independently selected from the group consisting of hydrogen, deuterium, halogen, a C₆-C₆₀ aryl group, a fluorenyl group, a C₂-C₆₀ heterocyclic group containing at least one heteroatom of O, N, S, Si, and P, a C₃-C₆₀ aliphatic ring, a fused ring group formed by a C₃-C₆₀ aliphatic ring with a C₆-C₆₀ aromatic ring, a C₁-C₅₀ alkyl group, a C₂-C₂₀ alkenyl group, a C₂-C₂₀ alkynyl group, a C₁-C₃₀ alkoxyl group, a C₆-C₃₀ aryloxy group and -L′-N(Rₐ)(R_{b}),
l is an integer of 0 to 4, and when l is an integer of 2 or more, a plurality of R₃s are the same or different from each other, and adjacent R₃ groups may be optionally linked to each other to form a ring, wherein the ring is a C₆-C₆₀ aromatic ring, a C₃-C₆₀ aliphatic ring, a C₂-C₆₀ heterocyclic group containing at least one heteroatom selected from the group consisting of O, N, S, Si, and P, or a combination thereof,
Z¹ to Z⁸ are each independently C(R) or N,
Ar⁴ is selected from the group consisting of a C₆-C₆₀ aryl group, a C₆-C₆₀ arylene group, a C₂-C₆₀ heterocyclic group containing at least one heteroatom selected from the group consisting of O, N, S, Si, and P, a C₃-C₆₀ aliphatic ring, a fused ring group formed by a C₃-C₆₀ aliphatic ring with a C₆-C₆₀ aromatic ring, a C₁-C₅₀ alkyl group, a C₁-C₅₀ alkylene group, a C₆-C₆₀ arylamine group, a fluorenyl group, and a fluorenylene group,
L² is selected from the group consisting of a single bond, a C₆-C₆₀ aryl group, a C₆-C₆₀ arylene group, a C₂-C₆₀ heterocyclic group containing at least one heteroatom selected from the group consisting of O, N, S, Si, and P, a C₃-C₆₀ aliphatic ring, a fluorenyl group, a fluorenylene group, and a combination thereof,
Ar¹ is selected from the group consisting of a C₆-C₆₀ aryl group, a fluorenyl group, a C₂-C₆₀ heterocyclic group containing at least one heteroatom selected from the group consisting of O, N, S, Si, and -L′-N(Rₐ)(R_{b}),
R is selected from the group consisting of hydrogen, a C₆-C₆₀ aryl group, a C₂-C₆₀ heterocyclic group containing at least one heteroatom selected from the group consisting of O, N, S, Si, a C₁-C₅₀ alkyl group, a C₆-C₆₀ arylamine group and a fluorenyl group, and adjacent groups may be optionally linked to each other to form a ring, and
L′, Rₐ and R_{b} are the same as defined in claim 1.

10. The organic electric element of claim 9, wherein Formula 1 is represented by Formula 5 or Formula 6.

11. The organic electric element of claim 9, wherein the light emitting layer further comprises at least one of the compounds represented by Formulas 2 to 4, and Formulas 2 to 4 comprised in the light emitting layer are comprised in Formula 1: 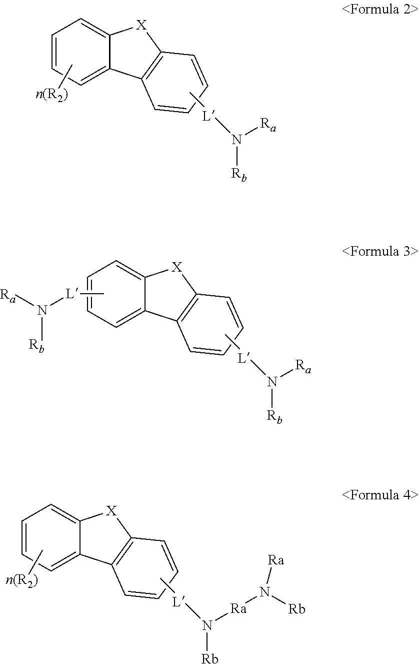
wherein, X, R₂, L′, Rₐ, R_{b} and n are the same as defined in claim 1, and Rₐs are the same or different from each other, and R_{b}s are the same or different from each other.

12. The organic electric element of claim 9, wherein Formula 1 is represented by one of Formulas 2 to 4, and the hole transport layer and the emission-auxiliary layer comprise the compound represented by Formulas 2 to 4: 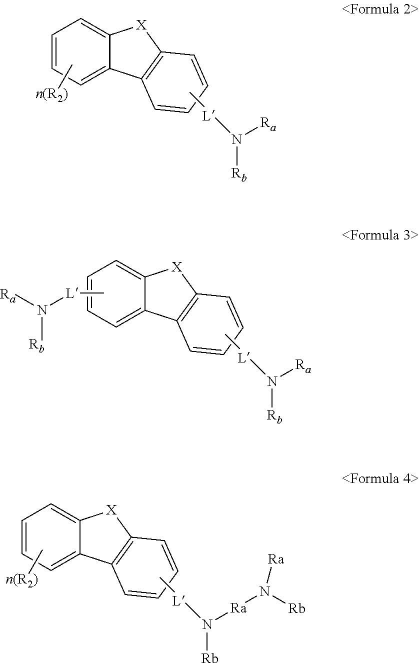
wherein, X, R₂, L′, Rₐ, R_{b} and n are the same as defined in claim 1, and Rₐs are the same or different from each other, and R_{b}s are the same or different from each other.

13. The organic electric element of claim 12, wherein the light emitting layer comprise the compound represented by Formula 5 and the compound represented by one of Formulas 2 to 4.

14. The organic electric element of claim 9, wherein Formula 1 is represented by Formula 3, the emission-auxiliary layer comprises the compound represented by Formulas 3, and the light emitting layer comprises the compound represented by Formulas 5: 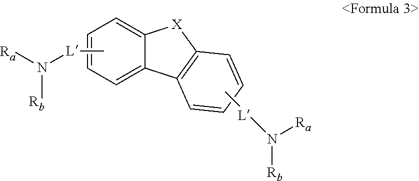
wherein, X, L′, Rₐ and R_{b} are the same as defined in claim 1, and Rₐs are the same or different from each other, and R_{b}s are the same or different from each other.

15. The organic electric element of claim 9, wherein Formula 1 is represented by Formula 3 or Formula 4, the hole transport layer comprises the compound represented by Formulas 4, the emission-auxiliary layer comprises the compound represented by Formulas 3, and the light emitting layer comprises the compound represented by Formulas 5: 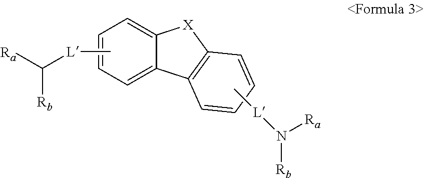
wherein X, R₂, n, L′, Rₐ and R_{b} the same as defined in claim 1, and Rₐs are the same or different from each other, and R_{b}s are the same or different from each other.

16. The organic electric element of claim 15, wherein X is O in Formulas 3 to 5.

17. The organic electric element of claim 15, wherein X is S in Formulas 3 to 5.

18. The organic electric element of claim 15, wherein X is O in Formula 4, and X is S in Formulas 3 and 5.

19. The organic electric element of claim 15, wherein X is O in Formula 4, and X is different from each other in Formulas 3 and 5.

20. The organic electric element of claim 15, wherein X is S in Formula 4, and X is O in Formulas 3 and 5.

21. The organic electric element of claim 15, wherein X is S in Formula 4, and X is different from each other in Formulas 3 and 5.

21. The compound of claim 1, wherein the compound represented by Formula 1 is one of the following compounds:

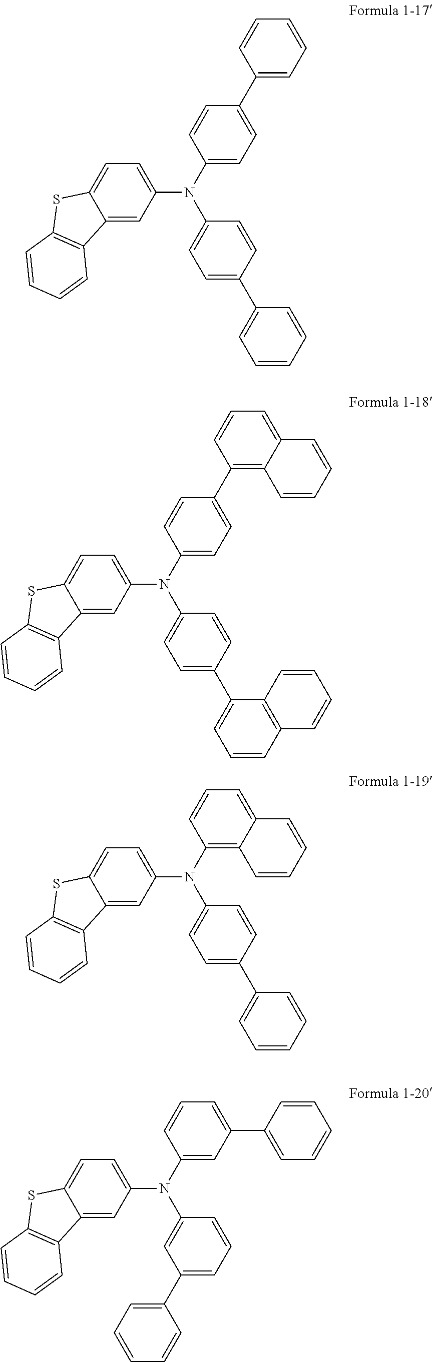

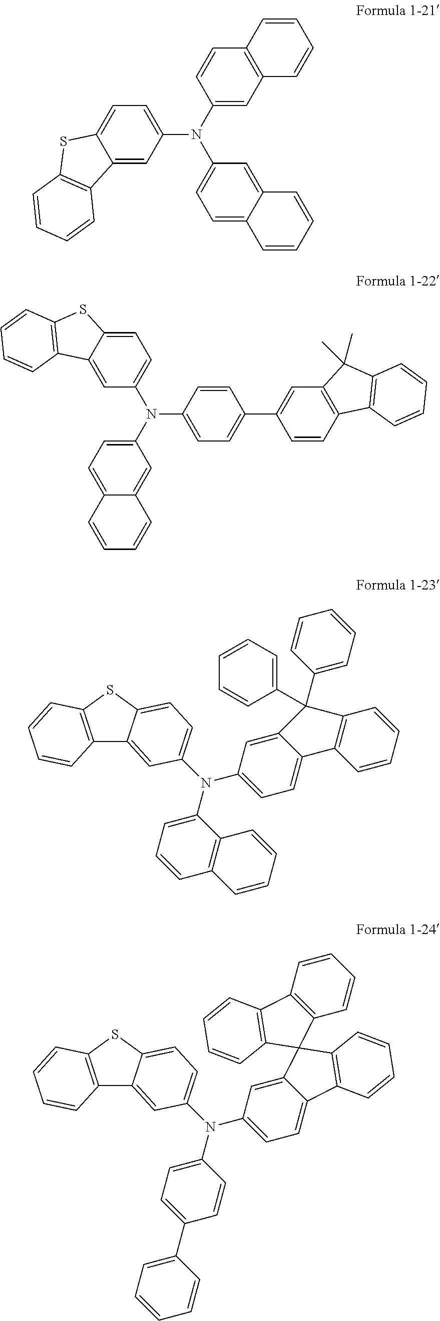 [SMILES: c1ccc2cc(N(c3ccc4ccccc4c3)c3ccc4sc5ccccc5c4c3)ccc2c1 ; CC1(C)c2ccccc2-c2ccc(-c3ccc(N(c4ccc5ccccc5c4)c4ccc5sc6ccccc6c5c4)cc3)cc21 ; c1ccc(C2(c3ccccc3)c3ccccc3-c3ccc(N(c4ccc5sc6ccccc6c5c4)c4cccc5ccccc45)cc32)cc1 ; c1ccc(-c2ccc(N(c3ccc4c(c3)C3(c5ccccc5-c5ccccc53)c3ccccc3-4)c3ccc4sc5ccccc5c4c3)cc2)cc1]

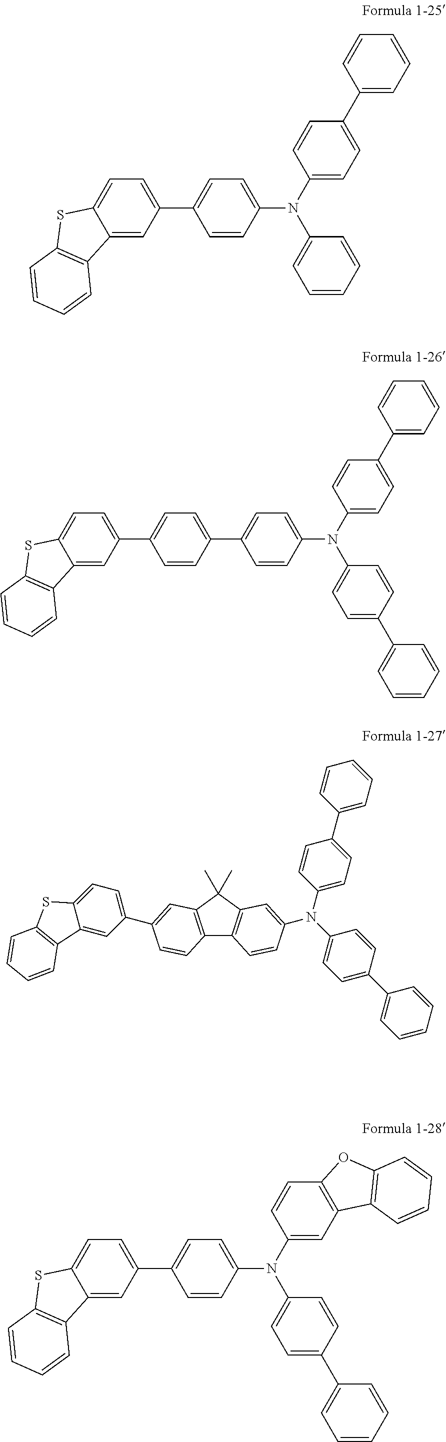 [SMILES: c1ccc(-c2ccc(N(c3ccccc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-c5ccc6sc7ccccc7c6c5)cc4)cc3)cc2)cc1 ; CC1(C)c2cc(-c3ccc4sc5ccccc5c4c3)ccc2-c2ccc(N(c3ccc(-c4ccccc4)cc3)c3ccc(-c4ccccc4)cc3)cc21 ; c1ccc(-c2ccc(N(c3ccc(-c4ccc5sc6ccccc6c5c4)cc3)c3ccc4oc5ccccc5c4c3)cc2)cc1]

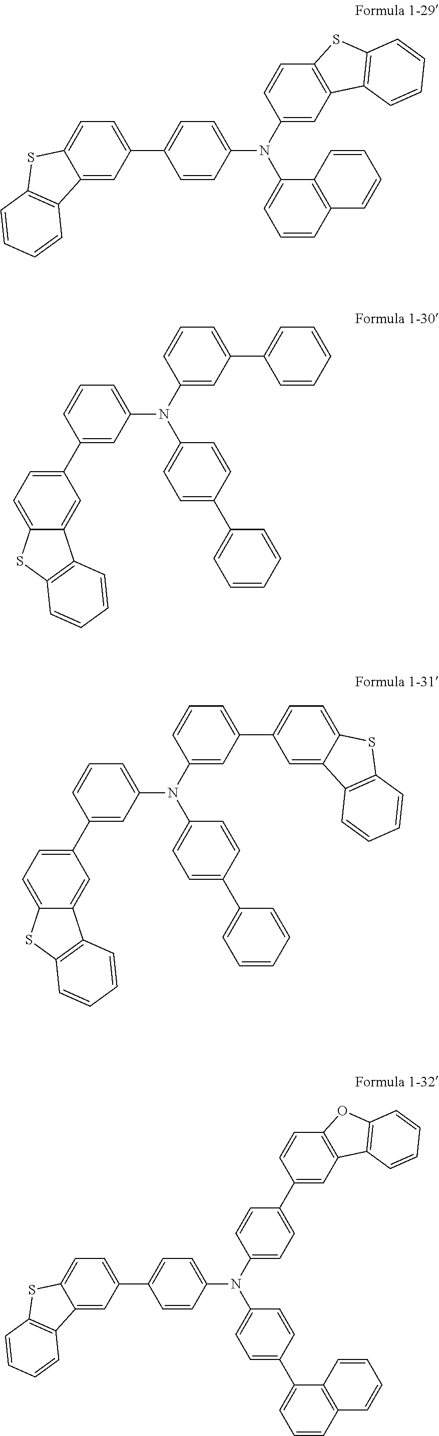 [SMILES: c1ccc2c(N(c3ccc(-c4ccc5sc6ccccc6c5c4)cc3)c3ccc4sc5ccccc5c4c3)cccc2c1 ; c1ccc(-c2ccc(N(c3cccc(-c4ccccc4)c3)c3cccc(-c4ccc5sc6ccccc6c5c4)c3)cc2)cc1 ; c1ccc(-c2ccc(N(c3cccc(-c4ccc5sc6ccccc6c5c4)c3)c3cccc(-c4ccc5sc6ccccc6c5c4)c3)cc2)cc1 ; c1ccc2c(-c3ccc(N(c4ccc(-c5ccc6oc7ccccc7c6c5)cc4)c4ccc(-c5ccc6sc7ccccc7c6c5)cc4)cc3)cccc2c1]

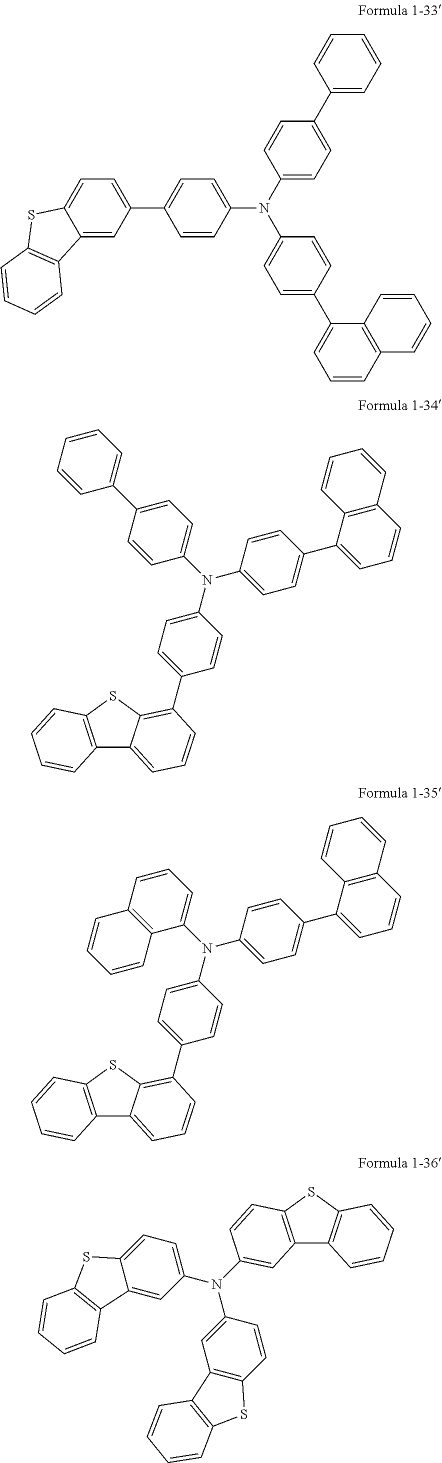 [SMILES: c1ccc(-c2ccc(N(c3ccc(-c4ccc5sc6ccccc6c5c4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5c4sc4ccccc45)cc3)cc2)cc1 ; c1ccc2c(-c3ccc(N(c4ccc(-c5cccc6c5sc5ccccc56)cc4)c4cccc5ccccc45)cc3)cccc2c1 ; c1ccc2c(c1)sc1ccc(N(c3ccc4sc5ccccc5c4c3)c3ccc4sc5ccccc5c4c3)cc12]

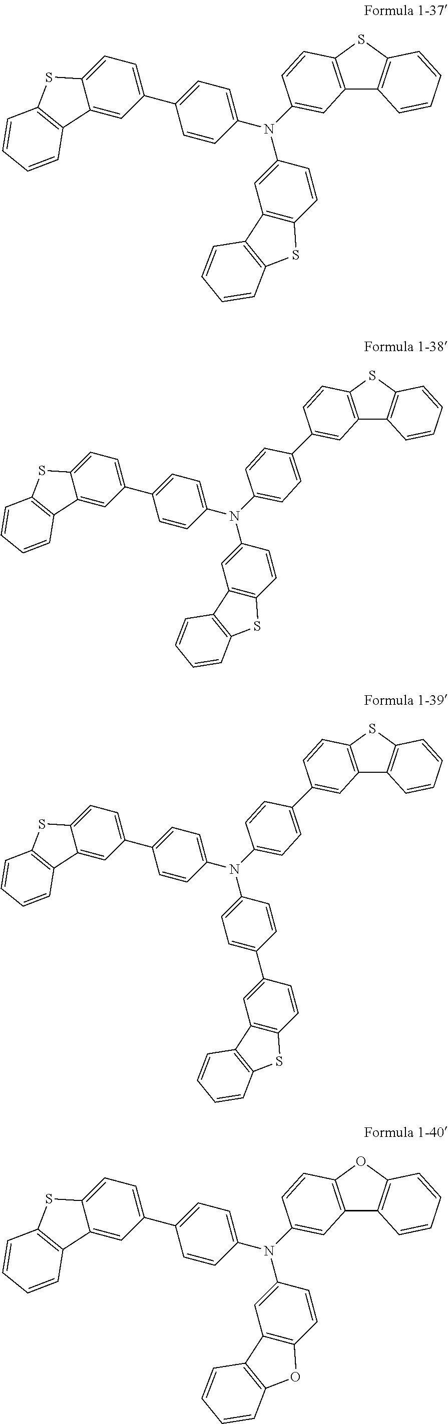 [SMILES: c1ccc2c(c1)sc1ccc(-c3ccc(N(c4ccc5sc6ccccc6c5c4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc(-c5ccc6sc7ccccc7c6c5)cc4)cc3)cc12 ; c1ccc2c(c1)oc1ccc(N(c3ccc(-c4ccc5sc6ccccc6c5c4)cc3)c3ccc4oc5ccccc5c4c3)cc12]

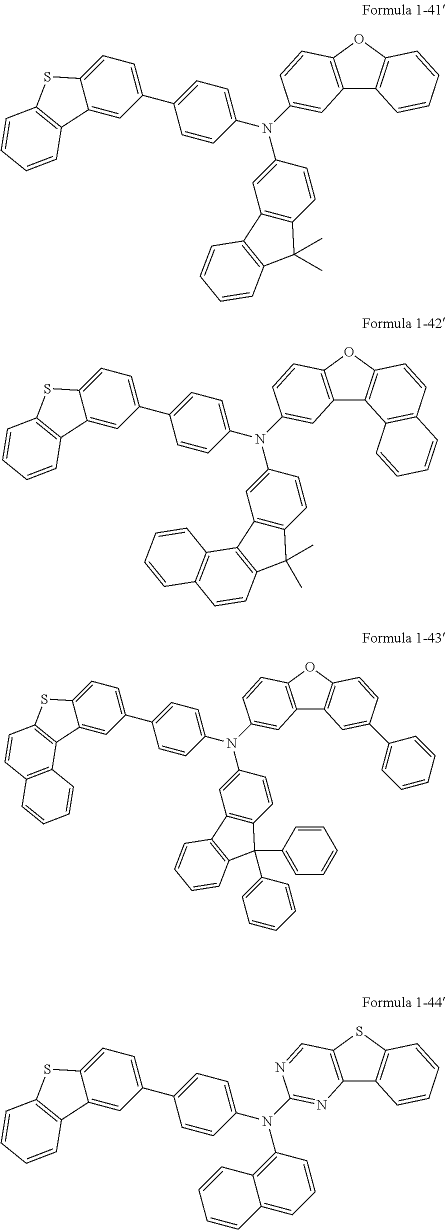 [SMILES: CC1(C)c2ccccc2-c2cc(N(c3ccc(-c4ccc5sc6ccccc6c5c4)cc3)c3ccc4oc5ccccc5c4c3)ccc21 ; CC1(C)c2ccc(N(c3ccc(-c4ccc5sc6ccccc6c5c4)cc3)c3ccc4oc5ccc6ccccc6c5c4c3)cc2-c2c1ccc1ccccc21 ; c1ccc(-c2ccc3oc4ccc(N(c5ccc(-c6ccc7sc8ccc9ccccc9c8c7c6)cc5)c5ccc6c(c5)-c5ccccc5C6(c5ccccc5)c5ccccc5)cc4c3c2)cc1 ; c1ccc2c(N(c3ccc(-c4ccc5sc6ccccc6c5c4)cc3)c3ncc4sc5ccccc5c4n3)cccc2c1]

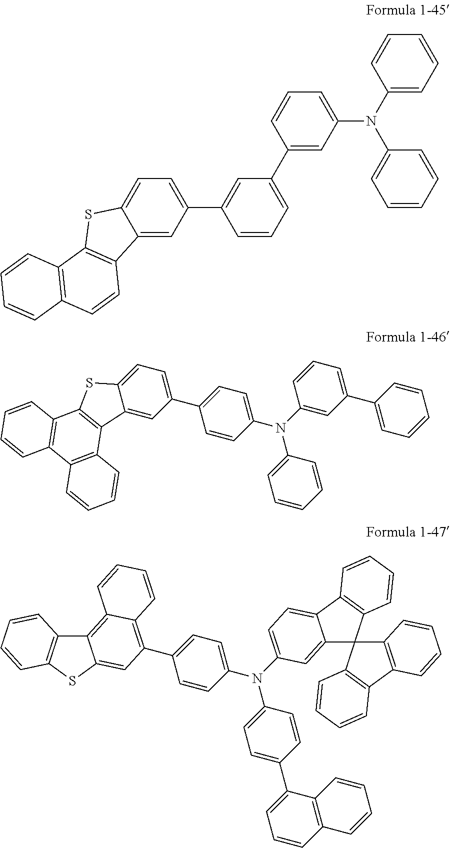 [SMILES: c1ccc(N(c2ccccc2)c2cccc(-c3cccc(-c4ccc5sc6c7ccccc7ccc6c5c4)c3)c2)cc1 ; c1ccc(-c2cccc(N(c3ccccc3)c3ccc(-c4ccc5sc6c7ccccc7c7ccccc7c6c5c4)cc3)c2)cc1 ; c1ccc2c(c1)-c1ccccc1C21c2ccccc2-c2ccc(N(c3ccc(-c4cccc5ccccc45)cc3)c3ccc(-c4cc5sc6ccccc6c5c5ccccc45)cc3)cc21]

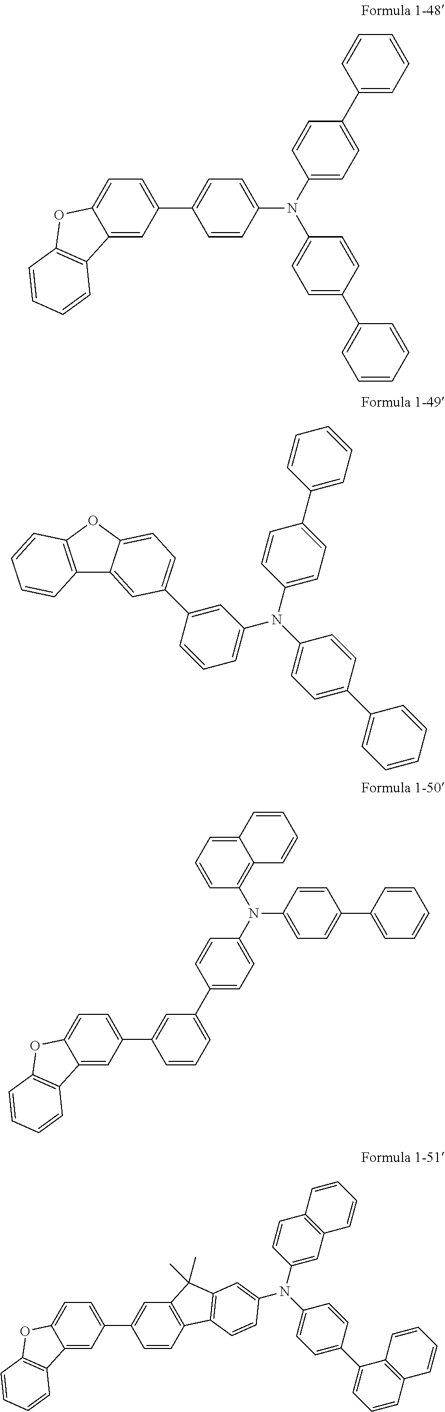 [SMILES: c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3cccc(-c4ccc5oc6ccccc6c5c4)c3)cc2)cc1 ; c1ccc(-c2ccc(N(c3ccc(-c4cccc(-c5ccc6oc7ccccc7c6c5)c4)cc3)c3cccc4ccccc34)cc2)cc1 ; CC1(C)c2cc(-c3ccc4oc5ccccc5c4c3)ccc2-c2ccc(N(c3ccc(-c4cccc5ccccc45)cc3)c3ccc4ccccc4c3)cc21]

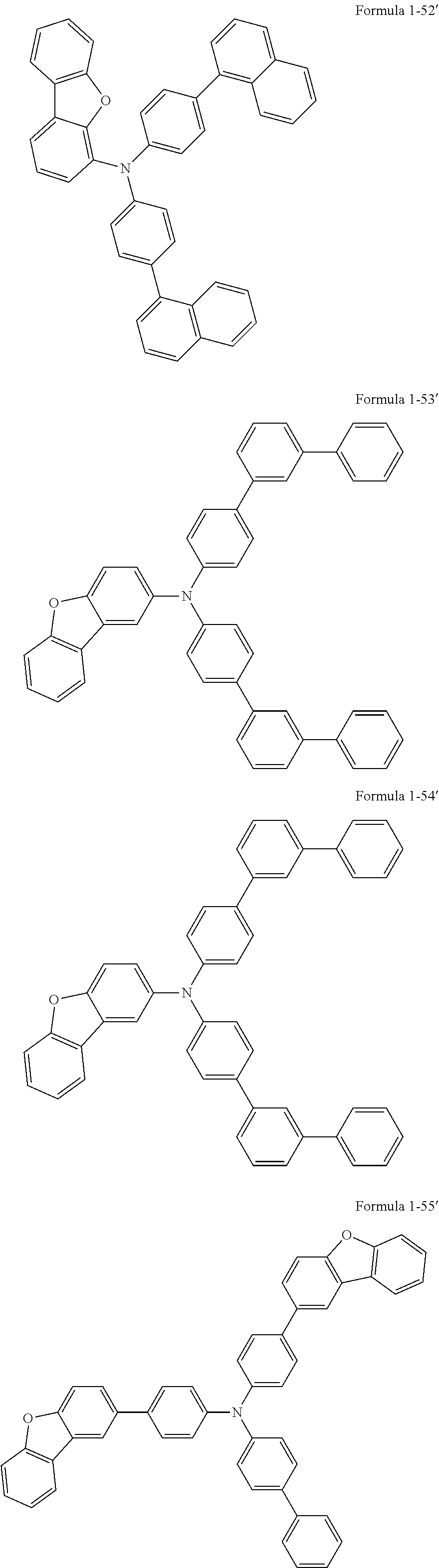 [SMILES: c1ccc2c(-c3ccc(N(c4ccc(-c5cccc6ccccc56)cc4)c4cccc5c4oc4ccccc45)cc3)cccc2c1 ; c1ccc(-c2cccc(-c3ccc(N(c4ccc(-c5cccc(-c6ccccc6)c5)cc4)c4ccc5oc6ccccc6c5c4)cc3)c2)cc1 ; c1ccc(-c2cccc(-c3ccc(N(c4ccc(-c5cccc(-c6ccccc6)c5)cc4)c4ccc5oc6ccccc6c5c4)cc3)c2)cc1 ; c1ccc(-c2ccc(N(c3ccc(-c4ccc5oc6ccccc6c5c4)cc3)c3ccc(-c4ccc5oc6ccccc6c5c4)cc3)cc2)cc1]

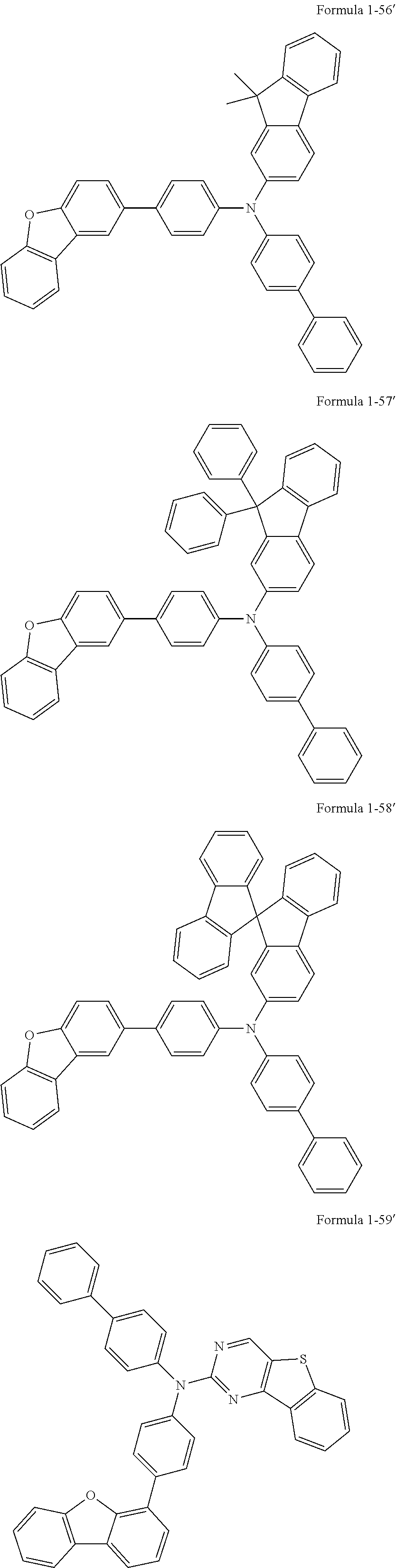

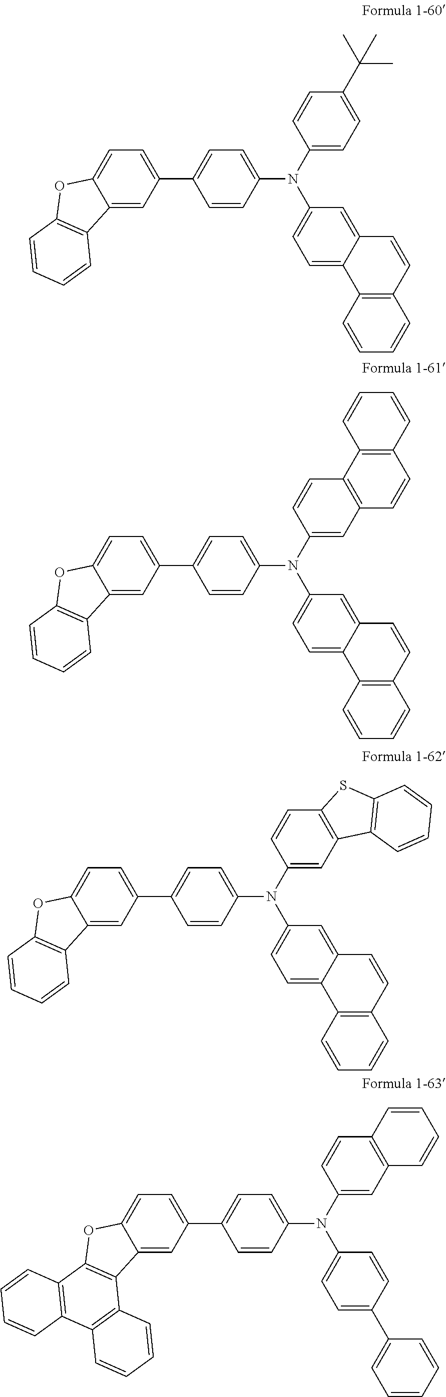

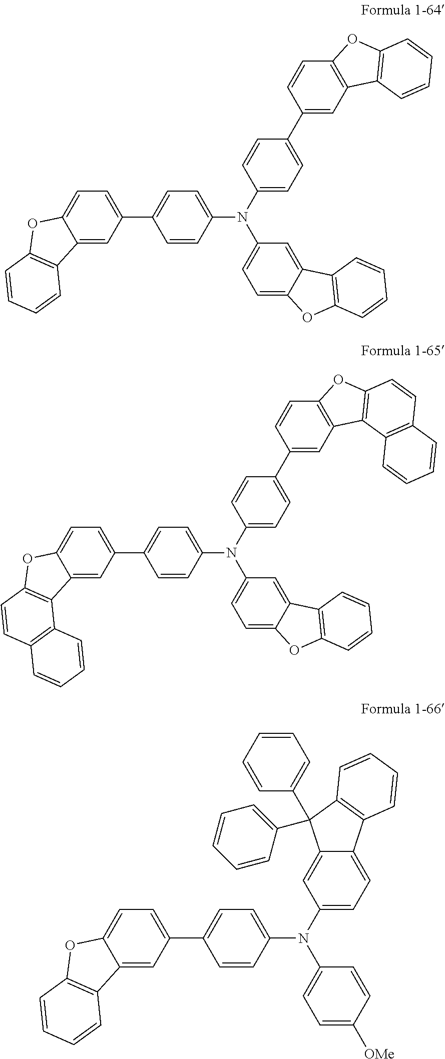 [SMILES: c1ccc2c(c1)oc1ccc(-c3ccc(N(c4ccc(-c5ccc6oc7ccccc7c6c5)cc4)c4ccc5oc6ccccc6c5c4)cc3)cc12 ; c1ccc2c(c1)ccc1oc3ccc(-c4ccc(N(c5ccc(-c6ccc7oc8ccc9ccccc9c8c7c6)cc5)c5ccc6oc7ccccc7c6c5)cc4)cc3c12 ; COc1ccc(N(c2ccc(-c3ccc4oc5ccccc5c4c3)cc2)c2ccc3c(c2)C(c2ccccc2)(c2ccccc2)c2ccccc2-3)cc1]

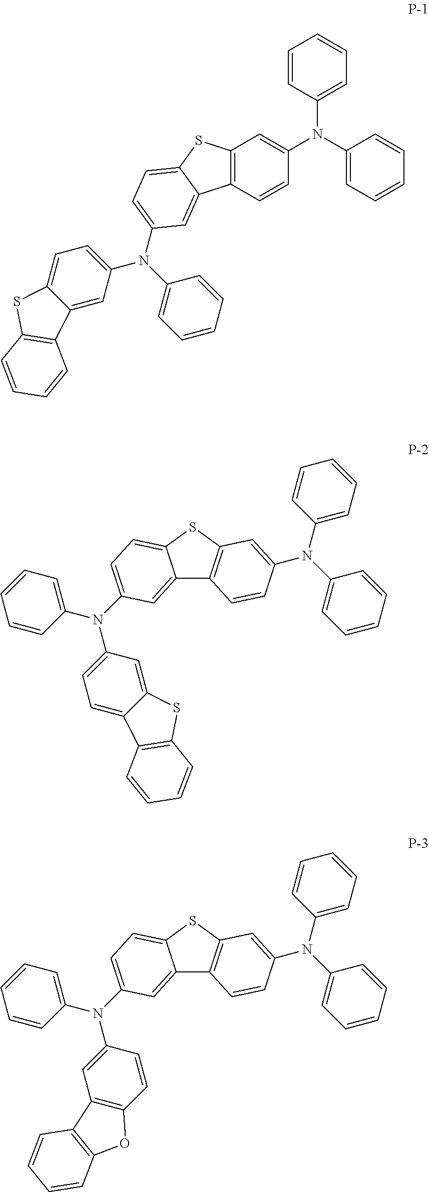 [SMILES: c1ccc(N(c2ccccc2)c2ccc3c(c2)sc2ccc(N(c4ccccc4)c4ccc5sc6ccccc6c5c4)cc23)cc1 ; c1ccc(N(c2ccccc2)c2ccc3c(c2)sc2ccc(N(c4ccccc4)c4ccc5c(c4)sc4ccccc45)cc23)cc1 ; c1ccc(N(c2ccccc2)c2ccc3c(c2)sc2ccc(N(c4ccccc4)c4ccc5oc6ccccc6c5c4)cc23)cc1]

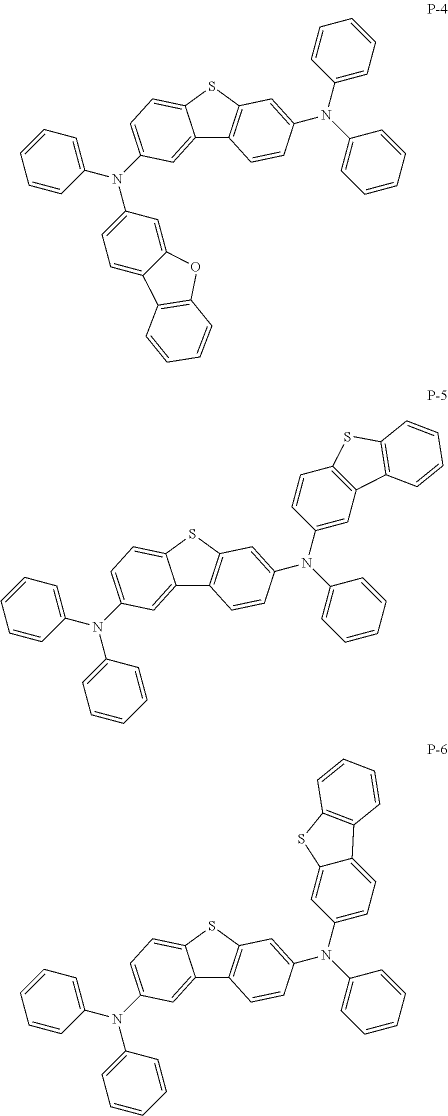 [SMILES: c1ccc(N(c2ccccc2)c2ccc3c(c2)sc2ccc(N(c4ccccc4)c4ccc5c(c4)oc4ccccc45)cc23)cc1 ; c1ccc(N(c2ccc3c(c2)sc2ccc(N(c4ccccc4)c4ccccc4)cc23)c2ccc3sc4ccccc4c3c2)cc1 ; c1ccc(N(c2ccc3c(c2)sc2ccccc23)c2ccc3c(c2)sc2ccc(N(c4ccccc4)c4ccccc4)cc23)cc1]

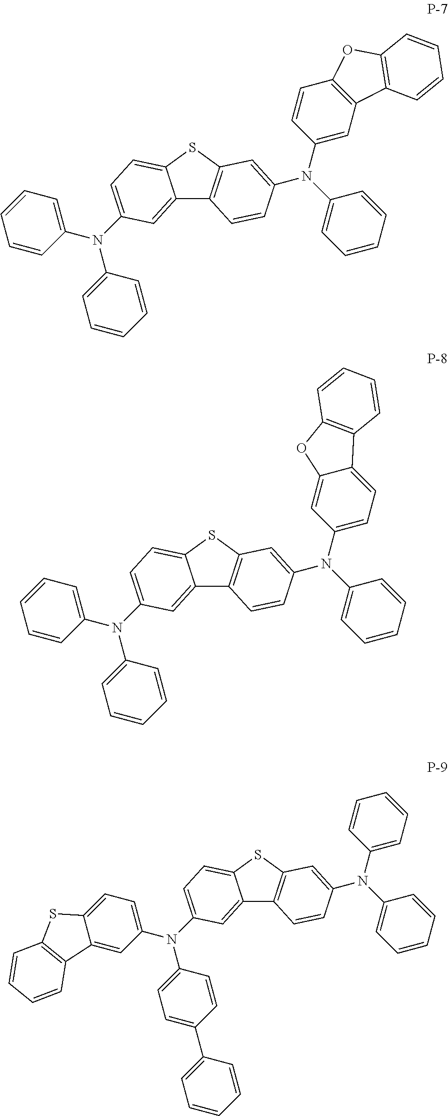 [SMILES: c1ccc(N(c2ccc3c(c2)sc2ccc(N(c4ccccc4)c4ccccc4)cc23)c2ccc3oc4ccccc4c3c2)cc1 ; c1ccc(N(c2ccc3c(c2)oc2ccccc23)c2ccc3c(c2)sc2ccc(N(c4ccccc4)c4ccccc4)cc23)cc1 ; c1ccc(-c2ccc(N(c3ccc4sc5ccccc5c4c3)c3ccc4sc5cc(N(c6ccccc6)c6ccccc6)ccc5c4c3)cc2)cc1]

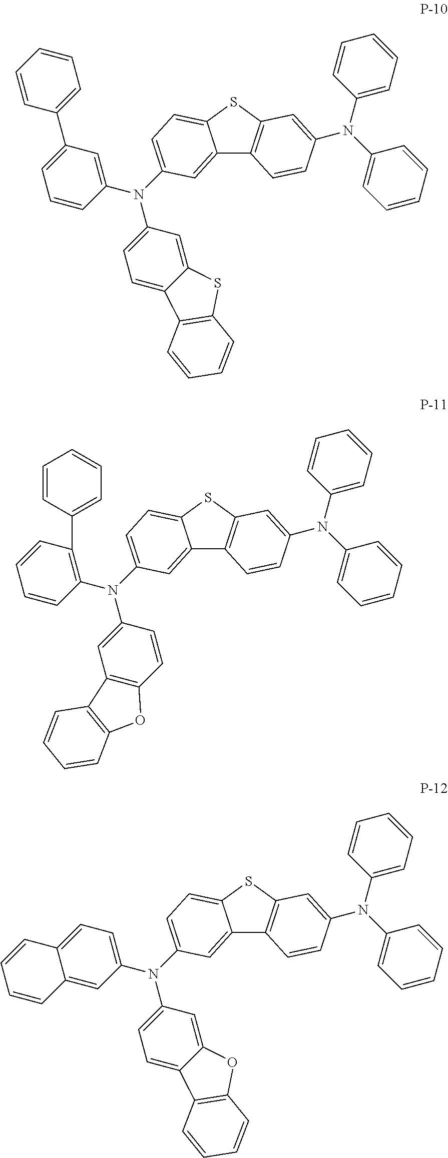 [SMILES: c1ccc(-c2cccc(N(c3ccc4c(c3)sc3ccccc34)c3ccc4sc5cc(N(c6ccccc6)c6ccccc6)ccc5c4c3)c2)cc1 ; c1ccc(-c2ccccc2N(c2ccc3oc4ccccc4c3c2)c2ccc3sc4cc(N(c5ccccc5)c5ccccc5)ccc4c3c2)cc1 ; c1ccc(N(c2ccccc2)c2ccc3c(c2)sc2ccc(N(c4ccc5ccccc5c4)c4ccc5c(c4)oc4ccccc45)cc23)cc1]

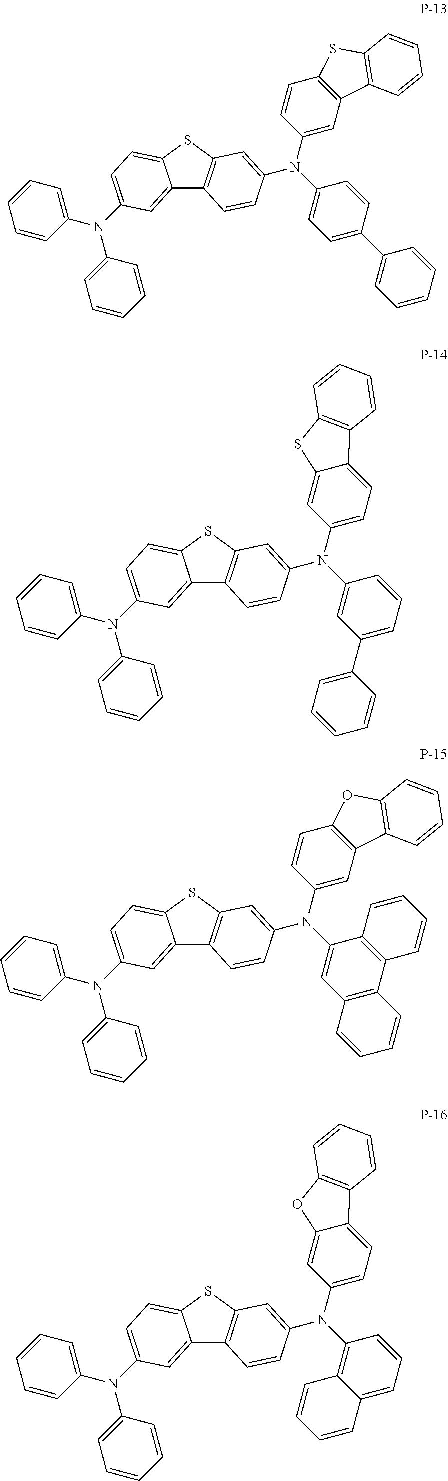 [SMILES: c1ccc(-c2ccc(N(c3ccc4c(c3)sc3ccc(N(c5ccccc5)c5ccccc5)cc34)c3ccc4sc5ccccc5c4c3)cc2)cc1 ; c1ccc(-c2cccc(N(c3ccc4c(c3)sc3ccccc34)c3ccc4c(c3)sc3ccc(N(c5ccccc5)c5ccccc5)cc34)c2)cc1 ; c1ccc(N(c2ccccc2)c2ccc3sc4cc(N(c5ccc6oc7ccccc7c6c5)c5cc6ccccc6c6ccccc56)ccc4c3c2)cc1 ; c1ccc(N(c2ccccc2)c2ccc3sc4cc(N(c5ccc6c(c5)oc5ccccc56)c5cccc6ccccc56)ccc4c3c2)cc1]

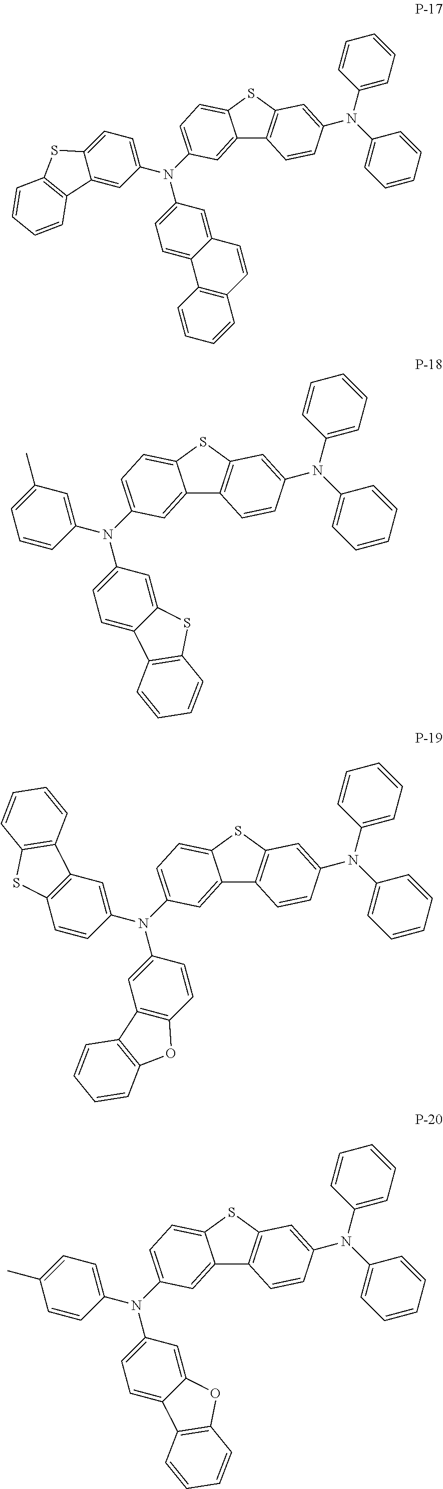 [SMILES: c1ccc(N(c2ccccc2)c2ccc3c(c2)sc2ccc(N(c4ccc5c(ccc6ccccc65)c4)c4ccc5sc6ccccc6c5c4)cc23)cc1 ; Cc1cccc(N(c2ccc3c(c2)sc2ccccc23)c2ccc3sc4cc(N(c5ccccc5)c5ccccc5)ccc4c3c2)c1 ; c1ccc(N(c2ccccc2)c2ccc3c(c2)sc2ccc(N(c4ccc5oc6ccccc6c5c4)c4ccc5sc6ccccc6c5c4)cc23)cc1 ; Cc1ccc(N(c2ccc3c(c2)oc2ccccc23)c2ccc3sc4cc(N(c5ccccc5)c5ccccc5)ccc4c3c2)cc1]

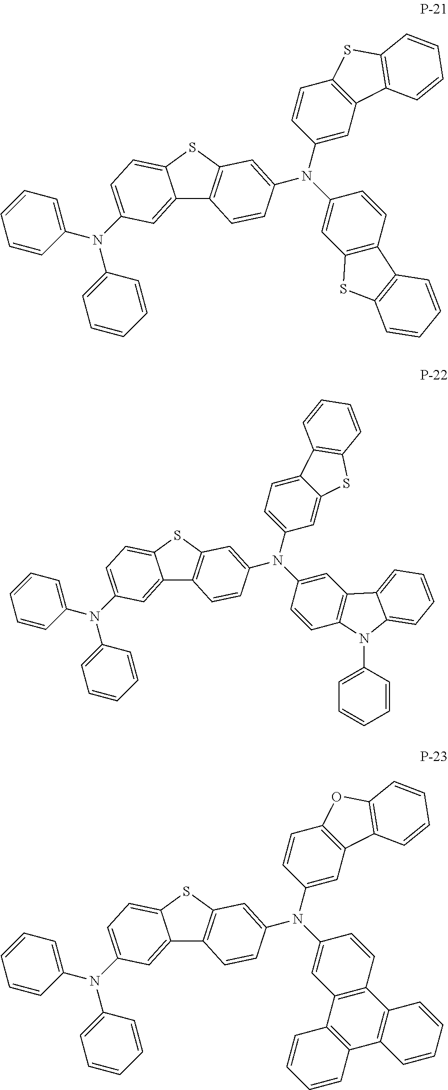 [SMILES: c1ccc(N(c2ccccc2)c2ccc3sc4cc(N(c5ccc6c(c5)sc5ccccc56)c5ccc6sc7ccccc7c6c5)ccc4c3c2)cc1 ; c1ccc(N(c2ccccc2)c2ccc3sc4cc(N(c5ccc6c(c5)sc5ccccc56)c5ccc6c(c5)c5ccccc5n6-c5ccccc5)ccc4c3c2)cc1 ; c1ccc(N(c2ccccc2)c2ccc3sc4cc(N(c5ccc6oc7ccccc7c6c5)c5ccc6c7ccccc7c7ccccc7c6c5)ccc4c3c2)cc1]

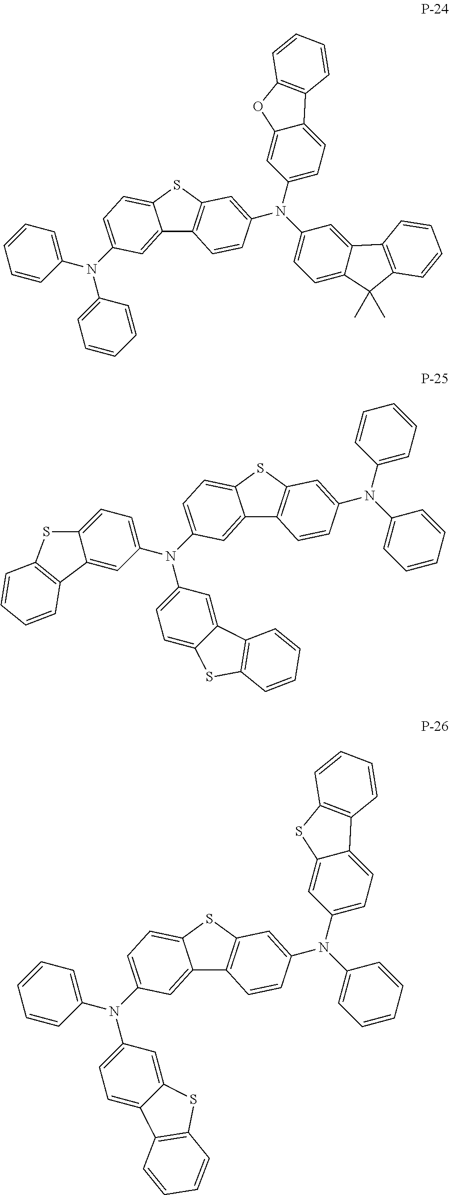 [SMILES: CC1(C)c2ccccc2-c2cc(N(c3ccc4c(c3)oc3ccccc34)c3ccc4c(c3)sc3ccc(N(c5ccccc5)c5ccccc5)cc34)ccc21 ; c1ccc(N(c2ccccc2)c2ccc3c(c2)sc2ccc(N(c4ccc5sc6ccccc6c5c4)c4ccc5sc6ccccc6c5c4)cc23)cc1 ; c1ccc(N(c2ccc3c(c2)sc2ccccc23)c2ccc3c(c2)sc2ccc(N(c4ccccc4)c4ccc5c(c4)sc4ccccc45)cc23)cc1]

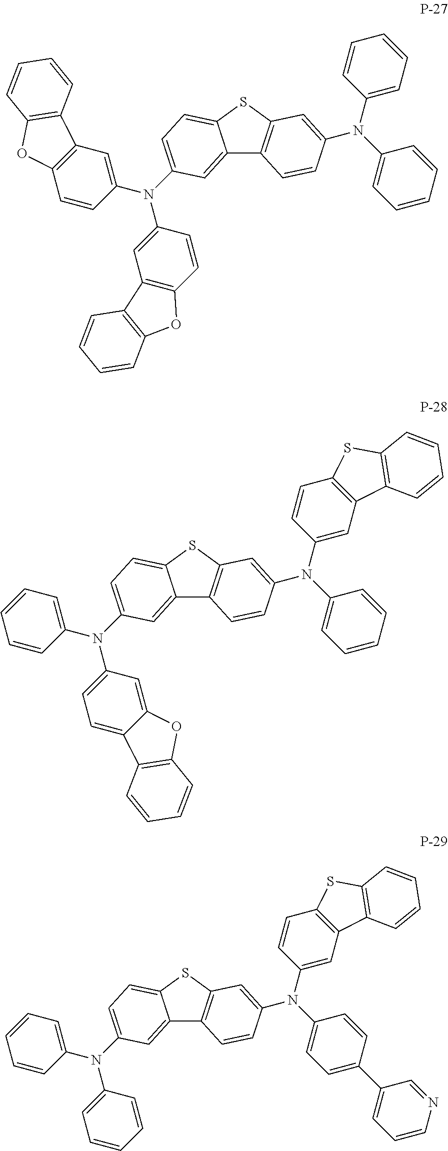 [SMILES: c1ccc(N(c2ccccc2)c2ccc3c(c2)sc2ccc(N(c4ccc5oc6ccccc6c5c4)c4ccc5oc6ccccc6c5c4)cc23)cc1 ; c1ccc(N(c2ccc3c(c2)sc2ccc(N(c4ccccc4)c4ccc5c(c4)oc4ccccc45)cc23)c2ccc3sc4ccccc4c3c2)cc1 ; c1ccc(N(c2ccccc2)c2ccc3sc4cc(N(c5ccc(-c6cccnc6)cc5)c5ccc6sc7ccccc7c6c5)ccc4c3c2)cc1]

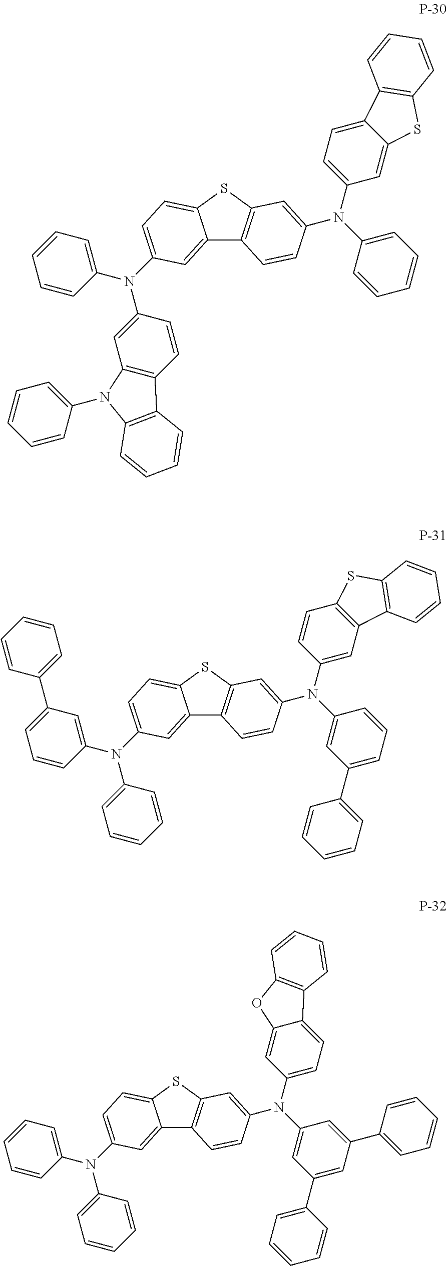 [SMILES: c1ccc(N(c2ccc3c(c2)sc2ccccc23)c2ccc3c(c2)sc2ccc(N(c4ccccc4)c4ccc5c6ccccc6n(-c6ccccc6)c5c4)cc23)cc1 ; c1ccc(-c2cccc(N(c3ccc4c(c3)sc3ccc(N(c5ccccc5)c5cccc(-c6ccccc6)c5)cc34)c3ccc4sc5ccccc5c4c3)c2)cc1 ; c1ccc(-c2cc(-c3ccccc3)cc(N(c3ccc4c(c3)oc3ccccc34)c3ccc4c(c3)sc3ccc(N(c5ccccc5)c5ccccc5)cc34)c2)cc1]

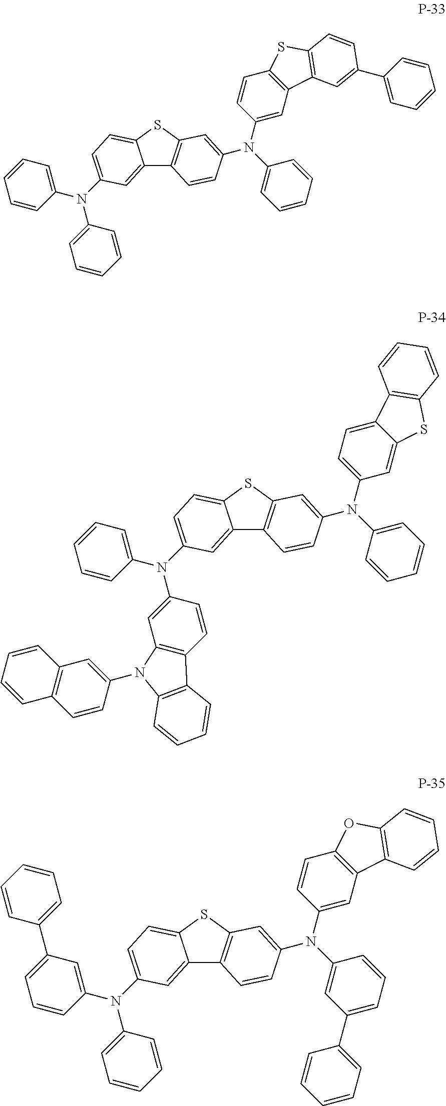 [SMILES: c1ccc(-c2ccc3sc4ccc(N(c5ccccc5)c5ccc6c(c5)sc5ccc(N(c7ccccc7)c7ccccc7)cc56)cc4c3c2)cc1 ; c1ccc(N(c2ccc3c(c2)sc2ccccc23)c2ccc3c(c2)sc2ccc(N(c4ccccc4)c4ccc5c6ccccc6n(-c6ccc7ccccc7c6)c5c4)cc23)cc1 ; c1ccc(-c2cccc(N(c3ccc4c(c3)sc3ccc(N(c5ccccc5)c5cccc(-c6ccccc6)c5)cc34)c3ccc4oc5ccccc5c4c3)c2)cc1]

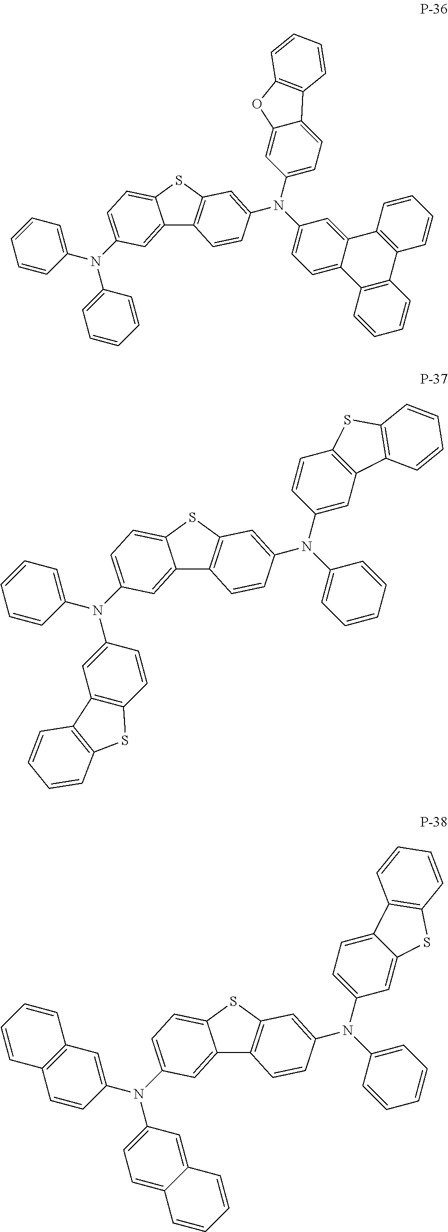 [SMILES: c1ccc(N(c2ccccc2)c2ccc3sc4cc(N(c5ccc6c(c5)oc5ccccc56)c5ccc6c7ccccc7c7ccccc7c6c5)ccc4c3c2)cc1 ; c1ccc(N(c2ccc3c(c2)sc2ccc(N(c4ccccc4)c4ccc5sc6ccccc6c5c4)cc23)c2ccc3sc4ccccc4c3c2)cc1 ; c1ccc(N(c2ccc3c(c2)sc2ccccc23)c2ccc3c(c2)sc2ccc(N(c4ccc5ccccc5c4)c4ccc5ccccc5c4)cc23)cc1]

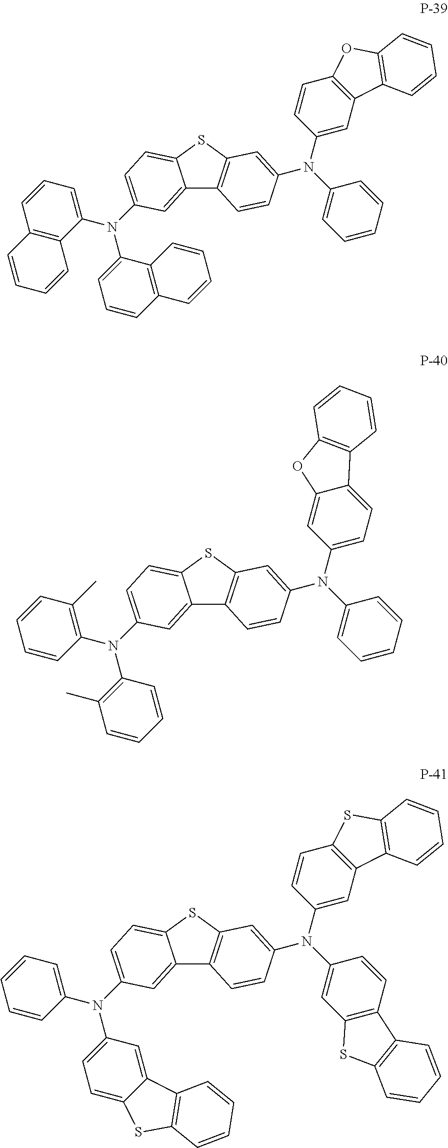 [SMILES: c1ccc(N(c2ccc3c(c2)sc2ccc(N(c4cccc5ccccc45)c4cccc5ccccc45)cc23)c2ccc3oc4ccccc4c3c2)cc1 ; Cc1ccccc1N(c1ccc2sc3cc(N(c4ccccc4)c4ccc5c(c4)oc4ccccc45)ccc3c2c1)c1ccccc1C ; c1ccc(N(c2ccc3sc4ccccc4c3c2)c2ccc3sc4cc(N(c5ccc6c(c5)sc5ccccc56)c5ccc6sc7ccccc7c6c5)ccc4c3c2)cc1]

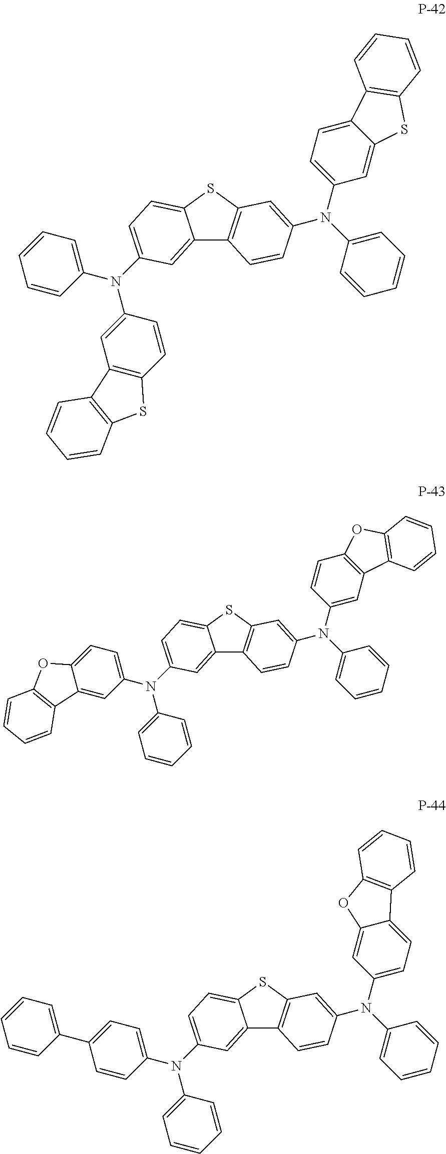 [SMILES: c1ccc(N(c2ccc3c(c2)sc2ccccc23)c2ccc3c(c2)sc2ccc(N(c4ccccc4)c4ccc5sc6ccccc6c5c4)cc23)cc1 ; c1ccc(N(c2ccc3c(c2)sc2ccc(N(c4ccccc4)c4ccc5oc6ccccc6c5c4)cc23)c2ccc3oc4ccccc4c3c2)cc1 ; c1ccc(-c2ccc(N(c3ccccc3)c3ccc4sc5cc(N(c6ccccc6)c6ccc7c(c6)oc6ccccc67)ccc5c4c3)cc2)cc1]

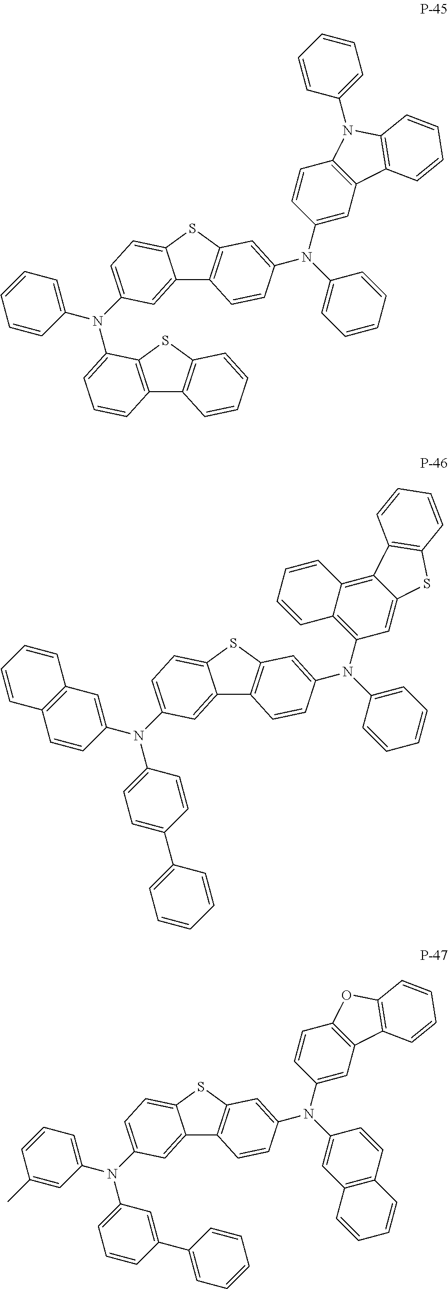 [SMILES: c1ccc(N(c2ccc3c(c2)sc2ccc(N(c4ccccc4)c4cccc5c4sc4ccccc45)cc23)c2ccc3c(c2)c2ccccc2n3-c2ccccc2)cc1 ; c1ccc(-c2ccc(N(c3ccc4ccccc4c3)c3ccc4sc5cc(N(c6ccccc6)c6cc7sc8ccccc8c7c7ccccc67)ccc5c4c3)cc2)cc1 ; Cc1cccc(N(c2cccc(-c3ccccc3)c2)c2ccc3sc4cc(N(c5ccc6ccccc6c5)c5ccc6oc7ccccc7c6c5)ccc4c3c2)c1]

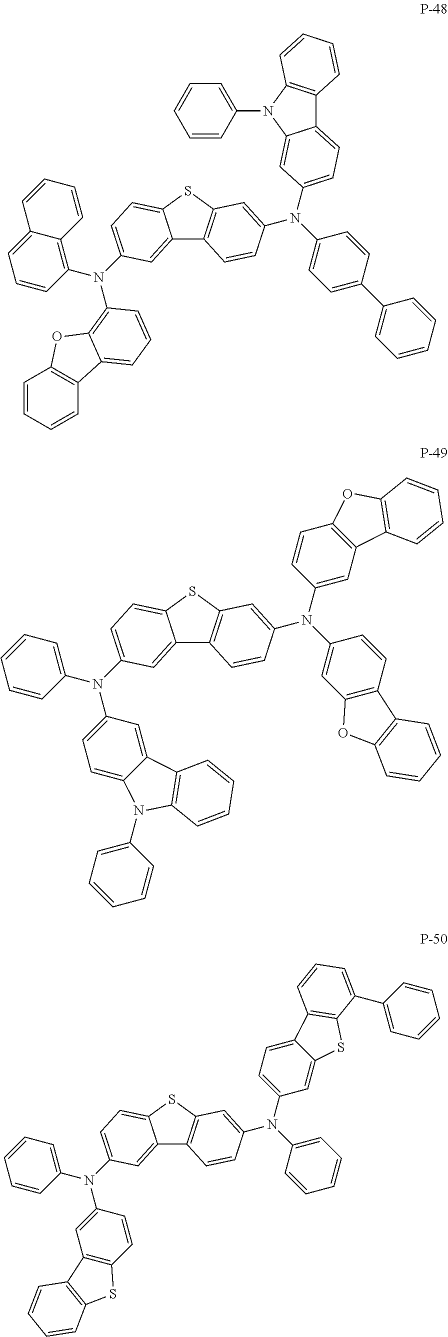 [SMILES: c1ccc(-c2ccc(N(c3ccc4c(c3)sc3ccc(N(c5cccc6ccccc56)c5cccc6c5oc5ccccc56)cc34)c3ccc4c5ccccc5n(-c5ccccc5)c4c3)cc2)cc1 ; c1ccc(N(c2ccc3sc4cc(N(c5ccc6c(c5)oc5ccccc56)c5ccc6oc7ccccc7c6c5)ccc4c3c2)c2ccc3c(c2)c2ccccc2n3-c2ccccc2)cc1 ; c1ccc(-c2cccc3c2sc2cc(N(c4ccccc4)c4ccc5c(c4)sc4ccc(N(c6ccccc6)c6ccc7sc8ccccc8c7c6)cc45)ccc23)cc1]

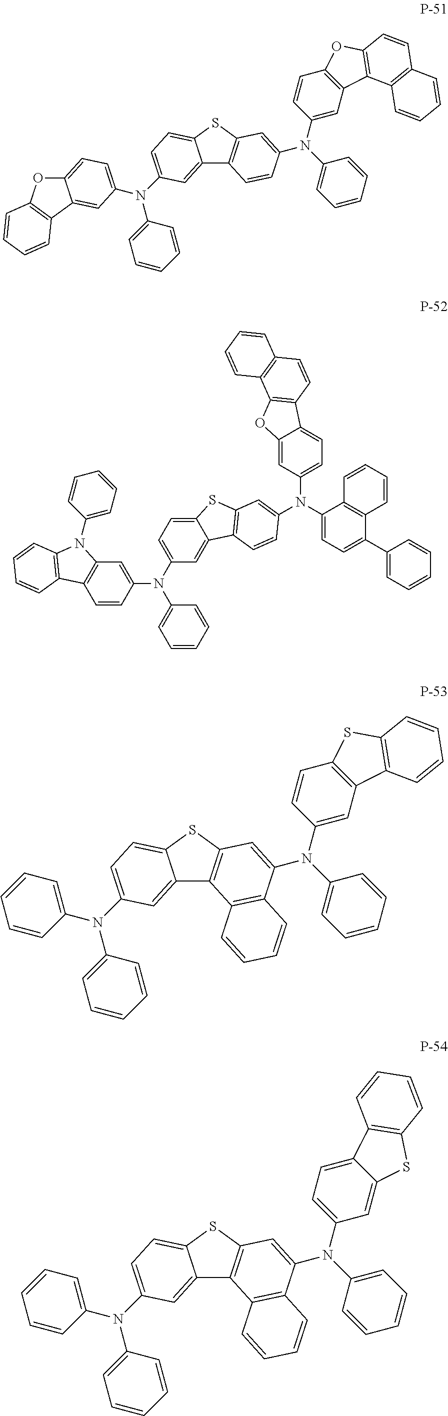 [SMILES: c1ccc(N(c2ccc3oc4ccccc4c3c2)c2ccc3sc4cc(N(c5ccccc5)c5ccc6oc7ccc8ccccc8c7c6c5)ccc4c3c2)cc1 ; c1ccc(-c2ccc(N(c3ccc4c(c3)oc3c5ccccc5ccc43)c3ccc4c(c3)sc3ccc(N(c5ccccc5)c5ccc6c7ccccc7n(-c7ccccc7)c6c5)cc34)c3ccccc23)cc1 ; c1ccc(N(c2ccccc2)c2ccc3sc4cc(N(c5ccccc5)c5ccc6sc7ccccc7c6c5)c5ccccc5c4c3c2)cc1 ; c1ccc(N(c2ccccc2)c2ccc3sc4cc(N(c5ccccc5)c5ccc6c(c5)sc5ccccc56)c5ccccc5c4c3c2)cc1]

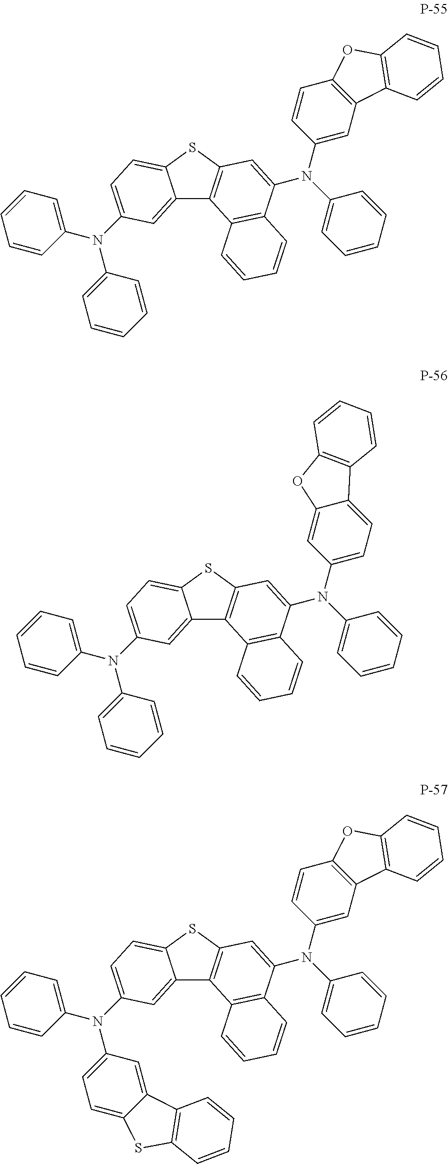 [SMILES: c1ccc(N(c2ccccc2)c2ccc3sc4cc(N(c5ccccc5)c5ccc6oc7ccccc7c6c5)c5ccccc5c4c3c2)cc1 ; c1ccc(N(c2ccccc2)c2ccc3sc4cc(N(c5ccccc5)c5ccc6c(c5)oc5ccccc56)c5ccccc5c4c3c2)cc1 ; c1ccc(N(c2ccc3sc4ccccc4c3c2)c2ccc3sc4cc(N(c5ccccc5)c5ccc6oc7ccccc7c6c5)c5ccccc5c4c3c2)cc1]

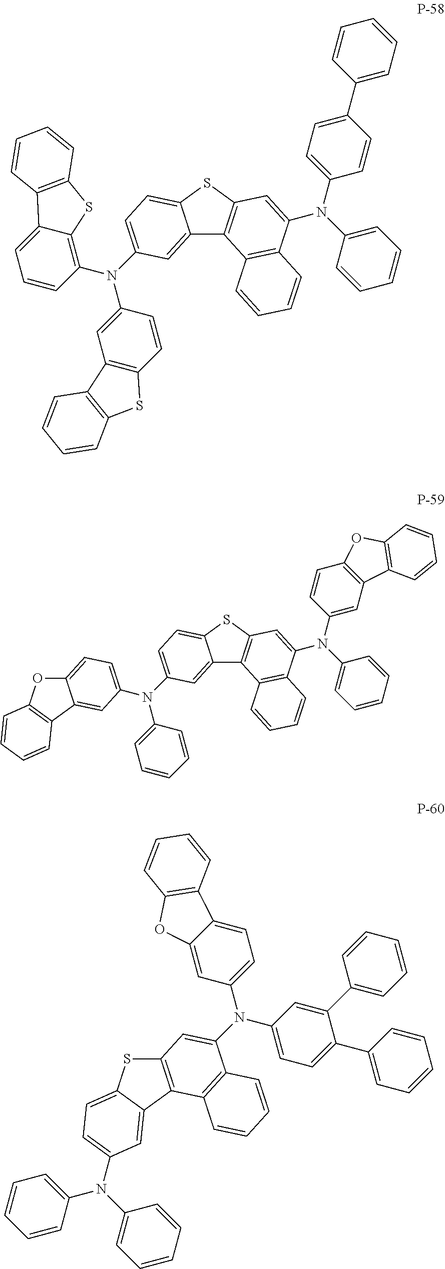 [SMILES: c1ccc(-c2ccc(N(c3ccccc3)c3cc4sc5ccc(N(c6ccc7sc8ccccc8c7c6)c6cccc7c6sc6ccccc67)cc5c4c4ccccc34)cc2)cc1 ; c1ccc(N(c2ccc3oc4ccccc4c3c2)c2ccc3sc4cc(N(c5ccccc5)c5ccc6oc7ccccc7c6c5)c5ccccc5c4c3c2)cc1 ; c1ccc(-c2ccc(N(c3ccc4c(c3)oc3ccccc34)c3cc4sc5ccc(N(c6ccccc6)c6ccccc6)cc5c4c4ccccc34)cc2-c2ccccc2)cc1]

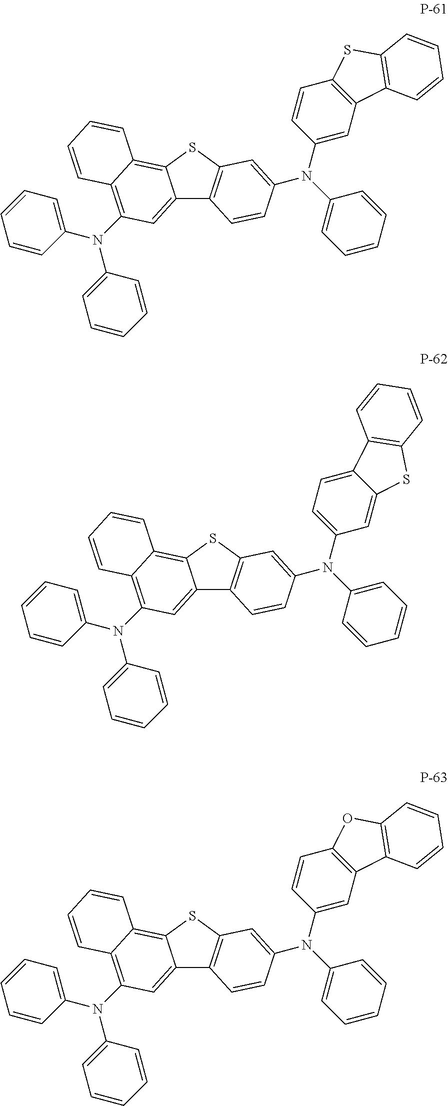 [SMILES: c1ccc(N(c2ccc3c(c2)sc2c4ccccc4c(N(c4ccccc4)c4ccccc4)cc32)c2ccc3sc4ccccc4c3c2)cc1 ; c1ccc(N(c2ccc3c(c2)sc2ccccc23)c2ccc3c(c2)sc2c4ccccc4c(N(c4ccccc4)c4ccccc4)cc32)cc1 ; c1ccc(N(c2ccc3c(c2)sc2c4ccccc4c(N(c4ccccc4)c4ccccc4)cc32)c2ccc3oc4ccccc4c3c2)cc1]

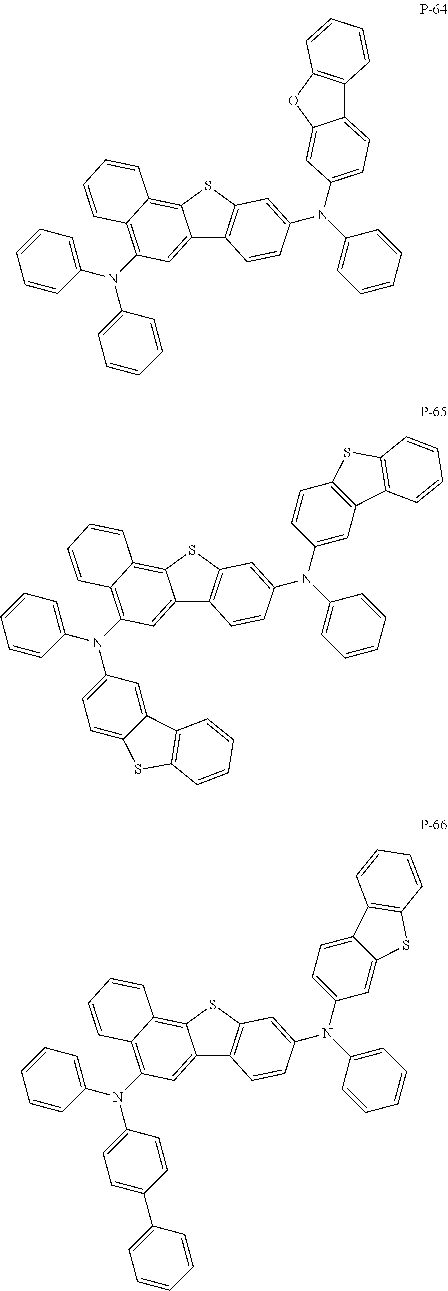 [SMILES: c1ccc(N(c2ccc3c(c2)oc2ccccc23)c2ccc3c(c2)sc2c4ccccc4c(N(c4ccccc4)c4ccccc4)cc32)cc1 ; c1ccc(N(c2ccc3c(c2)sc2c4ccccc4c(N(c4ccccc4)c4ccc5sc6ccccc6c5c4)cc32)c2ccc3sc4ccccc4c3c2)cc1 ; c1ccc(-c2ccc(N(c3ccccc3)c3cc4c5ccc(N(c6ccccc6)c6ccc7c(c6)sc6ccccc67)cc5sc4c4ccccc34)cc2)cc1]

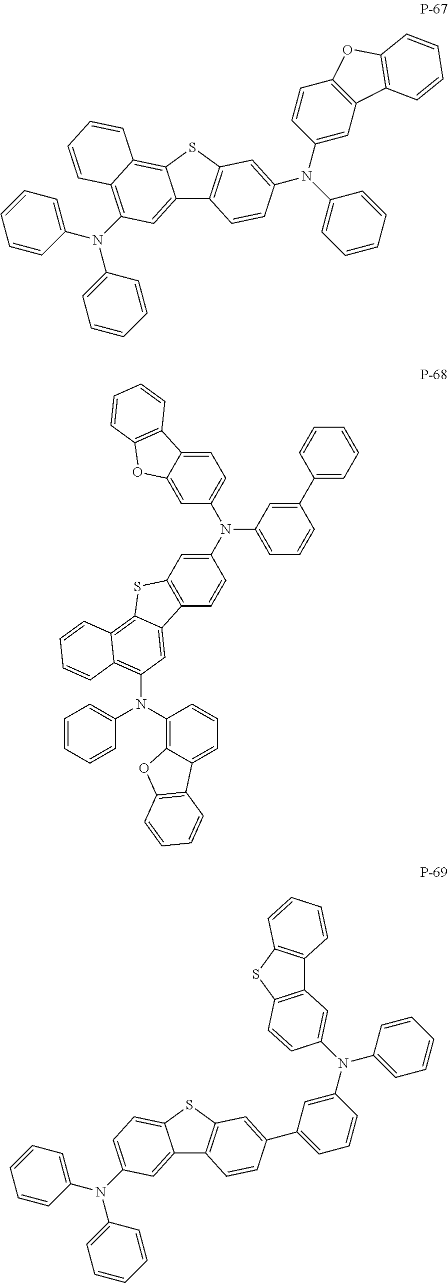 [SMILES: c1ccc(N(c2ccc3c(c2)sc2c4ccccc4c(N(c4ccccc4)c4ccccc4)cc32)c2ccc3oc4ccccc4c3c2)cc1 ; c1ccc(-c2cccc(N(c3ccc4c(c3)oc3ccccc34)c3ccc4c(c3)sc3c5ccccc5c(N(c5ccccc5)c5cccc6c5oc5ccccc56)cc43)c2)cc1 ; c1ccc(N(c2cccc(-c3ccc4c(c3)sc3ccc(N(c5ccccc5)c5ccccc5)cc34)c2)c2ccc3sc4ccccc4c3c2)cc1]

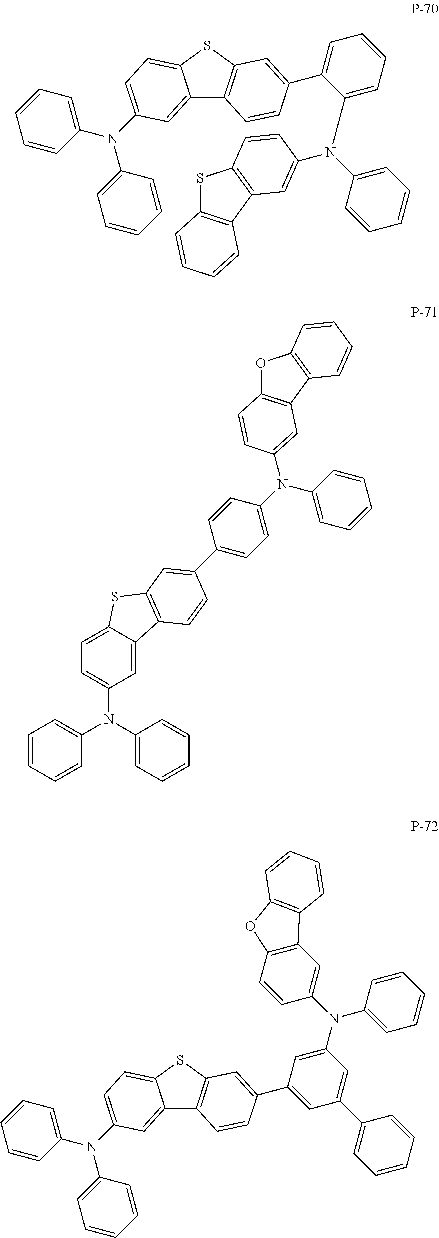 [SMILES: c1ccc(N(c2ccccc2)c2ccc3sc4cc(-c5ccccc5N(c5ccccc5)c5ccc6sc7ccccc7c6c5)ccc4c3c2)cc1 ; c1ccc(N(c2ccc(-c3ccc4c(c3)sc3ccc(N(c5ccccc5)c5ccccc5)cc34)cc2)c2ccc3oc4ccccc4c3c2)cc1 ; c1ccc(-c2cc(-c3ccc4c(c3)sc3ccc(N(c5ccccc5)c5ccccc5)cc34)cc(N(c3ccccc3)c3ccc4oc5ccccc5c4c3)c2)cc1]

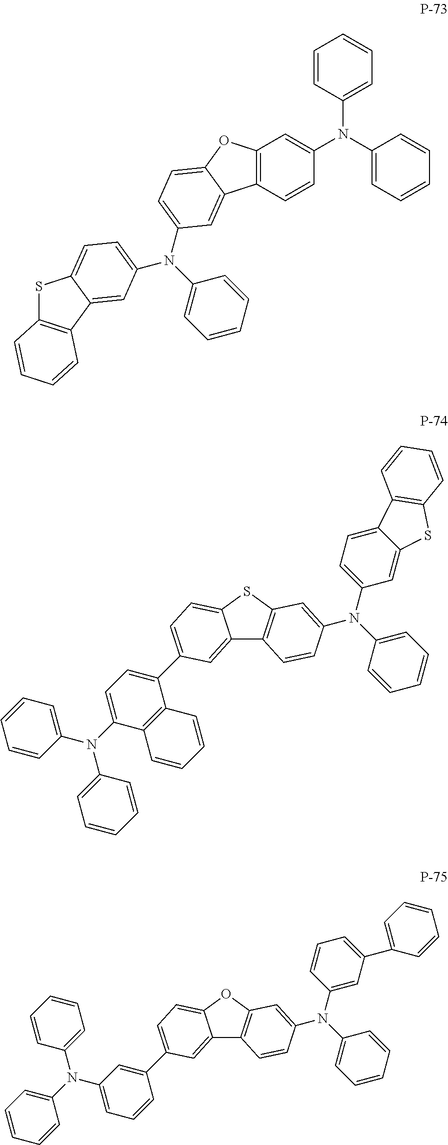 [SMILES: c1ccc(N(c2ccccc2)c2ccc3c(c2)oc2ccc(N(c4ccccc4)c4ccc5sc6ccccc6c5c4)cc23)cc1 ; c1ccc(N(c2ccc3c(c2)sc2ccccc23)c2ccc3c(c2)sc2ccc(-c4ccc(N(c5ccccc5)c5ccccc5)c5ccccc45)cc23)cc1 ; c1ccc(-c2cccc(N(c3ccccc3)c3ccc4c(c3)oc3ccc(-c5cccc(N(c6ccccc6)c6ccccc6)c5)cc34)c2)cc1]

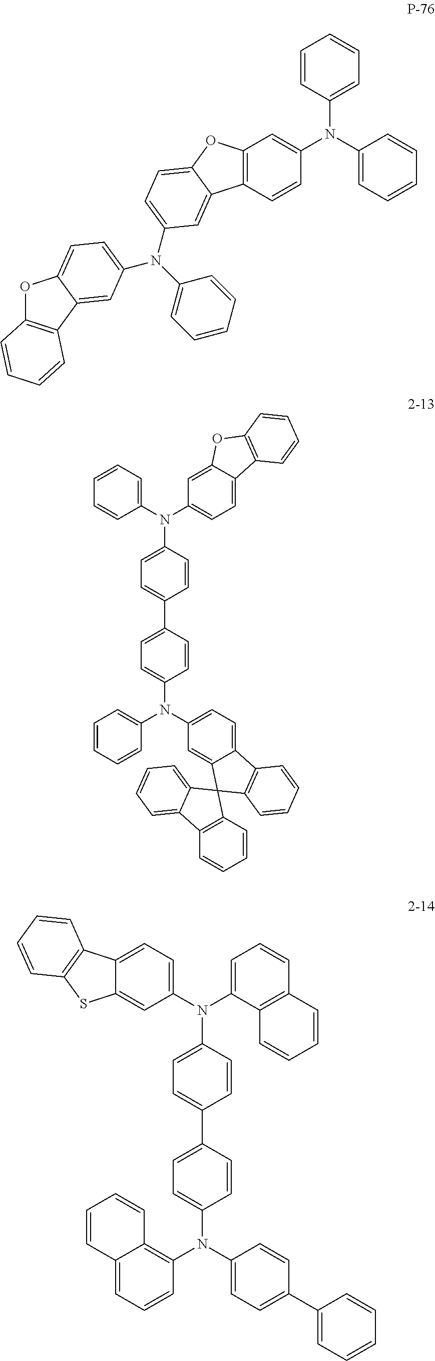 [SMILES: c1ccc(N(c2ccccc2)c2ccc3c(c2)oc2ccc(N(c4ccccc4)c4ccc5oc6ccccc6c5c4)cc23)cc1 ; c1ccc(N(c2ccc(-c3ccc(N(c4ccccc4)c4ccc5c(c4)oc4ccccc45)cc3)cc2)c2ccc3c(c2)C2(c4ccccc4-c4ccccc42)c2ccccc2-3)cc1 ; c1ccc(-c2ccc(N(c3ccc(-c4ccc(N(c5ccc6c(c5)sc5ccccc56)c5cccc6ccccc56)cc4)cc3)c3cccc4ccccc34)cc2)cc1]

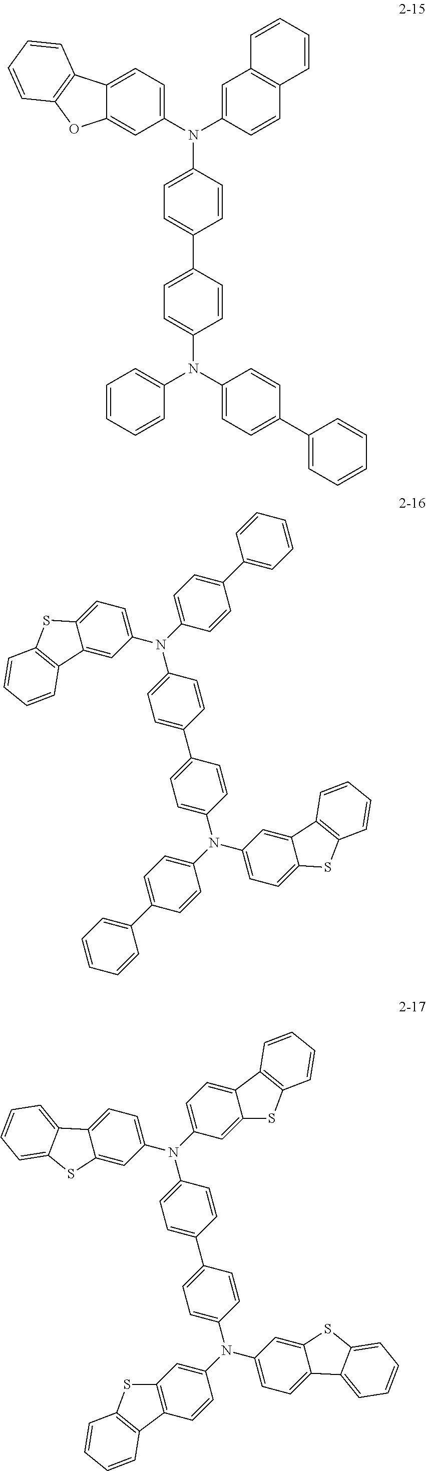 [SMILES: c1ccc(-c2ccc(N(c3ccccc3)c3ccc(-c4ccc(N(c5ccc6ccccc6c5)c5ccc6c(c5)oc5ccccc56)cc4)cc3)cc2)cc1 ; c1ccc(-c2ccc(N(c3ccc(-c4ccc(N(c5ccc(-c6ccccc6)cc5)c5ccc6sc7ccccc7c6c5)cc4)cc3)c3ccc4sc5ccccc5c4c3)cc2)cc1 ; c1ccc2c(c1)sc1cc(N(c3ccc(-c4ccc(N(c5ccc6c(c5)sc5ccccc56)c5ccc6c(c5)sc5ccccc56)cc4)cc3)c3ccc4c(c3)sc3ccccc34)ccc12]

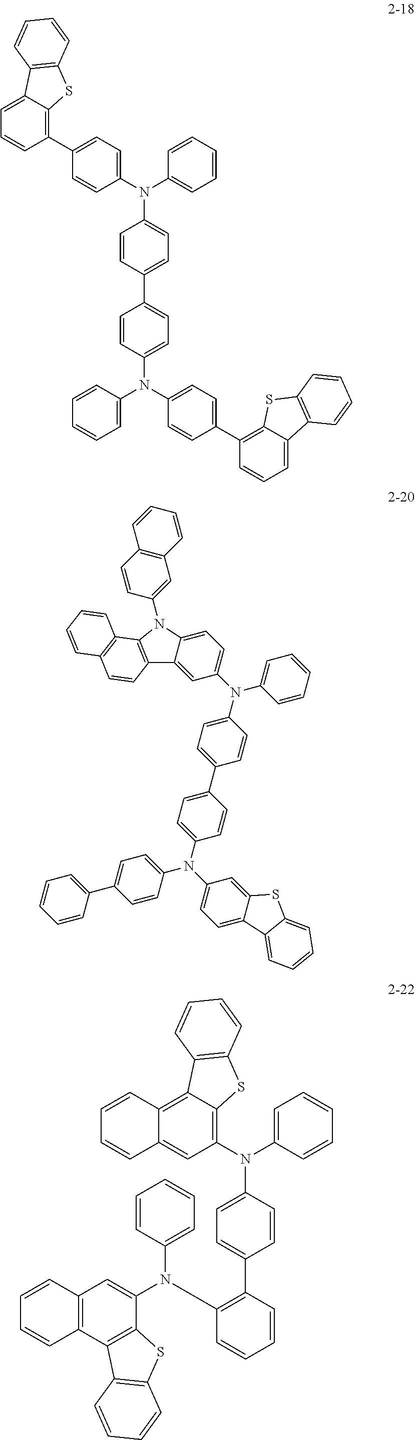 [SMILES: c1ccc(N(c2ccc(-c3ccc(N(c4ccccc4)c4ccc(-c5cccc6c5sc5ccccc56)cc4)cc3)cc2)c2ccc(-c3cccc4c3sc3ccccc34)cc2)cc1 ; c1ccc(-c2ccc(N(c3ccc(-c4ccc(N(c5ccccc5)c5ccc6c(c5)c5ccc7ccccc7c5n6-c5ccc6ccccc6c5)cc4)cc3)c3ccc4c(c3)sc3ccccc34)cc2)cc1 ; c1ccc(N(c2ccc(-c3ccccc3N(c3ccccc3)c3cc4ccccc4c4c3sc3ccccc34)cc2)c2cc3ccccc3c3c2sc2ccccc23)cc1]

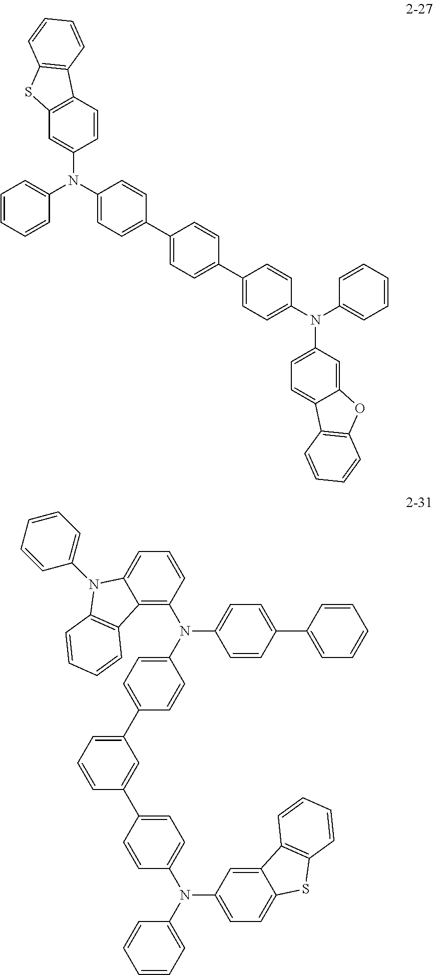 [SMILES: c1ccc(N(c2ccc(-c3ccc(-c4ccc(N(c5ccccc5)c5ccc6c(c5)sc5ccccc56)cc4)cc3)cc2)c2ccc3c(c2)oc2ccccc23)cc1 ; c1ccc(-c2ccc(N(c3ccc(-c4cccc(-c5ccc(N(c6ccccc6)c6ccc7sc8ccccc8c7c6)cc5)c4)cc3)c3cccc4c3c3ccccc3n4-c3ccccc3)cc2)cc1]

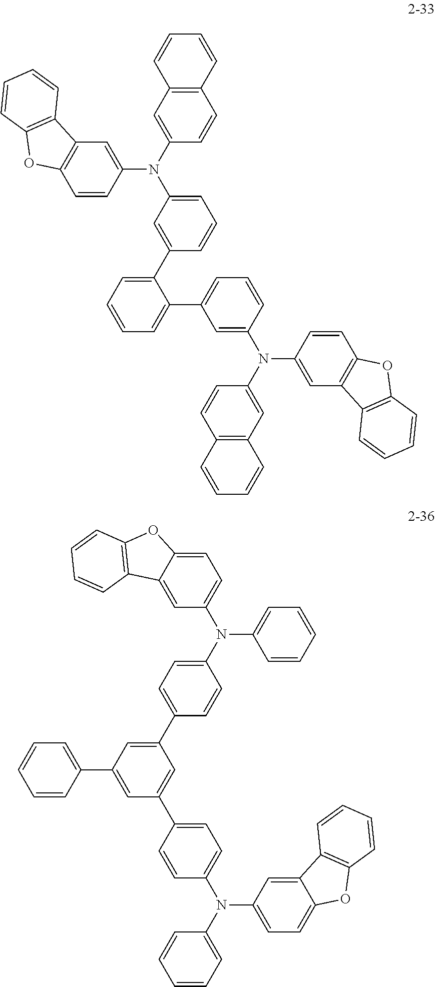 [SMILES: c1cc(-c2ccccc2-c2cccc(N(c3ccc4ccccc4c3)c3ccc4oc5ccccc5c4c3)c2)cc(N(c2ccc3ccccc3c2)c2ccc3oc4ccccc4c3c2)c1 ; c1ccc(-c2cc(-c3ccc(N(c4ccccc4)c4ccc5oc6ccccc6c5c4)cc3)cc(-c3ccc(N(c4ccccc4)c4ccc5oc6ccccc6c5c4)cc3)c2)cc1]

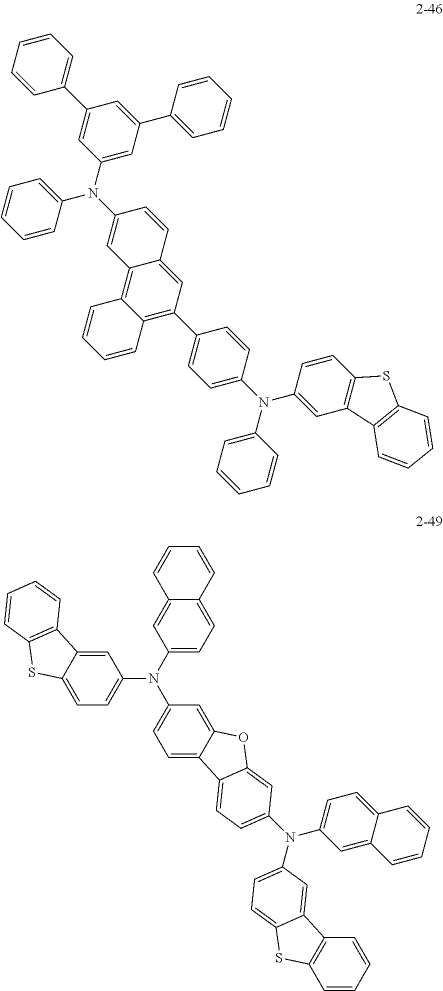 [SMILES: c1ccc(-c2cc(-c3ccccc3)cc(N(c3ccccc3)c3ccc4cc(-c5ccc(N(c6ccccc6)c6ccc7sc8ccccc8c7c6)cc5)c5ccccc5c4c3)c2)cc1 ; c1ccc2cc(N(c3ccc4c(c3)oc3cc(N(c5ccc6ccccc6c5)c5ccc6sc7ccccc7c6c5)ccc34)c3ccc4sc5ccccc5c4c3)ccc2c1]

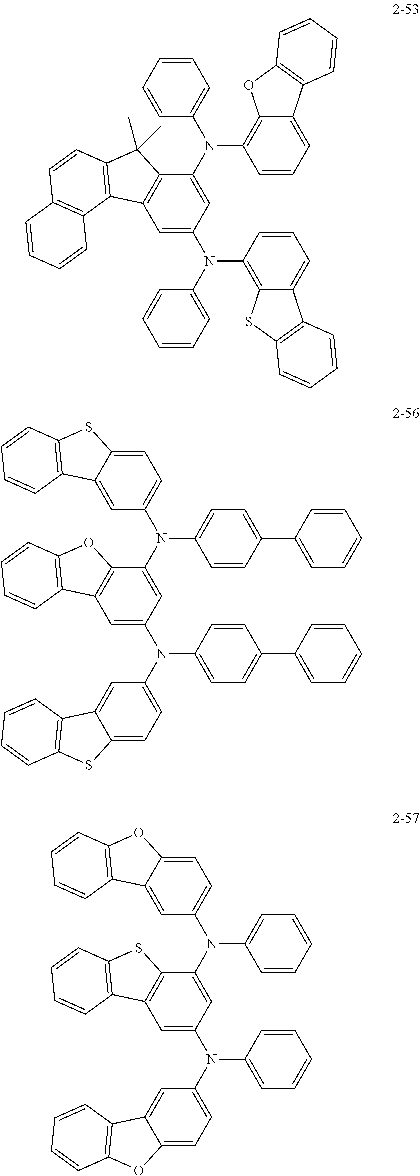 [SMILES: CC1(C)c2ccc3ccccc3c2-c2cc(N(c3ccccc3)c3cccc4c3sc3ccccc34)cc(N(c3ccccc3)c3cccc4c3oc3ccccc34)c21 ; c1ccc(-c2ccc(N(c3ccc4sc5ccccc5c4c3)c3cc(N(c4ccc(-c5ccccc5)cc4)c4ccc5sc6ccccc6c5c4)c4oc5ccccc5c4c3)cc2)cc1 ; c1ccc(N(c2ccc3oc4ccccc4c3c2)c2cc(N(c3ccccc3)c3ccc4oc5ccccc5c4c3)c3sc4ccccc4c3c2)cc1]

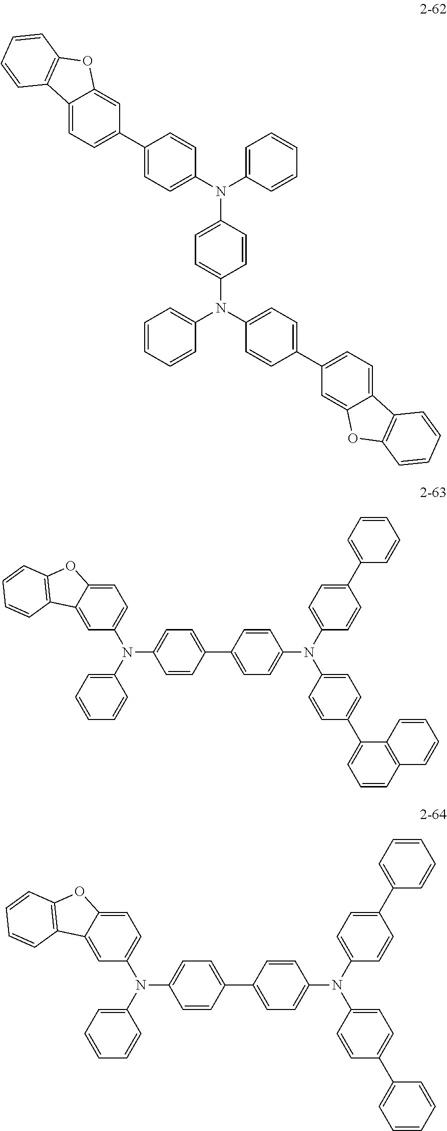 [SMILES: c1ccc(N(c2ccc(-c3ccc4c(c3)oc3ccccc34)cc2)c2ccc(N(c3ccccc3)c3ccc(-c4ccc5c(c4)oc4ccccc45)cc3)cc2)cc1 ; c1ccc(-c2ccc(N(c3ccc(-c4ccc(N(c5ccccc5)c5ccc6oc7ccccc7c6c5)cc4)cc3)c3ccc(-c4cccc5ccccc45)cc3)cc2)cc1 ; c1ccc(-c2ccc(N(c3ccc(-c4ccccc4)cc3)c3ccc(-c4ccc(N(c5ccccc5)c5ccc6oc7ccccc7c6c5)cc4)cc3)cc2)cc1]

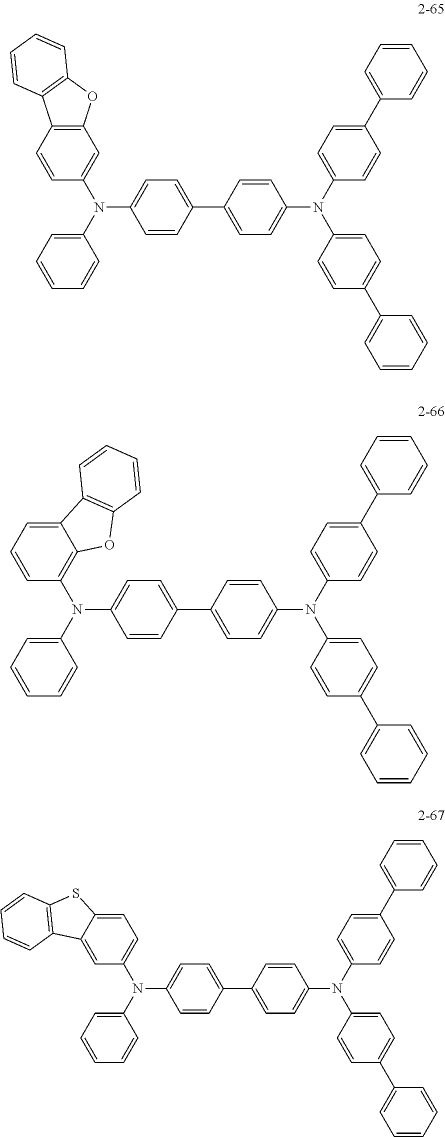 [SMILES: c1ccc(-c2ccc(N(c3ccc(-c4ccccc4)cc3)c3ccc(-c4ccc(N(c5ccccc5)c5ccc6c(c5)oc5ccccc56)cc4)cc3)cc2)cc1 ; c1ccc(-c2ccc(N(c3ccc(-c4ccccc4)cc3)c3ccc(-c4ccc(N(c5ccccc5)c5cccc6c5oc5ccccc56)cc4)cc3)cc2)cc1 ; c1ccc(-c2ccc(N(c3ccc(-c4ccccc4)cc3)c3ccc(-c4ccc(N(c5ccccc5)c5ccc6sc7ccccc7c6c5)cc4)cc3)cc2)cc1]

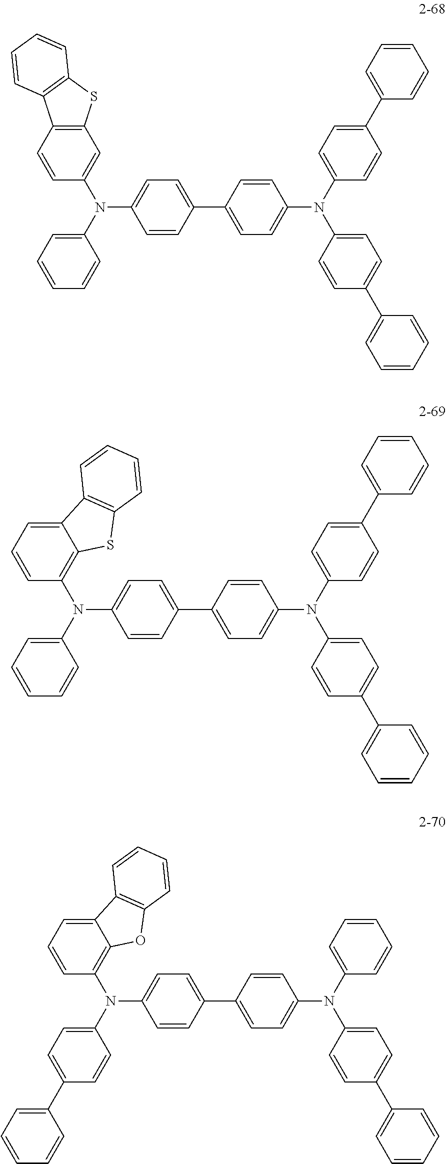 [SMILES: c1ccc(-c2ccc(N(c3ccc(-c4ccccc4)cc3)c3ccc(-c4ccc(N(c5ccccc5)c5ccc6c(c5)sc5ccccc56)cc4)cc3)cc2)cc1 ; c1ccc(-c2ccc(N(c3ccc(-c4ccccc4)cc3)c3ccc(-c4ccc(N(c5ccccc5)c5cccc6c5sc5ccccc56)cc4)cc3)cc2)cc1 ; c1ccc(-c2ccc(N(c3ccccc3)c3ccc(-c4ccc(N(c5ccc(-c6ccccc6)cc5)c5cccc6c5oc5ccccc56)cc4)cc3)cc2)cc1]

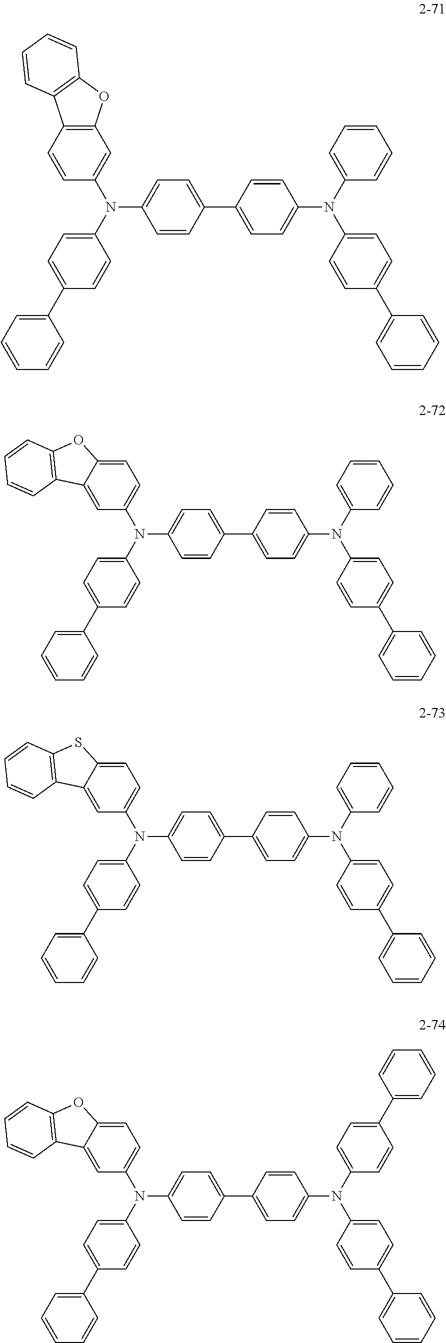 [SMILES: c1ccc(-c2ccc(N(c3ccccc3)c3ccc(-c4ccc(N(c5ccc(-c6ccccc6)cc5)c5ccc6c(c5)oc5ccccc56)cc4)cc3)cc2)cc1 ; c1ccc(-c2ccc(N(c3ccccc3)c3ccc(-c4ccc(N(c5ccc(-c6ccccc6)cc5)c5ccc6oc7ccccc7c6c5)cc4)cc3)cc2)cc1 ; c1ccc(-c2ccc(N(c3ccccc3)c3ccc(-c4ccc(N(c5ccc(-c6ccccc6)cc5)c5ccc6sc7ccccc7c6c5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc6oc7ccccc7c6c5)cc4)cc3)cc2)cc1]

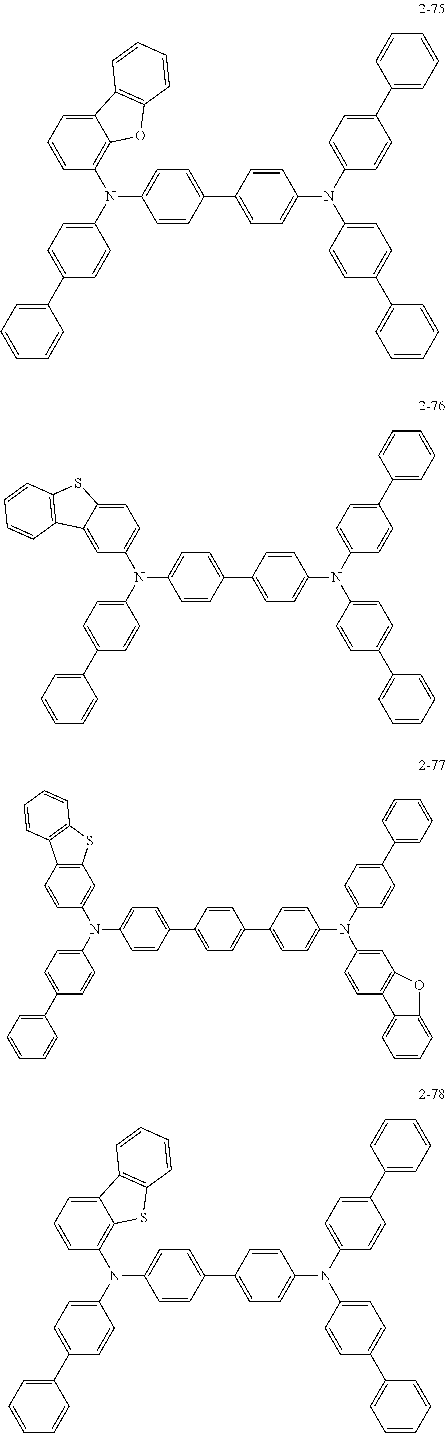 [SMILES: c1ccc(-c2ccc(N(c3ccc(-c4ccccc4)cc3)c3ccc(-c4ccc(N(c5ccc(-c6ccccc6)cc5)c5cccc6c5oc5ccccc56)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc6sc7ccccc7c6c5)cc4)cc3)cc2)cc1 ; c1ccc(-c2ccc(N(c3ccc(-c4ccc(-c5ccc(N(c6ccc(-c7ccccc7)cc6)c6ccc7c(c6)sc6ccccc67)cc5)cc4)cc3)c3ccc4c(c3)oc3ccccc34)cc2)cc1 ; c1ccc(-c2ccc(N(c3ccc(-c4ccccc4)cc3)c3ccc(-c4ccc(N(c5ccc(-c6ccccc6)cc5)c5cccc6c5sc5ccccc56)cc4)cc3)cc2)cc1]

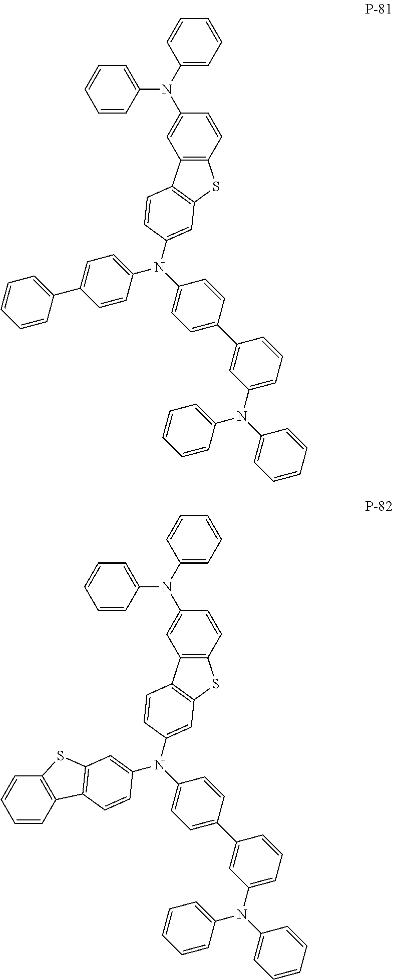 [SMILES: c1ccc(-c2ccc(N(c3ccc(-c4cccc(N(c5ccccc5)c5ccccc5)c4)cc3)c3ccc4c(c3)sc3ccc(N(c5ccccc5)c5ccccc5)cc34)cc2)cc1 ; c1ccc(N(c2ccccc2)c2cccc(-c3ccc(N(c4ccc5c(c4)sc4ccccc45)c4ccc5c(c4)sc4ccc(N(c6ccccc6)c6ccccc6)cc45)cc3)c2)cc1]

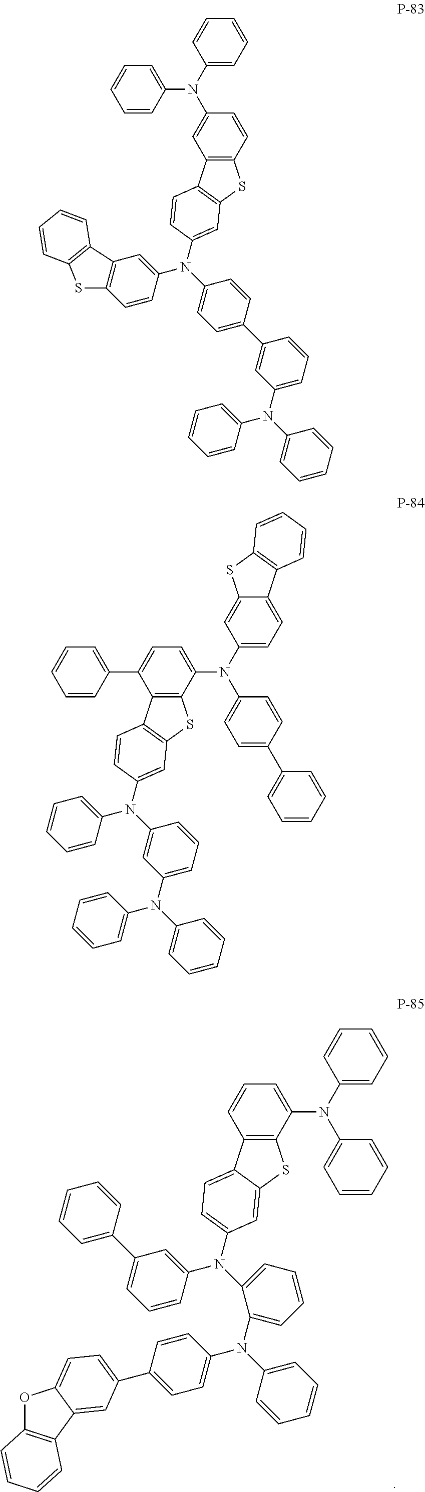

23. The compound of claim 9, wherein the compound represented by Formula 5 is one of the following compounds:

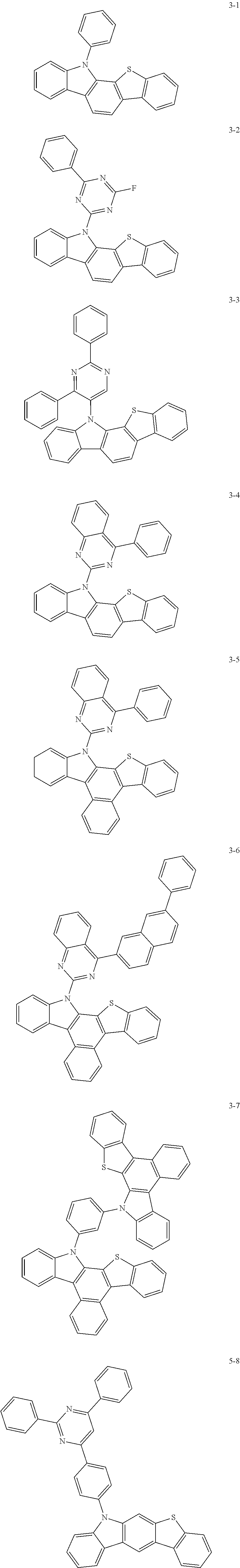

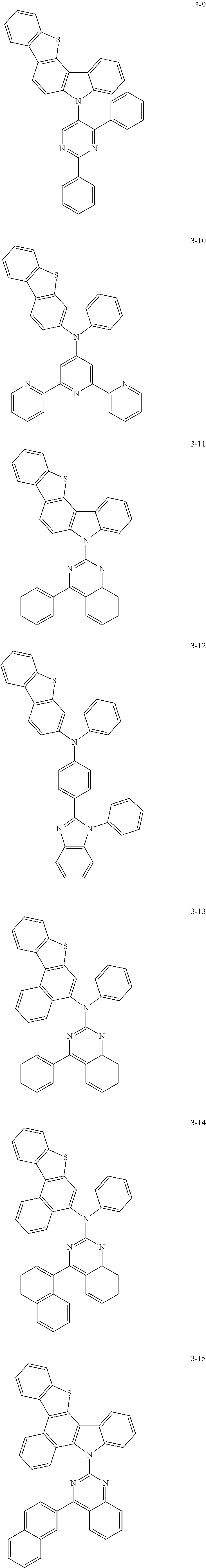

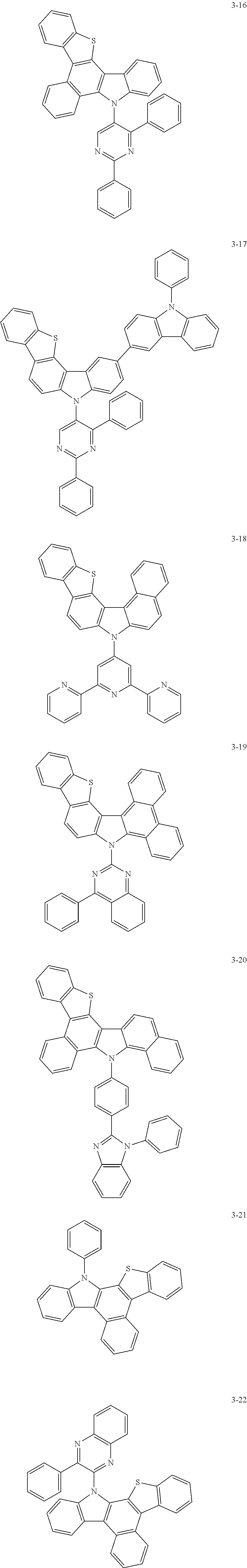

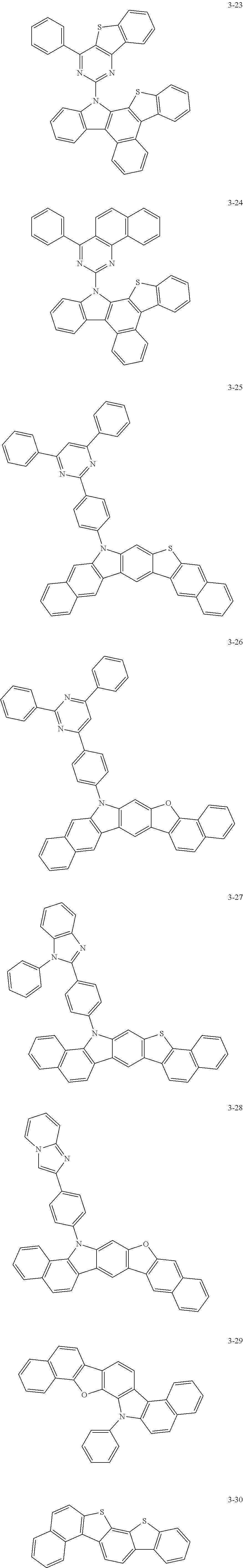

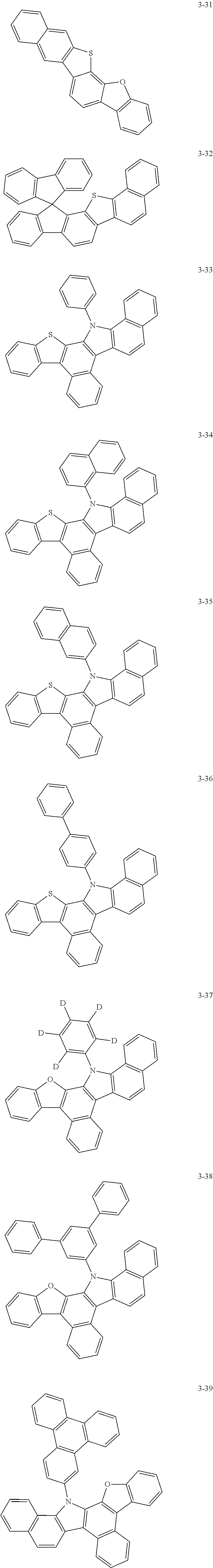

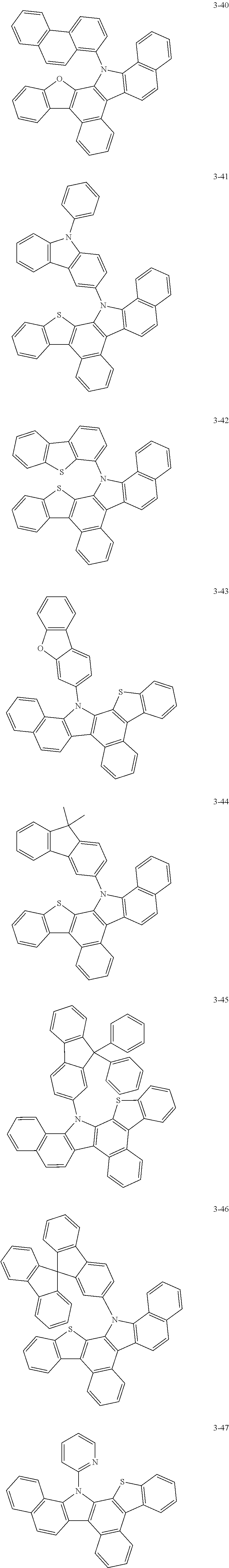

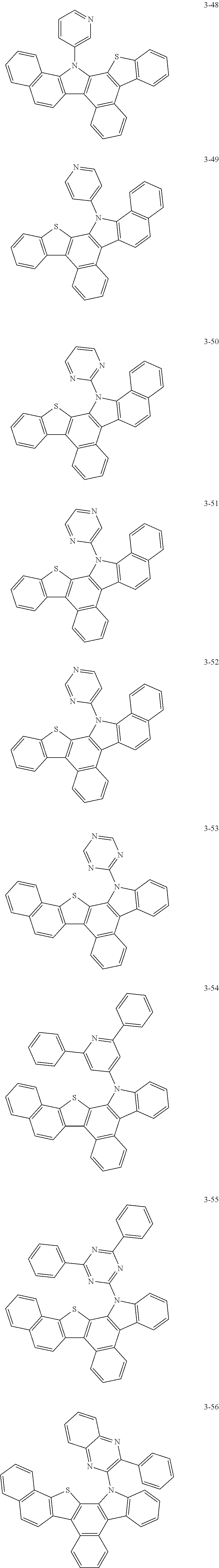

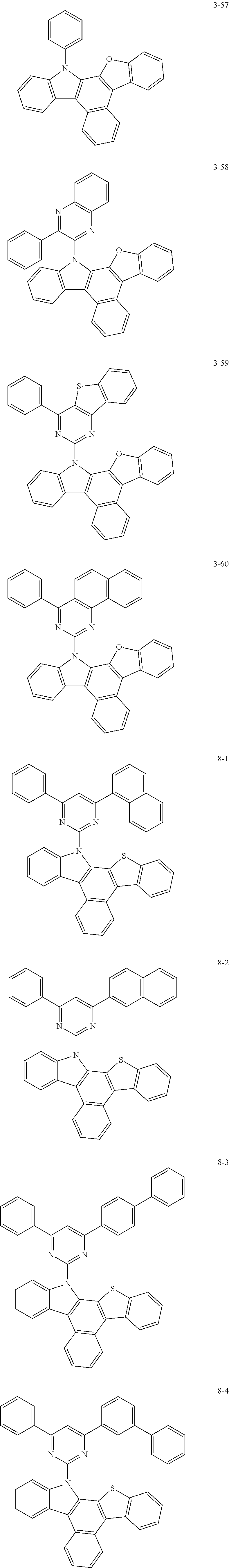

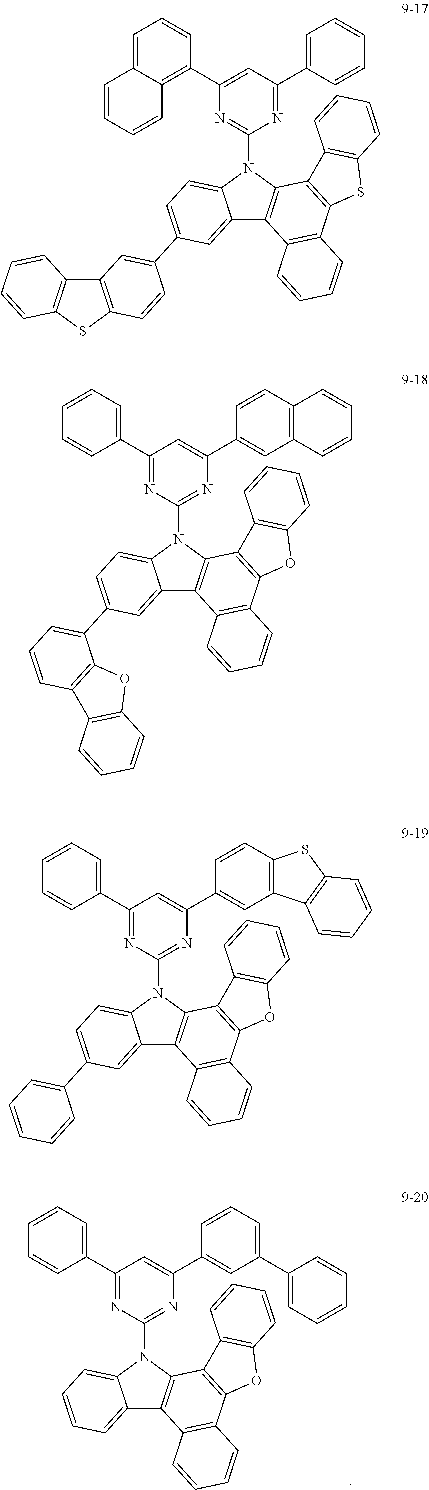 [SMILES: c1ccc(-c2cc(-c3cccc4ccccc34)nc(-n3c4ccc(-c5ccc6sc7ccccc7c6c5)cc4c4c5ccccc5c5sc6ccccc6c5c43)n2)cc1 ; c1ccc(-c2cc(-c3ccc4ccccc4c3)nc(-n3c4ccc(-c5cccc6c5oc5ccccc56)cc4c4c5ccccc5c5oc6ccccc6c5c43)n2)cc1 ; c1ccc(-c2ccc3c(c2)c2c4ccccc4c4oc5ccccc5c4c2n3-c2nc(-c3ccccc3)cc(-c3ccc4sc5ccccc5c4c3)n2)cc1 ; c1ccc(-c2cccc(-c3cc(-c4ccccc4)nc(-n4c5ccccc5c5c6ccccc6c6oc7ccccc7c6c54)n3)c2)cc1]

24. The compound of claim 9, wherein the compound represented by Formula 6 is one of the following compounds:

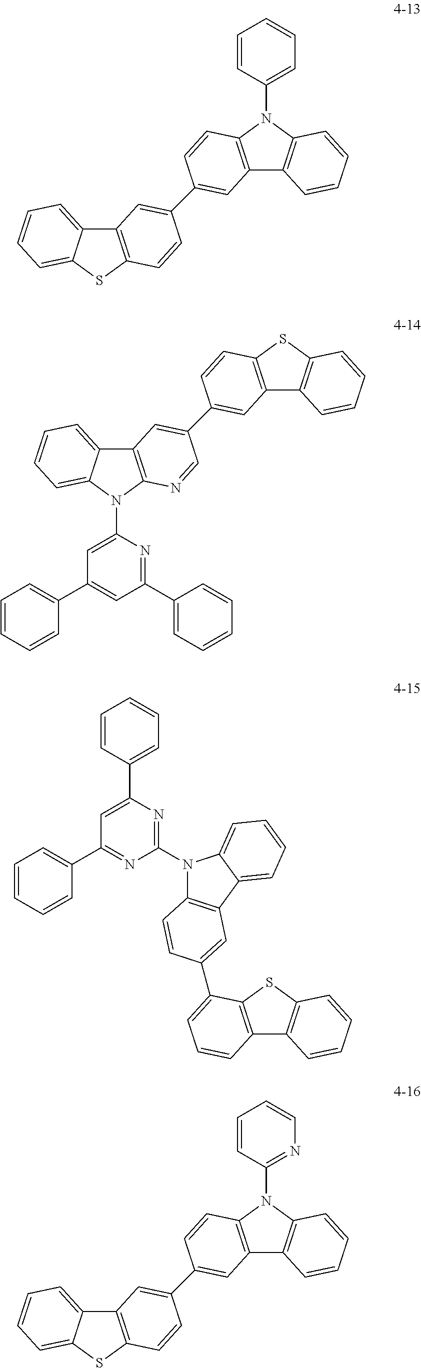 [SMILES: c1ccc(-n2c3ccccc3c3cc(-c4ccc5sc6ccccc6c5c4)ccc32)cc1 ; c1ccc(-c2cc(-c3ccccc3)nc(-n3c4ccccc4c4cc(-c5ccc6sc7ccccc7c6c5)cnc43)c2)cc1 ; c1ccc(-c2cc(-c3ccccc3)nc(-n3c4ccccc4c4cc(-c5cccc6c5sc5ccccc56)ccc43)n2)cc1 ; c1ccc(-n2c3ccccc3c3cc(-c4ccc5sc6ccccc6c5c4)ccc32)nc1]

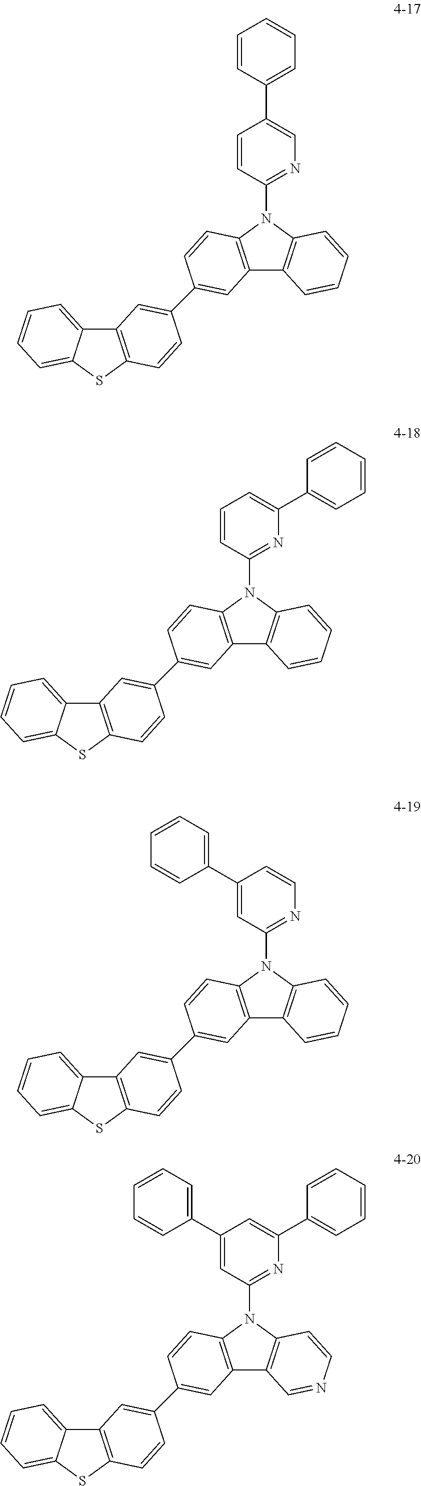 [SMILES: c1ccc(-c2ccc(-n3c4ccccc4c4cc(-c5ccc6sc7ccccc7c6c5)ccc43)nc2)cc1 ; c1ccc(-c2cccc(-n3c4ccccc4c4cc(-c5ccc6sc7ccccc7c6c5)ccc43)n2)cc1 ; c1ccc(-c2ccnc(-n3c4ccccc4c4cc(-c5ccc6sc7ccccc7c6c5)ccc43)c2)cc1 ; c1ccc(-c2cc(-c3ccccc3)nc(-n3c4ccncc4c4cc(-c5ccc6sc7ccccc7c6c5)ccc43)c2)cc1]

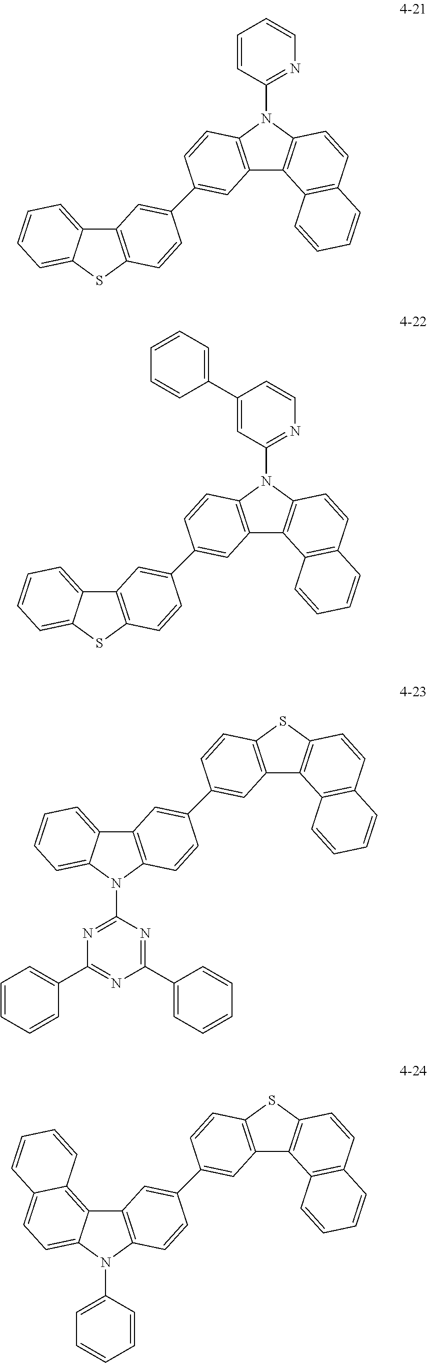 [SMILES: c1ccc(-n2c3ccc(-c4ccc5sc6ccccc6c5c4)cc3c3c4ccccc4ccc32)nc1 ; c1ccc(-c2ccnc(-n3c4ccc(-c5ccc6sc7ccccc7c6c5)cc4c4c5ccccc5ccc43)c2)cc1 ; c1ccc(-c2nc(-c3ccccc3)nc(-n3c4ccccc4c4cc(-c5ccc6sc7ccc8ccccc8c7c6c5)ccc43)n2)cc1 ; c1ccc(-n2c3ccc(-c4ccc5sc6ccc7ccccc7c6c5c4)cc3c3c4ccccc4ccc32)cc1]

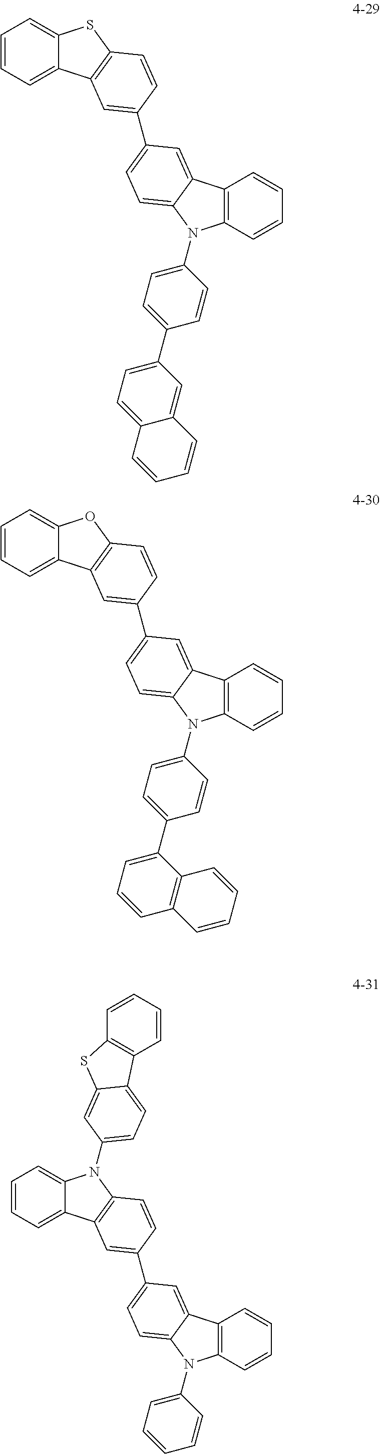 [SMILES: c1ccc2cc(-c3ccc(-n4c5ccccc5c5cc(-c6ccc7sc8ccccc8c7c6)ccc54)cc3)ccc2c1 ; c1ccc2c(-c3ccc(-n4c5ccccc5c5cc(-c6ccc7oc8ccccc8c7c6)ccc54)cc3)cccc2c1 ; c1ccc(-n2c3ccccc3c3cc(-c4ccc5c(c4)c4ccccc4n5-c4ccc5c(c4)sc4ccccc45)ccc32)cc1]

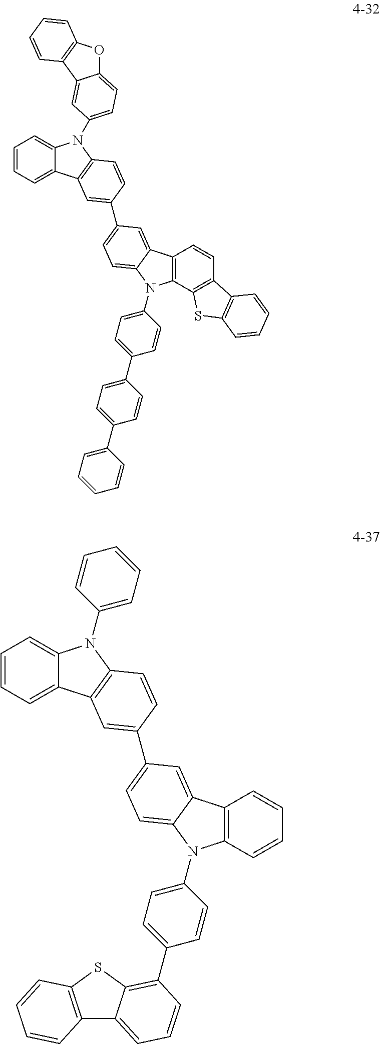 [SMILES: c1ccc(-c2ccc(-c3ccc(-n4c5ccc(-c6ccc7c(c6)c6ccccc6n7-c6ccc7oc8ccccc8c7c6)cc5c5ccc6c7ccccc7sc6c54)cc3)cc2)cc1 ; c1ccc(-n2c3ccccc3c3cc(-c4ccc5c(c4)c4ccccc4n5-c4ccc(-c5cccc6c5sc5ccccc56)cc4)ccc32)cc1]

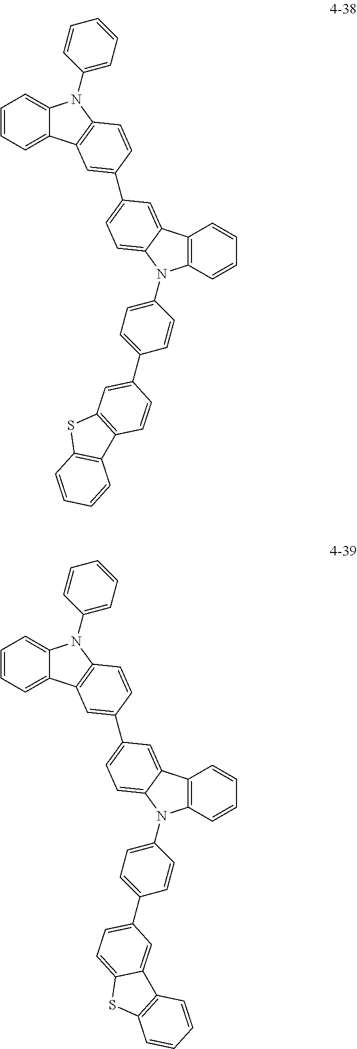 [SMILES: c1ccc(-n2c3ccccc3c3cc(-c4ccc5c(c4)c4ccccc4n5-c4ccc(-c5ccc6c(c5)sc5ccccc56)cc4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

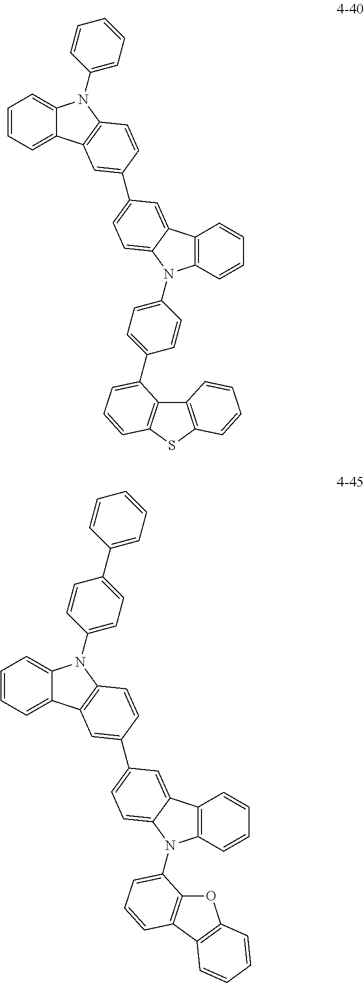 [SMILES: c1ccc(-n2c3ccccc3c3cc(-c4ccc5c(c4)c4ccccc4n5-c4ccc(-c5cccc6sc7ccccc7c56)cc4)ccc32)cc1 ; c1ccc(-c2ccc(-n3c4ccccc4c4cc(-c5ccc6c(c5)c5ccccc5n6-c5cccc6c5oc5ccccc56)ccc43)cc2)cc1]

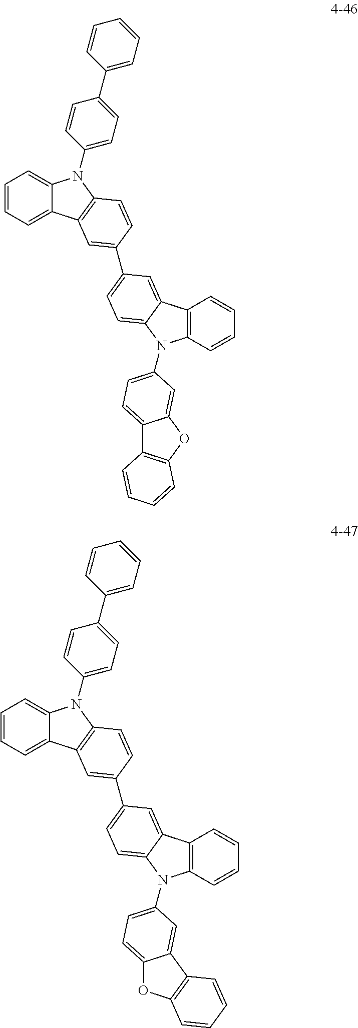 [SMILES: c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2ccc(-n3c4ccccc4c4cc(-c5ccc6c(c5)c5ccccc5n6-c5ccc6oc7ccccc7c6c5)ccc43)cc2)cc1]

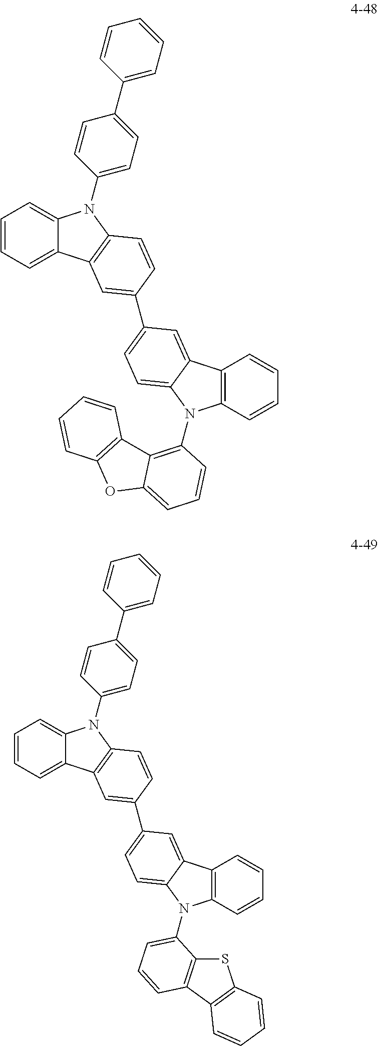 [SMILES: c1ccc(-c2ccc(-n3c4ccccc4c4cc(-c5ccc6c(c5)c5ccccc5n6-c5cccc6oc7ccccc7c56)ccc43)cc2)cc1 ; c1ccc(-c2ccc(-n3c4ccccc4c4cc(-c5ccc6c(c5)c5ccccc5n6-c5cccc6c5sc5ccccc56)ccc43)cc2)cc1]

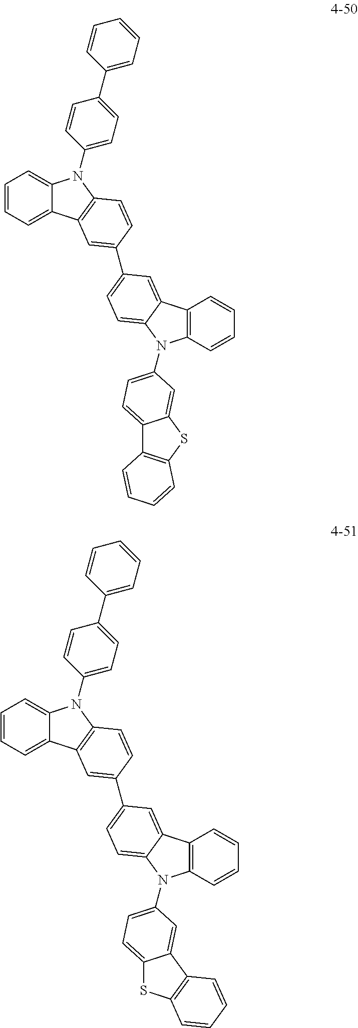 [SMILES: c1ccc(-c2ccc(-n3c4ccccc4c4cc(-c5ccc6c(c5)c5ccccc5n6-c5ccc6c(c5)sc5ccccc56)ccc43)cc2)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

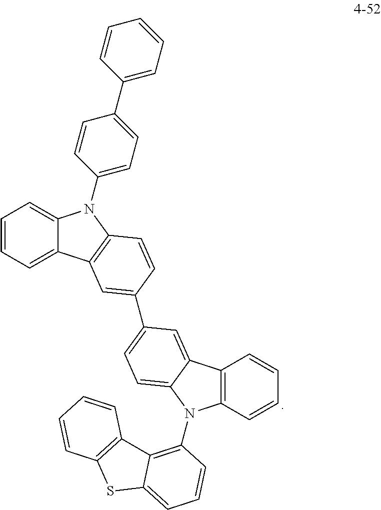 [SMILES: c1ccc(-c2ccc(-n3c4ccccc4c4cc(-c5ccc6c(c5)c5ccccc5n6-c5cccc6sc7ccccc7c56)ccc43)cc2)cc1]

25. An electronic device comprising a display device and a control unit for driving the display device, wherein the display device comprises the organic electric element of claim 1.

26. The electronic device of claim 25, wherein the organic electric element is one of an organic light emitting diode, an organic solar cell, an organic photo conductor, an organic transistor, an element for monochromatic and a quantum dot display.
